SEQUENCE LISTING (49 sequences)

SEQ ID NO: 1 - protein, 214 aa, Artificial Sequence
  Clone ID 11150 Trastuzumab VL  IgKCL
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 220 aa, Artificial Sequence
  Clone ID 21715 IgA2 hinge - IgA2m1 CH2 - IgA2m1 CH3
RVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAG

SEQ ID NO: 3 - protein, 443 aa, Artificial Sequence
  Clone ID 21755 Trastuzumab VH - IgG1 CH1 - IgG1/IgA2 hinge - IgA2m1 CH2 - IgA2m1 CH3
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAG

SEQ ID NO: 4 - protein, 220 aa, Artificial Sequence
  Clone ID 23767 IgA2 hinge - IgA2m1 CH2 - IgA2m1 CH3
RVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVRLTCRARGFSPKDVLVRWLQGSQELPREKYLTWASRQEPSQGTTTFAVTSILRVAAEDWKKGDTFSCMVGHEALPLAFTQKTIDRLAG

SEQ ID NO: 5 - protein, 220 aa, Artificial Sequence
  Clone ID 23768 IgA2 hinge - IgA2m1 CH2 - IgA2m1 CH3
RVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVRLTCKARGFSPKDVLVRWLQGSQELPREKYLTWASRQEPSQGTTTFAVTSILRVAAEDWKKGDTFSCMVGHEALPLAFTQKTIDRLAG

SEQ ID NO: 6 - protein, 220 aa, Artificial Sequence
  Clone ID 23769 IgA2 hinge - IgA2m1 CH2 - IgA2m1 CH3
RVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVRLTCKARGFSPKDVLVRWLQGSQELPREKYLTWASRQEPSQGTTTFAVTSRLRVAAEDWKKGDTFSCMVGHEALPLAFTQKTIDRLAG

SEQ ID NO: 7 - protein, 220 aa, Artificial Sequence
  Clone ID 23770 IgA2 hinge - IgA2m1 CH2 - IgA2m1 CH3
RVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVKLTCKAKGFSPKDVLVRWLQGSQELPREKYLTWASRQEPSQGTTTFAVTSRLRVAAEDWKKGDTFSCMVGHEALPLAFTQKTIDRLAG

SEQ ID NO: 8 - protein, 220 aa, Artificial Sequence
  Clone ID 23771 IgA2 hinge - IgA2m1 CH2 - IgA2m1 CH3
RVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLTCKAKGFSPKDVLVRWLQGSQELPREKYLTWASRQRPSQGTTTFAVTSKLRVAAEDWKKGDTFSCMVGHEALPLAFTQKTIDRLAG

SEQ ID NO: 9 - protein, 220 aa, Artificial Sequence
  Clone ID 23772 IgA2 hinge - IgA2m1 CH2 - IgA2m1 CH3
RVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLTCKAKGFSPKDVLVRWLQGSQELPREKYLTWASRQEPSQGTTTFAVTSKLRVAAEDWKKGDTFSCMVGHEALPLAFTQKTIDRLAG

SEQ ID NO: 10 - protein, 443 aa, Artificial Sequence
  Clone ID 23773 Trastuzumab VH - IgG1 CH1 - IgG1/IgA2 hinge - IgA2m1 CH2 - IgA2m1 CH3
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVDLTCDADGFSPKDVLVRWLQGSQELPREKYLTWASRQEPSQGTTTFAVTSELRVAAEDWKKGDTFSCMVGHEALPLAFTQKTIDRLAG

SEQ ID NO: 11 - protein, 443 aa, Artificial Sequence
  Clone ID 23774 Trastuzumab VH - IgG1 CH1 - IgG1/IgA2 hinge - IgA2m1 CH2 - IgA2m1 CH3
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVELTCEADGFSPKDVLVRWLQGSQELPREKYLTWASRQEPSQGTTTFAVTSDLEVAAEDWKKGDTFSCMVGHEALPLAFTQKTIDRLAG

SEQ ID NO: 12 - protein, 443 aa, Artificial Sequence
  Clone ID 23775 Trastuzumab VH - IgG1 CH1 - IgG1/IgA2 hinge - IgA2m1 CH2 - IgA2m1 CH3
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVELTCLADGFSPKDVLVRWLQGSQELPREKYLTWASRQEPSQGTTTFAVTSDLDVAAEDWKKGDTFSCMVGHEALPLAFTQKTIDRLAG

SEQ ID NO: 13 - protein, 443 aa, Artificial Sequence
  Clone ID 23776 Trastuzumab VH - IgG1 CH1 - IgG1/IgA2 hinge - IgA2m1 CH2 - IgA2m1 CH3
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVELTCLADGFSPKDVLVRWLQGSQELPREKYLTWASRQEPSQGTTTFAVTSELEVAAEDWKKGDTFSCMVGHEALPLAFTQKTIDRLAG

SEQ ID NO: 14 - protein, 443 aa, Artificial Sequence
  Clone ID 23777 Trastuzumab VH - IgG1 CH1 - IgG1/IgA2 hinge - IgA2m1 CH2 - IgA2m1 CH3
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLTCLADGFSPKDVLVRWLQGSQELPREKYLTWASRQDPSQGTTTFAVTSDLEVAAEDWKKGDTFSCMVGHEALPLAFTQKTIDRLAG

SEQ ID NO: 15 - protein, 443 aa, Artificial Sequence
  Clone ID 23778 Trastuzumab VH - IgG1 CH1 - IgG1/IgA2 hinge - IgA2m1 CH2 - IgA2m1 CH3
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLTCLARGFSPKDVLVRWLQGSQELPREKYLTWASRQEPSQGTTTFYVLSILRVAAEDWKKGDTFSCMVGHEALPLAFTQKTIDRLAG

SEQ ID NO: 16 - protein, 443 aa, Artificial Sequence
  Clone ID 23779 Trastuzumab VH - IgG1 CH1 - IgG1/IgA2 hinge - IgA2m1 CH2 - IgA2m1 CH3
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLTCLARGFSPKDVLVRWLQGSQELPREKYLTWASRQEPSQGTTTFYVYSILRVAAEDWKKGDTFSCMVGHEALPLAFTQKTIDRLAG

SEQ ID NO: 17 - protein, 443 aa, Artificial Sequence
  Clone ID 23780 Trastuzumab VH - IgG1 CH1 - IgG1/IgA2 hinge - IgA2m1 CH2 - IgA2m1 CH3
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLTCLARGFSPKDVLVRWLQGSQELPREKYLTWASRQEPSQGTTTFFVYSILRVAAEDWKKGDTFSCMVGHEALPLAFTQKTIDRLAG

SEQ ID NO: 18 - protein, 443 aa, Artificial Sequence
  Clone ID 23781 Trastuzumab VH - IgG1 CH1 - IgG1/IgA2 hinge - IgA2m1 CH2 - IgA2m1 CH3
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLTCMARGFSPKDVLVRWLQGSQELPREKYLTWASRQEPSQGTTTFFVWSILRVAAEDWKKGDTFSCMVGHEALPLAFTQKTIDRLAG

SEQ ID NO: 19 - protein, 443 aa, Artificial Sequence
  Clone ID 23782 Trastuzumab VH - IgG1 CH1 - IgG1/IgA2 hinge - IgA2m1 CH2 - IgA2m1 CH3
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLTCLARGFSPKDVLVRWLQGSQELPREKYLTWASRQEPSQGTTTFYVMSILRVAAEDWKKGDTFSCMVGHEALPLAFTQKTIDRLAG

SEQ ID NO: 20 - protein, 220 aa, Artificial Sequence
  Clone ID 23783 IgA2 hinge - IgA2m1 CH2 - IgA2m1 CH3
RVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLTCLARGFSPKDVLVRWLQGSQELPREKYTTLASRQEPSQGTTTFAVTSLLRVAAEDWKKGDTFSCMVGHEALPLAFTQKTIDRLAG

SEQ ID NO: 21 - protein, 220 aa, Artificial Sequence
  Clone ID 23784 IgA2 hinge - IgA2m1 CH2 - IgA2m1 CH3
RVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLTCLARGFSPKDVLVRWLQGSQELPREKYVTLASRQEPSQGTTTFAVTSLLRVAAEDWKKGDTFSCMVGHEALPLAFTQKTIDRLAG

SEQ ID NO: 22 - protein, 220 aa, Artificial Sequence
  Clone ID 23785 IgA2 hinge - IgA2m1 CH2 - IgA2m1 CH3
RVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLTCLARGFSPKDVLVRWLQGSQELPREKYLTLASRQEPSQGTTTFAVTSILRVAAEDWKKGDTFSCMVGHEALPLAFTQKTIDRLAG

SEQ ID NO: 23 - protein, 220 aa, Artificial Sequence
  Clone ID 23786 IgA2 hinge - IgA2m1 CH2 - IgA2m1 CH3
RVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLTCLARGFSPKDVLVRWLQGSQELPREKYVTTASRQEPSQGTTTFAVTSLLRVAAEDWKKGDTFSCMVGHEALPLAFTQKTIDRLAG

SEQ ID NO: 24 - protein, 443 aa, Artificial Sequence
  Clone ID 24674 Trastuzumab VH - IgG1 CH1 - IgG1/IgA2 hinge - IgA2m1 CH2 - IgA2m1 CH3
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLVCLARGFSPKDVLVRWLQGSQELPREKYLTWASRQEPSQGTTTFFVYSILRVAAEDWKKGDTFSCMVGHEALPLAFTQKTIDRLAG

SEQ ID NO: 25 - protein, 443 aa, Artificial Sequence
  Clone ID 24675 Trastuzumab VH - IgG1 CH1 - IgG1/IgA2 hinge - IgA2m1 CH2 - IgA2m1 CH3
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLLCLARGFSPKDVLVRWLQGSQELPREKYLTWASRQEPSQGTTTFFVYSILRVAAEDWKKGDTFSCMVGHEALPLAFTQKTIDRLAG

SEQ ID NO: 26 - protein, 443 aa, Artificial Sequence
  Clone ID 24676 Trastuzumab VH - IgG1 CH1 - IgG1/IgA2 hinge - IgA2m1 CH2 - IgA2m1 CH3
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLICLARGFSPKDVLVRWLQGSQELPREKYLTWASRQEPSQGTTTFFVYSILRVAAEDWKKGDTFSCMVGHEALPLAFTQKTIDRLAG

SEQ ID NO: 27 - protein, 220 aa, Artificial Sequence
  Clone ID 24677 IgA2 hinge - IgA2m1 CH2 - IgA2m1 CH3
RVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLFPPPSEELALNELVTLTCLARGFSPKDVLVRWLQGSQELPREKYVTTASRQEPSQGTTTFAVTSLLRVAAEDWKKGDTFSCMVGHEALPLAFTQKTIDRLAG

SEQ ID NO: 28 - protein, 443 aa, Artificial Sequence
  Clone ID 24678 Trastuzumab VH - IgG1 CH1 - IgG1/IgA2 hinge - IgA2m1 CH2 - IgA2m1 CH3
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVYLLPPPSEELALNELVTLTCLARGFSPKDVLVRWLQGSQELPREKYLTWASRQEPSQGTTTFFVYSILRVAAEDWKKGDTFSCMVGHEALPLAFTQKTIDRLAG

SEQ ID NO: 29 - protein, 220 aa, Artificial Sequence
  Clone ID 24679 IgA2 hinge - IgA2m1 CH2 - IgA2m1 CH3
RVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVYLLPPPSEELALNELVTLTCLARGFSPKDVLVRWLQGSQELPREKYVTTASRQEPSQGTTTFAVTSLLRVAAEDWKKGDTFSCMVGHEALPLAFTQKTIDRLAG

SEQ ID NO: 30 - protein, 443 aa, Artificial Sequence
  Clone ID 25880 Trastuzumab VH - IgG1 CH1 - IgG1/IgA2 hinge - IgA2m1 CH2 - IgA2m1 CH3
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVCLLPPPSEELALNELVTLTCLARGFSPKDVLVRWLQGSQELPREKYLTWASRQEPSQGTTTFFVYSILRVAAEDWKKGDTFSCMVGHEALPLAFTQKTIDRLAG

SEQ ID NO: 31 - protein, 220 aa, Artificial Sequence
  Clone ID 25881 IgA2 hinge - IgA2m1 CH2 - IgA2m1 CH3
RVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPCSEELALNELVTLTCLARGFSPKDVLVRWLQGSQELPREKYVTTASRQEPSQGTTTFAVTSLLRVAAEDWKKGDTFSCMVGHEALPLAFTQKTIDRLAG

SEQ ID NO: 32 - protein, 443 aa, Artificial Sequence
  Clone ID 25882 Trastuzumab VH - IgG1 CH1 - IgG1/IgA2 hinge - IgA2m1 CH2 - IgA2m1 CH3
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPCSEELALNELVTLTCLARGFSPKDVLVRWLQGSQELPREKYLTWASRQEPSQGTTTFFVYSILRVAAEDWKKGDTFSCMVGHEALPLAFTQKTIDRLAG

SEQ ID NO: 33 - protein, 220 aa, Artificial Sequence
  Clone ID 25883 IgA2 hinge - IgA2m1 CH2 - IgA2m1 CH3
RVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVCLLPPPSEELALNELVTLTCLARGFSPKDVLVRWLQGSQELPREKYVTTASRQEPSQGTTTFAVTSLLRVAAEDWKKGDTFSCMVGHEALPLAFTQKTIDRLAG

SEQ ID NO: 34 - protein, 443 aa, Artificial Sequence
  Clone ID 25884 Trastuzumab VH - IgG1 CH1 - IgG1/IgA2 hinge - IgA2m1 CH2 - IgA2m1 CH3
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVCLLPPCSEELALNELVTLTCLARGFSPKDVLVRWLQGSQELPREKYLTWASRQEPSQGTTTFFVYSILRVAAEDWKKGDTFSCMVGHEALPLAFTQKTIDRLAG

SEQ ID NO: 35 - protein, 220 aa, Artificial Sequence
  Clone ID 25885 IgA2 hinge - IgA2m1 CH2 - IgA2m1 CH3
RVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVCLLPPCSEELALNELVTLTCLARGFSPKDVLVRWLQGSQELPREKYVTTASRQEPSQGTTTFAVTSLLRVAAEDWKKGDTFSCMVGHEALPLAFTQKTIDRLAG

SEQ ID NO: 36 - protein, 443 aa, Artificial Sequence
  Clone ID 25886 Trastuzumab VH - IgG1 CH1 - IgG1/IgA2 hinge - IgA2m1 CH2 - IgA2m1 CH3
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVCLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAG

SEQ ID NO: 37 - protein, 220 aa, Artificial Sequence
  Clone ID 25887 IgA2 hinge - IgA2m1 CH2 - IgA2m1 CH3
RVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPCSEELALNELVTLTCLARGFSPKDVLVRWLQGSQELPREKYLTWASRQEPSQGTTTFAVTSILRVAAEDWKKGDTFSCMVGHEALPLAFTQKTIDRLAG

SEQ ID NO: 38 - protein, 218 aa, Artificial Sequence
  Anti-Her2 Fab HC
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 39 - protein, 214 aa, Artificial Sequence; identical to SEQ ID NO: 1
  Anti-Her2 Fab LC

SEQ ID NO: 40 - protein, 5 aa, Artificial Sequence
  IgG1 upper hinge
EPKSC

SEQ ID NO: 41 - protein, 7 aa, Artificial Sequence
  IgA2 hinge
RVPPPPP

SEQ ID NO: 42 - protein, 213 aa, Artificial Sequence
  IgA2m1 wild type
CCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAG

SEQ ID NO: 43 - protein, 213 aa, Artificial Sequence
  Modified IgA2m1
CCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAG

SEQ ID NO: 44 - protein, 213 aa, Artificial Sequence
  IgA1 wild type
CCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAG

SEQ ID NO: 45 - protein, 213 aa, Artificial Sequence
  IgA2m2 wild type
CCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRLAG

SEQ ID NO: 46 - protein, 19 aa, Artificial Sequence
  alpha-tailpiece IgA1/IgA2m1
KPTHVNVSVVMAEVDGTCY

SEQ ID NO: 47 - protein, 19 aa, Artificial Sequence
  alpha-tailpiece IgA2m2
KPTHINVSVVMAEADGTCY

SEQ ID NO: 48 - protein, 164 aa, Artificial Sequence
  J Chain
EFATMRPTWAWWLFLVLLLALWAPARGQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 49 - protein, 27 aa, Artificial Sequence
  Signal peptide
EFATMRPTWAWWLFLVLLLALWAPARG

(19) United States
(12) Patent Application Publication
(10) Pub. No.: US 2024/0294667 A9
(43) Pub. Date: Sep. 5, 2024

(54) HETERODIMERIC IGA FC CONSTRUCTS AND METHODS OF USE THEREOF

(71) Applicant: Zymeworks BC Inc., Vancouver (CA)

(72) Inventors: Eric Escobar-Cabrera, Vancouver (CA); Florian Heinkel, Vancouver (CA); Thomas Spreter Von Kreudenstein, Vancouver (CA); Meghan Marie Verstraete, Vancouver (CA); Surjit Bhimarao Dixit, Vancouver (CA)

(21) Appl. No.: 18/265,341

(22) PCT Filed: Dec. 3, 2021
(86) PCT No.: PCT/CA2021/051732

§ 371 Date: Jun. 5, 2023

Related U.S. Application Data
(60) Provisional application No. 63/194,828, filed on May 28, 2021.
(60) Provisional application No. 63/121,180, filed on Dec. 3, 2020.

(51) Int. Cl. C07K 16/32

(52) U.S. Cl. CPC C07K 16/32; C07K 2317/31; C07K 2317/526; C07K 2317/55; C07K 2317/622; C07K 2317/72; C07K 2317/732; C07K 2317/92; C07K 2317/94

(57) ABSTRACT

Heterodimeric IgA Fc (IgA HetFc) constructs comprising one or more amino acid mutations in the CH3 domain that allow for formation of a heterodimeric Fc having high purity and thermostability. The IgA HetFc constructs may comprise one or more target binding domains. Higher order IgA HetFc multimers comprising multiple IgA HetFc constructs may be prepared in which two of the IgA HetFc constructs are joined by a J chain.

### TECHNICAL FIELD

The present disclosure relates to the field of IgA-based immunotherapeutics and, in particular, to heterodimeric IgA Fe (IgA HetFc) constructs comprising one or more binding domain and the use of these constructs as therapeutics.

### BACKGROUND

Typically, antibody-based therapeutics contain an IgG-derived framework. The Ig subtype is stable, binds to targets with high affinity, has favourable pharmacokinetic behaviour and has a well understood functional impact on target and effector cells as a result of decades of focused research. However, there are limits to IgG-based functionality with respect to the effector cells it is able to activate and the valencies that can be obtained.

Neutrophils are an integral part of the immune system and are the most prevalent leukocyte found in human blood (see Table 1). IgA is the only Ig isotype that interacts with FcαRI on neutrophils via residues in the Cα2/Cα3 (IgA CH2/CH3) interface of the Fc. Interaction of IgA with FcαRI on neutrophils elicits a variety of pro-inflammatory responses including the release of Neutrophil Extracellular Traps (NETs), degranulation and chemokine release (Heineke, 2017, Eur J Clin Invest., 47(2):184-192). IgA also can mediate cytotoxicity ex vivo. Neutrophils activated by IgA have been shown to be capable of killing Her2⁺⁺⁺ BT474 cells (Borrok et al., 2015, MAbs 7:743-751). IgA mediated tumor cell killing via Her2 and other targets has been shown by neutrophils ex vivo (Brandsma et al., 2019, Front Immunol, 10:704). Moreover, IgA can mediate tumor growth inhibition in vivo. In particular, IgA has been shown to inhibit tumor growth in vivo in a FcαRI transgenic (Tg) mouse model (Boross et al., 2013, EMBO Mol Med, 5:1213-1226).

TABLE 1
Immune Cells in Human Blood*

| | Median | Median |
|---|---|---|
| | Concentration in | percentage of |
| | human blood | white blood |
| Cell type | [Cells × 10⁹/L] | cells |
| Neutrophils | 3.65 | 53.0 |
| Lymphocytes (incl. T cells, | 2.5 | 36.1 |
| B cells and NK cells) | | |
| Eosinophils | 0.15 | 3.2 |
| Basophils | 0.03 | 0.6 |
| Monocytes | 0.43 | 7.1 |

*Data collected from 291 adults (see Orfanakis, et al., 1970, Am J Clin Pathol, 53: 647-651)

Recruitment and activation of neutrophils via IgA affords new biological functions for antibody-based immunotherapies.

### SUMMARY

Described herein are heterodimeric IgA Fc constructs and methods of use thereof. One aspect of the present disclosure relates to an IgA heterodimeric Fc (IgA HetFc) construct comprising a first Fc polypeptide and a second Fc polypeptide, the first Fc polypeptide comprising a first CH3 domain sequence and the second Fc polypeptide comprising an second CH3 domain sequence, the first and second CH3 domain sequences forming a modified CH3 domain, wherein the first and second CH3 domain sequences comprise amino acid mutations that promote formation of a heterodimeric Fc over a homodimeric Fc, wherein: the amino acid mutations in the first CH3 domain sequence comprise an amino acid substitution at position A6085Y selected from A6085YF, A6085YY, A6085YM, A6085YW and A6085YH, and an amino acid substitution at position T6086 selected from T6086Y, T6086F, T6086M, T6086W and T6086H, and the amino acid mutations in the second CH3 domain sequence comprise an amino acid substitution at position W608I selected from W6081T, W6081L, W6081A, W6081V and W6081I, wherein the heterodimeric Fc is formed with a purity of 70% or higher, and wherein the numbering of amino acid positions is according to IMGT numbering.

Another aspect of the present disclosure relates to an IgA heterodimeric Fc (IgA HetFc) construct comprising a first Fc polypeptide and a second Fc polypeptide, the first Fc polypeptide comprising a first CH3 domain sequence and the second Fc polypeptide comprising an second CH3 domain sequence, the first and second CH3 domain sequences forming a modified CH3 domain, wherein the first and second CH3 domain sequences comprise amino acid mutations that promote formation of a heterodimeric Fc over a homodimeric Fc, wherein:
(a) the amino acid mutations in the first CH3 domain sequence comprise the amino acid substitutions: A6085YY and T6086L, and the amino acid mutations in the second CH3 domain sequence comprise the amino acid substitutions: L6079T, W6081L and I6088L; or
(b) the amino acid mutations in the first CH3 domain sequence comprise the amino acid substitutions: A6085YY and T6086Y, and the amino acid mutations in the second CH3 domain sequence comprise the amino acid substitutions: L6079T, W6081L and I6088L; or
(c) the amino acid mutations in the first CH3 domain sequence comprise the amino acid substitutions: A6085YF and T6086Y, and the amino acid mutations in the second CH3 domain sequence comprise the amino acid substitutions: L6079V, W6081L and I6088L; or
(d) the amino acid mutations in the first CH3 domain sequence comprise the amino acid substitutions: A6085YF and T6086Y, and the amino acid mutations in the second CH3 domain sequence comprise the amino acid substitutions: L6079V, W6081T and I6088L; or
(e) the amino acid mutations in the first CH3 domain sequence comprise the amino acid substitutions: T6022V, A6085YF and T6086Y, and the amino acid mutations in the second CH3 domain sequence comprise the amino acid substitutions: L6079V, W6081T and I6088L; or
(f) the amino acid mutations in the first CH3 domain sequence comprise the amino acid substitutions: T6022L, A6085YF and T6086Y, and the amino acid mutations in the second CH3 domain sequence comprise the amino acid substitutions: L6079V, W6081T and I6088L; or
(g) the amino acid mutations in the first CH3 domain sequence comprise the amino acid substitutions: T6022I, A6085YF and T6086Y, and the amino acid mutations in the second CH3 domain sequence comprise the amino acid substitutions: L6079V, W6081T and I6088L; or
(h) the amino acid mutations in the first CH3 domain sequence comprise the amino acid substitutions: A6085YF and T6086Y, and the amino acid mutations in the second CH3 domain sequence comprise the amino acid substitutions: L6007F, L6079V, W6081T and I6088L
(i) the amino acid mutations in the first CH3 domain sequence comprise the amino acid substitutions: H6005Y, A6085YF and T6086Y, and the amino acid mutations in the second CH3 domain sequence comprise the amino acid substitutions: H6005Y, L6079V, W6081T and I6088L; or
(j) the amino acid mutations in the first CH3 domain sequence comprise the amino acid substitutions: H6005C, A6085YF and T6086Y, and the amino acid mutations in the second CH3 domain sequence comprise the amino acid substitutions: P6010C, L6079V, W6081T and I6088L; or
(k) the amino acid mutations in the first CH3 domain sequence comprise the amino acid substitutions: P6010C, A6085YF and T6086Y, and the amino acid mutations in the second CH3 domain sequence comprise the amino acid substitutions: H6005C, L6079V, W6081T and I6088L; or
(l) the amino acid mutations in the first CH3 domain sequence comprise the amino acid substitutions: H6005C, P6010C, A6085YF and T6086Y, and the amino acid mutations in the second CH3 domain sequence comprise the amino acid substitutions: H6005C, P6010C, L6079V, W6081T and I6088L,
wherein the heterodimeric Fc is formed with a purity of 70% or higher, and wherein the numbering of amino acid positions is according to IMGT numbering.

Another aspect of the present disclosure relates to a conjugate comprising an IgA HetFc construct as described herein and one or more therapeutic, diagnostic or labeling agents.

Another aspect of the present disclosure relates to an IgA HetFc multimer comprising two or more IgA HetFc constructs as described herein and a J chain, wherein two of the IgA HetFc constructs are joined by the J chain.

Another aspect of the present disclosure relates to a pharmaceutical composition comprising an IgA HetFc construct as described herein and a pharmaceutically acceptable carrier or diluent.

Another aspect of the present disclosure relates to a pharmaceutical composition comprising a conjugate comprising an IgA HetFc construct and one or more therapeutic, diagnostic or labeling agents as described herein, and a pharmaceutically acceptable carrier or diluent.

Another aspect of the present disclosure relates to a pharmaceutical composition comprising an IgA HetFc multimer comprising two or more IgA HetFc constructs and a J chain as described herein, and a pharmaceutically acceptable carrier or diluent.

Another aspect of the present disclosure relates to an isolated polynucleotide or set of polynucleotides encoding an IgA HetFc construct as described herein.

Another aspect of the present disclosure relates to a vector set or set of vectors comprising one or more polynucleotides encoding an IgA HetFc as described herein.

Another aspect of the present disclosure relates to a host cell comprising one or more polynucleotides encoding an IgA HetFc as described herein.

Another aspect of the present disclosure relates to a method of preparing an IgA HetFc construct as described herein comprising transfecting a host cell with one or more polynucleotides encoding the IgA HetFc construct, and culturing the host cell under conditions suitable for expression of the IgA HetFc construct.

Another aspect of the present disclosure relates to a method of preparing an IgA HetFc multimer as described herein, comprising transfecting a host cell with one or more polynucleotides encoding an IgA HetFc construct comprising an α-tailpiece and a polynucleotide encoding a J chain, and culturing the host cell under conditions suitable for expression of the IgA HetFc construct and the J chain.

### BRIEF DESCRIPTION OF THE DRAWINGS

FIG. 1 presents a cartoon depicting negative and positive design concepts for mutations to drive heterodimerization of an IgA Fc.

FIG. 2 presents non-reducing CE-SDS profiles of IgA Fc one armed antibody (OAA) constructs after CaptureSelect™ IgA affinity purification: (A) IgA Fc OAA constructs comprising WT IgA CH3 (variant number 32595) or Steric Design 1, 2, 3, 4 or 6 (variant numbers 32516, 32517, 32518, 32519 and 32521, respectively), (B) IgA Fc OAA constructs comprising Steric Design 7, 8, 9, 10 or 11 (variant numbers 33330, 33331, 33332, 33333 and 33334, respectively).

FIG. 3 presents UPLC-SEC chromatograms of IgA Fc OAA constructs after CaptureSelect™ IgA affinity purification: (A) UPLC-SEC chromatogram of IgA Fc OAA construct comprising a WT IgA CH3 (variant number 32595); (B-K) UPLC-SEC chromatograms of IgA OAA constructs comprising Steric Design 1, 2, 3, 4, 6, 7, 8, 9, 10 or 11 (variant numbers 32516, 32517, 32518, 32519, 32521, 33330, 33331, 33332, 33333 and 33334, respectively).

FIG. 4 presents UPLC-SEC chromatograms of IgA Fc OAA constructs after purification by preparative SEC: (A) IgA OAA construct comprising WT IgA CH3 (variant number 32595), (B-J) IgA OAA constructs comprising Steric Design 1, 2, 3, 6, 7, 8, 9, 10 or 11 (variant numbers 32516, 32517, 32518, 32521, 33330, 33331, 33332, 33333 and 33334, respectively).

FIG. 5 presents non-reducing and reducing CE-SDS profiles of IgA Fc OAA constructs after purification by preparative SEC: (A) IgA OAA constructs comprising WT IgA CH3 (variant 32595) or Steric Design 1, 2, 3 or 6 (variant numbers 32516, 32517, 32518 and 32521, respectively), (B) IgA OAA constructs comprising Steric Design 7, 8, 9, 10 or 11 (variant numbers 33330, 33331, 33332, 33333 and 33334, respectively).

FIG. 6 presents an overlay of DSC thermograms for IgA Fc OAA constructs after purification by preparative SEC: (A) IgA Fc constructs comprising WT IgA CH3 (variant number 32595) or Steric Design 1, 2, 3 or 6 (variant numbers 32516, 32517, 32518 and 32521, respectively), (B) IgA Fc constructs comprising Steric Design 7-11 (variant numbers 33330-33334).

FIG. 7 depicts examples of components and configurations of IgA HetFc binding units: (A) the IgA HetFc scaffold to which binding domains are fused to form an IgA HetFc binding unit, (B) illustrative IgA HetFc binding unit showing the IgA HetFc scaffold with two exemplary binding domains attached; (C-H) illustrative IgA HetFc binding units having from one to four binding domains fused to the IgA HetFc scaffold in different configurations. Binding domains are shown as Fabs for illustrative purposes but may be various other binding domains (e.g. scFv) and combinations of binding domains. The formats provided are for illustrative purposes and does not limit the disclosure in any way.

FIG. 8 depicts illustrative higher order IgA HetFc multimers comprising two, four and five IgA HetFc binding units joined by a J chain (stippled). The two chains of the IgA HetFc are shown in grey and striped. The tailpiece assembly in the centre of each structure is indicated. A single orientation is shown for each assembly but many orientations are possible. Since the J chain and Fc:Fc interactions are not selective for chain A or chain B, the orientation of the binding domains of each binding unit can be reversed. (A) a dimeric IgA HetFc multimer comprising two bispecific IgA HetFc binding units joined by a J chain, (B) a tetrameric IgA HetFc multimer comprising four bispecific IgA HetFc binding units joined by a J chain, and (C) a pentameric IgA HetFc multimer comprising five bispecific IgA HetFc binding units joined by a J chain.

FIG. 9 presents structural representations of IgA HetFc design (Steric 6) with chain A and chain B indicated. The protein backbone is depicted in cartoon representation and side chains are shown as line representation. Non-polar hydrogens are not shown. (A) shows the full IgA heterodimeric Fc, and (B) presents a magnified view of the mutated residues centered around the core positions A6085, T6086 (both chain A) and W608I (chain B).

FIG. 10 presents an alignment of the amino acid sequences for the IgA1, IgA2 m1 and IgA2m2 Fc regions.

FIG. 11 presents IgA OAA variants based on an IgA HetFc with mutations eliminating binding of FcαR in one or both chains of the Fc.

FIG. 12 presents a modified IgA mAb based on an IgA HetFc that is capable of binding both FcαR and FcRn.

### DETAILED DESCRIPTION

The present disclosure relates to the engineering of IgA Fc regions to introduce amino acid mutations into the CH3 domain that promote formation of a heterodimeric IgA Fc (IgA HetFc). The IgA HetFc allows for construction of IgA-based bispecific or multispecific binding proteins, as well as IgA-based multimeric binding proteins. In accordance with the present disclosure, the one or more amino acid mutations comprised by the IgA HetFc constructs allow for formation of a heterodimeric Fc having a purity of at least about 70%. The IgA HetFc constructs of the present disclosure are also thermostable. For example, in certain embodiments, the CH3 domain of the IgA HetFc has a melting temperature (Tm) that is about 60° C. or higher. In some embodiments, the CH3 domain of the IgA Het Fc has a Tm that is within 10° C. (±10° C.) of the Tm of a wild-type IgA CH3 domain.

The IgA HetFc constructs of the present disclosure include IgA HetFc scaffolds, which comprise an IgA Fc region together with a hinge region; IgA HetFc binding units, which comprise an IgA scaffold and one or more binding domains; and IgA HetFc multimers, which comprise a plurality (e.g. two or more) IgA HetFc binding units.

The IgA HetFc constructs of the present disclosure introduce a multispecific potential to the IgA isotype with functionalities that are untapped by IgG. For example, in certain embodiments, the IgA HetFc facilitates the creation of multispecific and multimeric biologics capable of recruitment of neutrophils via the FcαRI. As neutrophils are an integral part of the immune system and are the most prevalent leukocyte found in human blood, recruitment and activation of neutrophils via IgA affords new biological functions for antibody-based immunotherapies. Certain embodiments of the present disclosure relate to methods of using IgA HetFc binding units and IgA HetFc multimers as therapeutics. Certain embodiments of the present disclosure relate to methods of using IgA HetFc binding units and IgA HetFc multimers as diagnostics.

### Definitions

Unless defined otherwise, all technical and scientific terms used herein have the same meaning as commonly understood by one of ordinary skill in the art.

As used herein, the term “about” refers to an approximately +/−10% variation from a given value, unless otherwise indicated. It is to be understood that such a variation is always included in any given value provided herein, whether or not it is specifically referred to.

The use of the word “a” or “an” when used herein in conjunction with the term “comprising” may mean “one,” but it is also consistent in certain embodiments with the meaning of “one or more,” “at least one” or “one or more than one.”

As used herein, the terms “comprising,” “having,” “including” and “containing,” and grammatical variations thereof, are inclusive or open-ended and do not exclude additional, unrecited elements and/or method steps. The term “consisting essentially of” when used herein in connection with a composition, use or method, denotes that additional elements and/or method steps may be present, but that these additions do not materially affect the manner in which the recited composition, method or use functions. The term “consisting of” when used herein in connection with a composition, use or method, excludes the presence of additional elements and/or method steps. A composition, use or method described herein as comprising certain elements and/or steps may also, in certain embodiments consist essentially of those elements and/or steps, and in other embodiments consist of those elements and/or steps, whether or not these embodiments are specifically referred to.

By “fused” is meant that the components of the multimers described herein (e.g. an antibody or antigen-binding fragment thereof and an Fc domain polypeptide) are linked by peptide bonds, either directly or via one or more peptide linkers.

As used herein, the term “single-chain” refers to a molecule comprising amino acid monomers linearly linked by peptide bonds. For example, an antigen-binding fragment of an antibody may comprise a single chain variable region (scFv).

As used herein an “IgA HetFc construct” is meant to include any of the IgA HetFc constructs described herein, including IgA HetFc scaffolds (heterodimeric IgA Fc), IgA HetFc binding units (heterodimeric IgA binding units) and IgA HetFc multimers.

The term “functional” in connection with a modified J chain means that the J chain retains the primary function of a native J chain, e.g., a native human J chain, in particular, the ability to enable efficient polymerization (dimerization, tetramerization) of IgA and binding of such polymers (dimers, tetramers) to the secretory component (SC)/polymeric (p)Ig.

The term “isolated,” as used herein with reference to a material, means that the material is removed from its original environment (for example, the natural environment if it is naturally occurring). For example, a naturally occurring polynucleotide or polypeptide present in a living animal is not isolated, but the same polynucleotide or polypeptide separated from some or all of the co-existing materials in the natural system, is isolated. Such polynucleotides could be part of a vector and/or such polynucleotides or polypeptides could be part of a composition, and still be isolated in that such vector or composition is not part of its natural environment.

The term “conservatively modified variant” when used herein with reference to an amino acid sequence, such as a peptide, polypeptide or protein sequence, means that the amino acid sequence has been altered by substitution, addition or deletion of a single amino acid or a small percentage of amino acids without significantly impact the function of the sequence. For example, a conservatively modified variant may be an amino acid sequence that has been altered by one or more conservative amino acid substitutions. Conservative substitution tables providing functionally similar amino acids are known to those of ordinary skill in the art. For example, the following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and [0139]8) Cysteine (C), Methionine (M) (see, for example, Creighton, Proteins: Structures and Molecular Properties (W H Freeman & Co.; 2nd edition (December 1993)). In certain embodiments, the IgA sequence used as a base sequence for the IgA HetFc constructs may be a conservatively modified variant.

The term “substantially identical” as used herein in relation to an amino acid sequence indicates that, when optimally aligned, for example using the methods described below, the sequence shares at least 70%, at least 75%, at least 80%, at least 85%, at least 90%, at least 95%, at least 96%, at least 97%, at least 98% or at least 99% sequence identity with a defined second amino acid sequence (or “reference sequence”). In certain embodiments, a substantially identical amino acid sequence has at least 95%, at least 96%, at least 97%, at least 98% or at least 99% sequence identity with the reference sequence. “Substantial identity” may be used to refer to various types and lengths of sequence, such as full-length sequence or a functional domain. Percent identity between two amino acid sequences can be determined in various ways well-known in the art, for example, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 10 (1981)) as incorporated into GeneMatcher Plus™ Schwarz and Dayhof (1979) Atlas of Protein Sequence and Structure, Dayhof, M. O., Ed pp 353-358; BLAST program (Basic Local Alignment Search Tool (Altschul, S. F., W. Gish, et al. (1990) J Mol Biol 215: 403-10), and variations thereof including BLAST-2, BLAST-P, BLAST-N, BLAST-X, WU-BLAST-2, ALIGN, ALIGN-2, CLUSTAL, and Megalign (DNASTAR) software. In addition, those skilled in the art can determine appropriate parameters for measuring alignment, including algorithms needed to achieve maximal alignment over the length of the sequences being compared. In general, for amino acid sequences, the length of comparison sequences will be at least 10 amino acids. One skilled in the art will understand that the actual length will depend on the overall length of the sequences being compared and may be at least 20, at least 30, at least 40, at least 50, at least 60, at least 70, at least 80, at least 90, at least 100, at least 110, at least 120, at least 130, at least 140, at least 150, or at least 200 amino acids, or it may be the full-length of the amino acid sequence. In certain embodiments, an IgA HetFc construct comprises an amino acid sequence that is at least 90%, at least 95%, at least 96%, at least 97%, at least 98%, or at least 99% identical to a reference amino acid sequence or fragment thereof as set forth in the Table(s) herein.

The terms “derived from” and “based on” when used with reference to a recombinant amino acid sequence mean that the recombinant amino acid sequence is substantially identical to the sequence of the corresponding wild-type amino acid sequence. For example, an IgA Fc amino acid sequence that is derived from (or based on) a wild-type IgA Fc sequence is substantially identical (e.g., shares at least 90%, at least 95%, at least 96%, at least 97%, at least 98% or at least 99% sequence identity) with the wild-type IgA Fc sequence.

The term “subject,” as used herein, refers to an animal, in some embodiments a mammal, which is the object of treatment, observation or experiment. An animal may be a human, a non-human primate, a companion animal (e.g., a dog, cat, and the like), a farm animal (e.g., a cow, sheep, pig, horse, and the like) or a laboratory animal (e.g., a rat, mouse, guinea pig, and the like).

The term “mammal,” as used herein, includes but is not limited to humans, non-human primates, canines, felines, murines, bovines, equines and porcines.

The term “knock-out or knockout” as used herein, refers to a mutation or a set of mutations within various locations in a variant resulting in eliminating or lessening binding to a binding target.

In the present description, any concentration range, percentage range, ratio range, or integer range is to be understood to include the value of any integer within the recited range and, when appropriate, fractions thereof (such as one tenth and one hundredth of an integer), unless otherwise indicated.

It is contemplated that any embodiment discussed herein can be implemented with respect to any method, use or composition disclosed herein.

Particular features, structures and/or characteristics described in connection with an embodiment disclosed herein may be combined with features, structures and/or characteristics described in connection with another embodiment disclosed herein in any suitable manner to provide one or more further embodiments.

It is also to be understood that the positive recitation of a feature in one embodiment, serves as a basis for excluding the feature in an alternative embodiment. For example, where a list of options is presented for a given embodiment or claim, it is to be understood that one or more option may be deleted from the list and the shortened list may form an alternative embodiment, whether or not such an alternative embodiment is specifically referred to.

Terms understood by those in the art of antibody technology are each given the meaning acquired in the art, unless expressly defined differently herein. Antibodies are known to have variable regions, a hinge region, and constant domains. Immunoglobulin structure and function are reviewed, for example, in Harlow et al (Eds.), Antibodies: A Laboratory Manual, Chapter 14 (Cold Spring Harbor Laboratory, Cold Spring Harbor, 1988).

Unless otherwise specified herein, numbering of amino acid residues in the IgA Fc region and IgA tailpiece is according to the IMGT numbering system (see Lefranc, et al., 2003, Dev Comp Immunol, 27:55-77; Lefranc, et al., 2005, Dev Comp Immunol, 29:185-203). Table 2 provides the IMGT numbering and amino acid sequence for the IgA2m1 Fc CH2 and CH3 domains, together with the equivalent EU numbering (by alignment). Numbering of other IgA Fc sequences can be readily determined by one skilled in the art by simple sequence alignment with the sequence shown in Table 2 using known techniques. Table 3 provides the IMGT numbering and amino acid sequence for the IgA tailpiece.

TABLE 2
IgA2m1* Fc CH2 and CH3 Domains
Sequence, IMGT and EU Numbering

| CH2 domain | | | CH3 domain | | |
|---|---|---|---|---|---|
| | | Amino | | | Amino |
| IMGT No. | EU No. | Acid | IMGT No. | EU No. | Acid |
| 5001 | 231 | C | 6001 | 341 | G |
| 5001A | 232 | C | 6001A | 342 | N |
| 5001Z | 237 | H | 6001B | 343 | T |
| 5002 | 238 | P | 6001Y | 344 | F |
| 5003 | 239 | R | 6001Z | 345 | R |
| 5004 | 240 | L | 6002 | 346 | P |
| 5005 | 241 | S | 6003 | 347 | E |
| 5006 | 242 | L | 6004 | 348 | V |
| 5007 | 243 | H | 6005 | 349 | H |
| 5008 | 244 | R | 6006 | 350 | L |
| 5009 | 245 | P | 6007 | 351 | L |
| 5010 | 246 | A | 6008 | 352 | P |
| 5011 | 247 | L | 6009 | 353 | P |
| 5012 | 248 | E | 6010 | 354 | P |
| 5013 | 249 | D | 6011 | 355 | S |
| 5014 | 250 | L | 6012 | 356 | E |
| 5015 | 251 | L | 6013 | 357 | E |
| 5015A | 252 | L | 6014 | 358 | L |
| 5016 | 254 | G | 6015 | 359 | A |
| 5017 | 255 | S | 6015A | 359A | L |
| 5018 | 256 | E | 6016 | 360 | N |
| 5019 | 257 | A | 6017 | 361 | E |
| 5020 | 258 | N | 6018 | 362 | L |
| 5021 | 259 | L | 6019 | 363 | V |
| 5022 | 260 | T | 6020 | 364 | T |
| 5023 | 261 | C | 6021 | 365 | L |
| 5024 | 262 | T | 6022 | 366 | T |
| 5025 | 263 | L | 6023 | 367 | C |
| 5026 | 264 | T | 6024 | 368 | L |
| 5027 | 265 | G | 6025 | 369 | A |
| 5028 | 266 | L | 6026 | 370 | R |
| 5029 | 267 | R | 6027 | 371 | G |
| 5030 | 268 | D | 6028 | 372 | F |
| 5031 | 269 | A | 6029 | 373 | S |
| 5035 | 271 | S | 6030 | 374 | P |
| 5036 | 272 | G | 6035 | 375 | K |
| 5037 | 273 | A | 6036 | 376 | D |
| 5038 | 274 | T | 6037 | 377 | V |
| 5039 | 275 | F | 6038 | 378 | L |
| 5040 | 276 | T | 6039 | 379 | V |
| 5041 | 277 | W | 6040 | 380 | R |
| 5042 | 278 | T | 6041 | 381 | W |
| 5043 | 279 | P | 6042 | 382 | L |
| 5044 | 280 | S | 6043 | 383 | Q |
| 5045 | 281 | S | 6044 | 384 | G |
| 5045A | 282 | G | 6045 | 385 | S |
| 5045B | 283 | K | 6045A | 386 | Q |
| 5045Z | 283A | S | 6045B | 387 | E |
| 5078 | 287 | A | 6045C | 388 | L |
| 5079 | 288 | V | 6045X | 389 | P |
| 5080 | 289 | Q | 6045Y | 389A | R |
| 5081 | 290 | G | 6045Z | 389B | E |
| 5082 | 291 | P | 6077 | 390 | K |
| 5083 | 292 | P | 6078 | 391 | Y |
| 5084 | 293 | E | 6079 | 392 | L |
| 5084A | 294 | R | 6080 | 393 | T |
| 5084B | 295 | D | 6081 | 394 | W |
| 5084C | 296 | L | 6082 | 395 | A |
| 5084Y | 297 | C | 6083 | 396 | S |
| 5084Z | 298 | G | 6084 | 397 | R |
| 5085 | 299 | C | 6084A | 398 | Q |
| 5085A | 300 | Y | 6084B | 399 | E |
| 5085B | 301 | S | 6084C | 400 | P |
| 5085Z | 302 | V | 6084X | 401 | S |
| 5086 | 303 | S | 6084Y | 401A | Q |
| 5087 | 304 | S | 6084Z | 401B | G |
| 5088 | 305 | V | 6085 | 401C | T |
| 5089 | 306 | L | 6085A | 402 | T |
| 5090 | 307 | P | 6085B | 403 | T |
| 5091 | 308 | G | 6085C | 404 | F |
| 5092 | 309 | C | 6085Y | 405 | A |
| 5093 | 310 | A | 6085Z | 406 | V |
| 5094 | 311 | Q | 6086 | 407 | T |
| 5095 | 312 | P | 6087 | 408 | S |
| 5096 | 313 | W | 6088 | 409 | I |
| 5097 | 314 | N | 6089 | 410 | L |
| 5098 | 315 | H | 6090 | 411 | R |
| 5099 | 316 | G | 6091 | 412 | V |
| 5100 | 317 | E | 6092 | 413 | A |
| 5101 | 318 | T | 6093 | 414 | A |
| 5102 | 319 | F | 6094 | 415 | E |
| 5103 | 320 | T | 6095 | 416 | D |
| 5104 | 321 | C | 6096 | 417 | W |
| 5105 | 322 | T | 6097 | 418 | K |
| 5106 | 323 | A | 6098 | 419 | K |
| 5107 | 324 | A | 6099 | 420 | G |
| 5108 | 325 | H | 6100 | 421 | D |
| 5109 | 326 | P | 6101 | 422 | T |
| 5110 | 327 | E | 6102 | 423 | F |
| 5113 | 328 | L | 6103 | 424 | S |
| 5114 | 329 | K | 6104 | 425 | C |
| 5115 | 330 | T | 6105 | 426 | M |
| 5116 | 331 | P | 6106 | 427 | V |
| 5117 | 332 | L | 6107 | 428 | G |
| 5118 | 333 | T | 6108 | 429 | H |
| 5119 | 334 | A | 6109 | 430 | E |
| 5120 | 335 | N | 6110 | 431 | A |
| 5121 | 336 | I | 6112 | 432 | L |
| 5122 | 337 | T | 6113 | 433 | P |
| 5123 | 338 | K | 6114 | 434 | L |
| 5124 | 339 | S | 6115 | 435 | A |
| | | | 6116 | 436 | F |
| | | | 6117 | 437 | T |
| | | | 6118 | 438 | Q |
| | | | 6119 | 439 | K |
| | | | 6120 | 440 | T |
| | | | 6121 | 441 | I |
| | | | 6122 | 442 | D |
| | | | 6123 | 443 | R |
| | | | 6124 | 444 | L |
| | | | 6125 | 445 | A |
| | | | 6129 | 446 | G |

*Chintalacharuvu, et al., 1994, J Immunol 152: 5299-5304

TABLE 3
IgA Tailpiece Sequence and IMGT Numbering

| IMGT No. | Amino Acid |
|---|---|
| 7001 | K |
| 7002 | P |
| 7003 | T |
| 7004 | H |
| 7005 | V |
| 7006 | N |
| 7007 | V |
| 7008 | S |
| 7009 | V |
| 7010 | V |
| 7011 | M |
| 7012 | A |
| 7013 | E |
| 7014 | V |
| 7015 | D |
| 7016 | G |
| 7017 | T |
| 7018 | C |
| 7019 | Y |

The section headings used herein are for organizational purposes only and are not to be construed as limiting the subject-matter described.

### Heterodimeric IgA Fc (IgA HetFc) Constructs

The present disclosure relates to heterodimeric IgA Fc (IgA HetFc) constructs. The IgA HetFc constructs comprise a heterodimer Fc region derived from an IgA Fc region. The heterodimer Fc region comprises a modified CH3 domain that includes one or more asymmetric amino acid mutations that promote heterodimer formation. In certain embodiments, the heterodimer Fc region comprised by the IgA HetFc construct may act as a scaffold (an IgA HetFc scaffold) to which one or more binding domains can be fused to provide an IgA HetFc binding unit. In certain embodiments, multiple (e.g. two or more) IgA binding units may be fused together, for example via a J-chain, to provide IgA HetFc multimers. Other agents (e.g., therapeutic or diagnostic agents) can optionally be conjugated to the IgA HetFc constructs in certain embodiments.

IgA exists as two subtypes, IgA1 and IgA2, as well as various allotypic variants (IgA2m1, IgA2m2, IgA2(n)). Of the two subtypes, IgA2 is more stable than IgA1 since its shorter hinge region renders it resistant to certain bacterial proteases. This shorter hinge also results in a rigid and non-planar structure which facilitates better multivalent binding of IgA2 to antigens on cell surfaces. For the purposes of the present disclosure, the heterodimer Fc region of an IgA HetFc construct may be derived from an IgA1 or IgA2 Fc region, including allotypic variants thereof. In certain embodiments, the heterodimer Fc region of an IgA HetFc construct may be derived from an IgA1 Fc region. In certain embodiments, the heterodimer Fc region of an IgA HetFc construct may be derived from an IgA2 Fc region or an allotypic variant thereof. In some embodiments, the heterodimer Fc region of an IgA HetFc construct may be derived from a human IgA Fc region. In some embodiments, the heterodimer Fc region of an IgA HetFc construct may be derived from a human IgA2 or IgA2m1 Fc region.

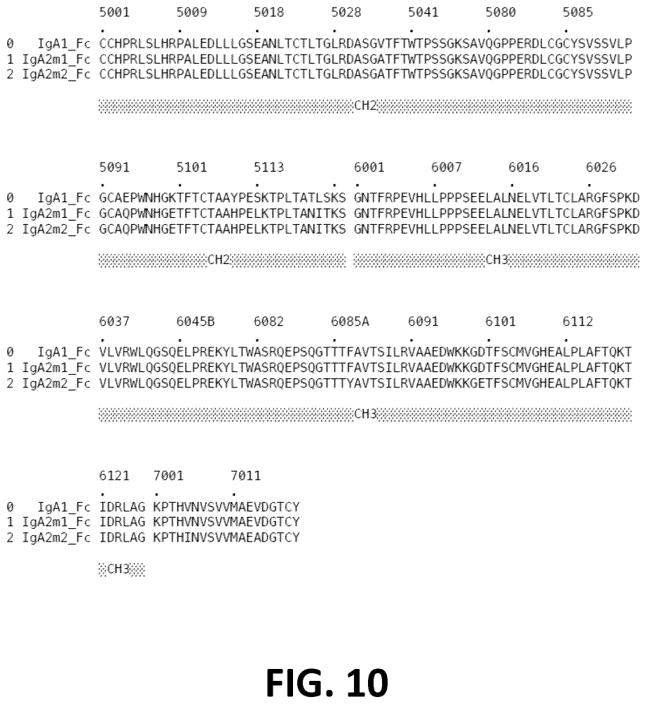

In some embodiments, the heterodimer Fc region of an IgA HetFc construct may be derived from a human IgA2m1 Fc region. Table 4 provides the amino acid sequence of the wild-type human IgA2m1 Fc sequence and of a modified form of IgA2m1 Fc sequence truncated to remove the tailpiece and mutated to remove a free cysteine and a glycosylation site. The Fc sequences correspond to IMGT numbering 5001-6129 of the human IgA2m1 heavy chain. The CH3 sequence of IgA2m1 (underlined) comprises amino acids 6097-6129 (IMGT numbering) of the full-length human IgA1 heavy chain (see e.g., Chintalacharuvu, et al., 1994, J Immunol, 152:5299-5304). The sequence of the IgA tailpiece is also shown. Amino acid sequences of the IgA1 and IgA2m2 Fc regions are provided in Sequence Table B as SEQ ID NOs:44 and 45. An alignment of the Fc sequences is provided in FIG. 10.

TABLE 4
IgA2m1 Fc Amino Acid Sequences

| | |
|---|---|
| IgA2m1 wild type | CCHPRLSLHRPALEDLLLGSEANLTCTLTGLRDASGAT |
| sequence¹ | FTWTPSSGKSAVQGPPERDLCGCYSVSSVLPGCAQPW |
| | NHGETFTCTAAHPELKTPLTANITKSGNTFRPEVHLLP |
| | PPSEELALNELVTLTCLARGFSPKDVLVRWLQGSQELP |
| | REKYLTWASRQEPSQGTTTFAVTSILRVAAEDWKKGD |
| | TFSCMVGHEALPLAFTQKTIDRLAG [SEQ ID NO: 42] |
| Modified IgA2m1 | CCHPRLSLHRPALEDLLLGSEANLTCTLTGLRDASGAT |
| sequence | FTWTPSSGKSAVQGPPERDLCGCYSVSSVLPGSAQPW |
| (C5092S/N5120T/I5121L/ | NHGETFTCTAAHPELKTPLTATLSKSGNTFRPEVHLLP |
| T5122S/Δ α-tailpiece²) | PPSEELALNELVTLTCLARGFSPKDVLVRWLQGSQELP |
| | REKYLTWASRQEPSQGTTTFAVTSILRVAAEDWKKGD |
| | TFSCMVGHEALPLAFTQKTIDRLAG [SEQ ID NO: 43] |
| IgA1/IgA2m1 Tailpiece | KPTHVNVSVVMAEVDGTCY [SEQ ID NO: 46] |

¹Chintalacharuvu, et al., 1994, J Immunol, 152:5299-5304
²Lohse et al., 2016, Cancer Res, 76:403-417. Mutations shown in bold and underline.

The terms “Fc region,” “Fc domain” and “Fc,” are used interchangeably herein to define a C-terminal region of an immunoglobulin heavy chain. An Fc region typically comprises a CH2 domain and a CH3 domain. The Fc region may also be considered to encompass the hinge region in certain embodiments. An “Fc polypeptide” of a dimeric Fc as used herein refers to one of the two polypeptides forming the dimeric Fc domain, i.e., a polypeptide comprising C-terminal constant regions of an immunoglobulin heavy chain, capable of stable self-association. For example, an Fc polypeptide of a dimeric IgA Fc comprises an IgA CH3 domain and may also comprise an IgA CH2 domain.

The Fc region of the IgA HetFc constructs is thus comprised of two Fc polypeptides: a first Fc polypeptide and a second Fc polypeptide, which may also be referred to herein as Chain A and Chain B. The terms first Fc polypeptide and second Fc polypeptide (or Chain A and Chain B) can be used interchangeably provided that each Fc region comprises one first Fc polypeptide and one second Fc polypeptide (or one Chain A polypeptide and one Chain B polypeptide). The first and second Fc polypeptides meet at an “interface.” The “interface” comprises “contact” amino acid residues in the first Fc polypeptide that interact with one or more “contact” amino acid residues in the second Fc polypeptide.

The CH3 domain of an Fc region comprises two CH3 domain sequences, one from each of the first and second Fc polypeptides of the dimeric Fc. The CH2 domain comprises two CH2 domain sequences, one from each of the first and second Fc polypeptides of the dimeric Fc.

The IgA HetFc constructs of the present disclosure comprise an IgA CH3 domain that has been asymmetrically modified to generate a heterodimer Fc region. Specifically, one or more amino acid mutations are introduced into the IgA CH3 domain in an asymmetric fashion resulting in a heterodimer Fc. As used herein, an asymmetric amino acid mutation is a mutation resulting in an amino acid at a specific position in one Fc polypeptide being different from the amino acid in the second Fc polypeptide at the same position. This can be a result of mutation of only one of the two amino acids in the first and second Fc polypeptides or mutation of both amino acids to two different amino acids. The IgA HetFc constructs disclosed herein comprise one or more asymmetric amino acid mutations in the CH3 domain.

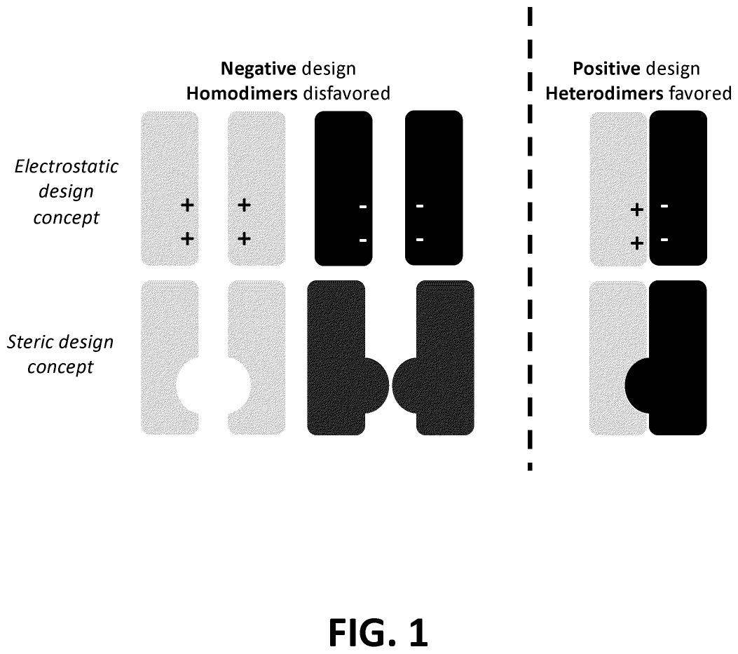

The design of IgA HetFc regions from wild-type homodimers is illustrated by the concept of positive and negative design in the context of protein engineering by balancing stability vs. specificity, wherein mutations are introduced with the goal of driving heterodimer formation over homodimer formation when the polypeptides are expressed in cell culture conditions. These general design concepts of positive and negative design are illustrated schematically in FIG. 1.

Negative design strategies maximize unfavorable interactions for the formation of homodimers, by either introducing bulky sidechains on one chain and small sidechains on the opposite, for example the knobs-into-holes strategy (Ridgway, et al., 1996, Protein Eng., 9(7):617-21; Atwell, et al., 1997, J Mol Biol., 270(1):26-35), or by electrostatic engineering that leads to repulsion of homodimer formation, for example the electrostatic steering strategy developed by Gunasekaran, et al. 21010, J Biol Chem., 285(25):19637-19646.

In positive design strategies, amino acid mutations are introduced into polypeptides to maximize favorable interactions within or between proteins. Such strategies assume that when introducing multiple mutations that specifically stabilize the desired heterodimer while neglecting the effect on the homodimers, the net effect will be better specificity for the desired heterodimer interactions over the homodimers and hence a greater heterodimer specificity. It is understood in the context of protein engineering that positive design strategies optimize the stability of the desired protein interactions, but rarely achieve greater than 90% specificity (Havranek & Harbury, 2003, Nat Struct Biol., 10(1):45-52; Bolon, et al., 2005, Proc Natl Acad Sci USA, 102(36):12724-9; Huang, et al., 2007, Protein Sci., 16(12):2770-4).

Disclosed herein is a method for designing IgA Fc heterodimers that results in stable and highly specific heterodimer formation. This design method combines both negative and positive design strategies along with structural and computational modeling guided protein engineering techniques (see Example 1 herein). The computational tools and structure-function analysis used in the method to generate the IgA HetFc constructs herein may include, for example, molecular dynamic analysis (MD), sidechain/backbone re-packing, Knowledge Base Potential (KBP), cavity (hydrophobic) packing analysis (LJ, AMBER, SASA, dSASA(carbon/all-atom)), electrostatic-GB calculations and coupling analysis. Computational methods for generating variant Fc regions are also described in International Patent Publication Nos. WO 2012/058768, WO 2015/021540, WO 2014/201566, WO 2014/138994, WO 2014/026296, WO 2013/188984, WO 2013/138923, WO 2012/040833, WO 2012/037659 and WO 2011/063518.

In certain embodiments, the IgA HetFc constructs resulting from the implementation of this method have a purity of 70% or higher, and a stability (as measured by melting temperature (Tm) of the CH3 domain) of 60° C. or higher. In certain embodiments, the IgA HetFc constructs resulting from the implementation of this method have a purity of 70% or higher, and a stability CH3 domain Tm (stability) within 10° C. of the CH3 domain Tm of the corresponding wild-type IgA Fc.

In accordance with the present disclosure, the amino acid mutations introduced into the CH3 domain of the IgA Fc promote heterodimer formation as compared to homodimer formation. This heterodimer formation as compared to homodimer formation is referred to herein interchangeably as “purity,” “specificity,” “heterodimer purity” or “heterodimer specificity.” It is understood that this heterodimer purity refers to the percentage of desired heterodimer formed as compared to homodimer species formed in solution under standard cell culture conditions. Heterodimer purity is assessed prior to selective purification of the heterodimer species. In certain embodiments, purity may be assessed after an IgA affinity purification step that is not selective for homodimer/heterodimer purification (e.g., after CaptureSelect™ IgA affinity purification). For instance, a heterodimer purity of 70% indicates that 70% of the Fc dimers isolated from cell culture after an IgA affinity purification step are the desired Fc heterodimer.

In certain embodiments, the IgA HetFc has a purity of greater than about 70%, for example, greater than about 71%, or greater than about 72%, or greater than about 73%, or greater than about 74%, or greater than about 75%, or greater than about 76%, or greater than about 77%, or greater than about 78%, or greater than about 79%. In some embodiments, the IgA HetFc has a purity of greater than about 80%, for example, greater than about 81%, or greater than about 82%, or greater than about 83%, or greater than about 84%, or greater than about 85%, or greater than about 86%, or greater than about 87%, or greater than about 88%, or greater than about 89%. In some embodiments, the IgA HetFc has a purity of greater than about 90%, for example, greater than about 91%, or greater than about 92%, or greater than about 93%, or greater than about 94%, or greater than about 95%, or greater than about 96%, or greater than about 97%, or greater than about 98%, or greater than about 99%.

In certain embodiments, the IgA HetFc has a purity of between about 70% and 100%. In some embodiments, the IgA HetFc has a purity of between about 70% and about 98%, or between about 70% and about 97%, or between about 70% and about 96%. In some embodiments, the IgA HetFc has a purity between about 72% and about 98%, or between about 74% and about 98%, or between about 75% and about 98%.

The relative amounts of heterodimer and homodimer in a sample of IgA HetFc, and thus the purity of the IgA HetFc, may be determined using various techniques known in the art including, but not limited to, size-exclusion chromatography (SEC), non-reducing sodium dodecyl sulfate polyacrylamide gel electrophoresis (SDS-PAGE), non-reducing capillary electrophoresis sodium dodecyl sulfate (CE-SDS) and liquid chromatography mass spectrometry (LC-MS).

In some embodiments, the IgA HetFc has a purity of greater than about 70% as determined by non-reducing CE-SDS. In some embodiments, the IgA HetFc has a purity of greater than about 70% as determined by non-reducing CE-SDS performed by running a High Throughput Protein Express assay using CE-SDS LabChip® GXII (Perkin Elmer, Waltham, MA). In some embodiment, the IgA HetFc has a purity of greater than about 70% as determined by non-reducing CE-SDS performed as described in Example 4 herein.

In some embodiments, the IgA HetFc has a purity of greater than about 70% as determined by UPLC-SEC. In some embodiments, the IgA HetFc has a purity of greater than about 70% as determined by UPLC-SEC performed on an Agilent Technologies 1260 Infinity LC system using an Agilent Technologies AdvanceBio SEC 300A column at 25° C. In some embodiments, the IgA HetFc has a purity of greater than about 70% as determined by UPLC-SEC performed as described in Example 4 herein.

The IgA HetFc constructs in accordance with the present disclosure are thermostable. In the context of the IgA HetFc constructs disclosed herein, “thermostable” means that the IgA HetFc construct has a CH3 domain melting temperature (Tm) that is about 60° C. or higher, or has a CH3 domain Tm that is within 10° C. (±10° C.) of the Tm of a corresponding wild-type IgA CH3 domain.

In certain embodiments, the IgA HetFc has a CH3 domain Tm of about 60° C. or higher. In some embodiments, the IgA HetFc has a CH3 domain Tm of about 62° C. or higher, for example, about 63° C. or higher, or about 64° C. or higher, or about 65° C. or higher, or about 66° C. or higher, or about 67° C. or higher, or about 68° C. or higher, or about 69° C. or higher. In some embodiments, the IgA HetFc has a CH3 domain Tm of about 70° C. or higher, for example, about 71° C. or higher, or about 72° C. or higher, or about 73° C. or higher.

In certain embodiments, the IgA HetFc has a CH3 domain Tm of between about 60° C. and about 74° C. In some embodiments, the IgA HetFc has a CH3 domain Tm of between about 62° C. and about 74° C., or between about 63° C. and about 74° C., or about 64° C. and about 74° C., or between about 65° C. and about 74° C.

In certain embodiments, the IgA HetFc construct has a CH3 domain Tm that is within 10° C. (±10° C.) of the Tm of a corresponding wild-type IgA CH3 domain. In some embodiments, the IgA HetFc construct has a CH3 domain Tm that is within 9° C. (±9° C.) of the Tm of a corresponding wild-type IgA CH3 domain, for example, within 8° C. (±8° C.), or within 7° C. (±7° C.), or within 6° C. (±6° C.), or within 5° C. (±5° C.) of the Tm of a corresponding wild-type IgA CH3 domain.

In certain embodiments, the IgA HetFc construct has a CH3 domain Tm that is about 60° C. or higher, or has a CH3 domain Tm that is within 10° C. (±10° C.) of the Tm of a corresponding wild-type IgA CH3 domain in the absence of any additional disulfide bonds in the CH3 domain. In certain embodiments, the IgA HetFc construct comprises one or more additional disulfide bonds in the CH3 domain as compared to wild-type IgA CH3 domain, but has a CH3 domain Tm that is about 60° C. or higher, or has a CH3 domain Tm that is within 10° C. (±10° C.) of the Tm of a corresponding wild-type IgA CH3 domain in the absence of the one or more disulfide bonds.

Stability measured as Tm can be determined using techniques known in the art, such as by differential scanning calorimetry (DSC), differential scanning fluorimetry (DSF), circular dichroism spectroscopy (CD) and hydrogen exchange (HX). In certain embodiments, Tm is determined by DSC.

In certain embodiments, the IgA HetFc construct has a CH3 domain Tm that is about 60° C. or higher, or has a CH3 domain Tm that is within 10° C. (i 10° C.) of the Tm of a corresponding wild-type IgA CH3 domain, where the Tm is determined by DSC. In some embodiments, the IgA HetFc construct has a CH3 domain Tm that is about 60° C. or higher, or has a CH3 domain Tm that is within 10° C. (±10° C.) of the Tm of a corresponding wild-type IgA CH3 domain, where the Tm is determined by DSC using a NanoDSC (TA Instruments, New Castle, DE, USA). In some embodiments, the IgA HetFc construct has a CH3 domain Tm that is about 60° C. or higher, or has a CH3 domain Tm that is within 10° C. (±10° C.) of the Tm of a corresponding wild-type IgA CH3 domain, where the Tm is determined by DSC following the protocol described in Example 6 herein.

In certain embodiments, the IgA HetFc:
(i) has a purity of greater than about 70%, for example, greater than about 71%, or greater than about 72%, or greater than about 73%, or greater than about 74%, or greater than about 75%, or greater than about 76%, or greater than about 77%, or greater than about 78%, or greater than about 79%, or greater than about 80%, or greater than about 81%, or greater than about 82%, or greater than about 83%, or greater than about 84%, or greater than about 85%, or greater than about 86%, or greater than about 87%, or greater than about 88%, or greater than about 89%, or greater than about 90%, or greater than about 91%, or greater than about 92%, or greater than about 93%, or greater than about 94%, or greater than about 95%, or greater than about 96%, or greater than about 97%, or greater than about 98%, or greater than about 99%, and
(ii) has a CH3 domain Tm that is between about 60° C. and about 74° C., for example, between about 62° C. and about 74° C., or between about 63° C. and about 74° C., or about 64° C. and about 74° C., or between about 65° C. and about 74° C.

In certain embodiments, the IgA HetFc:
(i) has a purity of greater than about 70%, for example, greater than about 71%, or greater than about 72%, or greater than about 73%, or greater than about 74%, or greater than about 75%, or greater than about 76%, or greater than about 77%, or greater than about 78%, or greater than about 79%, or greater than about 80%, or greater than about 81%, or greater than about 82%, or greater than about 83%, or greater than about 84%, or greater than about 85%, or greater than about 86%, or greater than about 87%, or greater than about 88%, or greater than about 89%, or greater than about 90%, or greater than about 91%, or greater than about 92%, or greater than about 93%, or greater than about 94%, or greater than about 95%, or greater than about 96%, or greater than about 97%, or greater than about 98%, or greater than about 99%, and
(ii) has a CH3 domain Tm that is within 10° C. (±10° C.) of the Tm of a corresponding wild-type IgA CH3 domain, for example, within 9° C. (±9° C.), or within 8° C. (±8° C.), or within 7° C. (±7° C.), or within 6° C. (±6° C.), or within 5° C. (±5° C.) of the Tm of a corresponding wild-type IgA CH3 domain.

In certain embodiments, the IgA HetFc construct comprises one or more mutations to either eliminate binding to a binding target, or one or more mutations to introduce binding to the Neonatal Fc Receptor (FcRn), or both.

### Modified CH3 Domains

The IgA HetFc constructs described herein comprise a modified CH3 domain comprising asymmetric amino acid mutations. Specifically, the IgA HetFc constructs comprise two Fc polypeptides: a first Fc polypeptide that comprises a first CH3 domain sequence comprising one or more amino acid mutations and a second Fc polypeptide that comprises a second CH3 domain sequence comprising one or more amino acid mutations, where at least one of the amino acid mutations in the first CH3 domain sequence is different to the amino acid mutations in the second CH3 domain sequence. The first and second CH3 domain sequences together form the modified CH3 domain. The amino acid mutations introduced asymmetrically into the first and second CH3 domain sequences result in formation of a heterodimeric Fc, rather than a homodimeric Fc, when the two CH3 domain sequences dimerize.

As noted above, an “asymmetric amino acid mutation” in this context refers to a mutation where an amino acid at a specific position in a first CH3 domain sequence is different from the amino acid in a second CH3 domain sequence at the same position. An asymmetric mutation can be a result of mutation of only one of the two amino acids at the same respective amino acid position in each CH3 domain sequence, or a different mutation of both amino acids at the same respective position on each of the first and second CH3 domain sequences. The CH3 domain sequences of an IgA HetFc can comprise one, or more than one, asymmetric amino acid mutation.

By employing the computational strategies disclosed herein, a core set of asymmetric mutations to the IgA CH3 domain were identified for providing the desired property of promoting formation of a heterodimer Fc. This core set of mutations is shown in Table 5.

TABLE 5
IgA HetFc Core Mutations

| Chain | Position (IMGT) | Amino Acid Substitution |
|---|---|---|
| A | A6085Y | F, Y, M, W, H |
| | T6086 | Y, F, M, W, H |
| B | W6081 | T, L, A, V, I |

In certain embodiments, the IgA HetFc construct comprises a modified CH3 domain in which the amino acid mutations in the first CH3 domain sequence comprise an amino acid substitution at position A6085Y selected from A6085YF, A6085YY, A6085YM, A6085YW and A6085YH, and an amino acid substitution at position T6086 selected from T6086Y, T6086F, T6086M, T6086W and T6086H; and the amino acid mutations in the second CH3 domain sequence comprise an amino acid substitution at position W608I selected from W6081T, W6081L, W6081A, W6081V and W6081I.

In certain embodiments, the IgA HetFc construct comprises a modified CH3 domain comprising the amino acid mutations as set forth for any one of the designs shown in Table 7.

In some embodiments, the amino acid substitution at position A6085Y in the first CH3 domain sequence is A6085YF, A6085YY or A6085YW. In some embodiments, the amino acid substitution at position A6085Y in the first CH3 domain sequence is A6085YF or A6085YY.

In some embodiments, the amino acid substitution at position T6086 in the first CH3 domain sequence is T6086Y, T6086F or T6086W. In some embodiments, the amino acid substitution at position T6086 in the first CH3 domain sequence is T6086Y.

In some embodiments, the amino acid substitution at position W608I in the second CH3 domain sequence is W6081T or W6081L.

In certain embodiments, the IgA HetFc construct comprises a modified CH3 domain in which the amino acid mutations in the first CH3 domain sequence comprise the amino acid substitutions A6085YF and T6086W, and the amino acid mutations in the second CH3 domain sequence comprise the amino acid substitution W6081T or W6081L.

In some embodiments, the IgA HetFc construct comprises a modified CH3 domain in which the amino acid mutations in the first CH3 domain sequence comprise the amino acid substitutions A6085YF and T6086W, and the amino acid mutations in the second CH3 domain sequence comprise the amino acid substitution W6081T.

In certain embodiments, the first CH3 domain sequence of the IgA HetFc construct may optionally further comprise one or more of:
(i) an amino acid substitution at position T6022 selected from T6022V, T6022I, T6022L and T6022A; and/or
(ii) an amino acid substitution at position H6005 selected from H6005Y, H6005F, H6005M and H6005W.

In certain embodiments, the second CH3 domain sequence of the IgA HetFc construct may optionally further comprise one or more of:
(i) an amino acid substitution at position H6005 selected from H6005Y, H6005F, H6005M and H6005W; and/or
(ii) an amino acid substitution at position L6079 selected from L6079V, L6079T, L6079A and L6079I; and/or
(iii) an amino acid substitution at position 16088 selected from I6088L, I6088A, I6088V and I6088T; and/or
(iv) an amino acid substitution at position L6007 selected from L6007F, L6007Y, L6007M, L6007W, L6007H and L6007I.

In certain embodiments, the IgA HetFc construct comprises a modified CH3 domain in which the amino acid mutations in the first CH3 domain sequence comprise an amino acid substitution at position A6085Y selected from A6085YF, A6085YY, A6085YM, A6085YW and A6085YH, and an amino acid substitution at position T6086 selected from T6086Y, T6086F, T6086M, T6086W and T6086H; and the amino acid mutations in the second CH3 domain sequence comprise an amino acid substitution at position W608I selected from W6081T, W6081L, W6081A, W6081V and W6081I; and
(i) the amino acid mutations in the first CH3 domain sequence further comprise an amino acid substitution at position T6022 selected from T6022V, T6022I, T6022L and T6022A; and/or
(ii) the amino acid mutations in the first CH3 domain sequence further comprise an amino acid substitution at position H6005 selected from H6005Y, H6005F, H6005M and H6005W; and/or
(iii) the amino acid mutations in the second CH3 domain sequence further comprise an amino acid substitution at position H6005 selected from H6005Y, H6005F, H6005M and H6005W; and/or
(iv) the amino acid mutations in the first CH3 domain sequence further comprise an amino acid substitution at position H6005 selected from H6005Y, H6005F, H6005M and H6005W, and the amino acid mutations in the second CH3 domain sequence further comprise an amino acid substitution at position H6005 selected from H6005Y, H6005F, H6005M and H6005W; and/or
(v) the amino acid mutations in the second CH3 domain sequence further comprise an amino acid substitution at position L6079 selected from L6079V, L6079T, L6079A and L6079I; and/or
(vi) the amino acid mutations in the second CH3 domain sequence further comprise an amino acid substitution at position 16088 selected from I6088L, I6088A, I6088V and I6088T; and/or
(vii) the amino acid mutations in the second CH3 domain sequence further comprise an amino acid substitution at position L6007 selected from L6007F, L6007Y, L6007M, L6007W, L6007H and L6007I.

In some embodiments, the amino acid mutation at position T6022 in the first CH3 domain sequence is selected from T6022V, T6022I and T6022L.

In some embodiments, the amino acid mutation at position H6005 in the first CH3 domain sequence is H6005Y.

In some embodiments, the amino acid mutation at position H6005 in the second CH3 domain sequence is H6005Y.

In some embodiments, the amino acid mutation at position L6079 in the second CH3 domain sequence is L6079V or L6079T.

In some embodiments, the amino acid mutation at position 16088 in the second CH3 domain sequence is I6088L.

In some embodiments, the amino acid mutation at position L6007 in the second CH3 domain sequence is L6007F.

In certain embodiments, the modified CH3 domain of the IgA HetFc construct further comprises amino acid substitutions to introduce cysteine residues capable of forming a disulfide bond. In some embodiments, the modified CH3 domain of the IgA HetFc construct further comprises two cysteine substitutions that introduce one disulfide bond into the CH3 domain. In some embodiments, the modified CH3 domain of the IgA HetFc construct further comprises four cysteine substitutions that introduce two disulfide bonds into the CH3 domain. In some embodiments, the cysteine substitutions comprise the mutation H6005C in one CH3 domain sequence and the mutation P6010C in the other CH3 domain sequence. In some embodiments, the cysteine substitutions comprise the mutations H6005C and P6010C in one CH3 domain sequence and the mutations P6010C and H6005C in the other CH3 domain sequence.

Accordingly, in certain embodiments, the IgA HetFc construct comprises a modified CH3 domain comprising either one or two introduced (i.e. non-natural) disulfide bonds in which:
(i) one CH3 domain sequence comprises the mutation H6005C and the other CH3 domain sequence comprises the mutation P6010C; or
(ii) one CH3 domain sequence comprises the mutations H6005C and P6010C, and the other CH3 domain sequence comprises the mutations P6010C and H6005C.

In certain embodiments, the IgA HetFc construct comprises a modified CH3 domain in which the amino acid mutations in the first CH3 domain sequence comprise an amino acid substitution at position A6085Y selected from A6085YF, A6085YY, A6085YM, A6085YW and A6085YH, and an amino acid substitution at position T6086 selected from T6086Y, T6086F, T6086M, T6086W and T6086H; and the amino acid mutations in the second CH3 domain sequence comprise an amino acid substitution at position W608I selected from W6081T, W6081L, W6081A, W6081V and W6081I; where
(i) the first CH3 domain of the IgA HetFc construct may optionally further comprise an amino acid substitution at position T6022 selected from T6022V, T6022I, T6022L and T6022A; and
(ii) the second CH3 domain of the IgA HetFc construct may optionally further comprise one or more of: an amino acid substitution at position L6079 selected from L6079V, L6079T, L6079A and L6079I; and/or an amino acid substitution at position 16088 selected from I6088L, I6088A, I6088V and I6088T; and/or an amino acid substitution at position L6007 selected from L6007F, L6007Y, L6007M, L6007W, L6007H and L6007I, and
(iii) the modified CH3 domain comprises either one or two introduced (i.e., non-natural) disulfide bonds as described above.

In certain embodiments, the IgA HetFc construct comprises a modified CH3 domain in which the amino acid mutations in the first CH3 domain sequence comprise an amino acid substitution at positions A6085Y and T6086, and the amino acid mutations in the second CH3 domain sequence comprise an amino acid substitution at position W608I and optionally an amino acid mutation at one or both of positions L6079 and 16088, where
the amino acid substitution at position A6085 is selected from A6085YF, A6085YY, A6085YM, A6085YW and A6085YH;
the amino acid substitution at position T6086 is selected from T6086Y, T6086F, T6086M, T6086W and T6086H;
the amino acid substitution at position W608I is selected from W6081T, W6081L, W6081A, W6081V and W6081I;
the optional amino acid substitution at position L6079 is selected from L6079V, L6079T, L6079A and L6079I; and
the optional amino acid substitution at position 16088 is selected from I6088L, I6088A, I6088V and I6088T.

In certain embodiments, the IgA HetFc construct comprises a modified CH3 domain comprising the amino acid mutations as set forth for any one of the designs shown in Table 8. In certain embodiments, the IgA HetFc construct comprises a modified CH3 domain comprising the amino acid mutations as set forth for any one of the designs shown in Table 9. In certain embodiments, the IgA HetFc construct comprises a modified CH3 domain comprising the amino acid mutations as set forth for any one of the designs shown in Table 10.

In some embodiments, the amino acid substitution at position A6085Y is A6085YF, A6085YY or A6085YW. In some embodiments, the amino acid substitution at position A6085Y is A6085YF or A6085YY. In some embodiments, the amino acid substitution at position T6086 is T6086Y, T6086F or T6086W. In some embodiments, the amino acid substitution at position T6086 is T6086Y. In some embodiments, the amino acid substitution at position W608I is W6081T or W6081L. In some embodiments, the optional amino acid substitution at position L6079 is L6079V or L6079T. In some embodiments, the optional amino acid substitution at position 16088 is I6088L.

In some embodiments, the the IgA HetFc construct comprises a modified CH3 domain in which the amino acid mutations in the first CH3 domain sequence comprise an amino acid substitution at positions A6085Y and T6086, and the amino acid mutations in the second CH3 domain sequence comprise an amino acid substitution at position W608I and optionally at one or both of positions L6079 and 16088, as described in any one of the embodiments above, and either the first CH3 domain sequence or the second CH3 domain sequence or both the first and second CH3 domain sequences further comprise an amino acid substitution at position H6005 selected from H6005Y, H6005F, H6005M and H6005W. In some embodiments, either the first CH3 domain sequence or the second CH3 domain sequence or both the first and second CH3 domain sequences further comprise the amino acid substitution H6005Y.

In certain embodiments, the IgA HetFc construct comprises a modified CH3 domain in which the amino acid mutations in the first CH3 domain sequence comprise an amino acid substitution at positions A6085Y and T6086, and the amino acid mutations in the second CH3 domain sequence comprise an amino acid substitution at position W608I and optionally at one or more of positions L6007, L6079 and 16088, where
the amino acid substitution at position A6085 is selected from A6085YF, A6085YY, A6085YM, A6085YW and A6085YH;
the amino acid substitution at position T6086 is selected from T6086Y, T6086F, T6086M, T6086W and T6086H;
the amino acid substitution at position W608I is selected from W6081T, W6081L, W6081A, W6081V and W6081I;
the optional amino acid substitution at position L6007 is selected from L6007F, L6007Y, L6007M, L6007W, L6007H and L6007I;
the optional amino acid substitution at position L6079 is selected from L6079V, L6079T, L6079A and L6079I; and
the optional amino acid substitution at position 16088 is selected from I6088L, I6088A, I6088V and I6088T.

In some embodiments, the amino acid substitution at position A6085Y is A6085YF, A6085YY or A6085YW. In some embodiments, the amino acid substitution at position A6085Y is A6085YF or A6085YY. In some embodiments, the amino acid substitution at position T6086 is T6086Y, T6086F or T6086W. In some embodiments, the amino acid substitution at position T6086 is T6086Y. In some embodiments, the amino acid substitution at position W608I is W6081T or W6081L. In some embodiments, the amino acid substitution at position L6007 is L6007F. In some embodiments, the amino acid substitution at position L6079 is L6079V or L6079T. In some embodiments, the amino acid substitution at position 16088 is I6088L.

In some embodiments, the the IgA HetFc construct comprises a modified CH3 domain in which the amino acid mutations in the first CH3 domain sequence comprise an amino acid substitution at positions A6085Y and T6086, and the amino acid mutations in the second CH3 domain sequence comprise an amino acid substitution at position W608I and optionally at one or more of positions L6007, L6079 and 16088, as described in any one of the embodiments above, and either the first CH3 domain sequence or the second CH3 domain sequence or both the first and second CH3 domain sequences further comprise an amino acid substitution at position H6005 selected from H6005Y, H6005F, H6005M and H6005W. In some embodiments, either the first CH3 domain sequence or the second CH3 domain sequence or both the first and second CH3 domain sequences further comprise the amino acid substitution H6005Y.

In certain embodiments, the IgA HetFc construct comprises a modified CH3 domain in which the amino acid mutations are the amino acid substitutions listed in Table 6 for any one of variants v32516, v32517, v32518, v32521, v33330, v33331, v33332, v33333, v33334, v34688, v34689 or v34690. In some embodiments, the IgA HetFc construct comprises a modified CH3 domain in which the amino acid mutations are the amino acid substitutions listed in Table 6 for any one of variants v32521, v33333 or v33334.

TABLE 6
Illustrative IgA HetFc Variants

| | | CH3 Domain Sequence Mutations | |
|---|---|---|---|
| Variant | Design | Chain A | Chain B |
| 32516 | Steric 1 | A6085YY_T6086L | L6079T_W6081L_I6088L |
| 32517 | Steric 2 | A6085YY_T6086Y | L6079T_W6081L_I6088L |
| 32518 | Steric 3 | A6085YF_T6086Y | L6079V_W6081L_I6088L |
| 32521 | Steric 6 | A6085YF_T6086Y | L6079V_W6081T_I6088L |
| 33330 | Steric 7 | T6022V_A6085YF_T6086Y | L6079V_W6081T_I6088L |
| 33331 | Steric 8 | T6022L_A6085YF_T6086Y | L6079V_W6081T_I6088L |
| 33332 | Steric 9 | T6022I_A6085YF_T6086Y | L6079V_W6081T_I6088L |
| 33333 | Steric 10 | A6085YF_T6086Y | L6007F_L6079V_W6081T_I6088L |
| 33334 | Steric 11 | H6005Y_A6085YF_T6086Y | H6005Y_L6079V_W6081T_I6088L |
| 34688 | Steric 6 + | H6005C_A6085YF_T6086Y | P6010C_L6079V_W6081T_I6088L |
| | Disulfide | | |
| 34689 | Steric 6 + | P6010C_A6085YF_T6086Y | H6005C_L6079V_W6081T_I6088L |
| | Disulfide | | |
| 34690 | Steric 6 + 2× | H6005C_P6010C_A6085YF_T6086Y | H6005C_P6010C_L6079V_W6081T_I6088L |
| | Disulfide | | |

In certain embodiments, the IgA HetFc construct of the present disclosure comprises a modified CH3 domain having an amino acid sequence as set forth in the CH3 domain sequence comprised by SEQ ID NOs. 15 and 20; SEQ ID NOs. 16 and 20; SEQ ID NOs. 17 and 21; SEQ ID NOs. 17 and 23; SEQ ID NOs. 24 and 23; SEQ ID NOs. 25 and 23; SEQ ID NOs. 26 and 23; SEQ ID NOs. 17 and 27; SEQ ID NOs. 28 and 29; SEQ TD NOs. 30 and 31; SEQ ID NOs. 32 and 33; or SEQ ID NOs. 34 and 35. IgA CH2 and CH3 domains can readily be identified within the noted SEQ ID NOs by comparison with the IgA sequences provided in Tables 2 and 4 herein.

### Modified CH2 Domains

In certain embodiments, the IgA HetFc construct further comprises a modified CH2 domain comprising one or more amino acid mutations, for example, mutations that alter one or more functions of the CH2 domain. Illustrative mutations include, but are not limited to, mutations at position C5092 (which attaches to the secretory compartment in WT IgA) and mutations at the glycosylation site at position N5120.

In certain embodiments, the modified CH2 comprises a mutation at position C5092. In some embodiments, the mutation at position C5092 is an amino acid substitution selected from C5092S, C5092A, C5092T, C5092N and C5092Q. In some embodiments, the mutation at position C5092 is C5092S. In certain embodiments, the modified CH2 domain comprises a mutation at the glycosylation site at position N5120, where the mutation prevents glycosylation. In some embodiments, the mutation at position N5120 is the amino acid substitution N5120T.

In certain embodiments, the HetFc IgA construct comprises a modified CH2 domain that comprises a mutation at one or more of positions C5092, N5120, 15121 and T5122. In some embodiments, the HetFc IgA construct comprises a modified CH2 domain that comprises one or more amino acid substitutions selected from C5092S, N5120T, I5121L and T5122S. In some embodiments, the HetFc IgA construct comprises a modified CH2 domain that comprises the amino acid substitutions C5092S, N5120T, I5121L and T5122S.

In some embodiments, the modified CH2 domain comprises asymmetric amino acid substitutions in the first and/or second Fc polypeptide chain. In some embodiments, the modified CH2 domain comprises asymmetric amino acid substitutions that allow one chain of the CH2 domain to selectively bind an Fc receptor. In certain embodiments, the modified CH2 domain comprises asymmetric amino acid mutations that promote selective binding to Fcα receptors.

One skilled in the art will understand that the IgA HetFc constructs of the present disclosure may have altered ligand (e.g. FcαRI) binding properties (examples of binding properties include but are not limited to, binding specificity, equilibrium dissociation constant (K_{D}), dissociation and association rates (k_{off} and kₒₙ respectively), binding affinity and/or avidity) and that certain alterations may be more or less desirable depending on the end use of the IgA HetFc construct. It is well known in the art that the equilibrium dissociation constant (K_{D}) is defined as k_{off}/kₒₙ. For certain applications, it generally understood that an IgA HetFc construct with a low K_{D} may be preferable to an IgA HetFc construct with a high K_{D}. However, in some instances the value of the kₒₙ or k_{off} may be more relevant than the value of the K_{D}. One skilled in the art can determine which kinetic parameter is most important for a given IgA HetFc construct application.

In certain embodiments, the IgA HetFc comprises substitutions that reduce or eliminate binding to the Fcα receptors (see for example, Carayannopoulos, 1996, JEM, 183:1579-1586; Bakema, 2006, J Immunol, 176:3603-3610, https://www.pnas.org/content/115/38/E8882). IgA HetFc constructs with reduced or eliminated binding to the Fcα receptors can be useful, for example, in a setting in which activation of neutrophils is not desired, such as in a setting of cytokine release syndrome where the IgA HetFc construct can bind and clear cytokines in a subject in need thereof while avoiding activation of neutrophils. An IgA HetFc with only one FcαRI binding site can be useful to investigate the dependency of IgA-dependent neutrophil activation on the valency of FcαRI engagement.

An IgA HetFc can be useful to create a molecule capable of binding to FcαRI as well as the Neonatal Fc Receptor (FcRn) in a single Fc. Since binding sites for FcαRI and FcRn are located in structurally equivalent regions of IgA and IgG, respectively (Kelton, W. et al., 2014, Chem Biol 21:1603-1609, https://www.sciencedirect.com/science/article/pii/S1074552114004098?via%3Dihub), their introduction on a chain in an Fc is mutually exclusive and a heterodimeric Fc is needed. An IgA HetFc with an FcRn binding site grafted onto one chain is useful as it able to activate neutrophils via the FcαRI as well as having an increased half-life due to the introduction of the interaction with FcRn, thus addressing the known half-life limitation when using IgA for the therapeutic benefit.

An IgA HetFc can further be useful to create a molecule capable of binding to receptors or purification resins or detection molecules in a monovalent fashion. Likewise, it can be useful to create IgA HetFc-based molecules with combinations of receptor binding sites, purification or detection sites that would otherwise lie in mutually exclusive regions of the Fc. One such example would be to equip previously described IgG/A hybrid molecules (Kelton, W. et al., 2014, Chem Biol 21:1603-1609, Borrok, M. J. et al., 2015, mAbs, 7:4, 743-751, DOI: 10.1080/19420862.2015.1047570) with differing Fey receptor binding sites on the two chains of the Fc to create an Fcγ receptor binding profile that has a unique biological activity. Receptor binding sites include FcαR, FcRn, Fcγ receptors, C1q, Secretory Component, SSL7, Streptococcal IgA binding protein, N. meningitidis type 2 IgA1 protease, H. influenzae type 2 IgA1 protease. Purification and detection sites include protein A, polyhistidine tags, FLAG tags and Myc tags. Introducing a protein A binding site, for example, can be used to purify the IgA HetFc based molecule using techniques established and widely used for IgG based therapeutics that are unsuitable for a WT IgA Fc due to the lack of protein A binding.

### Target Binding Domains

The IgA HetFc described herein may function as a heterodimeric scaffold to which a variety of different binding domains or other moieties can be fused. In certain embodiments, the present disclosure relates to IgA HetFc constructs which are IgA HetFc binding units comprising one or more target binding domains fused to the IgA HetFc. Target binding domains for use in the IgA HetFc binding units include various proteinaceous moieties that specifically bind to a target of interest. “Specifically binds,” in this context, means that the binding is selective for the desired target and can be distinguished from unwanted or non-specific interactions. The ability of a binding domain to specifically bind to a target can be measured by various techniques familiar to one of skill in the art, e.g. enzyme-linked immunosorbent assay (ELISA), surface plasmon resonance (SPR) technique (e.g. analyzed on a BIAcore™ instrument) (Liljeblad, et al., 2000, Glyco J., 17:323-329) or traditional binding assays (Heeley, 2002, Endocr Res., 28:217-229).

Examples of target binding domains include, but are not limited to, receptors, receptor fragments (such as extracellular portions), ligands, cytokines and antigen-binding fragments of antibodies. In certain embodiments, the IgA HetFc binding unit comprises one or more binding domains that are antigen-binding domains, for example, receptor or antibody fragments.

In certain embodiments, the IgA HetFc binding unit comprises one or more target binding domains that are antigen-binding antibody fragments. Such antigen-binding antibody fragments may be derived from IgA or from other antibody isotypes such as IgG, IgM, IgD, or IgE. In some embodiments, the antigen-binding antibody fragments may be synthetic, chimeric or humanized. Antigen-binding antibody fragments include, but are not limited to, variable or hypervariable regions of light and/or heavy chains of an antibody (V_{L}, V_{H}), variable fragments (Fv), Fab′ fragments, F(ab′) 2 fragments, Fab fragments, single chain antibodies (scAb), single chain variable regions (scFv), VHH, complementarity determining regions (CDRs), domain antibodies (dAbs), single domain heavy chain immunoglobulins and single domain light chain immunoglobulins. Antigen-binding sites of an antibody typically contain six CDRs which contribute in varying degrees to the affinity of the binding site for antigen. There are three heavy chain variable domain CDRs (CDRH1, CDRH2 and CDRH3) and three light chain variable domain CDRs (CDRL1, CDRL2 and CDRL3). The extent of CDR and framework regions (FRs) is determined by comparison to a compiled database of amino acid sequences in which those regions have been defined according to variability among the sequences and/or structural information from antibody/antigen complexes. Also included within the scope of this disclosure are functional antigen-binding sites comprised of fewer CDRs (i.e. where binding specificity is determined by three, four or five CDRs). Less than a complete set of 6 CDRs may be sufficient for binding to some binding targets. Thus, in some instances, the CDRs of a VH or a VL domain alone will be sufficient for specific binding. Furthermore, certain antibodies might have non-CDR-associated binding sites for an antigen. Such binding sites are specifically contemplated herein. Antigen-binding antibody fragments may be from a single species or may be chimeric or humanized.

In certain embodiments, the binding domain comprises an antigen-binding receptor fragment, for example, an MHC-peptide complex-binding fragment of a T cell receptor (TCR). TCR fragments for use in the IgA HetFc constructs herein may comprise antigen-binding fragments of αβTCR or γδTCR heterodimers. In some embodiments, IgA HetFc constructs herein may comprise an antigen-binding fragment of a αβTCR heterodimer that comprises at least a TCR α chain variable domain and a TCR β chain variable domain such that the αβTCR fragment is able to bind to its cognate MHC/peptide. In some embodiments, the antigen-binding TCR fragment is a single-chain TCR (scTCR) or a soluble TCR domain (see, for example, International Patent Publication Nos. WO 1999/018129 and WO 2009/117117). Other TCR antigen-binding fragments are known in the art and are described, for example, in Wilson & Garcia, 1997, Curr. Opin. Struct. Biol. 7:839-848; van Boxel, et al., 2009, J. Immunol. Methods, 350:14-21; Stone, et al., 2012, Methods Enzymol., 503:189-222 and Li, et al., 2005, Nat. Biotechnol., 23:349-354).

Other target binding domains include immunomodulatory Ig domains, non-Ig viral receptor decoys, non-immunoglobulin proteins that mimic antibody binding and structures such as anticalins, affilins, affibody molecules, affimers, affitins, alphabodies, avimers, DARPins, fynomers, kunitz domain peptides, monobodies, and binding domains based on other engineered scaffolds such as SpA, GroEL, fibronectin, lipocalin and CTLA4 scaffolds. Further examples of target binding domains include a ligand for a desired receptor, a ligand-binding portion of a receptor, a lectin and peptides that specifically bind to one or more target antigens.

In certain embodiments, the IgA HetFc binding unit comprises a binding domain that comprises an antigen-binding fragment of a therapeutic or diagnostic antibody. In some embodiments, a target binding domain comprised by the IgA HetFc binding unit specifically binds to a cell surface molecule, such as a protein, lipid or polysaccharide. In some embodiments, a binding domain comprised by the IgA HetFc binding unit specifically binds a target antigen expressed on a tumor cell, virally infected cell, bacterially infected cell, damaged red blood cell, arterial plaque cell, inflamed tissue cell or fibrotic tissue cell.

In certain embodiments, the target binding domain comprised by the IgA HetFc binding unit is an immune response modulator. In certain embodiments, the target binding domain comprised by the IgA HetFc binding unit specifically binds a cytokine receptor. In certain embodiments, the target binding domain comprised by the IgA HetFc binding unit specifically binds to a tumor antigen. In certain embodiments, the target binding domain comprised by the IgA HetFc binding unit is, or specifically binds to, an immune checkpoint protein.

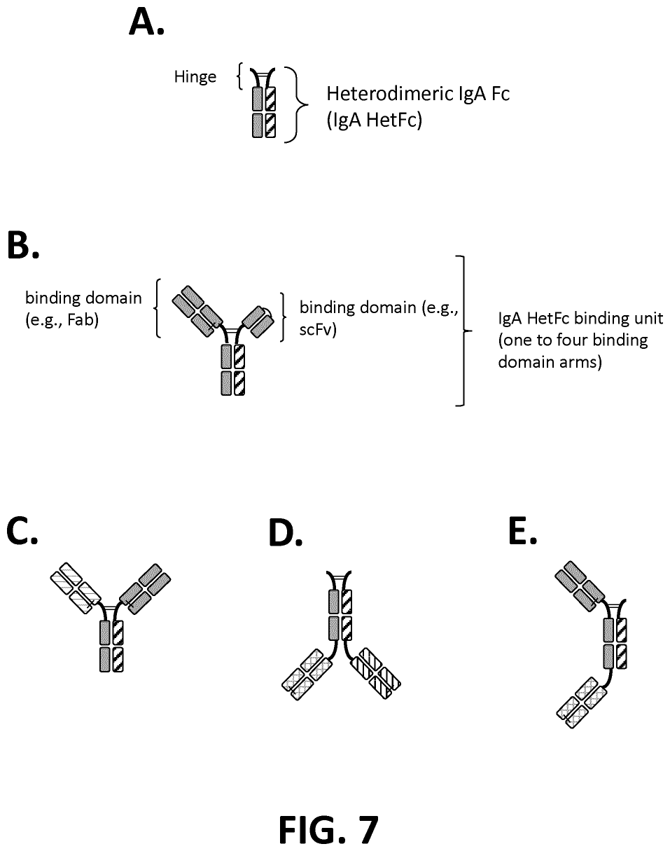

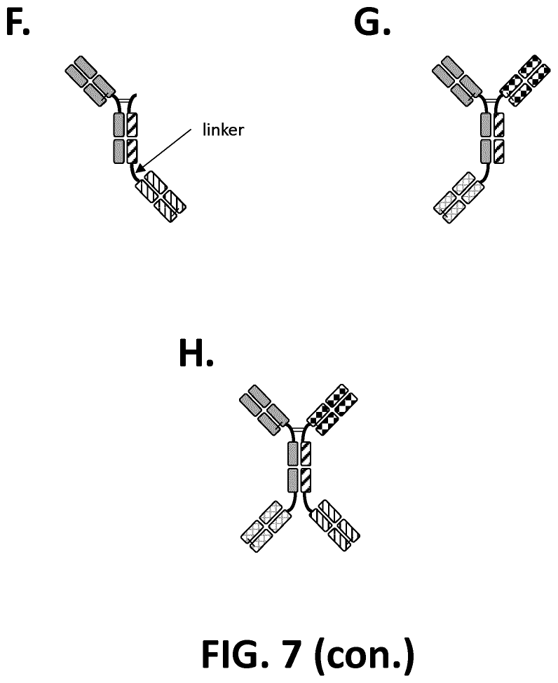

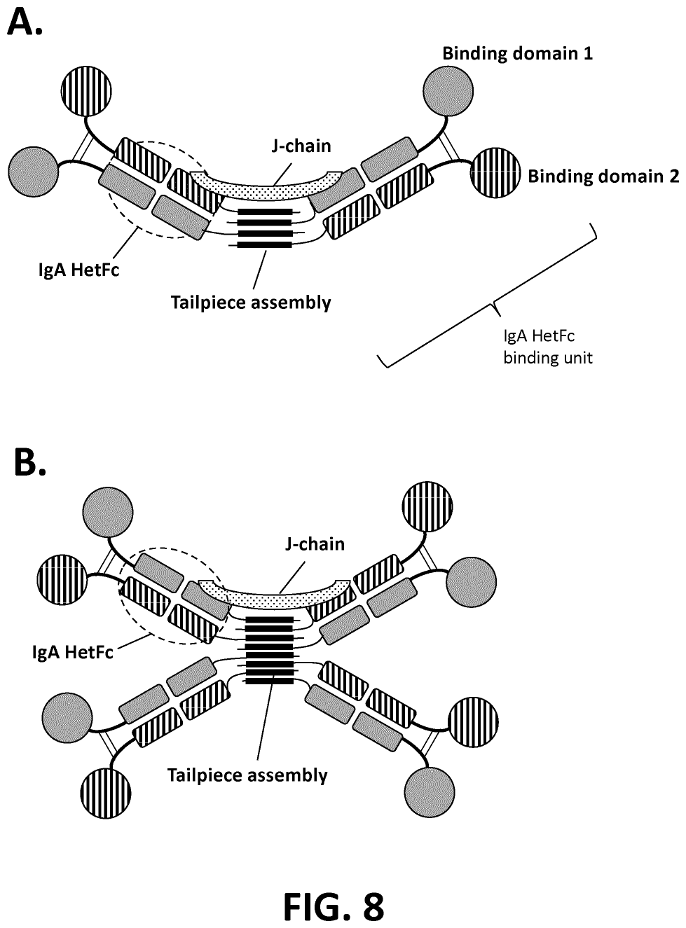

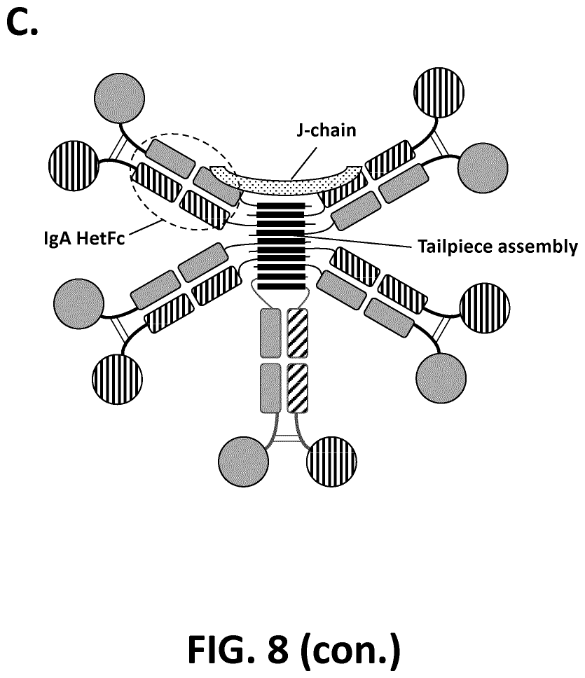

As a result of the heterodimeric nature of the IgA HetFc, different binding domains can be fused to one or both chains of the Fc heterodimer to generate a wide range of functional multispecific IgA HetFc binding units. Non-limiting illustrative examples of such multispecific IgA HetFc binding units are shown in FIG. 7. In addition, higher order IgA HetFc multimers may be generated by joining multiple IgA HetFc binding units together, for example, by joining with a J chain. Multimeric IgA structures typically comprise an IgA dimer in a tail-to-tail configuration linked by a J chain and tailpiece-to-tailpiece interactions, with additional IgA monomers linked to the dimer just via tailpiece-to-tailpiece mediated disulfide bonds and no direct contacts to the J chain in the complex (see, for example, Kumar, et al., 2020, Science, 10.1126/science.aaz5807). Non-limiting illustrative examples of such IgA HetFc multimers are shown in FIG. 8.

The IgA HetFc binding units according to the present disclosure may be monospecific, bispecific, trispecific, tetraspecific or have greater multispecificity. Multispecific IgA HetFc binding units may specifically bind to different epitopes of a desired target molecule or may specifically bind to different target molecules or may bind a target molecule as well as a heterologous epitope, such as a heterologous polypeptide or solid support material.

In some embodiments, the IgA HetFc binding unit comprises two or more target binding domains each having a different binding specificity. In this regard, the binding domains may bind the same target but bind to different epitopes on the same target or they may each bind to a different target.

In certain embodiments, the IgA Fc binding unit comprises a target binding domain fused to one Fc polypeptide (e.g., Chain A) and either no target binding domain or a different target binding domain fused to the other Fc polypeptide (e.g., Chain B). Thus, Chain A and Chain B of the IgA HetFc differ in their Fc regions (as described above, having mutations in the CH3 domain to drive heterodimer formation) and may also differ in their binding specificities.

The term IgA HetFc binding unit is used herein to refer to an IgA HetFc construct having a heterodimer Fc as described herein (e.g., a pair of IgA Fc polypeptides each comprising at least an IgA CH3 domain), where at least one IgA Fc polypeptide is fused to a target binding domain. In certain embodiments, both Fc polypeptides of the IgA HetFc construct are each independently fused to a target binding domain. As shown in FIG. 7, an IgA HetFc binding unit may comprise from one to four target binding domains fused to the HetFc in a variety of different configurations. In certain embodiments, additional target binding domains may be included in the IgA HetFc binding unit by fusing one or more additional target binding domains to a target binding domain fused to the IgA HetFc.

IgA HetFc binding units in accordance with the present disclosure may be derived from a single species, or may be chimeric or humanized. For example, the IgA Fc polypeptides may be human and the target binding domains may be derived from another species, such as another mammal (e.g., mouse, rat, rabbit, non-human primate, or the like).

FIG. 7 is a diagram showing illustrative configurations of IgA HetFc constructs comprising target binding domains (IgA HetFc binding units). In certain embodiments, an IgA HetFc binding unit comprises one, two, three or four target binding domains fused the IgA HetFc. In some embodiments, an IgA HetFc binding unit has a one-armed format in that one Fc polypeptide is fused to a target binding domain and the other Fc polypeptide is not.

In some embodiments, the IgA HetFc binding unit comprises one target binding domain fused to the N-terminal end of one Fc polypeptide (e.g., Chain A) and one target binding domain fused to the N-terminal end of the other Fc polypeptide (e.g., Chain B) (see, for example, FIG. 7B, FIG. 7C). In some embodiments, the IgA HetFc binding unit comprises one target binding domain fused to the N-terminal end of one Fc polypeptide (e.g. Chain A) and one target binding domain fused to the C-terminal end of the other Fc polypeptide (e.g., Chain B) (see, for example, FIG. 7F). In some embodiments, the IgA HetFc binding unit comprises one target binding domain fused to the C-terminal end of one Fc polypeptide (e.g., Chain A) and one target binding domain fused to the C-terminal end of the other Fc polypeptide (e.g. Chain B) (see, for example. FIG. 7D). In some embodiments, the IgA HetFc binding unit comprises target binding domains fused to both ends of one Fc polypeptide (e.g. to the N-terminal end and to the C-terminal end of Chain A) (see, for example, FIG. 7E). In some embodiments, the IgA HetFc binding unit comprises target binding domains fused to both ends of one Fc polypeptide (e.g. to the N-terminal end and to the C-terminal end of Chain A), and a target binding domain fused to one end (either the N-terminal or C-terminal end) of the other Fc polypeptide (e.g. Chain B) (see, for example, FIG. 7G). In some embodiments, the IgA HetFc binding unit comprises target binding domains fused to both ends of one Fe polypeptide (e.g. to the N-terminal end and to the C-terminal end of Chain A), and target binding domains fused to both ends of the other Fc polypeptide (e.g. to the N-terminal end and to the C-terminal end of Chain B) (see, for example, FIG. 7H). Other configurations including additional target binding units fused in tandem are also contemplated.

In some embodiments, the IgA HetFc binding unit is bispecific, i.e. comprises two target binding domains, each having a different specificity. In some embodiments, the IgA HetFc binding unit is trispecific, i.e. comprises three target binding domains, each having a different specificity. In some embodiments, the IgA HetFc binding unit is tetraspecific, i.e. comprises four target binding domains, each having a different specificity. Greater specificities may be achievable by including some target binding domains in tandem. In some embodiments, at least some of the target binding domains in bispecific, trispecific or tetraspecific IgA HetFc binding units bind to the same target but different epitopes on the target. In some embodiments, at least some of the target binding domains in bispecific, trispecific or tetraspecific IgA HetFc binding units bind to different target molecules.

It should be noted that the specificity of an IgA HetFc binding unit does not necessarily correlate to the number of target binding domains it contains, for example, an IgA HetFc binding unit may comprise two target binding domains but still be monospecific if both target binding domains bind the same target.

In certain embodiments, the present disclosure provides for higher order IgA HetFc multimers that comprise two or more IgA HetFc binding units. In certain embodiments, higher order IgA HetFc multimers of the present disclosure comprise two, four or five IgA HetFc binding units. In certain embodiments, at least two of the IgA HetFc binding units comprised by an IgA HetFc multimer are connected through their tailpieces by a J chain. In the IgA HetFc multimers disclosed herein, the J chain may be a full-length native J chain, but may also contain amino acid alterations, such as substitutions, insertions, deletions, truncations, specifically including J chain fragments, as long as the J chain remains functional. In certain embodiments, the J chain comprised by an IgA HetFc multimer is a modified J chain as described in International Patent Publication No. WO 2015/153912. In certain embodiments, the J chain has the amino acid sequence set forth in SEQ ID NO:48.

As noted above, the IgA HetFc binding units described herein allow for the assembly of IgA HetFc multimers, which are multimeric and multispecific. IgA Het Fc multimers have the potential for fine-tuning avidity effects that can increase the apparent affinity of low-affinity target binding domains and increase clustering and specificity and the associated functionality associated with increased valency. FIG. 8 is a diagram showing illustrative configurations of IgA HetFc multimers.

In some embodiments, an IgA HetFc multimer may be “dimeric” in that it comprises two IgA HetFc binding units joined by a J chain. The IgA HetFc binding units may be monospecific, or they may be bispecific (see, for example, FIG. 8A), or a combination thereof. In some embodiments, a dimeric IgA HetFc multimer of the present disclosure comprises two bispecific IgA HetFc binding units, each binding unit having the same binding specificity (AB, AB). In some embodiments, a dimeric IgA HetFc multimer of the present disclosure comprises two bispecific IgA HetFc binding units, where at least one of the two binding units has a different binding specificity (e.g. AB, BC or AC, BC or AB, CD). Thus, in certain embodiments, each of the two binding units has two specificities, which may be the same (AB, AB) or different (AB, CD or AB, AC, for example).

In some embodiments, the IgA HetFc multimer may be “tetrameric” in that it comprises four IgA HetFc binding units, at least two of which are joined by a J chain. The IgA HetFc binding units may be monospecific, or they may be bispecific (see, for example, FIG. 8B), or combinations thereof. In some embodiments, a tetrameric IgA HetFc multimer of the present disclosure comprises four bispecific binding units, each binding unit having the same binding specificity (AB, AB, AB, AB). Tetrameric IgA HetFc multimers comprising IgA HetFc binding units that are either monospecific or bispecific and have different binding specificities are also contemplated in some embodiments.

In some embodiments, the IgA HetFc multimer may be “pentameric” in that it comprises five IgA HetFc binding units, at least two of which are joined by a J chain. The IgA HetFc binding units may be monospecific, or they may be bispecific (see, for example, FIG. 8C), or combinations thereof. In some embodiments, a pentameric IgA HetFc multimer of the present disclosure comprises five bispecific binding units, each binding unit having the same binding specificity (AB, AB, AB, AB, AB). Pentameric IgA HetFc multimers comprising IgA HetFc binding units that are either monospecific or bispecific and have different binding specificities are also contemplated in some embodiments.

The term “valent,” as used herein, denotes the presence of a specified number of binding sites in the IgA HetFc constructs. For example, the terms “bivalent,” “tetravalent,” “hexavalent,” “octavalent” and “decavalent” denote the presence of two binding sites, four binding sites, six binding sites, eight binding sites and ten binding sites, respectively. Thus, in reference to FIG. 8 herein, the dimeric IgA HetFc multimer shown in FIG. 8A, comprising two bispecific binding units, is tetravalent; the tetrameric IgA HetFc multimer shown in FIG. 8B is octavalent (i.e. comprises four bispecific binding units), and the pentameric IgA HetFc multimer shown in FIG. 8C is decavalent (i.e. comprises five bispecific binding units). Similarly, in reference to FIG. 7, the IgA HetFc binding units shown in FIGS. 7B, C, D, E and F are bivalent, the IgA HetFc binding unit shown in FIG. 7G is trivalent, and the IgA HetFc binding unit shown in FIG. 7H is tetravalent.

In the IgA HetFc binding units and multimers, different components or domains may be fused directly to one another (i.e. without a linker) or one or more of the components or domains may be fused to an adjoining component or domain indirectly via a peptide linker. Peptide linkers suitable for linking components of multi-component proteins are well-known in the art and are selected to allow arrangement of the components such that each may still carry out its intended function.

Peptide linkers are typically between about 2 and about 150 amino acids in length. Useful linkers include glycine-serine (GlySer) linkers, which are well-known in the art and comprise glycine and serine units combined in various orders. Examples include, but are not limited to, (GS)ₙ, (GSGGS)ₙ, (GGGS)ₙ and (GGGGS)ₙ, where n is an integer of at least one, typically an integer between 1 and about 10, for example, between 1 and about 8, between 1 and about 6, or between 1 and about 5; (Gly₃Ser)ₙ(Gly₄Ser)₁, (Gly₃Ser)₁(Gly₄Ser)ₙ, (Gly₃Ser)ₙ(Gly₄Ser)ₙ, or (Gly₄Ser)ₙ, wherein n is an integer of 1 to 5. Other useful linkers include sequences derived from immunoglobulin hinge sequences. The linker may comprise all or part of a hinge sequence from any one of the four IgG classes or from a TCR and may optionally include additional sequences. For example, the linker may include a portion of an immunoglobulin hinge sequence and a glycine-serine sequence. A non-limiting example is a linker that includes approximately the first 15 residues of the IgG1 hinge followed by a GlySer linker sequence, such as those described above, that is about 10 amino acids in length.

### Conjugates

Certain embodiments of the present disclosure relate to conjugates comprising an IgA HetFc construct as described herein (e.g. an IgA HetFc scaffold, IgA HetFc binding unit or IgA HetFc multimer) conjugated to one or more active agents, such as therapeutic, diagnostic or labeling agents.

Examples of therapeutic agents include, but are not limited to, antimetabolites, alkylating agents, anthracyclines, antibiotics, anti-mitotic agents, toxins, apoptotic agents, thrombotic agents, anti-angiogenic agents, biological response modifiers, growth factors, radioactive materials and macrocyclic chelators useful for conjugating radiometal ions. Examples of diagnostic agents include, but are not limited to, various imaging agents such as fluorescent materials, luminescent materials and radioactive materials. Examples of labeling agents include, but are not limited to, enzymes, prosthetic groups, fluorescent materials, luminescent materials and radioactive materials.

Conjugation of the selected active agent to an IgA HetFc construct can be accomplished in a variety of ways and may be direct or via a linker. Linkers for conjugation of active agents are bifunctional or multifunctional moieties capable of linking one or more active agents to an IgA HetFc construct. A bifunctional (or monovalent) linker links a single active agent to a single site on the construct, whereas a multifunctional (or polyvalent) linker links more than one active agent to a single site on the construct. Linkers capable of linking one active agent to more than one site on the IgA HetFc construct may also be considered to be multifunctional.

Conjugation may be achieved, for example, through surface lysines on the IgA HetFc construct, reductive-coupling to oxidized carbohydrates on the IgA HetFc construct, or through cysteine residues on the IgA HetFc construct liberated by reducing interchain disulfide linkages. Alternatively, conjugation may be achieved by modification of the IgA HetFc construct to include additional cysteine residues (see, for example, U.S. Pat. Nos. 7,521,541; 8,455,622 and 9,000,130) or non-natural amino acids that provide reactive handles, such as selenomethionine, p-acetylphenylalanine, formylglycine or p-azidomethyl-L-phenylalanine (see, for example, Hofer et al., 2009, Biochemistry, 48:12047-12057; Axup et al., 2012, PNAS, 109:16101-16106; Wu et al., 2009, PNAS, 106:3000-3005; Zimmerman et al., 2014, Bioconj. Chem., 25:351-361) to allow for site-specific conjugation.

Methods for conjugating various agents to proteins, including immunoglobulins, are known in the art (see, for example, in Bioconjugate Techniques (G. T. Hermanson, 2013, Academic Press).

### Polynucleotides and Methods of Preparing IgA HetFc Constructs

The IgA HetFc constructs described herein may be prepared using standard recombinant methods. Recombinant production of an IgA HetFc construct generally involves synthesizing one or more polynucleotides encoding the IgA HetFc construct, cloning the one or more polynucleotides into an appropriate vector or vectors, and introducing the vector(s) into a suitable host cell for expression of the IgA HetFc construct. Recombinant production of proteins is well-known in the art and may be achieved using standard techniques as described, for example, in Sambrook et al., Molecular Cloning: A Laboratory Manual, 3rd ed., Cold Spring Harbor Laboratory Press, Cold Spring Harbor, N Y (2001); Ausubel et al., Current Protocols in Molecular Biology, (1987 & updates), John Wiley & Sons, New York, NY; and Harlow and Lane, Antibodies: A Laboratory Manual, Cold Spring Harbor Laboratory Press, Cold Spring Harbor, NY (1990).

Certain embodiments of the present disclosure thus relate to an isolated polynucleotide or set of polynucleotides encoding an IgA HetFc construct as described herein. A polynucleotide in this context may encode all or part of an IgA HetFc construct.

The terms “nucleic acid,” “nucleic acid molecule” and “polynucleotide” are used interchangeably herein and refer to a polymeric form of nucleotides of any length, either deoxyribonucleotides or ribonucleotides, or analogues thereof. The polynucleotide may be of genomic, cDNA, RNA, semisynthetic or synthetic origin, or any combination thereof.

A polynucleotide that “encodes” an IgA HetFc construct is a polynucleotide that is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. A transcription termination sequence may be located 3′ to the coding sequence.

The one or more polynucleotides encoding the IgA HetFc construct may be inserted into a suitable expression vector or vectors, either directly or after one or more subcloning steps, using standard ligation techniques. Examples of suitable vectors include, but are not limited to, plasmids, phagemids, cosmids, bacteriophage, baculoviruses, retroviruses or DNA viruses. The vector is typically selected to be functional in the particular host cell that will be employed, i.e. the vector is compatible with the host cell machinery, permitting amplification and/or expression of the polynucleotide(s). Selection of appropriate vector and host cell combinations in this regard is well within the ordinary skills of a worker in the art.

Certain embodiments of the present disclosure thus relate to vectors (such as expression vectors) comprising one or more polynucleotides encoding an IgA HetFc construct. The polynucleotide(s) may be comprised by a single vector or by more than one vector. In some embodiments, the polynucleotides are comprised by a multicistronic vector.

Typically, expression vectors will contain one or more regulatory elements for plasmid maintenance and for cloning and expression of exogenous polynucleotide sequences. Examples of such regulatory elements include promoters, enhancer sequences, origins of replication, transcriptional termination sequences, donor and acceptor splice sites, leader sequences for polypeptide secretion, ribosome binding sites, polyadenylation sequences, polylinker regions for inserting the polynucleotide encoding the polypeptide to be expressed, and selectable markers.

Regulatory elements may be homologous (i.e. from the same species and/or strain as the host cell), heterologous (i.e. from a species other than the host cell species or strain), hybrid (i.e. a combination of regulatory elements from more than one source) or synthetic. As such, the source of a regulatory element may be any prokaryotic or eukaryotic organism provided that the flanking sequence is functional in, and can be activated by, the machinery of the host cell being employed.

Optionally, the vector may also contain a “tag”-encoding sequence. A tag-encoding sequence is a nucleic acid sequence located at the 5′ or 3′ end of the coding sequence that encodes a heterologous peptide sequence, such as a polyHis (for example, 6×His), FLAG®, HA (hemaglutinin influenza virus), myc, metal-affinity, avidin/streptavidin, glutathione-S-transferase (GST) or biotin tag. This tag typically remains fused to the expressed polypeptide and can serve as a means for affinity purification or detection of the polypeptide. Optionally, the tag can subsequently be removed from the purified polypeptide by various means such as using certain peptidases for cleavage.

Various expression vectors are readily available from commercial sources. Alternatively, when a commercial vector containing all the desired regulatory elements is not available, an expression vector may be constructed using a commercially available vector as a starting vector.

Where one or more of the desired regulatory elements are not already present in the vector, they may be individually obtained and ligated into the vector. Methods and sources for obtaining various regulatory elements are well known to one skilled in the art.

Following construction of the expression vector(s) including the polynucleotide(s) encoding the IgA HetFc construct, the vector(s) may be inserted into a suitable host cell for amplification and/or protein expression. The transformation of an expression vector into a selected host cell may be accomplished by well-known methods including transfection, infection, calcium phosphate co-precipitation, electroporation, microinjection, lipofection, DEAE-dextran mediated transfection, and other known techniques. The method selected will in part be a function of the type of host cell to be used. These methods and other suitable methods are well known to the skilled person (see, for example, Sambrook, et al., ibid.).

A host cell, when cultured under appropriate conditions, expresses the polypeptide encoded by the vector and the polypeptide can subsequently be collected from the culture medium (if the host cell secretes the polypeptide) or directly from the host cell producing it (if the polypepitde is not secreted). The host cell may be prokaryotic (for example, a bacterial cell) or eukaryotic (for example, a yeast, fungi, plant or mammalian cell). The selection of an appropriate host cell can be readily made by the skilled person taking into account various factors, such as desired expression levels, polypeptide modifications that are desirable or necessary for activity (such as glycosylation or phosphorylation) and ease of folding into a biologically active molecule.

Certain embodiments of the present disclosure thus relate to host cells comprising polynucleotide(s) encoding the IgA HetFc construct, or one or more vectors comprising the polynucleotide(s). In certain embodiments, the host cell is a eukaryotic cell.

For example, eukaryotic microbes such as filamentous fungi or yeast may be employed as host cells, including fungi and yeast strains whose glycosylation pathways have been “humanized” (see, for example, Gerngross, 2004, Nat. Biotech., 22:1409-1414, and Li et al., 2006, Nat. Biotech., 24:210-215). Plant cells may also be utilized as host cells (see, for example, U.S. Pat. Nos. 5,959,177; 6,040,498; 6,420,548; 7,125,978 and 6,417,429, describing PLANTIBODIES™ technology).

In some embodiments, the eukaryotic host cell is a mammalian cell. Various mammalian cell lines may be used as host cells. Examples of useful mammalian host cell lines include, but are not limited to, monkey kidney CV1 line transformed by SV40 (COS-7), human embryonic kidney line 293 (HEK293 cells as described, for example, in Graham, et al., 1977, J. Gen Virol., 36:59), baby hamster kidney cells (BHK), mouse sertoli cells (TM4 cells as described, for example, in Mather, 1980, Biol. Reprod., 23:243-251), monkey kidney cells (CV1), African green monkey kidney cells (VERO-76), human cervical carcinoma cells (HeLa), canine kidney cells (MDCK), buffalo rat liver cells (BRL 3A), human lung cells (W138), human liver cells (Hep G2), mouse mammary tumour cells (MMT 060562), TRI cells (as described, for example, in Mather, et al., 1982, Annals N.Y. Acad. Sci., 383:44-68), MRC 5 cells, FS4 cells, Chinese hamster ovary (CHO) cells (including DHFR⁻ CHO cells as described in Urlaub, et al., 1980, Proc. Natl. Acad. Sci. USA, 77:4216) and myeloma cell lines (such as Y0, NS0 and Sp2/0). See also, Yazaki and Wu, 2003, Methods in Molecular Biology, Vol. 248, pp. 255-268 (B. K. C. Lo, ed., Humana Press, Totowa, N.J.).

Certain embodiments of the present disclosure relate to methods of preparing an IgA HetFc construct described herein, comprising transfecting a host cell with one or more polynucleotides encoding the IgA HetFc construct, for example in the form of one or more vectors comprising the polynucleotide(s), and culturing the host cell under conditions suitable for expression of the encoded IgA HetFc construct.

Typically, the IgA HetFc construct is isolated from the host cell after expression and may optionally be purified. Methods for isolating and purifying expressed proteins are well-known in the art. Standard purification methods include, for example, chromatographic techniques, such ion exchange, hydrophobic interaction, affinity, sizing, gel filtration or reversed-phase, which may be carried out at atmospheric pressure or at medium or high pressure using systems such as FPLC, MPLC and HPLC. Other purification methods include electrophoretic, immunological, precipitation, dialysis and chromatofocusing techniques. Ultrafiltration and diafiltration techniques, in conjunction with protein concentration, may also be useful.

A variety of natural proteins are known in the art to bind Fc regions of antibodies, and these proteins can therefore be used in the purification of Fc-containing proteins. For example, the bacterial proteins A and G bind to the Fc region. Purification can often be enabled by a particular fusion partner or affinity tag as described above. For example, antibodies may be purified using glutathione resin if a GST fusion is employed, Ni⁺² affinity chromatography if a His-tag is employed, or immobilized anti-flag antibody if a flag-tag is used. Examples of useful purification techniques are described in Harlow and Lane, Antibodies: A Laboratory Manual, Cold Spring Harbor Laboratory Press, Cold Spring Harbor, NY (1990), and Protein Purification: Principles and Practice, 3^{rd} Ed., Scopes, Springer-Verlag, NY (1994). The degree of purification necessary will vary depending on the use of the IgA HetFc construct. In some instances, no purification may be necessary.

In certain embodiments, the IgA HetFc constructs herein are purified using one or more purification methods known in the art, including but not limited to, affinity chromatography, affinity chromatography by non-reducing CE-SDS, affinity purification (protein A purification columns, CaptureSelect™ IgA affinity purification) and size exclusion chromatography, e.g. UPLC-SEC (see also Examples 1-6).

### Post-Translational Modifications

In certain embodiments, the IgA HetFc constructs described herein may be post-translationally modified.

The term “post-translationally modified” and grammatical variations thereof such as “post-translational modification,” refers to a modification of a natural or non-natural amino acid that occurs to such an amino acid after it has been incorporated into a polypeptide chain. The term encompasses, by way of example only, co-translational in vivo modifications, co-translational in vitro modifications (such as in a cell-free translation system), post-translational in vivo modifications and post-translational in vitro modifications.

Specific examples of post-translational modifications include, but are not limited to, glycosylation, acetylation, phosphorylation, amidation, derivatization by known protecting/blocking groups, proteolytic cleavage, or a combination thereof. Other examples include chemical modification by known techniques including, but not limited to, specific chemical cleavage by cyanogen bromide, trypsin, chymotrypsin, papain, V8 protease or NaBH₄; acetylation; formylation; oxidation; reduction or metabolic synthesis in the presence of tunicamycin.

Additional post-translational modifications include attachment of chemical moieties to the amino acid backbone, chemical modifications of N-linked or O-linked carbohydrate chains, and addition or deletion of an N-terminal methionine residue as a result of prokaryotic host cell expression.

In certain embodiments, IgA HetFc constructs described herein may optionally be modified with a detectable label, such as an enzymatic, fluorescent, isotopic or affinity label to allow for detection and isolation of the protein. Examples of suitable enzyme labels include horseradish peroxidase, alkaline phosphatase, beta-galactosidase and acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride and phycoerythrin; an example of a luminescent material includes luminol; examples of bioluminescent materials include luciferase, luciferin and aequorin; and examples of suitable radioactive materials include radioactive isotopes of iodine, carbon, sulfur, tritium, indium, technetium, thallium, gallium, palladium, molybdenum, xenon and fluorine.

In some embodiments, the IgA HetFc constructs described herein may optionally be attached to macrocyclic chelators that associate with radiometal ions.

In those embodiments in which the IgA HetFc constructs are modified, either by natural processes, such as post-translational processing, or by chemical modification techniques, the same type of modification may optionally be present in the same or varying degrees at several sites in a given polypeptide.

In certain embodiments, the IgA HetFc constructs may be attached to a solid support, which may be particularly useful for immunoassays or purification of polypeptides that are bound by, or bind to, or associate with proteins described herein. Such solid supports include, but are not limited to, glass, cellulose, polyacrylamide, nylon, polystyrene, polyvinyl chloride and polypropylene.

### Characterization of IgA HetFc Constructs

IgA HetFc constructs as described herein may be characterized in a variety of ways. For example, purity of the IgA HetFc constructs may be assessed using techniques well known in the art including, but not limited to, SDS-PAGE gels, western blots, densitometry, mass spectrometry, size-exclusion chromatography (SEC) or non-reducing capillary electrophoresis sodium dodecyl sulfate (CE-SDS). In certain embodiments, purity of the IgA HetFc constructs is assessed by SEC or CE-SDS.

Protein stability may also be characterized using an array of art-known techniques including, but not limited to, size exclusion chromatography (SEC); UV, visible or CD spectroscopy; mass spectroscopy; differential light scattering (DLS); bench top stability assay; freeze thawing coupled with other characterization techniques; differential scanning calorimetry (DSC); differential scanning fluorimetry (DSF); hydrophobic interaction chromatography (HIC); isoelectric focusing; receptor binding assays or relative protein expression levels. In certain embodiments, stability of the IgA HetFc constructs is assessed by measuring CH3 domain melting temperature (Tm), as compared to wild-type CH3 domain Tm, using techniques well known in the art such as DSC or DSF.

Where appropriate, IgA HetFc constructs of the present disclosure may also be assayed for the ability to specifically bind to a ligand, receptor or target antigen (e.g. to FcαRI, or to a target antigen of a binding domain comprised by the IgA HetFc construct). Various immunoassays known in the art may be employed to analyze specific binding and cross-reactivity including, but are not limited to, competitive and non-competitive assay systems using techniques such as western blots, radioimmunoassays, ELISA (enzyme-linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays. Such assays are routine and well known in the art (see, for example, Ausubel, et al., eds, 1994, Current Protocols in Molecular Biology, John Wiley and Sons, Inc., New York).

IgA HetFc constructs that are confirmed to specifically bind to the target ligand, receptor or antigen may optionally also be assayed for their affinity for the ligand, receptor or antigen. Binding affinity and parameters such as the on-rate and the off-rate of the interaction can be determined, for example, by competitive binding assays. The kinetic parameters of an IgA HetFc construct may also be determined using surface plasmon resonance (SPR) based assays known in the art, such as BIAcore™ kinetic analysis. Various SPR-based assays are known in the art (see, for example, Mullet, et al., 2000, Methods, 22:77-91; Dong, et al., 2002, Rev. Mol. Biotech., 82:303-23; Fivash, et al., 1998, Curr Opinion in Biotechnology, 9:97-101; Rich, et al., 2000, Curr Opinion in Biotechnology, 11:54-61, and U.S. Pat. Nos. 6,373,577; 6,289,286; 5,322,798; 5,341,215 and 6,268,125). Fluorescence activated cell sorting (FACS), using techniques known to those skilled in the art, may also be used for characterizing the binding of an IgA HetFc construct to a molecule expressed on the cell surface (e.g. an Fc receptor or a cell surface antigen). Flow cytometers for sorting and examining biological cells are well known in the art (see, for example, U.S. Pat. Nos. 4,347,935; 5,464,581; 5,483,469; 5,602,039; 5,643,796 and 6,211,477). Other known flow cytometers are the FACS Vantage™ system manufactured by Becton Dickinson and Company (Franklin Lakes, NJ) and the COPAS™ system manufactured by Union Biometrica (Holliston, MA). A detailed description of binding affinities and kinetics can be found in Paul, W. E., ed., 1999, Fundamental Immunology, 4th Ed., Lippincott-Raven, Philadelphia, which focuses on antibody-immunogen interactions.

Binding properties of the IgA HetFc constructs may also be characterized by in vitro functional assays for determining one or more FcαRI downstream functions (see, for example, Bakema, 2006, J Immunol, 176:3603-3610).

### Methods of Use

Certain embodiments of the present disclosure relate to the use of the IgA HetFc constructs described herein in therapeutic or diagnostic methods. For example, IgA constructs may be used in methods of engaging neutrophils via FcαRI, and methods of activating neutrophils via FcαRI.

IgA HetFc constructs comprising one or more binding domains and IgA HetFc constructs conjugated to a therapeutic agent may be used in methods of treatment, for example, treating a subject with cancer, autoimmune disease, immune or inflammatory disorders or an infectious disease. Similarly, IgA constructs comprising one or more binding domains and IgA HetFc constructs conjugated to a labeling or diagnostic agent may be used in methods of diagnosis, for example, diagnosing a subject with cancer, autoimmune disease, immune or inflammatory disorders or an infectious disease.

When used in methods of treatment, the IgA HetFc constructs are administered to the subject in a therapeutically effective amount. The term “therapeutically effective amount” as used herein refers to an amount of an IgA HetFc construct described herein or a composition comprising an IgA HetFc construct described herein being administered that will accomplish the goal of the recited method, for example, relieve to some extent one or more of the symptoms of the disease or disorder being treated. The amount of the composition described herein which will be effective in the treatment of the disease or disorder in question can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances.

In some embodiments in which the IgA HetFc construct is used in a method of treatment, the IgA HetFc construct may be administered in combination with a therapeutically effective amount of one or more additional therapeutic agents known to those skilled in the art for the treatment of the disease or disorder in question.

Desirable effects of treatment include, but are not limited to, one or more of alleviation of symptoms, diminishment of any direct or indirect pathological consequences of the disease or disorder, preventing metastasis, decreasing the rate of disease progression, amelioration or palliation of the disease state, improved survival, remission, improved prognosis or delaying the recurrence of disease.

### Pharmaceutical Compositions

For therapeutic or diagnostic use, the IgA HetFc constructs may be provided in the form of compositions which comprise the IgA HetFc construct and a pharmaceutically acceptable carrier or diluent. The compositions may be prepared by known procedures using well-known and readily available ingredients and may be formulated for administration to a subject by, for example, oral (including, for example, buccal or sublingual), topical, parenteral, rectal or vaginal routes, or by inhalation or spray. The term “parenteral” as used herein includes injection or infusion by subcutaneous, intradermal, intra-articular, intravenous, intramuscular, intravascular, intrasternal or intrathecal routes.

The composition will typically be formulated in a format suitable for administration to a subject by the chosen route, for example, as a syrup, elixir, tablet, troche, lozenge, hard or soft capsule, pill, suppository, oily or aqueous suspension, dispersible powder or granule, emulsion, injectable or solution. Compositions may be provided as unit dosage formulations.

Pharmaceutically acceptable carriers are generally non-toxic to recipients at the dosages and concentrations employed. Examples of such carriers include, but are not limited to, buffers such as phosphate, citrate, and other organic acids; antioxidants such as ascorbic acid and methionine; preservatives such as octadecyldimethylbenzyl ammonium chloride, hexamethonium chloride, benzalkonium chloride, benzethonium chloride, phenol, butyl alcohol, benzyl alcohol, alkyl parabens (such as methyl or propyl paraben), catechol, resorcinol, cyclohexanol, 3-pentanol and m-cresol; low molecular weight (less than about 10 amino acids) polypeptides; proteins such as serum albumin or gelatin; hydrophilic polymers such as polyvinylpyrrolidone; amino acids such as glycine, glutamine, asparagine, histidine, arginine or lysine; monosaccharides, disaccharides, and other carbohydrates such as glucose, mannose or dextrins; chelating agents such as EDTA; sugars such as sucrose, mannitol, trehalose or sorbitol; salt-forming counter-ions such as sodium; metal complexes such as Zn-protein complexes, and non-ionic surfactants such as polyethylene glycol (PEG).

In certain embodiments, the compositions may be in the form of a sterile injectable aqueous or oleaginous solution or suspension. Such solutions or suspensions may be formulated using suitable dispersing or wetting agents and/or suspending agents that are known in the art. The sterile injectable solution or suspension may comprise the IgA HetFc constructs in a non-toxic parentally acceptable diluent or solvent. Acceptable diluents and solvents that may be employed include, for example, 1,3-butanediol, water, Ringer's solution or isotonic sodium chloride solution. In addition, sterile, fixed oils may be employed as a solvent or suspending medium. For this purpose, various bland fixed oils may be employed, including synthetic mono- or diglycerides. In addition, fatty acids such as oleic acid find use in the preparation of injectables. Adjuvants such as local anaesthetics, preservatives and/or buffering agents as known in the art may also be included in the injectable solution or suspension.

Other pharmaceutical compositions and methods of preparing pharmaceutical compositions are known in the art and are described, for example, in “Remington: The Science and Practice of Pharmacy” (formerly “Remingtons Pharmaceutical Sciences”); Gennaro, A., Lippincott, Williams & Wilkins, Philadelphia, PA (2000).

### Kits and Articles of Manufacture

Certain embodiments of the present disclosure relate to kits comprising one or more IgA HetFc constructs described herein. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale. The kit may optionally contain instructions or directions outlining the method of use or administration regimen for the IgA HetFc constructs.

When one or more components of the kit are provided as solutions, for example an aqueous solution, or a sterile aqueous solution, the container means may itself be an inhalant, syringe, pipette, eye dropper, or other such like apparatus, from which the solution may be administered to a subject or applied to and mixed with the other components of the kit.

The components of the kit may also be provided in dried or lyophilized form and the kit can additionally contain a suitable solvent for reconstitution of the lyophilized components. Irrespective of the number or type of containers, the kits described herein also may comprise an instrument for assisting with the administration of the composition to a patient. Such an instrument may be an inhalant, nasal spray device, syringe, pipette, forceps, measured spoon, eye dropper or similar medically approved delivery vehicle.

Certain embodiments relate to an article of manufacture containing materials useful for treatment of a patient as described herein. The article of manufacture comprises a container and a label or package insert on or associated with the container. Suitable containers include, for example, bottles, vials, syringes, intravenous solution bags, and the like. The containers may be formed from a variety of materials such as glass or plastic. The container holds a composition comprising the IgA HetFc construct which is by itself or combined with another composition effective for treating the patient and may have a sterile access port (for example the container may be an intravenous solution bag or a vial having a stopper pierceable by a hypodermic injection needle). The label or package insert indicates that the composition is used for treating the condition of choice. The article of manufacture may further comprise a second container comprising a pharmaceutically acceptable buffer, such as bacteriostatic water for injection (BWFI), phosphate-buffered saline, Ringer's solution or dextrose solution. The article of manufacture may optionally further include other materials desirable from a commercial and user standpoint, including other buffers, diluents, filters, needles, and syringes.

The following Examples are provided for illustrative purposes and are not intended to limit the scope of the invention in any way.

TABLE 7
IgA HetFc Designs Comprising Core Mutations

CH3 Domain Mutations

| No. | Chain A | Chain B |
|---|---|---|
| D1 | A6085YF_T6086Y | W6081T |
| D2 | A6085YY_T6086Y | W6081T |
| D3 | A6085YM_T6086Y | W6081T |
| D4 | A6085YW_T6086Y | W6081T |
| D5 | A6085YH_T6086Y | W6081T |
| D6 | A6085YF_T6086F | W6081T |
| D7 | A6085YY_T6086F | W6081T |
| D8 | A6085YM_T6086F | W6081T |
| D9 | A6085YW_T6086F | W6081T |
| D10 | A6085YH_T6086F | W6081T |
| D11 | A6085YF_T6086M | W6081T |
| D12 | A6085YY_T6086M | W6081T |
| D13 | A6085YM_T6086M | W6081T |
| D14 | A6085YW_T6086M | W6081T |
| D15 | A6085YH_T6086M | W6081T |
| D16 | A6085YF_T6086W | W6081T |
| D17 | A6085YY_T6086W | W6081T |
| D18 | A6085YM_T6086W | W6081T |
| D19 | A6085YW_T6086W | W6081T |
| D20 | A6085YH_T6086W | W6081T |
| D21 | A6085YF_T6086H | W6081T |
| D22 | A6085YY_T6086H | W6081T |
| D23 | A6085YM_T6086H | W6081T |
| D24 | A6085YW_T6086H | W6081T |
| D25 | A6085YH_T6086H | W6081T |
| D26 | A6085YF_T6086Y | W6081L |
| D27 | A6085YY_T6086Y | W6081L |
| D28 | A6085YM_T6086Y | W6081L |
| D29 | A6085YW_T6086Y | W6081L |
| D30 | A6085YH_T6086Y | W6081L |
| D31 | A6085YF_T6086F | W6081L |
| D32 | A6085YY_T6086F | W6081L |
| D33 | A6085YM_T6086F | W6081L |
| D34 | A6085YW_T6086F | W6081L |
| D35 | A6085YH_T6086F | W6081L |
| D36 | A6085YF_T6086M | W6081L |
| D37 | A6085YY_T6086M | W6081L |
| D38 | A6085YM_T6086M | W6081L |
| D39 | A6085YW_T6086M | W6081L |
| D40 | A6085YH_T6086M | W6081L |
| D41 | A6085YF_T6086W | W6081L |
| D42 | A6085YY_T6086W | W6081L |
| D43 | A6085YM_T6086W | W6081L |
| D44 | A6085YW_T6086W | W6081L |
| D45 | A6085YH_T6086W | W6081L |
| D46 | A6085YF_T6086H | W6081L |
| D47 | A6085YY_T6086H | W6081L |
| D48 | A6085YM_T6086H | W6081L |
| D49 | A6085YW_T6086H | W6081L |
| D50 | A6085YH_T6086H | W6081L |
| D51 | A6085YF_T6086Y | W6081A |
| D52 | A6085YY_T6086Y | W6081A |
| D53 | A6085YM_T6086Y | W6081A |
| D54 | A6085YW_T6086Y | W6081A |
| D55 | A6085YH_T6086Y | W6081A |
| D56 | A6085YF_T6086F | W6081A |
| D57 | A6085YY_T6086F | W6081A |
| D58 | A6085YM_T6086F | W6081A |
| D59 | A6085YW_T6086F | W6081A |
| D60 | A6085YH_T6086F | W6081A |
| D61 | A6085YF_T6086M | W6081A |
| D62 | A6085YY_T6086M | W6081A |
| D63 | A6085YM_T6086M | W6081A |
| D64 | A6085YW_T6086M | W6081A |
| D65 | A6085YH_T6086M | W6081A |
| D66 | A6085YF_T6086W | W6081A |
| D67 | A6085YY_T6086W | W6081A |
| D68 | A6085YM_T6086W | W6081A |
| D69 | A6085YW_T6086W | W6081A |
| D70 | A6085YH_T6086W | W6081A |
| D71 | A6085YF_T6086H | W6081A |
| D72 | A6085YY_T6086H | W6081A |
| D73 | A6085YM_T6086H | W6081A |
| D74 | A6085YW_T6086H | W6081A |
| D75 | A6085YH_T6086H | W6081A |
| D76 | A6085YF_T6086Y | W6081V |
| D77 | A6085YY_T6086Y | W6081V |
| D78 | A6085YM_T6086Y | W6081V |
| D79 | A6085YW_T6086Y | W6081V |
| D80 | A6085YH_T6086Y | W6081V |
| D81 | A6085YF_T6086F | W6081V |
| D82 | A6085YY_T6086F | W6081V |
| D83 | A6085YM_T6086F | W6081V |
| D84 | A6085YW_T6086F | W6081V |
| D85 | A6085YH_T6086F | W6081V |
| D86 | A6085YF_T6086M | W6081V |
| D87 | A6085YY_T6086M | W6081V |
| D88 | A6085YM_T6086M | W6081V |
| D89 | A6085YW_T6086M | W6081V |
| D90 | A6085YH_T6086M | W6081V |
| D91 | A6085YF_T6086W | W6081V |
| D92 | A6085YY_T6086W | W6081V |
| D93 | A6085YM_T6086W | W6081V |
| D94 | A6085YW_T6086W | W6081V |
| D95 | A6085YH_T6086W | W6081V |
| D96 | A6085YF_T6086H | W6081V |
| D97 | A6085YY_T6086H | W6081V |
| D98 | A6085YM_T6086H | W6081V |
| D99 | A6085YW_T6086H | W6081V |
| D100 | A6085YH_T6086H | W6081V |
| D101 | A6085YF_T6086Y | W6081I |
| D102 | A6085YY_T6086Y | W6081I |
| D103 | A6085YM_T6086Y | W6081I |
| D104 | A6085YW_T6086Y | W6081I |
| D105 | A6085YH_T6086Y | W6081I |
| D106 | A6085YF_T6086F | W6081I |
| D107 | A6085YY_T6086F | W6081I |
| D108 | A6085YM_T6086F | W6081I |
| D109 | A6085YW_T6086F | W6081I |
| D110 | A6085YH_T6086F | W6081I |
| D111 | A6085YF_T6086M | W6081I |
| D112 | A6085YY_T6086M | W6081I |
| D113 | A6085YM_T6086M | W6081I |
| D114 | A6085YW_T6086M | W6081I |
| D115 | A6085YH_T6086M | W6081I |
| D116 | A6085YF_T6086W | W6081I |
| D117 | A6085YY_T6086W | W6081I |
| D118 | A6085YM_T6086W | W6081I |
| D119 | A6085YW_T6086W | W6081I |
| D120 | A6085YH_T6086W | W6081I |
| D121 | A6085YF_T6086H | W6081I |
| D122 | A6085YY_T6086H | W6081I |
| D123 | A6085YM_T6086H | W6081I |
| D124 | A6085YW_T6086H | W6081I |
| D125 | A6085YH_T6086H | W6081I |

TABLE 8
IgA HetFc Designs comprising Core Mutations in Combination
with Mutation at Position 6079 in Chain B

CH3 Domain Mutations

| No. | Chain A | Chain B |
|---|---|---|
| D126 | A6085YF_T6086Y | L6079V_W6081T |
| D127 | A6085YY_T6086Y | L6079V_W6081T |
| D128 | A6085YM_T6086Y | L6079V_W6081T |
| D129 | A6085YW_T6086Y | L6079V_W6081T |
| D130 | A6085YH_T6086Y | L6079V_W6081T |
| D131 | A6085YF_T6086F | L6079V_W6081T |
| D132 | A6085YY_T6086F | L6079V_W6081T |
| D133 | A6085YM_T6086F | L6079V_W6081T |
| D134 | A6085YW_T6086F | L6079V_W6081T |
| D135 | A6085YH_T6086F | L6079V_W6081T |
| D136 | A6085YF_T6086M | L6079V_W6081T |
| D137 | A6085YY_T6086M | L6079V_W6081T |
| D138 | A6085YM_T6086M | L6079V_W6081T |
| D139 | A6085YW_T6086M | L6079V_W6081T |
| D140 | A6085YH_T6086M | L6079V_W6081T |
| D141 | A6085YF_T6086W | L6079V_W6081T |
| D142 | A6085YY_T6086W | L6079V_W6081T |
| D143 | A6085YM_T6086W | L6079V_W6081T |
| D144 | A6085YW_T6086W | L6079V_W6081T |
| D145 | A6085YH_T6086W | L6079V_W6081T |
| D146 | A6085YF_T6086H | L6079V_W6081T |
| D147 | A6085YY_T6086H | L6079V_W6081T |
| D148 | A6085YM_T6086H | L6079V_W6081T |
| D149 | A6085YW_T6086H | L6079V_W6081T |
| D150 | A6085YH_T6086H | L6079V_W6081T |
| D151 | A6085YF_T6086Y | L6079V_W6081L |
| D152 | A6085YY_T6086Y | L6079V_W6081L |
| D153 | A6085YM_T6086Y | L6079V_W6081L |
| D154 | A6085YW_T6086Y | L6079V_W6081L |
| D155 | A6085YH_T6086Y | L6079V_W6081L |
| D156 | A6085YF_T6086F | L6079V_W6081L |
| D157 | A6085YY_T6086F | L6079V_W6081L |
| D158 | A6085YM_T6086F | L6079V_W6081L |
| D159 | A6085YW_T6086F | L6079V_W6081L |
| D160 | A6085YH_T6086F | L6079V_W6081L |
| D161 | A6085YF_T6086M | L6079V_W6081L |
| D162 | A6085YY_T6086M | L6079V_W6081L |
| D163 | A6085YM_T6086M | L6079V_W6081L |
| D164 | A6085YW_T6086M | L6079V_W6081L |
| D165 | A6085YH_T6086M | L6079V_W6081L |
| D166 | A6085YF_T6086W | L6079V_W6081L |
| D167 | A6085YY_T6086W | L6079V_W6081L |
| D168 | A6085YM_T6086W | L6079V_W6081L |
| D169 | A6085YW_T6086W | L6079V_W6081L |
| D170 | A6085YH_T6086W | L6079V_W6081L |
| D171 | A6085YF_T6086H | L6079V_W6081L |
| D172 | A6085YY_T6086H | L6079V_W6081L |
| D173 | A6085YM_T6086H | L6079V_W6081L |
| D174 | A6085YW_T6086H | L6079V_W6081L |
| D175 | A6085YH_T6086H | L6079V_W6081L |
| D176 | A6085YF_T6086Y | L6079V_W6081A |
| D177 | A6085YY_T6086Y | L6079V_W6081A |
| D178 | A6085YM_T6086Y | L6079V_W6081A |
| D179 | A6085YW_T6086Y | L6079V_W6081A |
| D180 | A6085YH_T6086Y | L6079V_W6081A |
| D181 | A6085YF_T6086F | L6079V_W6081A |
| D182 | A6085YY_T6086F | L6079V_W6081A |
| D183 | A6085YM_T6086F | L6079V_W6081A |
| D184 | A6085YW_T6086F | L6079V_W6081A |
| D185 | A6085YH_T6086F | L6079V_W6081A |
| D186 | A6085YF_T6086M | L6079V_W6081A |
| D187 | A6085YY_T6086M | L6079V_W6081A |
| D188 | A6085YM_T6086M | L6079V_W6081A |
| D189 | A6085YW_T6086M | L6079V_W6081A |
| D190 | A6085YH_T6086M | L6079V_W6081A |
| D191 | A6085YF_T6086W | L6079V_W6081A |
| D192 | A6085YY_T6086W | L6079V_W6081A |
| D193 | A6085YM_T6086W | L6079V_W6081A |
| D194 | A6085YW_T6086W | L6079V_W6081A |
| D195 | A6085YH_T6086W | L6079V_W6081A |
| D196 | A6085YF_T6086H | L6079V_W6081A |
| D197 | A6085YY_T6086H | L6079V_W6081A |
| D198 | A6085YM_T6086H | L6079V_W6081A |
| D199 | A6085YW_T6086H | L6079V_W6081A |
| D200 | A6085YH_T6086H | L6079V_W6081A |
| D201 | A6085YF_T6086Y | L6079V_W6081V |
| D202 | A6085YY_T6086Y | L6079V_W6081V |
| D203 | A6085YM_T6086Y | L6079V_W6081V |
| D204 | A6085YW_T6086Y | L6079V_W6081V |
| D205 | A6085YH_T6086Y | L6079V_W6081V |
| D206 | A6085YF_T6086F | L6079V_W6081V |
| D207 | A6085YY_T6086F | L6079V_W6081V |
| D208 | A6085YM_T6086F | L6079V_W6081V |
| D209 | A6085YW_T6086F | L6079V_W6081V |
| D210 | A6085YH_T6086F | L6079V_W6081V |
| D211 | A6085YF_T6086M | L6079V_W6081V |
| D212 | A6085YY_T6086M | L6079V_W6081V |
| D213 | A6085YM_T6086M | L6079V_W6081V |
| D214 | A6085YW_T6086M | L6079V_W6081V |
| D215 | A6085YH_T6086M | L6079V_W6081V |
| D216 | A6085YF_T6086W | L6079V_W6081V |
| D217 | A6085YY_T6086W | L6079V_W6081V |
| D218 | A6085YM_T6086W | L6079V_W6081V |
| D219 | A6085YW_T6086W | L6079V_W6081V |
| D220 | A6085YH_T6086W | L6079V_W6081V |
| D221 | A6085YF_T6086H | L6079V_W6081V |
| D222 | A6085YY_T6086H | L6079V_W6081V |
| D223 | A6085YM_T6086H | L6079V_W6081V |
| D224 | A6085YW_T6086H | L6079V_W6081V |
| D225 | A6085YH_T6086H | L6079V_W6081V |
| D226 | A6085YF_T6086Y | L6079V_W6081I |
| D227 | A6085YY_T6086Y | L6079V_W6081I |
| D228 | A6085YM_T6086Y | L6079V_W6081I |
| D229 | A6085YW_T6086Y | L6079V_W6081I |
| D230 | A6085YH_T6086Y | L6079V_W6081I |
| D231 | A6085YF_T6086F | L6079V_W6081I |
| D232 | A6085YY_T6086F | L6079V_W6081I |
| D233 | A6085YM_T6086F | L6079V_W6081I |
| D234 | A6085YW_T6086F | L6079V_W6081I |
| D235 | A6085YH_T6086F | L6079V_W6081I |
| D236 | A6085YF_T6086M | L6079V_W6081I |
| D237 | A6085YY_T6086M | L6079V_W6081I |
| D238 | A6085YM_T6086M | L6079V_W6081I |
| D239 | A6085YW_T6086M | L6079V_W6081I |
| D240 | A6085YH_T6086M | L6079V_W6081I |
| D241 | A6085YF_T6086W | L6079V_W6081I |
| D242 | A6085YY_T6086W | L6079V_W6081I |
| D243 | A6085YM_T6086W | L6079V_W6081I |
| D244 | A6085YW_T6086W | L6079V_W6081I |
| D245 | A6085YH_T6086W | L6079V_W6081I |
| D246 | A6085YF_T6086H | L6079V_W6081I |
| D247 | A6085YY_T6086H | L6079V_W6081I |
| D248 | A6085YM_T6086H | L6079V_W6081I |
| D249 | A6085YW_T6086H | L6079V_W6081I |
| D250 | A6085YH_T6086H | L6079V_W6081I |
| D251 | A6085YF_T6086Y | L6079T_W6081T |
| D252 | A6085YY_T6086Y | L6079T_W6081T |
| D253 | A6085YM_T6086Y | L6079T_W6081T |
| D254 | A6085YW_T6086Y | L6079T_W6081T |
| D255 | A6085YH_T6086Y | L6079T_W6081T |
| D256 | A6085YF_T6086F | L6079T_W6081T |
| D257 | A6085YY_T6086F | L6079T_W6081T |
| D258 | A6085YM_T6086F | L6079T_W6081T |
| D259 | A6085YW_T6086F | L6079T_W6081T |
| D260 | A6085YH_T6086F | L6079T_W6081T |
| D261 | A6085YF_T6086M | L6079T_W6081T |
| D262 | A6085YY_T6086M | L6079T_W6081T |
| D263 | A6085YM_T6086M | L6079T_W6081T |
| D264 | A6085YW_T6086M | L6079T_W6081T |
| D265 | A6085YH_T6086M | L6079T_W6081T |
| D266 | A6085YF_T6086W | L6079T_W6081T |
| D267 | A6085YY_T6086W | L6079T_W6081T |
| D268 | A6085YM_T6086W | L6079T_W6081T |
| D269 | A6085YW_T6086W | L6079T_W6081T |
| D270 | A6085YH_T6086W | L6079T_W6081T |
| D271 | A6085YF_T6086H | L6079T_W6081T |
| D272 | A6085YY_T6086H | L6079T_W6081T |
| D273 | A6085YM_T6086H | L6079T_W6081T |
| D274 | A6085YW_T6086H | L6079T_W6081T |
| D275 | A6085YH_T6086H | L6079T_W6081T |
| D276 | A6085YF_T6086Y | L6079T_W6081L |
| D277 | A6085YY_T6086Y | L6079T_W6081L |
| D278 | A6085YM_T6086Y | L6079T_W6081L |
| D279 | A6085YW_T6086Y | L6079T_W6081L |
| D280 | A6085YH_T6086Y | L6079T_W6081L |
| D281 | A6085YF_T6086F | L6079T_W6081L |
| D282 | A6085YY_T6086F | L6079T_W6081L |
| D283 | A6085YM_T6086F | L6079T_W6081L |
| D284 | A6085YW_T6086F | L6079T_W6081L |
| D285 | A6085YH_T6086F | L6079T_W6081L |
| D286 | A6085YF_T6086M | L6079T_W6081L |
| D287 | A6085YY_T6086M | L6079T_W6081L |
| D288 | A6085YM_T6086M | L6079T_W6081L |
| D289 | A6085YW_T6086M | L6079T_W6081L |
| D290 | A6085YH_T6086M | L6079T_W6081L |
| D291 | A6085YF_T6086W | L6079T_W6081L |
| D292 | A6085YY_T6086W | L6079T_W6081L |
| D293 | A6085YM_T6086W | L6079T_W6081L |
| D294 | A6085YW_T6086W | L6079T_W6081L |
| D295 | A6085YH_T6086W | L6079T_W6081L |
| D296 | A6085YF_T6086H | L6079T_W6081L |
| D297 | A6085YY_T6086H | L6079T_W6081L |
| D298 | A6085YM_T6086H | L6079T_W6081L |
| D299 | A6085YW_T6086H | L6079T_W6081L |
| D300 | A6085YH_T6086H | L6079T_W6081L |
| D301 | A6085YF_T6086Y | L6079T_W6081A |
| D302 | A6085YY_T6086Y | L6079T_W6081A |
| D303 | A6085YM_T6086Y | L6079T_W6081A |
| D304 | A6085YW_T6086Y | L6079T_W6081A |
| D305 | A6085YH_T6086Y | L6079T_W6081A |
| D306 | A6085YF_T6086F | L6079T_W6081A |
| D307 | A6085YY_T6086F | L6079T_W6081A |
| D308 | A6085YM_T6086F | L6079T_W6081A |
| D309 | A6085YW_T6086F | L6079T_W6081A |
| D310 | A6085YH_T6086F | L6079T_W6081A |
| D311 | A6085YF_T6086M | L6079T_W6081A |
| D312 | A6085YY_T6086M | L6079T_W6081A |
| D313 | A6085YM_T6086M | L6079T_W6081A |
| D314 | A6085YW_T6086M | L6079T_W6081A |
| D315 | A6085YH_T6086M | L6079T_W6081A |
| D316 | A6085YF_T6086W | L6079T_W6081A |
| D317 | A6085YY_T6086W | L6079T_W6081A |
| D318 | A6085YM_T6086W | L6079T_W6081A |
| D319 | A6085YW_T6086W | L6079T_W6081A |
| D320 | A6085YH_T6086W | L6079T_W6081A |
| D321 | A6085YF_T6086H | L6079T_W6081A |
| D322 | A6085YY_T6086H | L6079T_W6081A |
| D323 | A6085YM_T6086H | L6079T_W6081A |
| D324 | A6085YW_T6086H | L6079T_W6081A |
| D325 | A6085YH_T6086H | L6079T_W6081A |
| D326 | A6085YF_T6086Y | L6079T_W6081V |
| D327 | A6085YY_T6086Y | L6079T_W6081V |
| D328 | A6085YM_T6086Y | L6079T_W6081V |
| D329 | A6085YW_T6086Y | L6079T_W6081V |
| D330 | A6085YH_T6086Y | L6079T_W6081V |
| D331 | A6085YF_T6086F | L6079T_W6081V |
| D332 | A6085YY_T6086F | L6079T_W6081V |
| D333 | A6085YM_T6086F | L6079T_W6081V |
| D334 | A6085YW_T6086F | L6079T_W6081V |
| D335 | A6085YH_T6086F | L6079T_W6081V |
| D336 | A6085YF_T6086M | L6079T_W6081V |
| D337 | A6085YY_T6086M | L6079T_W6081V |
| D338 | A6085YM_T6086M | L6079T_W6081V |
| D339 | A6085YW_T6086M | L6079T_W6081V |
| D340 | A6085YH_T6086M | L6079T_W6081V |
| D341 | A6085YF_T6086W | L6079T_W6081V |
| D342 | A6085YY_T6086W | L6079T_W6081V |
| D343 | A6085YM_T6086W | L6079T_W6081V |
| D344 | A6085YW_T6086W | L6079T_W6081V |
| D345 | A6085YH_T6086W | L6079T_W6081V |
| D346 | A6085YF_T6086H | L6079T_W6081V |
| D347 | A6085YY_T6086H | L6079T_W6081V |
| D348 | A6085YM_T6086H | L6079T_W6081V |
| D349 | A6085YW_T6086H | L6079T_W6081V |
| D350 | A6085YH_T6086H | L6079T_W6081V |
| D351 | A6085YF_T6086Y | L6079T_W6081I |
| D352 | A6085YY_T6086Y | L6079T_W6081I |
| D353 | A6085YM_T6086Y | L6079T_W6081I |
| D354 | A6085YW_T6086Y | L6079T_W6081I |
| D355 | A6085YH_T6086Y | L6079T_W6081I |
| D356 | A6085YF_T6086F | L6079T_W6081I |
| D357 | A6085YY_T6086F | L6079T_W6081I |
| D358 | A6085YM_T6086F | L6079T_W6081I |
| D359 | A6085YW_T6086F | L6079T_W6081I |
| D360 | A6085YH_T6086F | L6079T_W6081I |
| D361 | A6085YF_T6086M | L6079T_W6081I |
| D362 | A6085YY_T6086M | L6079T_W6081I |
| D363 | A6085YM_T6086M | L6079T_W6081I |
| D364 | A6085YW_T6086M | L6079T_W6081I |
| D365 | A6085YH_T6086M | L6079T_W6081I |
| D366 | A6085YF_T6086W | L6079T_W6081I |
| D367 | A6085YY_T6086W | L6079T_W6081I |
| D368 | A6085YM_T6086W | L6079T_W6081I |
| D369 | A6085YW_T6086W | L6079T_W6081I |
| D370 | A6085YH_T6086W | L6079T_W6081I |
| D371 | A6085YF_T6086H | L6079T_W6081I |
| D372 | A6085YY_T6086H | L6079T_W6081I |
| D373 | A6085YM_T6086H | L6079T_W6081I |
| D374 | A6085YW_T6086H | L6079T_W6081I |
| D375 | A6085YH_T6086H | L6079T_W6081I |
| D376 | A6085YF_T6086Y | L6079A_W6081T |
| D377 | A6085YY_T6086Y | L6079A_W6081T |
| D378 | A6085YM_T6086Y | L6079A_W6081T |
| D379 | A6085YW_T6086Y | L6079A_W6081T |
| D380 | A6085YH_T6086Y | L6079A_W6081T |
| D381 | A6085YF_T6086F | L6079A_W6081T |
| D382 | A6085YY_T6086F | L6079A_W6081T |
| D383 | A6085YM_T6086F | L6079A_W6081T |
| D384 | A6085YW_T6086F | L6079A_W6081T |
| D385 | A6085YH_T6086F | L6079A_W6081T |
| D386 | A6085YF_T6086M | L6079A_W6081T |
| D387 | A6085YY_T6086M | L6079A_W6081T |
| D388 | A6085YM_T6086M | L6079A_W6081T |
| D389 | A6085YW_T6086M | L6079A_W6081T |
| D390 | A6085YH_T6086M | L6079A_W6081T |
| D391 | A6085YF_T6086W | L6079A_W6081T |
| D392 | A6085YY_T6086W | L6079A_W6081T |
| D393 | A6085YM_T6086W | L6079A_W6081T |
| D394 | A6085YW_T6086W | L6079A_W6081T |
| D395 | A6085YH_T6086W | L6079A_W6081T |
| D396 | A6085YF_T6086H | L6079A_W6081T |
| D397 | A6085YY_T6086H | L6079A_W6081T |
| D398 | A6085YM_T6086H | L6079A_W6081T |
| D399 | A6085YW_T6086H | L6079A_W6081T |
| D400 | A6085YH_T6086H | L6079A_W6081T |
| D401 | A6085YF_T6086Y | L6079A_W6081L |
| D402 | A6085YY_T6086Y | L6079A_W6081L |
| D403 | A6085YM_T6086Y | L6079A_W6081L |
| D405 | A6085YH_T6086Y | L6079A_W6081L |
| D406 | A6085YF_T6086F | L6079A_W6081L |
| D407 | A6085YY_T6086F | L6079A_W6081L |
| D408 | A6085YM_T6086F | L6079A_W6081L |
| D409 | A6085YW_T6086F | L6079A_W6081L |
| D410 | A6085YH_T6086F | L6079A_W6081L |
| D411 | A6085YF_T6086M | L6079A_W6081L |
| D412 | A6085YY_T6086M | L6079A_W6081L |
| D413 | A6085YM_T6086M | L6079A_W6081L |
| D414 | A6085YW_T6086M | L6079A_W6081L |
| D415 | A6085YH_T6086M | L6079A_W6081L |
| D416 | A6085YF_T6086W | L6079A_W6081L |
| D417 | A6085YY_T6086W | L6079A_W6081L |
| D418 | A6085YM_T6086W | L6079A_W6081L |
| D419 | A6085YW_T6086W | L6079A_W6081L |
| D420 | A6085YH_T6086W | L6079A_W6081L |
| D421 | A6085YF_T6086H | L6079A_W6081L |
| D422 | A6085YY_T6086H | L6079A_W6081L |
| D423 | A6085YM_T6086H | L6079A_W6081L |
| D424 | A6085YW_T6086H | L6079A_W6081L |
| D425 | A6085YH_T6086H | L6079A_W6081L |
| D426 | A6085YF_T6086Y | L6079A_W6081A |
| D427 | A6085YY_T6086Y | L6079A_W6081A |
| D428 | A6085YM_T6086Y | L6079A_W6081A |
| D429 | A6085YW_T6086Y | L6079A_W6081A |
| D430 | A6085YH_T6086Y | L6079A_W6081A |
| D431 | A6085YF_T6086F | L6079A_W6081A |
| D432 | A6085YY_T6086F | L6079A_W6081A |
| D433 | A6085YM_T6086F | L6079A_W6081A |
| D434 | A6085YW_T6086F | L6079A_W6081A |
| D435 | A6085YH_T6086F | L6079A_W6081A |
| D436 | A6085YF_T6086M | L6079A_W6081A |
| D437 | A6085YY_T6086M | L6079A_W6081A |
| D438 | A6085YM_T6086M | L6079A_W6081A |
| D439 | A6085YW_T6086M | L6079A_W6081A |
| D440 | A6085YH_T6086M | L6079A_W6081A |
| D441 | A6085YF_T6086W | L6079A_W6081A |
| D442 | A6085YY_T6086W | L6079A_W6081A |
| D443 | A6085YM_T6086W | L6079A_W6081A |
| D444 | A6085YW_T6086W | L6079A_W6081A |
| D445 | A6085YH_T6086W | L6079A_W6081A |
| D446 | A6085YF_T6086H | L6079A_W6081A |
| D447 | A6085YY_T6086H | L6079A_W6081A |
| D448 | A6085YM_T6086H | L6079A_W6081A |
| D449 | A6085YW_T6086H | L6079A_W6081A |
| D450 | A6085YH_T6086H | L6079A_W6081A |
| D451 | A6085YF_T6086Y | L6079A_W6081V |
| D452 | A6085YY_T6086Y | L6079A_W6081V |
| D453 | A6085YM_T6086Y | L6079A_W6081V |
| D454 | A6085YW_T6086Y | L6079A_W6081V |
| D455 | A6085YH_T6086Y | L6079A_W6081V |
| D456 | A6085YF_T6086F | L6079A_W6081V |
| D457 | A6085YY_T6086F | L6079A_W6081V |
| D458 | A6085YM_T6086F | L6079A_W6081V |
| D459 | A6085YW_T6086F | L6079A_W6081V |
| D460 | A6085YH_T6086F | L6079A_W6081V |
| D461 | A6085YF_T6086M | L6079A_W6081V |
| D462 | A6085YY_T6086M | L6079A_W6081V |
| D463 | A6085YM_T6086M | L6079A_W6081V |
| D464 | A6085YW_T6086M | L6079A_W6081V |
| D465 | A6085YH_T6086M | L6079A_W6081V |
| D466 | A6085YF_T6086W | L6079A_W6081V |
| D467 | A6085YY_T6086W | L6079A_W6081V |
| D468 | A6085YM_T6086W | L6079A_W6081V |
| D469 | A6085YW_T6086W | L6079A_W6081V |
| D470 | A6085YH_T6086W | L6079A_W6081V |
| D471 | A6085YF_T6086H | L6079A_W6081V |
| D472 | A6085YY_T6086H | L6079A_W6081V |
| D473 | A6085YM_T6086H | L6079A_W6081V |
| D474 | A6085YW_T6086H | L6079A_W6081V |
| D475 | A6085YH_T6086H | L6079A_W6081V |
| D476 | A6085YF_T6086Y | L6079A_W6081I |
| D477 | A6085YY_T6086Y | L6079A_W6081I |
| D478 | A6085YM_T6086Y | L6079A_W6081I |
| D479 | A6085YW_T6086Y | L6079A_W6081I |
| D480 | A6085YH_T6086Y | L6079A_W6081I |
| D481 | A6085YF_T6086F | L6079A_W6081I |
| D482 | A6085YY_T6086F | L6079A_W6081I |
| D483 | A6085YM_T6086F | L6079A_W6081I |
| D484 | A6085YW_T6086F | L6079A_W6081I |
| D485 | A6085YH_T6086F | L6079A_W6081I |
| D486 | A6085YF_T6086M | L6079A_W6081I |
| D487 | A6085YY_T6086M | L6079A_W6081I |
| D488 | A6085YM_T6086M | L6079A_W6081I |
| D489 | A6085YW_T6086M | L6079A_W6081I |
| D490 | A6085YH_T6086M | L6079A_W6081I |
| D491 | A6085YF_T6086W | L6079A_W6081I |
| D492 | A6085YY_T6086W | L6079A_W6081I |
| D493 | A6085YM_T6086W | L6079A_W6081I |
| D494 | A6085YW_T6086W | L6079A_W6081I |
| D495 | A6085YH_T6086W | L6079A_W6081I |
| D496 | A6085YF_T6086H | L6079A_W6081I |
| D497 | A6085YY_T6086H | L6079A_W6081I |
| D498 | A6085YM_T6086H | L6079A_W6081I |
| D499 | A6085YW_T6086H | L6079A_W6081I |
| D500 | A6085YH_T6086H | L6079A_W6081I |
| D501 | A6085YF_T6086Y | L6079I_W6081T |
| D502 | A6085YY_T6086Y | L6079I_W6081T |
| D503 | A6085YM_T6086Y | L6079I_W6081T |
| D504 | A6085YW_T6086Y | L6079I_W6081T |
| D505 | A6085YH_T6086Y | L6079I_W6081T |
| D506 | A6085YF_T6086F | L6079I_W6081T |
| D507 | A6085YY_T6086F | L6079I_W6081T |
| D508 | A6085YM_T6086F | L6079I_W6081T |
| D509 | A6085YW_T6086F | L6079I_W6081T |
| D510 | A6085YH_T6086F | L6079I_W6081T |
| D511 | A6085YF_T6086M | L6079I_W6081T |
| D512 | A6085YY_T6086M | L6079I_W6081T |
| D513 | A6085YM_T6086M | L6079I_W6081T |
| D514 | A6085YW_T6086M | L6079I_W6081T |
| D515 | A6085YH_T6086M | L6079I_W6081T |
| D516 | A6085YF_T6086W | L6079I_W6081T |
| D517 | A6085YY_T6086W | L6079I_W6081T |
| D518 | A6085YM_T6086W | L6079I_W6081T |
| D519 | A6085YW_T6086W | L6079I_W6081T |
| D520 | A6085YH_T6086W | L6079I_W6081T |
| D521 | A6085YF_T6086H | L6079I_W6081T |
| D522 | A6085YY_T6086H | L6079I_W6081T |
| D523 | A6085YM_T6086H | L6079I_W6081T |
| D524 | A6085YW_T6086H | L6079I_W6081T |
| D525 | A6085YH_T6086H | L6079I_W6081T |
| D526 | A6085YF_T6086Y | L6079I_W6081L |
| D527 | A6085YY_T6086Y | L6079I_W6081L |
| D528 | A6085YM_T6086Y | L6079I_W6081L |
| D529 | A6085YW_T6086Y | L6079I_W6081L |
| D530 | A6085YH_T6086Y | L6079I_W6081L |
| D531 | A6085YF_T6086F | L6079I_W6081L |
| D532 | A6085YY_T6086F | L6079I_W6081L |
| D533 | A6085YM_T6086F | L6079I_W6081L |
| D534 | A6085YW_T6086F | L6079I_W6081L |
| D535 | A6085YH_T6086F | L6079I_W6081L |
| D536 | A6085YF_T6086M | L6079I_W6081L |
| D537 | A6085YY_T6086M | L6079I_W6081L |
| D538 | A6085YM_T6086M | L6079I_W6081L |
| D539 | A6085YW_T6086M | L6079I_W6081L |
| D540 | A6085YH_T6086M | L6079I_W6081L |
| D541 | A6085YF_T6086W | L6079I_W6081L |
| D542 | A6085YY_T6086W | L6079I_W6081L |
| D543 | A6085YM_T6086W | L6079I_W6081L |
| D544 | A6085YW_T6086W | L6079I_W6081L |
| D545 | A6085YH_T6086W | L6079I_W6081L |
| D546 | A6085YF_T6086H | L6079I_W6081L |
| D547 | A6085YY_T6086H | L6079I_W6081L |
| D548 | A6085YM_T6086H | L6079I_W6081L |
| D549 | A6085YW_T6086H | L6079I_W6081L |
| D550 | A6085YH_T6086H | L6079I_W6081L |
| D551 | A6085YF_T6086Y | L6079I_W6081A |
| D552 | A6085YY_T6086Y | L6079I_W6081A |
| D553 | A6085YM_T6086Y | L6079I_W6081A |
| D554 | A6085YW_T6086Y | L6079I_W6081A |
| D555 | A6085YH_T6086Y | L6079I_W6081A |
| D556 | A6085YF_T6086F | L6079I_W6081A |
| D557 | A6085YY_T6086F | L6079I_W6081A |
| D558 | A6085YM_T6086F | L6079I_W6081A |
| D559 | A6085YW_T6086F | L6079I_W6081A |
| D560 | A6085YH_T6086F | L6079I_W6081A |
| D561 | A6085YF_T6086M | L6079I_W6081A |
| D562 | A6085YY_T6086M | L6079I_W6081A |
| D563 | A6085YM_T6086M | L6079I_W6081A |
| D564 | A6085YW_T6086M | L6079I_W6081A |
| D565 | A6085YH_T6086M | L6079I_W6081A |
| D566 | A6085YF_T6086W | L6079I_W6081A |
| D567 | A6085YY_T6086W | L6079I_W6081A |
| D568 | A6085YM_T6086W | L6079I_W6081A |
| D569 | A6085YW_T6086W | L6079I_W6081A |
| D570 | A6085YH_T6086W | L6079I_W6081A |
| D571 | A6085YF_T6086H | L6079I_W6081A |
| D572 | A6085YY_T6086H | L6079I_W6081A |
| D573 | A6085YM_T6086H | L6079I_W6081A |
| D574 | A6085YW_T6086H | L6079I_W6081A |
| D575 | A6085YH_T6086H | L6079I_W6081A |
| D576 | A6085YF_T6086Y | L6079I_W6081V |
| D577 | A6085YY_T6086Y | L6079I_W6081V |
| D578 | A6085YM_T6086Y | L6079I_W6081V |
| D579 | A6085YW_T6086Y | L6079I_W6081V |
| D580 | A6085YH_T6086Y | L6079I_W6081V |
| D581 | A6085YF_T6086F | L6079I_W6081V |
| D582 | A6085YY_T6086F | L6079I_W6081V |
| D583 | A6085YM_T6086F | L6079I_W6081V |
| D584 | A6085YW_T6086F | L6079I_W6081V |
| D585 | A6085YH_T6086F | L6079I_W6081V |
| D586 | A6085YF_T6086M | L6079I_W6081V |
| D587 | A6085YY_T6086M | L6079I_W6081V |
| D588 | A6085YM_T6086M | L6079I_W6081V |
| D589 | A6085YW_T6086M | L6079I_W6081V |
| D590 | A6085YH_T6086M | L6079I_W6081V |
| D591 | A6085YF_T6086W | L6079I_W6081V |
| D592 | A6085YY_T6086W | L6079I_W6081V |
| D593 | A6085YM_T6086W | L6079I_W6081V |
| D594 | A6085YW_T6086W | L6079I_W6081V |
| D595 | A6085YH_T6086W | L6079I_W6081V |
| D596 | A6085YF_T6086H | L6079I_W6081V |
| D597 | A6085YY_T6086H | L6079I_W6081V |
| D598 | A6085YM_T6086H | L6079I_W6081V |
| D599 | A6085YW_T6086H | L6079I_W6081V |
| D600 | A6085YH_T6086H | L6079I_W6081V |
| D601 | A6085YF_T6086Y | L6079I_W6081I |
| D602 | A6085YY_T6086Y | L6079I_W6081I |
| D603 | A6085YM_T6086Y | L6079I_W6081I |
| D604 | A6085YW_T6086Y | L6079I_W6081I |
| D605 | A6085YH_T6086Y | L6079I_W6081I |
| D606 | A6085YF_T6086F | L6079I_W6081I |
| D607 | A6085YY_T6086F | L6079I_W6081I |
| D608 | A6085YM_T6086F | L6079I_W6081I |
| D609 | A6085YW_T6086F | L6079I_W6081I |
| D610 | A6085YH_T6086F | L6079I_W6081I |
| D611 | A6085YF_T6086M | L6079I_W6081I |
| D612 | A6085YY_T6086M | L6079I_W6081I |
| D613 | A6085YM_T6086M | L6079I_W6081I |
| D614 | A6085YW_T6086M | L6079I_W6081I |
| D615 | A6085YH_T6086M | L6079I_W6081I |
| D616 | A6085YF_T6086W | L6079I_W6081I |
| D617 | A6085YY_T6086W | L6079I_W6081I |
| D618 | A6085YM_T6086W | L6079I_W6081I |
| D619 | A6085YW_T6086W | L6079I_W6081I |
| D620 | A6085YH_T6086W | L6079I_W6081I |
| D621 | A6085YF_T6086H | L6079I_W6081I |
| D622 | A6085YY_T6086H | L6079I_W6081I |
| D623 | A6085YM_T6086H | L6079I_W6081I |
| D624 | A6085YW_T6086H | L6079I_W6081I |
| D625 | A6085YH_T6086H | L6079I_W6081I |

TABLE 9
IgA HetFc Designs comprising Core Mutations in Combination
with Mutation at Position 6088 in Chain B

CH3 Domain Mutations

| No. | Chain A | Chain B |
|---|---|---|
| D626 | A6085YF_T6086Y | W6081T_I6088L |
| D627 | A6085YY_T6086Y | W6081T_I6088L |
| D628 | A6085YM_T6086Y | W6081T_I6088L |
| D629 | A6085YW_T6086Y | W6081T_I6088L |
| D630 | A6085YH_T6086Y | W6081T_I6088L |
| D631 | A6085YF_T6086F | W6081T_I6088L |
| D632 | A6085YY_T6086F | W6081T_I6088L |
| D633 | A6085YM_T6086F | W6081T_I6088L |
| D634 | A6085YW_T6086F | W6081T_I6088L |
| D635 | A6085YH_T6086F | W6081T_I6088L |
| D636 | A6085YF_T6086M | W6081T_I6088L |
| D637 | A6085YY_T6086M | W6081T_I6088L |
| D638 | A6085YM_T6086M | W6081T_I6088L |
| D639 | A6085YW_T6086M | W6081T_I6088L |
| D640 | A6085YH_T6086M | W6081T_I6088L |
| D641 | A6085YF_T6086W | W6081T_I6088L |
| D642 | A6085YY_T6086W | W6081T_I6088L |
| D643 | A6085YM_T6086W | W6081T_I6088L |
| D644 | A6085YW_T6086W | W6081T_I6088L |
| D645 | A6085YH_T6086W | W6081T_I6088L |
| D646 | A6085YF_T6086H | W6081T_I6088L |
| D647 | A6085YY_T6086H | W6081T_I6088L |
| D648 | A6085YM_T6086H | W6081T_I6088L |
| D649 | A6085YW_T6086H | W6081T_I6088L |
| D650 | A6085YH_T6086H | W6081T_I6088L |
| D651 | A6085YF_T6086Y | W6081L_I6088L |
| D652 | A6085YY_T6086Y | W6081L_I6088L |
| D653 | A6085YM_T6086Y | W6081L_I6088L |
| D654 | A6085YW_T6086Y | W6081L_I6088L |
| D655 | A6085YH_T6086Y | W6081L_I6088L |
| D656 | A6085YF_T6086F | W6081L_I6088L |
| D657 | A6085YY_T6086F | W6081L_I6088L |
| D658 | A6085YM_T6086F | W6081L_I6088L |
| D659 | A6085YW_T6086F | W6081L_I6088L |
| D660 | A6085YH_T6086F | W6081L_I6088L |
| D661 | A6085YF_T6086M | W6081L_I6088L |
| D662 | A6085YY_T6086M | W6081L_I6088L |
| D663 | A6085YM_T6086M | W6081L_I6088L |
| D664 | A6085YW_T6086M | W6081L_I6088L |
| D665 | A6085YH_T6086M | W6081L_I6088L |
| D666 | A6085YF_T6086W | W6081L_I6088L |
| D667 | A6085YY_T6086W | W6081L_I6088L |
| D668 | A6085YM_T6086W | W6081L_I6088L |
| D669 | A6085YW_T6086W | W6081L_I6088L |
| D670 | A6085YH_T6086W | W6081L_I6088L |
| D671 | A6085YF_T6086H | W6081L_I6088L |
| D672 | A6085YY_T6086H | W6081L_I6088L |
| D673 | A6085YM_T6086H | W6081L_I6088L |
| D674 | A6085YW_T6086H | W6081L_I6088L |
| D675 | A6085YH_T6086H | W6081L_I6088L |
| D676 | A6085YF_T6086Y | W6081A_I6088L |
| D677 | A6085YY_T6086Y | W6081A_I6088L |
| D678 | A6085YM_T6086Y | W6081A_I6088L |
| D679 | A6085YW_T6086Y | W6081A_I6088L |
| D680 | A6085YH_T6086Y | W6081A_I6088L |
| D681 | A6085YF_T6086F | W6081A_I6088L |
| D682 | A6085YY_T6086F | W6081A_I6088L |
| D683 | A6085YM_T6086F | W6081A_I6088L |
| D684 | A6085YW_T6086F | W6081A_I6088L |
| D685 | A6085YH_T6086F | W6081A_I6088L |
| D686 | A6085YF_T6086M | W6081A_I6088L |
| D687 | A6085YY_T6086M | W6081A_I6088L |
| D688 | A6085YM_T6086M | W6081A_I6088L |
| D689 | A6085YW_T6086M | W6081A_I6088L |
| D690 | A6085YH_T6086M | W6081A_I6088L |
| D691 | A6085YF_T6086W | W6081A_I6088L |
| D692 | A6085YY_T6086W | W6081A_I6088L |
| D693 | A6085YM_T6086W | W6081A_I6088L |
| D694 | A6085YW_T6086W | W6081A_I6088L |
| D695 | A6085YH_T6086W | W6081A_I6088L |
| D696 | A6085YF_T6086H | W6081A_I6088L |
| D697 | A6085YY_T6086H | W6081A_I6088L |
| D698 | A6085YM_T6086H | W6081A_I6088L |
| D699 | A6085YW_T6086H | W6081A_I6088L |
| D700 | A6085YH_T6086H | W6081A_I6088L |
| D701 | A6085YF_T6086Y | W6081V_I6088L |
| D702 | A6085YY_T6086Y | W6081V_I6088L |
| D703 | A6085YM_T6086Y | W6081V_I6088L |
| D704 | A6085YW_T6086Y | W6081V_I6088L |
| D705 | A6085YH_T6086Y | W6081V_I6088L |
| D706 | A6085YF_T6086F | W6081V_I6088L |
| D707 | A6085YY_T6086F | W6081V_I6088L |
| D708 | A6085YM_T6086F | W6081V_I6088L |
| D709 | A6085YW_T6086F | W6081V_I6088L |
| D710 | A6085YH_T6086F | W6081V_I6088L |
| D711 | A6085YF_T6086M | W6081V_16088L |
| D712 | A6085YY_T6086M | W6081V_I6088L |
| D713 | A6085YM_T6086M | W6081V_I6088L |
| D714 | A6085YW_T6086M | W6081V_I6088L |
| D715 | A6085YH_T6086M | W6081V_I6088L |
| D716 | A6085YF_T6086W | W6081V_I6088L |
| D717 | A6085YY_T6086W | W6081V_I6088L |
| D718 | A6085YM_T6086W | W6081V_I6088L |
| D719 | A6085YW_T6086W | W6081V_I6088L |
| D720 | A6085YH_T6086W | W6081V_I6088L |
| D721 | A6085YF_T6086H | W6081V_I6088L |
| D722 | A6085YY_T6086H | W6081V_I6088L |
| D723 | A6085YM_T6086H | W6081V_I6088L |
| D724 | A6085YW_T6086H | W6081V_I6088L |
| D725 | A6085YH_T6086H | W6081V_I6088L |
| D726 | A6085YF_T6086Y | W6081I_I6088L |
| D727 | A6085YY_T6086Y | W6081I_I6088L |
| D728 | A6085YM_T6086Y | W6081I_I6088L |
| D729 | A6085YW_T6086Y | W6081I_I6088L |
| D730 | A6085YH_T6086Y | W6081I_I6088L |
| D731 | A6085YF_T6086F | W6081I_I6088L |
| D732 | A6085YY_T6086F | W6081I_I6088L |
| D733 | A6085YM_T6086F | W6081I_I6088L |
| D734 | A6085YW_T6086F | W6081I_I6088L |
| D735 | A6085YH_T6086F | W6081I_I6088L |
| D736 | A6085YF_T6086M | W6081I_I6088L |
| D737 | A6085YY_T6086M | W6081I_I6088L |
| D738 | A6085YM_T6086M | W6081I_I6088L |
| D739 | A6085YW_T6086M | W6081I_I6088L |
| D740 | A6085YH_T6086M | W6081I_I6088L |
| D741 | A6085YF_T6086W | W6081I_I6088L |
| D742 | A6085YY_T6086W | W6081I_I6088L |
| D743 | A6085YM_T6086W | W6081I_I6088L |
| D744 | A6085YW_T6086W | W6081I_I6088L |
| D745 | A6085YH_T6086W | W6081I_I6088L |
| D746 | A6085YF_T6086H | W6081I_I6088L |
| D747 | A6085YY_T6086H | W6081I_I6088L |
| D748 | A6085YM_T6086H | W6081I_I6088L |
| D749 | A6085YW_T6086H | W6081I_I6088L |
| D750 | A6085YH_T6086H | W6081I_I6088L |
| D751 | A6085YF_T6086Y | W6081T_I6088A |
| D752 | A6085YY_T6086Y | W6081T_I6088A |
| D753 | A6085YM_T6086Y | W6081T_I6088A |
| D754 | A6085YW_T6086Y | W6081T_I6088A |
| D755 | A6085YH_T6086Y | W6081T_I6088A |
| D756 | A6085YF_T6086F | W6081T_I6088A |
| D757 | A6085YY_T6086F | W6081T_I6088A |
| D758 | A6085YM_T6086F | W6081T_I6088A |
| D759 | A6085YW_T6086F | W6081T_I6088A |
| D760 | A6085YH_T6086F | W6081T_I6088A |
| D761 | A6085YF_T6086M | W6081T_I6088A |
| D762 | A6085YY_T6086M | W6081T_I6088A |
| D763 | A6085YM_T6086M | W6081T_I6088A |
| D764 | A6085YW_T6086M | W6081T_I6088A |
| D765 | A6085YH_T6086M | W6081T_I6088A |
| D766 | A6085YF_T6086W | W6081T_I6088A |
| D767 | A6085YY_T6086W | W6081T_I6088A |
| D768 | A6085YM_T6086W | W6081T_I6088A |
| D769 | A6085YW_T6086W | W6081T_I6088A |
| D770 | A6085YH_T6086W | W6081T_I6088A |
| D771 | A6085YF_T6086H | W6081T_I6088A |
| D772 | A6085YY_T6086H | W6081T_I6088A |
| D773 | A6085YM_T6086H | W6081T_I6088A |
| D774 | A6085YW_T6086H | W6081T_I6088A |
| D775 | A6085YH_T6086H | W6081T_I6088A |
| D776 | A6085YF_T6086Y | W6081L_I6088A |
| D777 | A6085YY_T6086Y | W6081L_I6088A |
| D778 | A6085YM_T6086Y | W6081L_I6088A |
| D779 | A6085YW_T6086Y | W6081L_I6088A |
| D780 | A6085YH_T6086Y | W6081L_I6088A |
| D781 | A6085YF_T6086F | W6081L_I6088A |
| D782 | A6085YY_T6086F | W6081L_I6088A |
| D783 | A6085YM_T6086F | W6081L_I6088A |
| D784 | A6085YW_T6086F | W6081L_I6088A |
| D785 | A6085YH_T6086F | W6081L_I6088A |
| D786 | A6085YF_T6086M | W6081L_I6088A |
| D787 | A6085YY_T6086M | W6081L_I6088A |
| D788 | A6085YM_T6086M | W6081L_I6088A |
| D789 | A6085YW_T6086M | W6081L_I6088A |
| D790 | A6085YH_T6086M | W6081L_I6088A |
| D791 | A6085YF_T6086W | W6081L_I6088A |
| D792 | A6085YY_T6086W | W6081L_I6088A |
| D793 | A6085YM_T6086W | W6081L_I6088A |
| D794 | A6085YW_T6086W | W6081L_I6088A |
| D795 | A6085YH_T6086W | W6081L_I6088A |
| D796 | A6085YF_T6086H | W6081L_I6088A |
| D797 | A6085YY_T6086H | W6081L_I6088A |
| D798 | A6085YM_T6086H | W6081L_I6088A |
| D799 | A6085YW_T6086H | W6081L_I6088A |
| D800 | A6085YH_T6086H | W6081L_I6088A |
| D801 | A6085YF_T6086Y | W6081A_I6088A |
| D802 | A6085YY_T6086Y | W6081A_I6088A |
| D803 | A6085YM_T6086Y | W6081A_I6088A |
| D804 | A6085YW_T6086Y | W6081A_I6088A |
| D805 | A6085YH_T6086Y | W6081A_I6088A |
| D806 | A6085YF_T6086F | W6081A_I6088A |
| D807 | A6085YY_T6086F | W6081A_I6088A |
| D808 | A6085YM_T6086F | W6081A_I6088A |
| D809 | A6085YW_T6086F | W6081A_I6088A |
| D810 | A6085YH_T6086F | W6081A_I6088A |
| D811 | A6085YF_T6086M | W6081A_I6088A |
| D812 | A6085YY_T6086M | W6081A_I6088A |
| D813 | A6085YM_T6086M | W6081A_I6088A |
| D814 | A6085YW_T6086M | W6081A_I6088A |
| D815 | A6085YH_T6086M | W6081A_I6088A |
| D816 | A6085YF_T6086W | W6081A_I6088A |
| D817 | A6085YY_T6086W | W6081A_I6088A |
| D818 | A6085YM_T6086W | W6081A_I6088A |
| D819 | A6085YW_T6086W | W6081A_I6088A |
| D820 | A6085YH_T6086W | W6081A_I6088A |
| D821 | A6085YF_T6086H | W6081A_I6088A |
| D822 | A6085YY_T6086H | W6081A_I6088A |
| D823 | A6085YM_T6086H | W6081A_I6088A |
| D824 | A6085YW_T6086H | W6081A_I6088A |
| D825 | A6085YH_T6086H | W6081A_I6088A |
| D826 | A6085YF_T6086Y | W6081V_I6088A |
| D827 | A6085YY_T6086Y | W6081V_I6088A |
| D828 | A6085YM_T6086Y | W6081V_I6088A |
| D829 | A6085YW_T6086Y | W6081V_I6088A |
| D830 | A6085YH_T6086Y | W6081V_I6088A |
| D831 | A6085YF_T6086F | W6081V_I6088A |
| D832 | A6085YY_T6086F | W6081V_I6088A |
| D833 | A6085YM_T6086F | W6081V_I6088A |
| D834 | A6085YW_T6086F | W6081V_I6088A |
| D835 | A6085YH_T6086F | W6081V_I6088A |
| D836 | A6085YF_T6086M | W6081V_I6088A |
| D837 | A6085YY_T6086M | W6081V_I6088A |
| D838 | A6085YM_T6086M | W6081V_I6088A |
| D839 | A6085YW_T6086M | W6081V_I6088A |
| D840 | A6085YH_T6086M | W6081V_I6088A |
| D841 | A6085YF_T6086W | W6081V_I6088A |
| D842 | A6085YY_T6086W | W6081V_I6088A |
| D843 | A6085YM_T6086W | W6081V_I6088A |
| D844 | A6085YW_T6086W | W6081V_I6088A |
| D845 | A6085YH_T6086W | W6081V_I6088A |
| D846 | A6085YF_T6086H | W6081V_I6088A |
| D847 | A6085YY_T6086H | W6081V_I6088A |
| D848 | A6085YM_T6086H | W6081V_I6088A |
| D849 | A6085YW_T6086H | W6081V_I6088A |
| D850 | A6085YH_T6086H | W6081V_I6088A |
| D851 | A6085YF_T6086Y | W6081I_I6088A |
| D852 | A6085YY_T6086Y | W6081I_I6088A |
| D853 | A6085YM_T6086Y | W6081I_I6088A |
| D854 | A6085YW_T6086Y | W6081I_I6088A |
| D855 | A6085YH_T6086Y | W6081I_I6088A |
| D856 | A6085YF_T6086F | W6081I_I6088A |
| D857 | A6085YY_T6086F | W6081I_I6088A |
| D858 | A6085YM_T6086F | W6081I_I6088A |
| D859 | A6085YW_T6086F | W6081I_I6088A |
| D860 | A6085YH_T6086F | W6081I_I6088A |
| D861 | A6085YF_T6086M | W6081I_I6088A |
| D862 | A6085YY_T6086M | W6081I_I6088A |
| D863 | A6085YM_T6086M | W6081I_I6088A |
| D864 | A6085YW_T6086M | W6081I_I6088A |
| D865 | A6085YH_T6086M | W6081I_I6088A |
| D866 | A6085YF_T6086W | W6081I_I6088A |
| D867 | A6085YY_T6086W | W6081I_I6088A |
| D868 | A6085YM_T6086W | W6081I_I6088A |
| D869 | A6085YW_T6086W | W6081I_I6088A |
| D870 | A6085YH_T6086W | W6081I_I6088A |
| D871 | A6085YF_T6086H | W6081I_I6088A |
| D872 | A6085YY_T6086H | W6081I_I6088A |
| D873 | A6085YM_T6086H | W6081I_I6088A |
| D874 | A6085YW_T6086H | W6081I_I6088A |
| D875 | A6085YH_T6086H | W6081I_I6088A |
| D876 | A6085YF_T6086Y | W6081T_I6088V |
| D877 | A6085YY_T6086Y | W6081T_I6088V |
| D878 | A6085YM_T6086Y | W6081T_I6088V |
| D879 | A6085YW_T6086Y | W6081T_I6088V |
| D880 | A6085YH_T6086Y | W6081T_I6088V |
| D881 | A6085YF_T6086F | W6081T_I6088V |
| D882 | A6085YY_T6086F | W6081T_I6088V |
| D883 | A6085YM_T6086F | W6081T_I6088V |
| D884 | A6085YW_T6086F | W6081T_I6088V |
| D885 | A6085YH_T6086F | W6081T_I6088V |
| D886 | A6085YF_T6086M | W6081T_I6088V |
| D887 | A6085YY_T6086M | W6081T_I6088V |
| D888 | A6085YM_T6086M | W6081T_I6088V |
| D889 | A6085YW_T6086M | W6081T_I6088V |
| D890 | A6085YH_T6086M | W6081T_I6088V |
| D891 | A6085YF_T6086W | W6081T_I6088V |
| D892 | A6085YY_T6086W | W6081T_I6088V |
| D893 | A6085YM_T6086W | W6081T_I6088V |
| D894 | A6085YW_T6086W | W6081T_I6088V |
| D895 | A6085YH_T6086W | W6081T_I6088V |
| D896 | A6085YF_T6086H | W6081T_I6088V |
| D898 | A6085YM_T6086H | W6081T_I6088V |
| D899 | A6085YW_T6086H | W6081T_I6088V |
| D900 | A6085YH_T6086H | W6081T_I6088V |
| D901 | A6085YF_T6086Y | W6081L_I6088V |
| D902 | A6085YY_T6086Y | W6081L_I6088V |
| D903 | A6085YM_T6086Y | W6081L_I6088V |
| D904 | A6085YW_T6086Y | W6081L_I6088V |
| D905 | A6085YH_T6086Y | W6081L_I6088V |
| D906 | A6085YF_T6086F | W6081L_I6088V |
| D907 | A6085YY_T6086F | W6081L_I6088V |
| D908 | A6085YM_T6086F | W6081L_I6088V |
| D909 | A6085YW_T6086F | W6081L_I6088V |
| D910 | A6085YH_T6086F | W6081L_I6088V |
| D911 | A6085YF_T6086M | W6081L_I6088V |
| D912 | A6085YY_T6086M | W6081L_I6088V |
| D913 | A6085YM_T6086M | W6081L_I6088V |
| D914 | A6085YW_T6086M | W6081L_I6088V |
| D915 | A6085YH_T6086M | W6081L_I6088V |
| D916 | A6085YF_T6086W | W6081L_I6088V |
| D917 | A6085YY_T6086W | W6081L_I6088V |
| D918 | A6085YM_T6086W | W6081L_I6088V |
| D919 | A6085YW_T6086W | W6081L_I6088V |
| D920 | A6085YH_T6086W | W6081L_I6088V |
| D921 | A6085YF_T6086H | W6081L_I6088V |
| D922 | A6085YY_T6086H | W6081L_I6088V |
| D923 | A6085YM_T6086H | W6081L_I6088V |
| D924 | A6085YW_T6086H | W6081L_I6088V |
| D925 | A6085YH_T6086H | W6081L_I6088V |
| D926 | A6085YF_T6086Y | W6081A_I6088V |
| D927 | A6085YY_T6086Y | W6081A_I6088V |
| D928 | A6085YM_T6086Y | W6081A_I6088V |
| D929 | A6085YW_T6086Y | W6081A_I6088V |
| D930 | A6085YH_T6086Y | W6081A_I6088V |
| D931 | A6085YF_T6086F | W6081A_I6088V |
| D932 | A6085YY_T6086F | W6081A_I6088V |
| D933 | A6085YM_T6086F | W6081A_I6088V |
| D934 | A6085YW_T6086F | W6081A_I6088V |
| D935 | A6085YH_T6086F | W6081A_I6088V |
| D936 | A6085YF_T6086M | W6081A_I6088V |
| D937 | A6085YY_T6086M | W6081A_I6088V |
| D938 | A6085YM_T6086M | W6081A_I6088V |
| D939 | A6085YW_T6086M | W6081A_I6088V |
| D940 | A6085YH_T6086M | W6081A_I6088V |
| D941 | A6085YF_T6086W | W6081A_I6088V |
| D942 | A6085YY_T6086W | W6081A_I6088V |
| D943 | A6085YM_T6086W | W6081A_I6088V |
| D944 | A6085YW_T6086W | W6081A_I6088V |
| D945 | A6085YH_T6086W | W6081A_I6088V |
| D946 | A6085YF_T6086H | W6081A_I6088V |
| D947 | A6085YY_T6086H | W6081A_I6088V |
| D948 | A6085YM_T6086H | W6081A_I6088V |
| D949 | A6085YW_T6086H | W6081A_I6088V |
| D950 | A6085YH_T6086H | W6081A_I6088V |
| D951 | A6085YF_T6086Y | W6081V_I6088V |
| D952 | A6085YY_T6086Y | W6081V_I6088V |
| D953 | A6085YM_T6086Y | W6081V_I6088V |
| D954 | A6085YW_T6086Y | W6081V_I6088V |
| D955 | A6085YH_T6086Y | W6081V_I6088V |
| D956 | A6085YF_T6086F | W6081V_I6088V |
| D957 | A6085YY_T6086F | W6081V_I6088V |
| D958 | A6085YM_T6086F | W6081V_I6088V |
| D959 | A6085YW_T6086F | W6081V_I6088V |
| D960 | A6085YH_T6086F | W6081V_I6088V |
| D961 | A6085YF_T6086M | W6081V_I6088V |
| D962 | A6085YY_T6086M | W6081V_I6088V |
| D963 | A6085YM_T6086M | W6081V_I6088V |
| D964 | A6085YW_T6086M | W6081V_I6088V |
| D965 | A6085YH_T6086M | W6081V_I6088V |
| D966 | A6085YF_T6086W | W6081V_I6088V |
| D967 | A6085YY_T6086W | W6081V_I6088V |
| D968 | A6085YM_T6086W | W6081V_I6088V |
| D969 | A6085YW_T6086W | W6081V_I6088V |
| D970 | A6085YH_T6086W | W6081V_I6088V |
| D971 | A6085YF_T6086H | W6081V_I6088V |
| D972 | A6085YY_T6086H | W6081V_I6088V |
| D973 | A6085YM_T6086H | W6081V_I6088V |
| D974 | A6085YW_T6086H | W6081V_I6088V |
| D975 | A6085YH_T6086H | W6081V_I6088V |
| D976 | A6085YF_T6086Y | W6081I_I6088V |
| D977 | A6085YY_T6086Y | W6081I_I6088V |
| D978 | A6085YM_T6086Y | W6081I_I6088V |
| D979 | A6085YW_T6086Y | W6081I_I6088V |
| D980 | A6085YH_T6086Y | W6081I_I6088V |
| D981 | A6085YF_T6086F | W6081I_I6088V |
| D982 | A6085YY_T6086F | W6081I_I6088V |
| D983 | A6085YM_T6086F | W6081I_I6088V |
| D984 | A6085YW_T6086F | W6081I_I6088V |
| D985 | A6085YH_T6086F | W6081I_I6088V |
| D986 | A6085YF_T6086M | W6081I_I6088V |
| D987 | A6085YY_T6086M | W6081I_I6088V |
| D988 | A6085YM_T6086M | W6081I_I6088V |
| D989 | A6085YW_T6086M | W6081I_I6088V |
| D990 | A6085YH_T6086M | W6081I_I6088V |
| D991 | A6085YF_T6086W | W6081I_I6088V |
| D992 | A6085YY_T6086W | W6081I_I6088V |
| D993 | A6085YM_T6086W | W6081I_I6088V |
| D994 | A6085YW_T6086W | W6081I_I6088V |
| D995 | A6085YH_T6086W | W6081I_I6088V |
| D996 | A6085YF_T6086H | W6081I_I6088V |
| D997 | A6085YY_T6086H | W6081I_I6088V |
| D998 | A6085YM_T6086H | W6081I_I6088V |
| D999 | A6085YW_T6086H | W6081I_I6088V |
| D1000 | A6085YH_T6086H | W6081I_I6088V |
| D1001 | A6085YF_T6086Y | W6081T_I6088T |
| D1002 | A6085YY_T6086Y | W6081T_I6088T |
| D1003 | A6085YM_T6086Y | W6081T_I6088T |
| D1004 | A6085YW_T6086Y | W6081T_I6088T |
| D1005 | A6085YH_T6086Y | W6081T_I6088T |
| D1006 | A6085YF_T6086F | W6081T_I6088T |
| D1007 | A6085YY_T6086F | W6081T_I6088T |
| D1008 | A6085YM_T6086F | W6081T_I6088T |
| D1009 | A6085YW_T6086F | W6081T_I6088T |
| D1010 | A6085YH_T6086F | W6081T_I6088T |
| D1011 | A6085YF_T6086M | W6081T_I6088T |
| D1012 | A6085YY_T6086M | W6081T_I6088T |
| D1013 | A6085YM_T6086M | W6081T_I6088T |
| D1014 | A6085YW_T6086M | W6081T_I6088T |
| D1015 | A6085YH_T6086M | W6081T_I6088T |
| D1016 | A6085YF_T6086W | W6081T_I6088T |
| D1017 | A6085YY_T6086W | W6081T_I6088T |
| D1018 | A6085YM_T6086W | W6081T_I6088T |
| D1019 | A6085YW_T6086W | W6081T_I6088T |
| D1020 | A6085YH_T6086W | W6081T_I6088T |
| D1021 | A6085YF_T6086H | W6081T_I6088T |
| D1022 | A6085YY_T6086H | W6081T_I6088T |
| D1023 | A6085YM_T6086H | W6081T_I6088T |
| D1024 | A6085YW_T6086H | W6081T_I6088T |
| D1025 | A6085YH_T6086H | W6081T_I6088T |
| D1026 | A6085YF_T6086Y | W6081L_I6088T |
| D1027 | A6085YY_T6086Y | W6081L_I6088T |
| D1028 | A6085YM_T6086Y | W6081L_I6088T |
| D1029 | A6085YW_T6086Y | W6081L_I6088T |
| D1030 | A6085YH_T6086Y | W6081L_I6088T |
| D1031 | A6085YF_T6086F | W6081L_I6088T |
| D1032 | A6085YY_T6086F | W6081L_I6088T |
| D1033 | A6085YM_T6086F | W6081L_I6088T |
| D1034 | A6085YW_T6086F | W6081L_I6088T |
| D1035 | A6085YH_T6086F | W6081L_I6088T |
| D1036 | A6085YF_T6086M | W6081L_I6088T |
| D1037 | A6085YY_T6086M | W6081L_I6088T |
| D1038 | A6085YM_T6086M | W6081L_I6088T |
| D1039 | A6085YW_T6086M | W6081L_I6088T |
| D1040 | A6085YH_T6086M | W6081L_I6088T |
| D1041 | A6085YF_T6086W | W6081L_I6088T |
| D1042 | A6085YY_T6086W | W6081L_I6088T |
| D1043 | A6085YM_T6086W | W6081L_I6088T |
| D1044 | A6085YW_T6086W | W6081L_I6088T |
| D1045 | A6085YH_T6086W | W6081L_I6088T |
| D1046 | A6085YF_T6086H | W6081L_I6088T |
| D1047 | A6085YY_T6086H | W6081L_I6088T |
| D1048 | A6085YM_T6086H | W6081L_I6088T |
| D1049 | A6085YW_T6086H | W6081L_I6088T |
| D1050 | A6085YH_T6086H | W6081L_I6088T |
| D1051 | A6085YF_T6086Y | W6081A_I6088T |
| D1052 | A6085YY_T6086Y | W6081A_I6088T |
| D1053 | A6085YM_T6086Y | W6081A_I6088T |
| D1054 | A6085YW_T6086Y | W6081A_I6088T |
| D1055 | A6085YH_T6086Y | W6081A_I6088T |
| D1056 | A6085YF_T6086F | W6081A_I6088T |
| D1057 | A6085YY_T6086F | W6081A_I6088T |
| D1058 | A6085YM_T6086F | W6081A_I6088T |
| D1059 | A6085YW_T6086F | W6081A_I6088T |
| D1060 | A6085YH_T6086F | W6081A_I6088T |
| D1061 | A6085YF_T6086M | W6081A_I6088T |
| D1062 | A6085YY_T6086M | W6081A_I6088T |
| D1063 | A6085YM_T6086M | W6081A_I6088T |
| D1064 | A6085YW_T6086M | W6081A_I6088T |
| D1065 | A6085YH_T6086M | W6081A_I6088T |
| D1066 | A6085YF_T6086W | W6081A_I6088T |
| D1067 | A6085YY_T6086W | W6081A_I6088T |
| D1068 | A6085YM_T6086W | W6081A_I6088T |
| D1069 | A6085YW_T6086W | W6081A_I6088T |
| D1070 | A6085YH_T6086W | W6081A_I6088T |
| D1071 | A6085YF_T6086H | W6081A_I6088T |
| D1072 | A6085YY_T6086H | W6081A_I6088T |
| D1073 | A6085YM_T6086H | W6081A_I6088T |
| D1074 | A6085YW_T6086H | W6081A_I6088T |
| D1075 | A6085YH_T6086H | W6081A_I6088T |
| D1076 | A6085YF_T6086Y | W6081V_I6088T |
| D1077 | A6085YY_T6086Y | W6081V_I6088T |
| D1078 | A6085YM_T6086Y | W6081V_I6088T |
| D1079 | A6085YW_T6086Y | W6081V_I6088T |
| D1080 | A6085YH_T6086Y | W6081V_I6088T |
| D1081 | A6085YF_T6086F | W6081V_I6088T |
| D1082 | A6085YY_T6086F | W6081V_I6088T |
| D1083 | A6085YM_T6086F | W6081V_I6088T |
| D1084 | A6085YW_T6086F | W6081V_I6088T |
| D1085 | A6085YH_T6086F | W6081V_I6088T |
| D1086 | A6085YF_T6086M | W6081V_I6088T |
| D1087 | A6085YY_T6086M | W6081V_I6088T |
| D1088 | A6085YM_T6086M | W6081V_I6088T |
| D1089 | A6085YW_T6086M | W6081V_I6088T |
| D1090 | A6085YH_T6086M | W6081V_I6088T |
| D1091 | A6085YF_T6086W | W6081V_I6088T |
| D1092 | A6085YY_T6086W | W6081V_I6088T |
| D1093 | A6085YM_T6086W | W6081V_I6088T |
| D1094 | A6085YW_T6086W | W6081V_I6088T |
| D1095 | A6085YH_T6086W | W6081V_I6088T |
| D1096 | A6085YF_T6086H | W6081V_I6088T |
| D1097 | A6085YY_T6086H | W6081V_I6088T |
| D1098 | A6085YM_T6086H | W6081V_I6088T |
| D1099 | A6085YW_T6086H | W6081V_I6088T |
| D1100 | A6085YH_T6086H | W6081V_I6088T |
| D1101 | A6085YF_T6086Y | W6081I_I6088T |
| D1102 | A6085YY_T6086Y | W6081I_I6088T |
| D1103 | A6085YM_T6086Y | W6081I_I6088T |
| D1104 | A6085YW_T6086Y | W6081I_I6088T |
| D1105 | A6085YH_T6086Y | W6081I_I6088T |
| D1106 | A6085YF_T6086F | W6081I_I6088T |
| D1107 | A6085YY_T6086F | W6081I_I6088T |
| D1108 | A6085YM_T6086F | W6081I_I6088T |
| D1109 | A6085YW_T6086F | W6081I_I6088T |
| D1110 | A6085YH_T6086F | W6081I_I6088T |
| D1111 | A6085YF_T6086M | W6081I_I6088T |
| D1112 | A6085YY_T6086M | W6081I_I6088T |
| D1113 | A6085YM_T6086M | W6081I_I6088T |
| D1114 | A6085YW_T6086M | W6081I_I6088T |
| D1115 | A6085YH_T6086M | W6081I_I6088T |
| D1116 | A6085YF_T6086W | W6081I_I6088T |
| D1117 | A6085YY_T6086W | W6081I_I6088T |
| D1118 | A6085YM_T6086W | W6081I_I6088T |
| D1119 | A6085YW_T6086W | W6081I_I6088T |
| D1120 | A6085YH_T6086W | W6081I_I6088T |
| D1121 | A6085YF_T6086H | W6081I_I6088T |
| D1122 | A6085YY_T6086H | W6081I_I6088T |
| D1123 | A6085YM_T6086H | W6081I_I6088T |
| D1124 | A6085YW_T6086H | W6081I_I6088T |
| D1125 | A6085YH_T6086H | W6081I_I6088T |

TABLE 10
IgA HetFc Designs comprising Core Mutations in Combination
with Mutations at Positions 6079 and 6088 in Chain B

CH3 Domain Mutations

| No. | Chain A | Chain B |
|---|---|---|
| D1126 | A6085YF_T6086Y | L6079V_W6081T_I6088L |
| D1127 | A6085YY_T6086Y | L6079V_W6081T_I6088L |
| D1128 | A6085YM_T6086Y | L6079V_W6081T_I6088L |
| D1129 | A6085YW_T6086Y | L6079V_W6081T_I6088L |
| D1130 | A6085YH_T6086Y | L6079V_W6081T_I6088L |
| D1131 | A6085YF_T6086F | L6079V_W6081T_I6088L |
| D1132 | A6085YY_T6086F | L6079V_W6081T_I6088L |
| D1133 | A6085YM_T6086F | L6079V_W6081T_I6088L |
| D1134 | A6085YW_T6086F | L6079V_W6081T_I6088L |
| D1135 | A6085YH_T6086F | L6079V_W6081T_I6088L |
| D1136 | A6085YF_T6086M | L6079V_W6081T_I6088L |
| D1137 | A6085YY_T6086M | L6079V_W6081T_I6088L |
| D1138 | A6085YM_T6086M | L6079V_W6081T_I6088L |
| D1139 | A6085YW_T6086M | L6079V_W6081T_I6088L |
| D1140 | A6085YH_T6086M | L6079V_W6081T_I6088L |
| D1141 | A6085YF_T6086W | L6079V_W6081T_I6088L |
| D1142 | A6085YY_T6086W | L6079V_W6081T_I6088L |
| D1143 | A6085YM_T6086W | L6079V_W6081T_I6088L |
| D1144 | A6085YW_T6086W | L6079V_W6081T_I6088L |
| D1145 | A6085YH_T6086W | L6079V_W6081T_I6088L |
| D1146 | A6085YF_T6086H | L6079V_W6081T_I6088L |
| D1147 | A6085YY_T6086H | L6079V_W6081T_I6088L |
| D1148 | A6085YM_T6086H | L6079V_W6081T_I6088L |
| D1149 | A6085YW_T6086H | L6079V_W6081T_I6088L |
| D1150 | A6085YH_T6086H | L6079V_W6081T_I6088L |
| D1151 | A6085YF_T6086Y | L6079V_W6081L_I6088L |
| D1152 | A6085YY_T6086Y | L6079V_W6081L_I6088L |
| D1153 | A6085YM_T6086Y | L6079V_W6081L_I6088L |
| D1154 | A6085YW_T6086Y | L6079V_W6081L_I6088L |
| D1155 | A6085YH_T6086Y | L6079V_W6081L_I6088L |
| D1156 | A6085YF_T6086F | L6079V_W6081L_I6088L |
| D1157 | A6085YY_T6086F | L6079V_W6081L_I6088L |
| D1158 | A6085YM_T6086F | L6079V_W6081L_I6088L |
| D1159 | A6085YW_T6086F | L6079V_W6081L_I6088L |
| D1160 | A6085YH_T6086F | L6079V_W6081L_I6088L |
| D1161 | A6085YF_T6086M | L6079V_W6081L_I6088L |
| D1162 | A6085YY_T6086M | L6079V_W6081L_I6088L |
| D1163 | A6085YM_T6086M | L6079V_W6081L_I6088L |
| D1164 | A6085YW_T6086M | L6079V_W6081L_I6088L |
| D1165 | A6085YH_T6086M | L6079V_W6081L_I6088L |
| D1166 | A6085YF_T6086W | L6079V_W6081L_I6088L |
| D1167 | A6085YY_T6086W | L6079V_W6081L_I6088L |
| D1168 | A6085YM_T6086W | L6079V_W6081L_I6088L |
| D1169 | A6085YW_T6086W | L6079V_W6081L_I6088L |
| D1170 | A6085YH_T6086W | L6079V_W6081L_I6088L |
| D1171 | A6085YF_T6086H | L6079V_W6081L_I6088L |
| D1172 | A6085YY_T6086H | L6079V_W6081L_I6088L |
| D1173 | A6085YM_T6086H | L6079V_W6081L_I6088L |
| D1174 | A6085YW_T6086H | L6079V_W6081L_I6088L |
| D1175 | A6085YH_T6086H | L6079V_W6081L_I6088L |
| D1176 | A6085YF_T6086Y | L6079V_W6081A_I6088L |
| D1177 | A6085YY_T6086Y | L6079V_W6081A_I6088L |
| D1178 | A6085YM_T6086Y | L6079V_W6081A_I6088L |
| D1179 | A6085YW_T6086Y | L6079V_W6081A_I6088L |
| D1180 | A6085YH_T6086Y | L6079V_W6081A_I6088L |
| D1181 | A6085YF_T6086F | L6079V_W6081A_I6088L |
| D1182 | A6085YY_T6086F | L6079V_W6081A_I6088L |
| D1183 | A6085YM_T6086F | L6079V_W6081A_I6088L |
| D1184 | A6085YW_T6086F | L6079V_W6081A_I6088L |
| D1185 | A6085YH_T6086F | L6079V_W6081A_I6088L |
| D1186 | A6085YF_T6086M | L6079V_W6081A_I6088L |
| D1187 | A6085YY_T6086M | L6079V_W6081A_I6088L |
| D1188 | A6085YM_T6086M | L6079V_W6081A_I6088L |
| D1189 | A6085YW_T6086M | L6079V_W6081A_I6088L |
| D1190 | A6085YH_T6086M | L6079V_W6081A_I6088L |
| D1191 | A6085YF_T6086W | L6079V_W6081A_I6088L |
| D1192 | A6085YY_T6086W | L6079V_W6081A_I6088L |
| D1193 | A6085YM_T6086W | L6079V_W6081A_I6088L |
| D1194 | A6085YW_T6086W | L6079V_W6081A_I6088L |
| D1195 | A6085YH_T6086W | L6079V_W6081A_I6088L |
| D1196 | A6085YF_T6086H | L6079V_W6081A_I6088L |
| D1197 | A6085YY_T6086H | L6079V_W6081A_I6088L |
| D1198 | A6085YM_T6086H | L6079V_W6081A_I6088L |
| D1199 | A6085YW_T6086H | L6079V_W6081A_I6088L |
| D1200 | A6085YH_T6086H | L6079V_W6081A_I6088L |
| D1201 | A6085YF_T6086Y | L6079V_W6081V_I6088L |
| D1202 | A6085YY_T6086Y | L6079V_W6081V_I6088L |
| D1203 | A6085YM_T6086Y | L6079V_W6081V_I6088L |
| D1204 | A6085YW_T6086Y | L6079V_W6081V_I6088L |
| D1205 | A6085YH_T6086Y | L6079V_W6081V_I6088L |
| D1206 | A6085YF_T6086F | L6079V_W6081V_I6088L |
| D1207 | A6085YY_T6086F | L6079V_W6081V_I6088L |
| D1208 | A6085YM_T6086F | L6079V_W6081V_I6088L |
| D1209 | A6085YW_T6086F | L6079V_W6081V_I6088L |
| D1210 | A6085YH_T6086F | L6079V_W6081V_I6088L |
| D1211 | A6085YF_T6086M | L6079V_W6081V_I6088L |
| D1212 | A6085YY_T6086M | L6079V_W6081V_I6088L |
| D1213 | A6085YM_T6086M | L6079V_W6081V_I6088L |
| D1214 | A6085YW_T6086M | L6079V_W6081V_I6088L |
| D1215 | A6085YH_T6086M | L6079V_W6081V_I6088L |
| D1216 | A6085YF_T6086W | L6079V_W6081V_I6088L |
| D1217 | A6085YY_T6086W | L6079V_W6081V_I6088L |
| D1218 | A6085YM_T6086W | L6079V_W6081V_I6088L |
| D1219 | A6085YW_T6086W | L6079V_W6081V_I6088L |
| D1220 | A6085YH_T6086W | L6079V_W6081V_I6088L |
| D1221 | A6085YF_T6086H | L6079V_W6081V_I6088L |
| D1222 | A6085YY_T6086H | L6079V_W6081V_I6088L |
| D1223 | A6085YM_T6086H | L6079V_W6081V_I6088L |
| D1224 | A6085YW_T6086H | L6079V_W6081V_I6088L |
| D1225 | A6085YH_T6086H | L6079V_W6081V_I6088L |
| D1226 | A6085YF_T6086Y | L6079V_W6081I_I6088L |
| D1227 | A6085YY_T6086Y | L6079V_W6081I_I6088L |
| D1228 | A6085YM_T6086Y | L6079V_W6081I_I6088L |
| D1229 | A6085YW_T6086Y | L6079V_W6081I_I6088L |
| D1230 | A6085YH_T6086Y | L6079V_W6081I_I6088L |
| D1231 | A6085YF_T6086F | L6079V_W6081I_I6088L |
| D1232 | A6085YY_T6086F | L6079V_W6081I_I6088L |
| D1233 | A6085YM_T6086F | L6079V_W6081I_I6088L |
| D1234 | A6085YW_T6086F | L6079V_W6081I_I6088L |
| D1235 | A6085YH_T6086F | L6079V_W6081I_I6088L |
| D1236 | A6085YF_T6086M | L6079V_W6081I_I6088L |
| D1237 | A6085YY_T6086M | L6079V_W6081I_I6088L |
| D1238 | A6085YM_T6086M | L6079V_W6081I_I6088L |
| D1239 | A6085YW_T6086M | L6079V_W6081I_I6088L |
| D1240 | A6085YH_T6086M | L6079V_W6081I_I6088L |
| D1241 | A6085YF_T6086W | L6079V_W6081I_I6088L |
| D1242 | A6085YY_T6086W | L6079V_W6081I_I6088L |
| D1243 | A6085YM_T6086W | L6079V_W6081I_I6088L |
| D1244 | A6085YW_T6086W | L6079V_W6081I_I6088L |
| D1245 | A6085YH_T6086W | L6079V_W6081I_I6088L |
| D1246 | A6085YF_T6086H | L6079V_W6081I_I6088L |
| D1247 | A6085YY_T6086H | L6079V_W6081I_I6088L |
| D1248 | A6085YM_T6086H | L6079V_W6081I_I6088L |
| D1249 | A6085YW_T6086H | L6079V_W6081I_I6088L |
| D1250 | A6085YH_T6086H | L6079V_W6081I_I6088L |
| D1251 | A6085YF_T6086Y | L6079T_W6081T_I6088L |
| D1252 | A6085YY_T6086Y | L6079T_W6081T_I6088L |
| D1253 | A6085YM_T6086Y | L6079T_W6081T_I6088L |
| D1254 | A6085YW_T6086Y | L6079T_W6081T_I6088L |
| D1255 | A6085YH_T6086Y | L6079T_W6081T_I6088L |
| D1256 | A6085YF_T6086F | L6079T_W6081T_I6088L |
| D1257 | A6085YY_T6086F | L6079T_W6081T_I6088L |
| D1258 | A6085YM_T6086F | L6079T_W6081T_I6088L |
| D1259 | A6085YW_T6086F | L6079T_W6081T_I6088L |
| D1260 | A6085YH_T6086F | L6079T_W6081T_I6088L |
| D1261 | A6085YF_T6086M | L6079T_W6081T_I6088L |
| D1262 | A6085YY_T6086M | L6079T_W6081T_I6088L |
| D1263 | A6085YM_T6086M | L6079T_W6081T_I6088L |
| D1264 | A6085YW_T6086M | L6079T_W6081T_I6088L |
| D1265 | A6085YH_T6086M | L6079T_W6081T_I6088L |
| D1266 | A6085YF_T6086W | L6079T_W6081T_I6088L |
| D1267 | A6085YY_T6086W | L6079T_W6081T_I6088L |
| D1268 | A6085YM_T6086W | L6079T_W6081T_I6088L |
| D1269 | A6085YW_T6086W | L6079T_W6081T_I6088L |
| D1270 | A6085YH_T6086W | L6079T_W6081T_I6088L |
| D1271 | A6085YF_T6086H | L6079T_W6081T_I6088L |
| D1272 | A6085YY_T6086H | L6079T_W6081T_I6088L |
| D1273 | A6085YM_T6086H | L6079T_W6081T_I6088L |
| D1274 | A6085YW_T6086H | L6079T_W6081T_I6088L |
| D1275 | A6085YH_T6086H | L6079T_W6081T_I6088L |
| D1276 | A6085YF_T6086Y | L6079T_W6081L_I6088L |
| D1277 | A6085YY_T6086Y | L6079T_W6081L_I6088L |
| D1278 | A6085YM_T6086Y | L6079T_W6081L_I6088L |
| D1279 | A6085YW_T6086Y | L6079T_W6081L_I6088L |
| D1280 | A6085YH_T6086Y | L6079T_W6081L_I6088L |
| D1281 | A6085YF_T6086F | L6079T_W6081L_I6088L |
| D1282 | A6085YY_T6086F | L6079T_W6081L_I6088L |
| D1283 | A6085YM_T6086F | L6079T_W6081L_I6088L |
| D1284 | A6085YW_T6086F | L6079T_W6081L_I6088L |
| D1285 | A6085YH_T6086F | L6079T_W6081L_I6088L |
| D1286 | A6085YF_T6086M | L6079T_W6081L_I6088L |
| D1287 | A6085YY_T6086M | L6079T_W6081L_I6088L |
| D1288 | A6085YM_T6086M | L6079T_W6081L_I6088L |
| D1289 | A6085YW_T6086M | L6079T_W6081L_I6088L |
| D1290 | A6085YH_T6086M | L6079T_W6081L_I6088L |
| D1291 | A6085YF_T6086W | L6079T_W6081L_I6088L |
| D1292 | A6085YY_T6086W | L6079T_W6081L_I6088L |
| D1293 | A6085YM_T6086W | L6079T_W6081L_I6088L |
| D1294 | A6085YW_T6086W | L6079T_W6081L_I6088L |
| D1295 | A6085YH_T6086W | L6079T_W6081L_I6088L |
| D1296 | A6085YF_T6086H | L6079T_W6081L_I6088L |
| D1297 | A6085YY_T6086H | L6079T_W6081L_I6088L |
| D1298 | A6085YM_T6086H | L6079T_W6081L_I6088L |
| D1299 | A6085YW_T6086H | L6079T_W6081L_I6088L |
| D1300 | A6085YH_T6086H | L6079T_W6081L_I6088L |
| D1301 | A6085YF_T6086Y | L6079T_W6081A_I6088L |
| D1302 | A6085YY_T6086Y | L6079T_W6081A_I6088L |
| D1303 | A6085YM_T6086Y | L6079T_W6081A_I6088L |
| D1304 | A6085YW_T6086Y | L6079T_W6081A_I6088L |
| D1305 | A6085YH_T6086Y | L6079T_W6081A_I6088L |
| D1306 | A6085YF_T6086F | L6079T_W6081A_I6088L |
| D1307 | A6085YY_T6086F | L6079T_W6081A_I6088L |
| D1308 | A6085YM_T6086F | L6079T_W6081A_I6088L |
| D1309 | A6085YW_T6086F | L6079T_W6081A_I6088L |
| D1310 | A6085YH_T6086F | L6079T_W6081A_I6088L |
| D1311 | A6085YF_T6086M | L6079T_W6081A_I6088L |
| D1312 | A6085YY_T6086M | L6079T_W6081A_I6088L |
| D1313 | A6085YM_T6086M | L6079T_W6081A_I6088L |
| D1314 | A6085YW_T6086M | L6079T_W6081A_I6088L |
| D1315 | A6085YH_T6086M | L6079T_W6081A_I6088L |
| D1316 | A6085YF_T6086W | L6079T_W6081A_I6088L |
| D1317 | A6085YY_T6086W | L6079T_W6081A_I6088L |
| D1318 | A6085YM_T6086W | L6079T_W6081A_I6088L |
| D1319 | A6085YW_T6086W | L6079T_W6081A_I6088L |
| D1320 | A6085YH_T6086W | L6079T_W6081A_I6088L |
| D1321 | A6085YF_T6086H | L6079T_W6081A_I6088L |
| D1322 | A6085YY_T6086H | L6079T_W6081A_I6088L |
| D1323 | A6085YM_T6086H | L6079T_W6081A_I6088L |
| D1324 | A6085YW_T6086H | L6079T_W6081A_I6088L |
| D1325 | A6085YH_T6086H | L6079T_W6081A_I6088L |
| D1326 | A6085YF_T6086Y | L6079T_W6081V_I6088L |
| D1327 | A6085YY_T6086Y | L6079T_W6081V_I6088L |
| D1328 | A6085YM_T6086Y | L6079T_W6081V_I6088L |
| D1329 | A6085YW_T6086Y | L6079T_W6081V_I6088L |
| D1330 | A6085YH_T6086Y | L6079T_W6081V_I6088L |
| D1331 | A6085YF_T6086F | L6079T_W6081V_I6088L |
| D1332 | A6085YY_T6086F | L6079T_W6081V_I6088L |
| D1333 | A6085YM_T6086F | L6079T_W6081V_I6088L |
| D1334 | A6085YW_T6086F | L6079T_W6081V_I6088L |
| D1335 | A6085YH_T6086F | L6079T_W6081V_I6088L |
| D1336 | A6085YF_T6086M | L6079T_W6081V_I6088L |
| D1337 | A6085YY_T6086M | L6079T_W6081V_I6088L |
| D1338 | A6085YM_T6086M | L6079T_W6081V_I6088L |
| D1339 | A6085YW_T6086M | L6079T_W6081V_I6088L |
| D1340 | A6085YH_T6086M | L6079T_W6081V_I6088L |
| D1341 | A6085YF_T6086W | L6079T_W6081V_I6088L |
| D1342 | A6085YY_T6086W | L6079T_W6081V_I6088L |
| D1343 | A6085YM_T6086W | L6079T_W6081V_I6088L |
| D1344 | A6085YW_T6086W | L6079T_W6081V_I6088L |
| D1345 | A6085YH_T6086W | L6079T_W6081V_I6088L |
| D1346 | A6085YF_T6086H | L6079T_W6081V_I6088L |
| D1347 | A6085YY_T6086H | L6079T_W6081V_I6088L |
| D1348 | A6085YM_T6086H | L6079T_W6081V_I6088L |
| D1349 | A6085YW_T6086H | L6079T_W6081V_I6088L |
| D1350 | A6085YH_T6086H | L6079T_W6081V_I6088L |
| D1351 | A6085YF_T6086Y | L6079T_W6081I_I6088L |
| D1352 | A6085YY_T6086Y | L6079T_W6081I_I6088L |
| D1353 | A6085YM_T6086Y | L6079T_W6081I_I6088L |
| D1354 | A6085YW_T6086Y | L6079T_W6081I_I6088L |
| D1355 | A6085YH_T6086Y | L6079T_W6081I_I6088L |
| D1356 | A6085YF_T6086F | L6079T_W6081I_I6088L |
| D1357 | A6085YY_T6086F | L6079T_W6081I_I6088L |
| D1358 | A6085YM_T6086F | L6079T_W6081I_I6088L |
| D1359 | A6085YW_T6086F | L6079T_W6081I_I6088L |
| D1360 | A6085YH_T6086F | L6079T_W6081I_I6088L |
| D1361 | A6085YF_T6086M | L6079T_W6081I_I6088L |
| D1362 | A6085YY_T6086M | L6079T_W6081I_I6088L |
| D1363 | A6085YM_T6086M | L6079T_W6081I_I6088L |
| D1364 | A6085YW_T6086M | L6079T_W6081I_I6088L |
| D1365 | A6085YH_T6086M | L6079T_W6081I_I6088L |
| D1366 | A6085YF_T6086W | L6079T_W6081I_I6088L |
| D1367 | A6085YY_T6086W | L6079T_W6081I_I6088L |
| D1368 | A6085YM_T6086W | L6079T_W6081I_I6088L |
| D1369 | A6085YW_T6086W | L6079T_W6081I_I6088L |
| D1370 | A6085YH_T6086W | L6079T_W6081I_I6088L |
| D1371 | A6085YF_T6086H | L6079T_W6081I_I6088L |
| D1372 | A6085YY_T6086H | L6079T_W6081I_I6088L |
| D1373 | A6085YM_T6086H | L6079T_W6081I_I6088L |
| D1374 | A6085YW_T6086H | L6079T_W6081I_I6088L |
| D1375 | A6085YH_T6086H | L6079T_W6081I_I6088L |
| D1376 | A6085YF_T6086Y | L6079A_W6081T_I6088L |
| D1377 | A6085YY_T6086Y | L6079A_W6081T_I6088L |
| D1378 | A6085YM_T6086Y | L6079A_W6081T_I6088L |
| D1379 | A6085YW_T6086Y | L6079A_W6081T_I6088L |
| D1380 | A6085YH_T6086Y | L6079A_W6081T_I6088L |
| D1381 | A6085YF_T6086F | L6079A_W6081T_I6088L |
| D1382 | A6085YY_T6086F | L6079A_W6081T_I6088L |
| D1383 | A6085YM_T6086F | L6079A_W6081T_I6088L |
| D1384 | A6085YW_T6086F | L6079A_W6081T_I6088L |
| D1385 | A6085YH_T6086F | L6079A_W6081T_I6088L |
| D1386 | A6085YF_T6086M | L6079A_W6081T_I6088L |
| D1387 | A6085YY_T6086M | L6079A_W6081T_I6088L |
| D1388 | A6085YM_T6086M | L6079A_W6081T_I6088L |
| D1389 | A6085YW_T6086M | L6079A_W6081T_I6088L |
| D1390 | A6085YH_T6086M | L6079A_W6081T_I6088L |
| D1391 | A6085YF_T6086W | L6079A_W6081T_I6088L |
| D1392 | A6085YY_T6086W | L6079A_W6081T_I6088L |
| D1393 | A6085YM_T6086W | L6079A_W6081T_I6088L |
| D1394 | A6085YW_T6086W | L6079A_W6081T_I6088L |
| D1395 | A6085YH_T6086W | L6079A_W6081T_I6088L |
| D1396 | A6085YF_T6086H | L6079A_W6081T_I6088L |
| D1397 | A6085YY_T6086H | L6079A_W6081T_I6088L |
| D1398 | A6085YM_T6086H | L6079A_W6081T_I6088L |
| D1399 | A6085YW_T6086H | L6079A_W6081T_I6088L |
| D1400 | A6085YH_T6086H | L6079A_W6081T_I6088L |
| D1401 | A6085YF_T6086Y | L6079A_W6081L_I6088L |
| D1402 | A6085YY_T6086Y | L6079A_W6081L_I6088L |
| D1403 | A6085YM_T6086Y | L6079A_W6081L_I6088L |
| D1404 | A6085YW_T6086Y | L6079A_W6081L_I6088L |
| D1405 | A6085YH_T6086Y | L6079A_W6081L_I6088L |
| D1406 | A6085YF_T6086F | L6079A_W6081L_I6088L |
| D1407 | A6085YY_T6086F | L6079A_W6081L_I6088L |
| D1408 | A6085YM_T6086F | L6079A_W6081L_I6088L |
| D1409 | A6085YW_T6086F | L6079A_W6081L_I6088L |
| D1410 | A6085YH_T6086F | L6079A_W6081L_I6088L |
| D1411 | A6085YF_T6086M | L6079A_W6081L_I6088L |
| D1412 | A6085YY_T6086M | L6079A_W6081L_I6088L |
| D1413 | A6085YM_T6086M | L6079A_W6081L_I6088L |
| D1414 | A6085YW_T6086M | L6079A_W6081L_I6088L |
| D1415 | A6085YH_T6086M | L6079A_W6081L_I6088L |
| D1416 | A6085YF_T6086W | L6079A_W6081L_I6088L |
| D1417 | A6085YY_T6086W | L6079A_W6081L_I6088L |
| D1418 | A6085YM_T6086W | L6079A_W6081L_I6088L |
| D1419 | A6085YW_T6086W | L6079A_W6081L_I6088L |
| D1420 | A6085YH_T6086W | L6079A_W6081L_I6088L |
| D1421 | A6085YF_T6086H | L6079A_W6081L_I6088L |
| D1422 | A6085YY_T6086H | L6079A_W6081L_I6088L |
| D1423 | A6085YM_T6086H | L6079A_W6081L_I6088L |
| D1424 | A6085YW_T6086H | L6079A_W6081L_I6088L |
| D1425 | A6085YH_T6086H | L6079A_W6081L_I6088L |
| D1426 | A6085YF_T6086Y | L6079A_W6081A_I6088L |
| D1427 | A6085YY_T6086Y | L6079A_W6081A_I6088L |
| D1428 | A6085YM_T6086Y | L6079A_W6081A_I6088L |
| D1429 | A6085YW_T6086Y | L6079A_W6081A_I6088L |
| D1430 | A6085YH_T6086Y | L6079A_W6081A_I6088L |
| D1431 | A6085YF_T6086F | L6079A_W6081A_I6088L |
| D1432 | A6085YY_T6086F | L6079A_W6081A_I6088L |
| D1433 | A6085YM_T6086F | L6079A_W6081A_I6088L |
| D1434 | A6085YW_T6086F | L6079A_W6081A_I6088L |
| D1435 | A6085YH_T6086F | L6079A_W6081A_I6088L |
| D1436 | A6085YF_T6086M | L6079A_W6081A_I6088L |
| D1437 | A6085YY_T6086M | L6079A_W6081A_I6088L |
| D1438 | A6085YM_T6086M | L6079A_W6081A_I6088L |
| D1439 | A6085YW_T6086M | L6079A_W6081A_I6088L |
| D1440 | A6085YH_T6086M | L6079A_W6081A_I6088L |
| D1441 | A6085YF_T6086W | L6079A_W6081A_I6088L |
| D1442 | A6085YY_T6086W | L6079A_W6081A_I6088L |
| D1443 | A6085YM_T6086W | L6079A_W6081A_I6088L |
| D1444 | A6085YW_T6086W | L6079A_W6081A_I6088L |
| D1445 | A6085YH_T6086W | L6079A_W6081A_I6088L |
| D1446 | A6085YF_T6086H | L6079A_W6081A_I6088L |
| D1447 | A6085YY_T6086H | L6079A_W6081A_I6088L |
| D1448 | A6085YM_T6086H | L6079A_W6081A_I6088L |
| D1449 | A6085YW_T6086H | L6079A_W6081A_I6088L |
| D1450 | A6085YH_T6086H | L6079A_W6081A_I6088L |
| D1451 | A6085YF_T6086Y | L6079A_W6081V_I6088L |
| D1452 | A6085YY_T6086Y | L6079A_W6081V_I6088L |
| D1453 | A6085YM_T6086Y | L6079A_W6081V_I6088L |
| D1454 | A6085YW_T6086Y | L6079A_W6081V_I6088L |
| D1455 | A6085YH_T6086Y | L6079A_W6081V_I6088L |
| D1456 | A6085YF_T6086F | L6079A_W6081V_I6088L |
| D1457 | A6085YY_T6086F | L6079A_W6081V_I6088L |
| D1458 | A6085YM_T6086F | L6079A_W6081V_I6088L |
| D1459 | A6085YW_T6086F | L6079A_W6081V_I6088L |
| D1460 | A6085YH_T6086F | L6079A_W6081V_I6088L |
| D1461 | A6085YF_T6086M | L6079A_W6081V_I6088L |
| D1462 | A6085YY_T6086M | L6079A_W6081V_I6088L |
| D1463 | A6085YM_T6086M | L6079A_W6081V_I6088L |
| D1464 | A6085YW_T6086M | L6079A_W6081V_I6088L |
| D1465 | A6085YH_T6086M | L6079A_W6081V_I6088L |
| D1466 | A6085YF_T6086W | L6079A_W6081V_I6088L |
| D1467 | A6085YY_T6086W | L6079A_W6081V_I6088L |
| D1468 | A6085YM_T6086W | L6079A_W6081V_I6088L |
| D1469 | A6085YW_T6086W | L6079A_W6081V_I6088L |
| D1470 | A6085YH_T6086W | L6079A_W6081V_I6088L |
| D1471 | A6085YF_T6086H | L6079A_W6081V_I6088L |
| D1472 | A6085YY_T6086H | L6079A_W6081V_I6088L |
| D1473 | A6085YM_T6086H | L6079A_W6081V_I6088L |
| D1474 | A6085YW_T6086H | L6079A_W6081V_I6088L |
| D1475 | A6085YH_T6086H | L6079A_W6081V_I6088L |
| D1476 | A6085YF_T6086Y | L6079A_W6081I_I6088L |
| D1477 | A6085YY_T6086Y | L6079A_W6081I_I6088L |
| D1478 | A6085YM_T6086Y | L6079A_W6081I_I6088L |
| D1479 | A6085YW_T6086Y | L6079A_W6081I_I6088L |
| D1480 | A6085YH_T6086Y | L6079A_W6081I_I6088L |
| D1481 | A6085YF_T6086F | L6079A_W6081I_I6088L |
| D1482 | A6085YY_T6086F | L6079A_W6081I_I6088L |
| D1483 | A6085YM_T6086F | L6079A_W6081I_I6088L |
| D1484 | A6085YW_T6086F | L6079A_W6081I_I6088L |
| D1485 | A6085YH_T6086F | L6079A_W6081I_I6088L |
| D1486 | A6085YF_T6086M | L6079A_W6081I_I6088L |
| D1487 | A6085YY_T6086M | L6079A_W6081I_I6088L |
| D1488 | A6085YM_T6086M | L6079A_W6081I_I6088L |
| D1489 | A6085YW_T6086M | L6079A_W6081I_I6088L |
| D1490 | A6085YH_T6086M | L6079A_W6081I_I6088L |
| D1491 | A6085YF_T6086W | L6079A_W6081I_I6088L |
| D1492 | A6085YY_T6086W | L6079A_W6081I_I6088L |
| D1493 | A6085YM_T6086W | L6079A_W6081I_I6088L |
| D1494 | A6085YW_T6086W | L6079A_W6081I_I6088L |
| D1495 | A6085YH_T6086W | L6079A_W6081I_I6088L |
| D1496 | A6085YF_T6086H | L6079A_W6081I_I6088L |
| D1497 | A6085YY_T6086H | L6079A_W6081I_I6088L |
| D1498 | A6085YM_T6086H | L6079A_W6081I_I6088L |
| D1499 | A6085YW_T6086H | L6079A_W6081I_I6088L |
| D1500 | A6085YH_T6086H | L6079A_W6081I_I6088L |
| D1501 | A6085YF_T6086Y | L6079I_W6081T_I6088L |
| D1502 | A6085YY_T6086Y | L6079I_W6081T_I6088L |
| D1503 | A6085YM_T6086Y | L6079I_W6081T_I6088L |
| D1504 | A6085YW_T6086Y | L6079I_W6081T_I6088L |
| D1505 | A6085YH_T6086Y | L6079I_W6081T_I6088L |
| D1506 | A6085YF_T6086F | L6079I_W6081T_I6088L |
| D1507 | A6085YY_T6086F | L6079I_W6081T_I6088L |
| D1508 | A6085YM_T6086F | L6079I_W6081T_I6088L |
| D1509 | A6085YW_T6086F | L6079I_W6081T_I6088L |
| D1510 | A6085YH_T6086F | L6079I_W6081T_I6088L |
| D1511 | A6085YF_T6086M | L6079I_W6081T_I6088L |
| D1512 | A6085YY_T6086M | L6079I_W6081T_I6088L |
| D1513 | A6085YM_T6086M | L6079I_W6081T_I6088L |
| D1514 | A6085YW_T6086M | L6079I_W6081T_I6088L |
| D1515 | A6085YH_T6086M | L6079I_W6081T_I6088L |
| D1516 | A6085YF_T6086W | L6079I_W6081T_I6088L |
| D1517 | A6085YY_T6086W | L6079I_W6081T_I6088L |
| D1518 | A6085YM_T6086W | L6079I_W6081T_I6088L |
| D1519 | A6085YW_T6086W | L6079I_W6081T_I6088L |
| D1520 | A6085YH_T6086W | L6079I_W6081T_I6088L |
| D1521 | A6085YF_T6086H | L6079I_W6081T_I6088L |
| D1522 | A6085YY_T6086H | L6079I_W6081T_I6088L |
| D1523 | A6085YM_T6086H | L6079I_W6081T_I6088L |
| D1524 | A6085YW_T6086H | L6079I_W6081T_I6088L |
| D1525 | A6085YH_T6086H | L6079I_W6081T_I6088L |
| D1526 | A6085YF_T6086Y | L6079I_W6081L_I6088L |
| D1527 | A6085YY_T6086Y | L6079I_W6081L_I6088L |
| D1528 | A6085YM_T6086Y | L6079I_W6081L_I6088L |
| D1529 | A6085YW_T6086Y | L6079I_W6081L_I6088L |
| D1530 | A6085YH_T6086Y | L6079I_W6081L_I6088L |
| D1531 | A6085YF_T6086F | L6079I_W6081L_I6088L |
| D1532 | A6085YY_T6086F | L6079I_W6081L_I6088L |
| D1533 | A6085YM_T6086F | L6079I_W6081L_I6088L |
| D1534 | A6085YW_T6086F | L6079I_W6081L_I6088L |
| D1535 | A6085YH_T6086F | L6079I_W6081L_I6088L |
| D1536 | A6085YF_T6086M | L6079I_W6081L_I6088L |
| D1537 | A6085YY_T6086M | L6079I_W6081L_I6088L |
| D1538 | A6085YM_T6086M | L6079I_W6081L_I6088L |
| D1539 | A6085YW_T6086M | L6079I_W6081L_I6088L |
| D1540 | A6085YH_T6086M | L6079I_W6081L_I6088L |
| D1541 | A6085YF_T6086W | L6079I_W6081L_I6088L |
| D1542 | A6085YY_T6086W | L6079I_W6081L_I6088L |
| D1543 | A6085YM_T6086W | L6079I_W6081L_I6088L |
| D1544 | A6085YW_T6086W | L6079I_W6081L_I6088L |
| D1545 | A6085YH_T6086W | L6079I_W6081L_I6088L |
| D1546 | A6085YF_T6086H | L6079I_W6081L_I6088L |
| D1547 | A6085YY_T6086H | L6079I_W6081L_I6088L |
| D1548 | A6085YM_T6086H | L6079I_W6081L_I6088L |
| D1549 | A6085YW_T6086H | L6079I_W6081L_I6088L |
| D1550 | A6085YH_T6086H | L6079I_W6081L_I6088L |
| D1551 | A6085YF_T6086Y | L6079I_W6081A_I6088L |
| D1552 | A6085YY_T6086Y | L6079I_W6081A_I6088L |
| D1553 | A6085YM_T6086Y | L6079I_W6081A_I6088L |
| D1554 | A6085YW_T6086Y | L6079I_W6081A_I6088L |
| D1555 | A6085YH_T6086Y | L6079I_W6081A_I6088L |
| D1556 | A6085YF_T6086F | L6079I_W6081A_I6088L |
| D1557 | A6085YY_T6086F | L6079I_W6081A_I6088L |
| D1558 | A6085YM_T6086F | L6079I_W6081A_I6088L |
| D1559 | A6085YW_T6086F | L6079I_W6081A_I6088L |
| D1560 | A6085YH_T6086F | L6079I_W6081A_I6088L |
| D1561 | A6085YF_T6086M | L6079I_W6081A_I6088L |
| D1562 | A6085YY_T6086M | L6079I_W6081A_I6088L |
| D1563 | A6085YM_T6086M | L6079I_W6081A_I6088L |
| D1564 | A6085YW_T6086M | L6079I_W6081A_I6088L |
| D1565 | A6085YH_T6086M | L6079I_W6081A_I6088L |
| D1566 | A6085YF_T6086W | L6079I_W6081A_I6088L |
| D1567 | A6085YY_T6086W | L6079I_W6081A_I6088L |
| D1568 | A6085YM_T6086W | L6079I_W6081A_I6088L |
| D1569 | A6085YW_T6086W | L6079I_W6081A_I6088L |
| D1570 | A6085YH_T6086W | L6079I_W6081A_I6088L |
| D1571 | A6085YF_T6086H | L6079I_W6081A_I6088L |
| D1572 | A6085YY_T6086H | L6079I_W6081A_I6088L |
| D1573 | A6085YM_T6086H | L6079I_W6081A_I6088L |
| D1574 | A6085YW_T6086H | L6079I_W6081A_I6088L |
| D1575 | A6085YH_T6086H | L6079I_W6081A_I6088L |
| D1576 | A6085YF_T6086Y | L6079I_W6081V_I6088L |
| D1577 | A6085YY_T6086Y | L6079I_W6081V_I6088L |
| D1578 | A6085YM_T6086Y | L6079I_W6081V_I6088L |
| D1579 | A6085YW_T6086Y | L6079I_W6081V_I6088L |
| D1580 | A6085YH_T6086Y | L6079I_W6081V_I6088L |
| D1581 | A6085YF_T6086F | L6079I_W6081V_I6088L |
| D1582 | A6085YY_T6086F | L6079I_W6081V_I6088L |
| D1583 | A6085YM_T6086F | L6079I_W6081V_I6088L |
| D1584 | A6085YW_T6086F | L6079I_W6081V_I6088L |
| D1585 | A6085YH_T6086F | L6079I_W6081V_I6088L |
| D1586 | A6085YF_T6086M | L6079I_W6081V_I6088L |
| D1587 | A6085YY_T6086M | L6079I_W6081V_I6088L |
| D1588 | A6085YM_T6086M | L6079I_W6081V_I6088L |
| D1589 | A6085YW_T6086M | L6079I_W6081V_I6088L |
| D1590 | A6085YH_T6086M | L6079I_W6081V_I6088L |
| D1591 | A6085YF_T6086W | L6079I_W6081V_I6088L |
| D1592 | A6085YY_T6086W | L6079I_W6081V_I6088L |
| D1593 | A6085YM_T6086W | L6079I_W6081V_I6088L |
| D1594 | A6085YW_T6086W | L6079I_W6081V_I6088L |
| D1595 | A6085YH_T6086W | L6079I_W6081V_I6088L |
| D1596 | A6085YF_T6086H | L6079I_W6081V_I6088L |
| D1597 | A6085YY_T6086H | L6079I_W6081V_I6088L |
| D1598 | A6085YM_T6086H | L6079I_W6081V_I6088L |
| D1599 | A6085YW_T6086H | L6079I_W6081V_I6088L |
| D1600 | A6085YH_T6086H | L6079I_W6081V_16088L |
| D1601 | A6085YF_T6086Y | L6079I_W6081I_I6088L |
| D1602 | A6085YY_T6086Y | L6079I_W6081I_I6088L |
| D1603 | A6085YM_T6086Y | L6079I_W6081I_I6088L |
| D1604 | A6085YW_T6086Y | L6079I_W6081I_I6088L |
| D1605 | A6085YH_T6086Y | L6079I_W6081I_I6088L |
| D1606 | A6085YF_T6086F | L6079I_W6081I_I6088L |
| D1607 | A6085YY_T6086F | L6079I_W6081I_I6088L |
| D1608 | A6085YM_T6086F | L6079I_W6081I_I6088L |
| D1609 | A6085YW_T6086F | L6079I_W6081I_I6088L |
| D1610 | A6085YH_T6086F | L6079I_W6081I_I6088L |
| D1611 | A6085YF_T6086M | L6079I_W6081I_I6088L |
| D1612 | A6085YY_T6086M | L6079I_W6081I_I6088L |
| D1613 | A6085YM_T6086M | L6079I_W6081I_I6088L |
| D1614 | A6085YW_T6086M | L6079I_W6081I_I6088L |
| D1615 | A6085YH_T6086M | L6079I_W6081I_I6088L |
| D1616 | A6085YF_T6086W | L6079I_W6081I_I6088L |
| D1617 | A6085YY_T6086W | L6079I_W6081I_I6088L |
| D1618 | A6085YM_T6086W | L6079I_W6081I_I6088L |
| D1619 | A6085YW_T6086W | L6079I_W6081I_I6088L |
| D1620 | A6085YH_T6086W | L6079I_W6081I_I6088L |
| D1621 | A6085YF_T6086H | L6079I_W6081I_I6088L |
| D1622 | A6085YY_T6086H | L6079I_W6081I_I6088L |
| D1623 | A6085YM_T6086H | L6079I_W6081I_I6088L |
| D1624 | A6085YW_T6086H | L6079I_W6081I_I6088L |
| D1625 | A6085YH_T6086H | L6079I_W6081I_I6088L |
| D1626 | A6085YF_T6086Y | L6079V_W6081T_I6088A |
| D1627 | A6085YY_T6086Y | L6079V_W6081T_I6088A |
| D1628 | A6085YM_T6086Y | L6079V_W6081T_I6088A |
| D1629 | A6085YW_T6086Y | L6079V_W6081T_I6088A |
| D1630 | A6085YH_T6086Y | L6079V_W6081T_I6088A |
| D1631 | A6085YF_T6086F | L6079V_W6081T_I6088A |
| D1632 | A6085YY_T6086F | L6079V_W6081T_I6088A |
| D1633 | A6085YM_T6086F | L6079V_W6081T_I6088A |
| D1634 | A6085YW_T6086F | L6079V_W6081T_I6088A |
| D1635 | A6085YH_T6086F | L6079V_W6081T_I6088A |
| D1636 | A6085YF_T6086M | L6079V_W6081T_I6088A |
| D1637 | A6085YY_T6086M | L6079V_W6081T_I6088A |
| D1638 | A6085YM_T6086M | L6079V_W6081T_I6088A |
| D1639 | A6085YW_T6086M | L6079V_W6081T_I6088A |
| D1640 | A6085YH_T6086M | L6079V_W6081T_I6088A |
| D1641 | A6085YF_T6086W | L6079V_W6081T_I6088A |
| D1642 | A6085YY_T6086W | L6079V_W6081T_I6088A |
| D1643 | A6085YM_T6086W | L6079V_W6081T_I6088A |
| D1644 | A6085YW_T6086W | L6079V_W6081T_I6088A |
| D1645 | A6085YH_T6086W | L6079V_W6081T_I6088A |
| D1646 | A6085YF_T6086H | L6079V_W6081T_I6088A |
| D1647 | A6085YY_T6086H | L6079V_W6081T_I6088A |
| D1648 | A6085YM_T6086H | L6079V_W6081T_I6088A |
| D1649 | A6085YW_T6086H | L6079V_W6081T_I6088A |
| D1650 | A6085YH_T6086H | L6079V_W6081T_I6088A |
| D1651 | A6085YF_T6086Y | L6079V_W6081L_I6088A |
| D1652 | A6085YY_T6086Y | L6079V_W6081L_I6088A |
| D1653 | A6085YM_T6086Y | L6079V_W6081L_I6088A |
| D1654 | A6085YW_T6086Y | L6079V_W6081L_I6088A |
| D1655 | A6085YH_T6086Y | L6079V_W6081L_I6088A |
| D1656 | A6085YF_T6086F | L6079V_W6081L_I6088A |
| D1657 | A6085YY_T6086F | L6079V_W6081L_I6088A |
| D1658 | A6085YM_T6086F | L6079V_W6081L_I6088A |
| D1659 | A6085YW_T6086F | L6079V_W6081L_I6088A |
| D1660 | A6085YH_T6086F | L6079V_W6081L_I6088A |
| D1661 | A6085YF_T6086M | L6079V_W6081L_I6088A |
| D1662 | A6085YY_T6086M | L6079V_W6081L_I6088A |
| D1663 | A6085YM_T6086M | L6079V_W6081L_I6088A |
| D1664 | A6085YW_T6086M | L6079V_W6081L_I6088A |
| D1665 | A6085YH_T6086M | L6079V_W6081L_I6088A |
| D1666 | A6085YF_T6086W | L6079V_W6081L_I6088A |
| D1667 | A6085YY_T6086W | L6079V_W6081L_I6088A |
| D1668 | A6085YM_T6086W | L6079V_W6081L_I6088A |
| D1669 | A6085YW_T6086W | L6079V_W6081L_I6088A |
| D1670 | A6085YH_T6086W | L6079V_W6081L_I6088A |
| D1671 | A6085YF_T6086H | L6079V_W6081L_I6088A |
| D1672 | A6085YY_T6086H | L6079V_W6081L_I6088A |
| D1673 | A6085YM_T6086H | L6079V_W6081L_I6088A |
| D1674 | A6085YW_T6086H | L6079V_W6081L_I6088A |
| D1675 | A6085YH_T6086H | L6079V_W6081L_I6088A |
| D1676 | A6085YF_T6086Y | L6079V_W6081A_I6088A |
| D1677 | A6085YY_T6086Y | L6079V_W6081A_I6088A |
| D1678 | A6085YM_T6086Y | L6079V_W6081A_I6088A |
| D1679 | A6085YW_T6086Y | L6079V_W6081A_I6088A |
| D1680 | A6085YH_T6086Y | L6079V_W6081A_I6088A |
| D1681 | A6085YF_T6086F | L6079V_W6081A_I6088A |
| D1682 | A6085YY_T6086F | L6079V_W6081A_I6088A |
| D1683 | A6085YM_T6086F | L6079V_W6081A_I6088A |
| D1684 | A6085YW_T6086F | L6079V_W6081A_I6088A |
| D1685 | A6085YH_T6086F | L6079V_W6081A_I6088A |
| D1686 | A6085YF_T6086M | L6079V_W6081A_I6088A |
| D1687 | A6085YY_T6086M | L6079V_W6081A_I6088A |
| D1688 | A6085YM_T6086M | L6079V_W6081A_I6088A |
| D1689 | A6085YW_T6086M | L6079V_W6081A_I6088A |
| D1690 | A6085YH_T6086M | L6079V_W6081A_I6088A |
| D1691 | A6085YF_T6086W | L6079V_W6081A_I6088A |
| D1692 | A6085YY_T6086W | L6079V_W6081A_I6088A |
| D1693 | A6085YM_T6086W | L6079V_W6081A_I6088A |
| D1694 | A6085YW_T6086W | L6079V_W6081A_I6088A |
| D1695 | A6085YH_T6086W | L6079V_W6081A_I6088A |
| D1696 | A6085YF_T6086H | L6079V_W6081A_I6088A |
| D1697 | A6085YY_T6086H | L6079V_W6081A_I6088A |
| D1698 | A6085YM_T6086H | L6079V_W6081A_I6088A |
| D1699 | A6085YW_T6086H | L6079V_W6081A_I6088A |
| D1700 | A6085YH_T6086H | L6079V_W6081A_I6088A |
| D1701 | A6085YF_T6086Y | L6079V_W6081V_I6088A |
| D1702 | A6085YY_T6086Y | L6079V_W6081V_I6088A |
| D1703 | A6085YM_T6086Y | L6079V_W6081V_I6088A |
| D1704 | A6085YW_T6086Y | L6079V_W6081V_I6088A |
| D1705 | A6085YH_T6086Y | L6079V_W6081V_I6088A |
| D1706 | A6085YF_T6086F | L6079V_W6081V_I6088A |
| D1707 | A6085YY_T6086F | L6079V_W6081V_I6088A |
| D1708 | A6085YM_T6086F | L6079V_W6081V_I6088A |
| D1709 | A6085YW_T6086F | L6079V_W6081V_I6088A |
| D1710 | A6085YH_T6086F | L6079V_W6081V_I6088A |
| D1711 | A6085YF_T6086M | L6079V_W6081V_I6088A |
| D1712 | A6085YY_T6086M | L6079V_W6081V_I6088A |
| D1713 | A6085YM_T6086M | L6079V_W6081V_I6088A |
| D1714 | A6085YW_T6086M | L6079V_W6081V_I6088A |
| D1715 | A6085YH_T6086M | L6079V_W6081V_I6088A |
| D1716 | A6085YF_T6086W | L6079V_W6081V_I6088A |
| D1717 | A6085YY_T6086W | L6079V_W6081V_I6088A |
| D1718 | A6085YM_T6086W | L6079V_W6081V_I6088A |
| D1719 | A6085YW_T6086W | L6079V_W6081V_I6088A |
| D1720 | A6085YH_T6086W | L6079V_W6081V_I6088A |
| D1721 | A6085YF_T6086H | L6079V_W6081V_I6088A |
| D1722 | A6085YY_T6086H | L6079V_W6081V_I6088A |
| D1723 | A6085YM_T6086H | L6079V_W6081V_I6088A |
| D1724 | A6085YW_T6086H | L6079V_W6081V_I6088A |
| D1725 | A6085YH_T6086H | L6079V_W6081V_I6088A |
| D1726 | A6085YF_T6086Y | L6079V_W6081I_I6088A |
| D1727 | A6085YY_T6086Y | L6079V_W6081I_I6088A |
| D1728 | A6085YM_T6086Y | L6079V_W6081I_I6088A |
| D1729 | A6085YW_T6086Y | L6079V_W6081I_I6088A |
| D1730 | A6085YH_T6086Y | L6079V_W6081I_I6088A |
| D1731 | A6085YF_T6086F | L6079V_W6081I_I6088A |
| D1732 | A6085YY_T6086F | L6079V_W6081I_I6088A |
| D1733 | A6085YM_T6086F | L6079V_W6081I_I6088A |
| D1734 | A6085YW_T6086F | L6079V_W6081I_I6088A |
| D1735 | A6085YH_T6086F | L6079V_W6081I_I6088A |
| D1736 | A6085YF_T6086M | L6079V_W6081I_I6088A |
| D1737 | A6085YY_T6086M | L6079V_W6081I_I6088A |
| D1738 | A6085YM_T6086M | L6079V_W6081I_I6088A |
| D1739 | A6085YW_T6086M | L6079V_W6081I_I6088A |
| D1740 | A6085YH_T6086M | L6079V_W6081I_I6088A |
| D1741 | A6085YF_T6086W | L6079V_W6081I_I6088A |
| D1742 | A6085YY_T6086W | L6079V_W6081I_I6088A |
| D1743 | A6085YM_T6086W | L6079V_W6081I_I6088A |
| D1744 | A6085YW_T6086W | L6079V_W6081I_I6088A |
| D1745 | A6085YH_T6086W | L6079V_W6081I_I6088A |
| D1746 | A6085YF_T6086H | L6079V_W6081I_I6088A |
| D1747 | A6085YY_T6086H | L6079V_W6081I_I6088A |
| D1748 | A6085YM_T6086H | L6079V_W6081I_I6088A |
| D1749 | A6085YW_T6086H | L6079V_W6081I_I6088A |
| D1750 | A6085YH_T6086H | L6079V_W6081I_I6088A |
| D1751 | A6085YF_T6086Y | L6079T_W6081T_I6088A |
| D1752 | A6085YY_T6086Y | L6079T_W6081T_I6088A |
| D1753 | A6085YM_T6086Y | L6079T_W6081T_I6088A |
| D1754 | A6085YW_T6086Y | L6079T_W6081T_I6088A |
| D1755 | A6085YH_T6086Y | L6079T_W6081T_I6088A |
| D1756 | A6085YF_T6086F | L6079T_W6081T_I6088A |
| D1757 | A6085YY_T6086F | L6079T_W6081T_I6088A |
| D1758 | A6085YM_T6086F | L6079T_W6081T_I6088A |
| D1759 | A6085YW_T6086F | L6079T_W6081T_I6088A |
| D1760 | A6085YH_T6086F | L6079T_W6081T_I6088A |
| D1761 | A6085YF_T6086M | L6079T_W6081T_I6088A |
| D1762 | A6085YY_T6086M | L6079T_W6081T_I6088A |
| D1763 | A6085YM_T6086M | L6079T_W6081T_I6088A |
| D1764 | A6085YW_T6086M | L6079T_W6081T_I6088A |
| D1765 | A6085YH_T6086M | L6079T_W6081T_I6088A |
| D1766 | A6085YF_T6086W | L6079T_W6081T_I6088A |
| D1767 | A6085YY_T6086W | L6079T_W6081T_I6088A |
| D1768 | A6085YM_T6086W | L6079T_W6081T_I6088A |
| D1769 | A6085YW_T6086W | L6079T_W6081T_I6088A |
| D1770 | A6085YH_T6086W | L6079T_W6081T_I6088A |
| D1771 | A6085YF_T6086H | L6079T_W6081T_I6088A |
| D1772 | A6085YY_T6086H | L6079T_W6081T_I6088A |
| D1773 | A6085YM_T6086H | L6079T_W6081T_I6088A |
| D1774 | A6085YW_T6086H | L6079T_W6081T_I6088A |
| D1775 | A6085YH_T6086H | L6079T_W6081T_I6088A |
| D1776 | A6085YF_T6086Y | L6079T_W6081L_I6088A |
| D1777 | A6085YY_T6086Y | L6079T_W6081L_I6088A |
| D1778 | A6085YM_T6086Y | L6079T_W6081L_I6088A |
| D1779 | A6085YW_T6086Y | L6079T_W6081L_I6088A |
| D1780 | A6085YH_T6086Y | L6079T_W6081L_I6088A |
| D1781 | A6085YF_T6086F | L6079T_W6081L_I6088A |
| D1782 | A6085YY_T6086F | L6079T_W6081L_I6088A |
| D1783 | A6085YM_T6086F | L6079T_W6081L_I6088A |
| D1784 | A6085YW_T6086F | L6079T_W6081L_I6088A |
| D1785 | A6085YH_T6086F | L6079T_W6081L_I6088A |
| D1786 | A6085YF_T6086M | L6079T_W6081L_I6088A |
| D1787 | A6085YY_T6086M | L6079T_W6081L_I6088A |
| D1788 | A6085YM_T6086M | L6079T_W6081L_I6088A |
| D1789 | A6085YW_T6086M | L6079T_W6081L_I6088A |
| D1790 | A6085YH_T6086M | L6079T_W6081L_I6088A |
| D1791 | A6085YF_T6086W | L6079T_W6081L_I6088A |
| D1792 | A6085YY_T6086W | L6079T_W6081L_I6088A |
| D1793 | A6085YM_T6086W | L6079T_W6081L_I6088A |
| D1794 | A6085YW_T6086W | L6079T_W6081L_I6088A |
| D1795 | A6085YH_T6086W | L6079T_W6081L_I6088A |
| D1796 | A6085YF_T6086H | L6079T_W6081L_I6088A |
| D1797 | A6085YY_T6086H | L6079T_W6081L_I6088A |
| D1798 | A6085YM_T6086H | L6079T_W6081L_I6088A |
| D1799 | A6085YW_T6086H | L6079T_W6081L_I6088A |
| D1800 | A6085YH_T6086H | L6079T_W6081L_I6088A |
| D1801 | A6085YF_T6086Y | L6079T_W6081A_I6088A |
| D1802 | A6085YY_T6086Y | L6079T_W6081A_I6088A |
| D1803 | A6085YM_T6086Y | L6079T_W6081A_I6088A |
| D1804 | A6085YW_T6086Y | L6079T_W6081A_I6088A |
| D1805 | A6085YH_T6086Y | L6079T_W6081A_I6088A |
| D1806 | A6085YF_T6086F | L6079T_W6081A_I6088A |
| D1807 | A6085YY_T6086F | L6079T_W6081A_I6088A |
| D1808 | A6085YM_T6086F | L6079T_W6081A_I6088A |
| D1809 | A6085YW_T6086F | L6079T_W6081A_I6088A |
| D1810 | A6085YH_T6086F | L6079T_W6081A_I6088A |
| D1811 | A6085YF_T6086M | L6079T_W6081A_I6088A |
| D1812 | A6085YY_T6086M | L6079T_W6081A_I6088A |
| D1813 | A6085YM_T6086M | L6079T_W6081A_I6088A |
| D1814 | A6085YW_T6086M | L6079T_W6081A_I6088A |
| D1815 | A6085YH_T6086M | L6079T_W6081A_I6088A |
| D1816 | A6085YF_T6086W | L6079T_W6081A_I6088A |
| D1817 | A6085YY_T6086W | L6079T_W6081A_I6088A |
| D1818 | A6085YM_T6086W | L6079T_W6081A_I6088A |
| D1819 | A6085YW_T6086W | L6079T_W6081A_I6088A |
| D1820 | A6085YH_T6086W | L6079T_W6081A_I6088A |
| D1821 | A6085YF_T6086H | L6079T_W6081A_I6088A |
| D1822 | A6085YY_T6086H | L6079T_W6081A_I6088A |
| D1823 | A6085YM_T6086H | L6079T_W6081A_I6088A |
| D1824 | A6085YW_T6086H | L6079T_W6081A_I6088A |
| D1825 | A6085YH_T6086H | L6079T_W6081A_I6088A |
| D1826 | A6085YF_T6086Y | L6079T_W6081V_I6088A |
| D1827 | A6085YY_T6086Y | L6079T_W6081V_I6088A |
| D1828 | A6085YM_T6086Y | L6079T_W6081V_I6088A |
| D1829 | A6085YW_T6086Y | L6079T_W6081V_I6088A |
| D1830 | A6085YH_T6086Y | L6079T_W6081V_I6088A |
| D1831 | A6085YF_T6086F | L6079T_W6081V_I6088A |
| D1832 | A6085YY_T6086F | L6079T_W6081V_I6088A |
| D1833 | A6085YM_T6086F | L6079T_W6081V_I6088A |
| D1834 | A6085YW_T6086F | L6079T_W6081V_I6088A |
| D1835 | A6085YH_T6086F | L6079T_W6081V_I6088A |
| D1836 | A6085YF_T6086M | L6079T_W6081V_I6088A |
| D1837 | A6085YY_T6086M | L6079T_W6081V_I6088A |
| D1838 | A6085YM_T6086M | L6079T_W6081V_I6088A |
| D1839 | A6085YW_T6086M | L6079T_W6081V_I6088A |
| D1840 | A6085YH_T6086M | L6079T_W6081V_I6088A |
| D1841 | A6085YF_T6086W | L6079T_W6081V_I6088A |
| D1842 | A6085YY_T6086W | L6079T_W6081V_I6088A |
| D1843 | A6085YM_T6086W | L6079T_W6081V_I6088A |
| D1844 | A6085YW_T6086W | L6079T_W6081V_I6088A |
| D1845 | A6085YH_T6086W | L6079T_W6081V_I6088A |
| D1846 | A6085YF_T6086H | L6079T_W6081V_I6088A |
| D1847 | A6085YY_T6086H | L6079T_W6081V_I6088A |
| D1848 | A6085YM_T6086H | L6079T_W6081V_I6088A |
| D1849 | A6085YW_T6086H | L6079T_W6081V_I6088A |
| D1850 | A6085YH_T6086H | L6079T_W6081V_I6088A |
| D1851 | A6085YF_T6086Y | L6079T_W6081I_I6088A |
| D1852 | A6085YY_T6086Y | L6079T_W6081I_I6088A |
| D1853 | A6085YM_T6086Y | L6079T_W6081I_I6088A |
| D1854 | A6085YW_T6086Y | L6079T_W6081I_I6088A |
| D1855 | A6085YH_T6086Y | L6079T_W6081I_I6088A |
| D1856 | A6085YF_T6086F | L6079T_W6081I_I6088A |
| D1857 | A6085YY_T6086F | L6079T_W6081I_I6088A |
| D1858 | A6085YM_T6086F | L6079T_W6081I_I6088A |
| D1859 | A6085YW_T6086F | L6079T_W6081I_I6088A |
| D1860 | A6085YH_T6086F | L6079T_W6081I_I6088A |
| D1861 | A6085YF_T6086M | L6079T_W6081I_I6088A |
| D1862 | A6085YY_T6086M | L6079T_W6081I_I6088A |
| D1863 | A6085YM_T6086M | L6079T_W6081I_I6088A |
| D1864 | A6085YW_T6086M | L6079T_W6081I_I6088A |
| D1865 | A6085YH_T6086M | L6079T_W6081I_I6088A |
| D1866 | A6085YF_T6086W | L6079T_W6081I_I6088A |
| D1867 | A6085YY_T6086W | L6079T_W6081I_I6088A |
| D1868 | A6085YM_T6086W | L6079T_W6081I_I6088A |
| D1869 | A6085YW_T6086W | L6079T_W6081I_I6088A |
| D1870 | A6085YH_T6086W | L6079T_W6081I_I6088A |
| D1871 | A6085YF_T6086H | L6079T_W6081I_I6088A |
| D1872 | A6085YY_T6086H | L6079T_W6081I_I6088A |
| D1873 | A6085YM_T6086H | L6079T_W6081I_I6088A |
| D1874 | A6085YW_T6086H | L6079T_W6081I_I6088A |
| D1875 | A6085YH_T6086H | L6079T_W6081I_I6088A |
| D1876 | A6085YF_T6086Y | L6079A_W6081T_I6088A |
| D1877 | A6085YY_T6086Y | L6079A_W6081T_I6088A |
| D1878 | A6085YM_T6086Y | L6079A_W6081T_I6088A |
| D1879 | A6085YW_T6086Y | L6079A_W6081T_I6088A |
| D1880 | A6085YH_T6086Y | L6079A_W6081T_I6088A |
| D1881 | A6085YF_T6086F | L6079A_W6081T_I6088A |
| D1882 | A6085YY_T6086F | L6079A_W6081T_I6088A |
| D1883 | A6085YM_T6086F | L6079A_W6081T_I6088A |
| D1884 | A6085YW_T6086F | L6079A_W6081T_I6088A |
| D1885 | A6085YH_T6086F | L6079A_W6081T_I6088A |
| D1886 | A6085YF_T6086M | L6079A_W6081T_I6088A |
| D1887 | A6085YY_T6086M | L6079A_W6081T_I6088A |
| D1888 | A6085YM_T6086M | L6079A_W6081T_I6088A |
| D1889 | A6085YW_T6086M | L6079A_W6081T_I6088A |
| D1890 | A6085YH_T6086M | L6079A_W6081T_I6088A |
| D1891 | A6085YF_T6086W | L6079A_W6081T_I6088A |
| D1892 | A6085YY_T6086W | L6079A_W6081T_I6088A |
| D1893 | A6085YM_T6086W | L6079A_W6081T_I6088A |
| D1894 | A6085YW_T6086W | L6079A_W6081T_I6088A |
| D1895 | A6085YH_T6086W | L6079A_W6081T_I6088A |
| D1896 | A6085YF_T6086H | L6079A_W6081T_I6088A |
| D1897 | A6085YY_T6086H | L6079A_W6081T_I6088A |
| D1898 | A6085YM_T6086H | L6079A_W6081T_I6088A |
| D1899 | A6085YW_T6086H | L6079A_W6081T_I6088A |
| D1900 | A6085YH_T6086H | L6079A_W6081T_I6088A |
| D1901 | A6085YF_T6086Y | L6079A_W6081L_I6088A |
| D1902 | A6085YY_T6086Y | L6079A_W6081L_I6088A |
| D1903 | A6085YM_T6086Y | L6079A_W6081L_I6088A |
| D1904 | A6085YW_T6086Y | L6079A_W6081L_I6088A |
| D1905 | A6085YH_T6086Y | L6079A_W6081L_I6088A |
| D1906 | A6085YF_T6086F | L6079A_W6081L_I6088A |
| D1907 | A6085YY_T6086F | L6079A_W6081L_I6088A |
| D1908 | A6085YM_T6086F | L6079A_W6081L_I6088A |
| D1909 | A6085YW_T6086F | L6079A_W6081L_I6088A |
| D1910 | A6085YH_T6086F | L6079A_W6081L_I6088A |
| D1911 | A6085YF_T6086M | L6079A_W6081L_I6088A |
| D1912 | A6085YY_T6086M | L6079A_W6081L_I6088A |
| D1913 | A6085YM_T6086M | L6079A_W6081L_I6088A |
| D1914 | A6085YW_T6086M | L6079A_W6081L_I6088A |
| D1915 | A6085YH_T6086M | L6079A_W6081L_I6088A |
| D1916 | A6085YF_T6086W | L6079A_W6081L_I6088A |
| D1917 | A6085YY_T6086W | L6079A_W6081L_I6088A |
| D1918 | A6085YM_T6086W | L6079A_W6081L_I6088A |
| D1919 | A6085YW_T6086W | L6079A_W6081L_I6088A |
| D1920 | A6085YH_T6086W | L6079A_W6081L_I6088A |
| D1921 | A6085YF_T6086H | L6079A_W6081L_I6088A |
| D1922 | A6085YY_T6086H | L6079A_W6081L_I6088A |
| D1923 | A6085YM_T6086H | L6079A_W6081L_I6088A |
| D1924 | A6085YW_T6086H | L6079A_W6081L_I6088A |
| D1925 | A6085YH_T6086H | L6079A_W6081L_I6088A |
| D1926 | A6085YF_T6086Y | L6079A_W6081A_I6088A |
| D1927 | A6085YY_T6086Y | L6079A_W6081A_I6088A |
| D1928 | A6085YM_T6086Y | L6079A_W6081A_I6088A |
| D1929 | A6085YW_T6086Y | L6079A_W6081A_I6088A |
| D1930 | A6085YH_T6086Y | L6079A_W6081A_I6088A |
| D1931 | A6085YF_T6086F | L6079A_W6081A_I6088A |
| D1932 | A6085YY_T6086F | L6079A_W6081A_I6088A |
| D1933 | A6085YM_T6086F | L6079A_W6081A_I6088A |
| D1934 | A6085YW_T6086F | L6079A_W6081A_I6088A |
| D1935 | A6085YH_T6086F | L6079A_W6081A_I6088A |
| D1936 | A6085YF_T6086M | L6079A_W6081A_I6088A |
| D1937 | A6085YY_T6086M | L6079A_W6081A_I6088A |
| D1938 | A6085YM_T6086M | L6079A_W6081A_I6088A |
| D1939 | A6085YW_T6086M | L6079A_W6081A_I6088A |
| D1940 | A6085YH_T6086M | L6079A_W6081A_I6088A |
| D1941 | A6085YF_T6086W | L6079A_W6081A_I6088A |
| D1942 | A6085YY_T6086W | L6079A_W6081A_I6088A |
| D1943 | A6085YM_T6086W | L6079A_W6081A_I6088A |
| D1944 | A6085YW_T6086W | L6079A_W6081A_I6088A |
| D1945 | A6085YH_T6086W | L6079A_W6081A_I6088A |
| D1946 | A6085YF_T6086H | L6079A_W6081A_I6088A |
| D1947 | A6085YY_T6086H | L6079A_W6081A_I6088A |
| D1948 | A6085YM_T6086H | L6079A_W6081A_I6088A |
| D1949 | A6085YW_T6086H | L6079A_W6081A_I6088A |
| D1950 | A6085YH_T6086H | L6079A_W6081A_I6088A |
| D1951 | A6085YF_T6086Y | L6079A_W6081V_I6088A |
| D1952 | A6085YY_T6086Y | L6079A_W6081V_I6088A |
| D1953 | A6085YM_T6086Y | L6079A_W6081V_I6088A |
| D1954 | A6085YW_T6086Y | L6079A_W6081V_I6088A |
| D1955 | A6085YH_T6086Y | L6079A_W6081V_I6088A |
| D1956 | A6085YF_T6086F | L6079A_W6081V_I6088A |
| D1957 | A6085YY_T6086F | L6079A_W6081V_I6088A |
| D1958 | A6085YM_T6086F | L6079A_W6081V_I6088A |
| D1959 | A6085YW_T6086F | L6079A_W6081V_I6088A |
| D1960 | A6085YH_T6086F | L6079A_W6081V_I6088A |
| D1961 | A6085YF_T6086M | L6079A_W6081V_I6088A |
| D1962 | A6085YY_T6086M | L6079A_W6081V_I6088A |
| D1963 | A6085YM_T6086M | L6079A_W6081V_I6088A |
| D1964 | A6085YW_T6086M | L6079A_W6081V_I6088A |
| D1965 | A6085YH_T6086M | L6079A_W6081V_I6088A |
| D1966 | A6085YF_T6086W | L6079A_W6081V_I6088A |
| D1967 | A6085YY_T6086W | L6079A_W6081V_I6088A |
| D1968 | A6085YM_T6086W | L6079A_W6081V_I6088A |
| D1969 | A6085YW_T6086W | L6079A_W6081V_I6088A |
| D1970 | A6085YH_T6086W | L6079A_W6081V_I6088A |
| D1971 | A6085YF_T6086H | L6079A_W6081V_I6088A |
| D1972 | A6085YY_T6086H | L6079A_W6081V_I6088A |
| D1973 | A6085YM_T6086H | L6079A_W6081V_I6088A |
| D1974 | A6085YW_T6086H | L6079A_W6081V_I6088A |
| D1975 | A6085YH_T6086H | L6079A_W6081V_I6088A |
| D1976 | A6085YF_T6086Y | L6079A_W6081I_I6088A |
| D1977 | A6085YY_T6086Y | L6079A_W6081I_I6088A |
| D1978 | A6085YM_T6086Y | L6079A_W6081I_I6088A |
| D1979 | A6085YW_T6086Y | L6079A_W6081I_I6088A |
| D1980 | A6085YH_T6086Y | L6079A_W6081I_I6088A |
| D1981 | A6085YF_T6086F | L6079A_W6081I_I6088A |
| D1982 | A6085YY_T6086F | L6079A_W6081I_I6088A |
| D1983 | A6085YM_T6086F | L6079A_W6081I_I6088A |
| D1984 | A6085YW_T6086F | L6079A_W6081I_I6088A |
| D1985 | A6085YH_T6086F | L6079A_W6081I_I6088A |
| D1986 | A6085YF_T6086M | L6079A_W6081I_I6088A |
| D1987 | A6085YY_T6086M | L6079A_W6081I_I6088A |
| D1988 | A6085YM_T6086M | L6079A_W6081I_I6088A |
| D1989 | A6085YW_T6086M | L6079A_W6081I_I6088A |
| D1990 | A6085YH_T6086M | L6079A_W6081I_I6088A |
| D1991 | A6085YF_T6086W | L6079A_W6081I_I6088A |
| D1992 | A6085YY_T6086W | L6079A_W6081I_I6088A |
| D1993 | A6085YM_T6086W | L6079A_W6081I_I6088A |
| D1994 | A6085YW_T6086W | L6079A_W6081I_I6088A |
| D1995 | A6085YH_T6086W | L6079A_W6081I_I6088A |
| D1996 | A6085YF_T6086H | L6079A_W6081I_I6088A |
| D1997 | A6085YY_T6086H | L6079A_W6081I_I6088A |
| D1998 | A6085YM_T6086H | L6079A_W6081I_I6088A |
| D1999 | A6085YW_T6086H | L6079A_W6081I_I6088A |
| D2000 | A6085YH_T6086H | L6079A_W6081I_I6088A |
| D2001 | A6085YF_T6086Y | L6079I_W6081T_I6088A |
| D2002 | A6085YY_T6086Y | L6079I_W6081T_I6088A |
| D2003 | A6085YM_T6086Y | L6079I_W6081T_I6088A |
| D2004 | A6085YW_T6086Y | L6079I_W6081T_I6088A |
| D2005 | A6085YH_T6086Y | L6079I_W6081T_I6088A |
| D2006 | A6085YF_T6086F | L6079I_W6081T_I6088A |
| D2007 | A6085YY_T6086F | L6079I_W6081T_I6088A |
| D2008 | A6085YM_T6086F | L6079I_W6081T_I6088A |
| D2009 | A6085YW_T6086F | L6079I_W6081T_I6088A |
| D2010 | A6085YH_T6086F | L6079I_W6081T_I6088A |
| D2011 | A6085YF_T6086M | L6079I_W6081T_I6088A |
| D2012 | A6085YY_T6086M | L6079I_W6081T_I6088A |
| D2013 | A6085YM_T6086M | L6079I_W6081T_I6088A |
| D2014 | A6085YW_T6086M | L6079I_W6081T_I6088A |
| D2015 | A6085YH_T6086M | L6079I_W6081T_I6088A |
| D2016 | A6085YF_T6086W | L6079I_W6081T_I6088A |
| D2017 | A6085YY_T6086W | L6079I_W6081T_I6088A |
| D2018 | A6085YM_T6086W | L6079I_W6081T_I6088A |
| D2019 | A6085YW_T6086W | L6079I_W6081T_I6088A |
| D2020 | A6085YH_T6086W | L6079I_W6081T_I6088A |
| D2021 | A6085YF_T6086H | L6079I_W6081T_I6088A |
| D2022 | A6085YY_T6086H | L6079I_W6081T_I6088A |
| D2023 | A6085YM_T6086H | L6079I_W6081T_I6088A |
| D2024 | A6085YW_T6086H | L6079I_W6081T_I6088A |
| D2025 | A6085YH_T6086H | L6079I_W6081T_I6088A |
| D2026 | A6085YF_T6086Y | L6079I_W6081L_I6088A |
| D2027 | A6085YY_T6086Y | L6079I_W6081L_I6088A |
| D2028 | A6085YM_T6086Y | L6079I_W6081L_I6088A |
| D2029 | A6085YW_T6086Y | L6079I_W6081L_I6088A |
| D2030 | A6085YH_T6086Y | L6079I_W6081L_I6088A |
| D2031 | A6085YF_T6086F | L6079I_W6081L_I6088A |
| D2032 | A6085YY_T6086F | L6079I_W6081L_I6088A |
| D2033 | A6085YM_T6086F | L6079I_W6081L_I6088A |
| D2034 | A6085YW_T6086F | L6079I_W6081L_I6088A |
| D2035 | A6085YH_T6086F | L6079I_W6081L_I6088A |
| D2036 | A6085YF_T6086M | L6079I_W6081L_I6088A |
| D2037 | A6085YY_T6086M | L6079I_W6081L_I6088A |
| D2038 | A6085YM_T6086M | L6079I_W6081L_I6088A |
| D2039 | A6085YW_T6086M | L6079I_W6081L_I6088A |
| D2040 | A6085YH_T6086M | L6079I_W6081L_I6088A |
| D2041 | A6085YF_T6086W | L6079I_W6081L_I6088A |
| D2042 | A6085YY_T6086W | L6079I_W6081L_I6088A |
| D2043 | A6085YM_T6086W | L6079I_W6081L_I6088A |
| D2044 | A6085YW_T6086W | L6079I_W6081L_I6088A |
| D2045 | A6085YH_T6086W | L6079I_W6081L_I6088A |
| D2046 | A6085YF_T6086H | L6079I_W6081L_I6088A |
| D2047 | A6085YY_T6086H | L6079I_W6081L_I6088A |
| D2048 | A6085YM_T6086H | L6079I_W6081L_I6088A |
| D2049 | A6085YW_T6086H | L6079I_W6081L_I6088A |
| D2050 | A6085YH_T6086H | L6079I_W6081L_I6088A |
| D2051 | A6085YF_T6086Y | L6079I_W6081A_I6088A |
| D2052 | A6085YY_T6086Y | L6079I_W6081A_I6088A |
| D2053 | A6085YM_T6086Y | L6079I_W6081A_I6088A |
| D2054 | A6085YW_T6086Y | L6079I_W6081A_I6088A |
| D2055 | A6085YH_T6086Y | L6079I_W6081A_I6088A |
| D2056 | A6085YF_T6086F | L6079I_W6081A_I6088A |
| D2057 | A6085YY_T6086F | L6079I_W6081A_I6088A |
| D2058 | A6085YM_T6086F | L6079I_W6081A_I6088A |
| D2059 | A6085YW_T6086F | L6079I_W6081A_I6088A |
| D2060 | A6085YH_T6086F | L6079I_W6081A_I6088A |
| D2061 | A6085YF_T6086M | L6079I_W6081A_I6088A |
| D2062 | A6085YY_T6086M | L6079I_W6081A_I6088A |
| D2063 | A6085YM_T6086M | L6079I_W6081A_I6088A |
| D2064 | A6085YW_T6086M | L6079I_W6081A_I6088A |
| D2065 | A6085YH_T6086M | L6079I_W6081A_I6088A |
| D2066 | A6085YF_T6086W | L6079I_W6081A_I6088A |
| D2067 | A6085YY_T6086W | L6079I_W6081A_I6088A |
| D2068 | A6085YM_T6086W | L6079I_W6081A_I6088A |
| D2069 | A6085YW_T6086W | L6079I_W6081A_I6088A |
| D2070 | A6085YH_T6086W | L6079I_W6081A_I6088A |
| D2071 | A6085YF_T6086H | L6079I_W6081A_I6088A |
| D2072 | A6085YY_T6086H | L6079I_W6081A_I6088A |
| D2073 | A6085YM_T6086H | L6079I_W6081A_I6088A |
| D2074 | A6085YW_T6086H | L6079I_W6081A_I6088A |
| D2075 | A6085YH_T6086H | L6079I_W6081A_I6088A |
| D2076 | A6085YF_T6086Y | L6079I_W6081V_I6088A |
| D2077 | A6085YY_T6086Y | L6079I_W6081V_I6088A |
| D2078 | A6085YM_T6086Y | L6079I_W6081V_I6088A |
| D2079 | A6085YW_T6086Y | L6079I_W6081V_I6088A |
| D2080 | A6085YH_T6086Y | L6079I_W6081V_I6088A |
| D2081 | A6085YF_T6086F | L6079I_W6081V_I6088A |
| D2082 | A6085YY_T6086F | L6079I_W6081V_I6088A |
| D2083 | A6085YM_T6086F | L6079I_W6081V_I6088A |
| D2084 | A6085YW_T6086F | L6079I_W6081V_I6088A |
| D2085 | A6085YH_T6086F | L6079I_W6081V_I6088A |
| D2086 | A6085YF_T6086M | L6079I_W6081V_I6088A |
| D2087 | A6085YY_T6086M | L6079I_W6081V_I6088A |
| D2088 | A6085YM_T6086M | L6079I_W6081V_I6088A |
| D2089 | A6085YW_T6086M | L6079I_W6081V_I6088A |
| D2090 | A6085YH_T6086M | L6079I_W6081V_I6088A |
| D2091 | A6085YF_T6086W | L6079I_W6081V_I6088A |
| D2092 | A6085YY_T6086W | L6079I_W6081V_I6088A |
| D2093 | A6085YM_T6086W | L6079I_W6081V_I6088A |
| D2094 | A6085YW_T6086W | L6079I_W6081V_I6088A |
| D2095 | A6085YH_T6086W | L6079I_W6081V_I6088A |
| D2096 | A6085YF_T6086H | L6079I_W6081V_I6088A |
| D2097 | A6085YY_T6086H | L6079I_W6081V_I6088A |
| D2098 | A6085YM_T6086H | L6079I_W6081V_I6088A |
| D2099 | A6085YW_T6086H | L6079I_W6081V_I6088A |
| D2100 | A6085YH_T6086H | L6079I_W6081V_I6088A |
| D2101 | A6085YF_T6086Y | L6079I_W6081I_I6088A |
| D2102 | A6085YY_T6086Y | L6079I_W6081I_I6088A |
| D2103 | A6085YM_T6086Y | L6079I_W6081I_I6088A |
| D2104 | A6085YW_T6086Y | L6079I_W6081I_I6088A |
| D2105 | A6085YH_T6086Y | L6079I_W6081I_I6088A |
| D2106 | A6085YF_T6086F | L6079I_W6081I_I6088A |
| D2107 | A6085YY_T6086F | L6079I_W6081I_I6088A |
| D2108 | A6085YM_T6086F | L6079I_W6081I_I6088A |
| D2109 | A6085YW_T6086F | L6079I_W6081I_I6088A |
| D2110 | A6085YH_T6086F | L6079I_W6081I_I6088A |
| D2111 | A6085YF_T6086M | L6079I_W6081I_I6088A |
| D2112 | A6085YY_T6086M | L6079I_W6081I_I6088A |
| D2113 | A6085YM_T6086M | L6079I_W6081I_I6088A |
| D2114 | A6085YW_T6086M | L6079I_W6081I_I6088A |
| D2115 | A6085YH_T6086M | L6079I_W6081I_I6088A |
| D2116 | A6085YF_T6086W | L6079I_W6081I_I6088A |
| D2117 | A6085YY_T6086W | L6079I_W6081I_I6088A |
| D2118 | A6085YM_T6086W | L6079I_W6081I_I6088A |
| D2119 | A6085YW_T6086W | L6079I_W6081I_I6088A |
| D2120 | A6085YH_T6086W | L6079I_W6081I_I6088A |
| D2121 | A6085YF_T6086H | L6079I_W6081I_I6088A |
| D2122 | A6085YY_T6086H | L6079I_W6081I_I6088A |
| D2123 | A6085YM_T6086H | L6079I_W6081I_I6088A |
| D2124 | A6085YW_T6086H | L6079I_W6081I_I6088A |
| D2125 | A6085YH_T6086H | L6079I_W6081I_I6088A |
| D2126 | A6085YF_T6086Y | L6079V_W6081T_I6088V |
| D2127 | A6085YY_T6086Y | L6079V_W6081T_I6088V |
| D2128 | A6085YM_T6086Y | L6079V_W6081T_I6088V |
| D2129 | A6085YW_T6086Y | L6079V_W6081T_I6088V |
| D2130 | A6085YH_T6086Y | L6079V_W6081T_I6088V |
| D2131 | A6085YF_T6086F | L6079V_W6081T_I6088V |
| D2132 | A6085YY_T6086F | L6079V_W6081T_I6088V |
| D2133 | A6085YM_T6086F | L6079V_W6081T_I6088V |
| D2134 | A6085YW_T6086F | L6079V_W6081T_I6088V |
| D2135 | A6085YH_T6086F | L6079V_W6081T_I6088V |
| D2136 | A6085YF_T6086M | L6079V_W6081T_I6088V |
| D2137 | A6085YY_T6086M | L6079V_W6081T_I6088V |
| D2138 | A6085YM_T6086M | L6079V_W6081T_I6088V |
| D2139 | A6085YW_T6086M | L6079V_W6081T_I6088V |
| D2140 | A6085YH_T6086M | L6079V_W6081T_I6088V |
| D2141 | A6085YF_T6086W | L6079V_W6081T_I6088V |
| D2142 | A6085YY_T6086W | L6079V_W6081T_I6088V |
| D2143 | A6085YM_T6086W | L6079V_W6081T_I6088V |
| D2144 | A6085YW_T6086W | L6079V_W6081T_I6088V |
| D2145 | A6085YH_T6086W | L6079V_W6081T_I6088V |
| D2146 | A6085YF_T6086H | L6079V_W6081T_I6088V |
| D2147 | A6085YY_T6086H | L6079V_W6081T_I6088V |
| D2148 | A6085YM_T6086H | L6079V_W6081T_I6088V |
| D2149 | A6085YW_T6086H | L6079V_W6081T_I6088V |
| D2150 | A6085YH_T6086H | L6079V_W6081T_I6088V |
| D2151 | A6085YF_T6086Y | L6079V_W6081L_I6088V |
| D2152 | A6085YY_T6086Y | L6079V_W6081L_I6088V |
| D2153 | A6085YM_T6086Y | L6079V_W6081L_I6088V |
| D2154 | A6085YW_T6086Y | L6079V_W6081L_I6088V |
| D2155 | A6085YH_T6086Y | L6079V_W6081L_I6088V |
| D2156 | A6085YF_T6086F | L6079V_W6081L_I6088V |
| D2157 | A6085YY_T6086F | L6079V_W6081L_I6088V |
| D2158 | A6085YM_T6086F | L6079V_W6081L_I6088V |
| D2159 | A6085YW_T6086F | L6079V_W6081L_I6088V |
| D2160 | A6085YH_T6086F | L6079V_W6081L_I6088V |
| D2161 | A6085YF_T6086M | L6079V_W6081L_I6088V |
| D2162 | A6085YY_T6086M | L6079V_W6081L_I6088V |
| D2163 | A6085YM_T6086M | L6079V_W6081L_I6088V |
| D2164 | A6085YW_T6086M | L6079V_W6081L_I6088V |
| D2165 | A6085YH_T6086M | L6079V_W6081L_I6088V |
| D2166 | A6085YF_T6086W | L6079V_W6081L_I6088V |
| D2167 | A6085YY_T6086W | L6079V_W6081L_I6088V |
| D2168 | A6085YM_T6086W | L6079V_W6081L_I6088V |
| D2169 | A6085YW_T6086W | L6079V_W6081L_I6088V |
| D2170 | A6085YH_T6086W | L6079V_W6081L_I6088V |
| D2171 | A6085YF_T6086H | L6079V_W6081L_I6088V |
| D2172 | A6085YY_T6086H | L6079V_W6081L_I6088V |
| D2173 | A6085YM_T6086H | L6079V_W6081L_I6088V |
| D2174 | A6085YW_T6086H | L6079V_W6081L_I6088V |
| D2175 | A6085YH_T6086H | L6079V_W6081L_I6088V |
| D2176 | A6085YF_T6086Y | L6079V_W6081A_I6088V |
| D2177 | A6085YY_T6086Y | L6079V_W6081A_I6088V |
| D2178 | A6085YM_T6086Y | L6079V_W6081A_I6088V |
| D2179 | A6085YW_T6086Y | L6079V_W6081A_I6088V |
| D2180 | A6085YH_T6086Y | L6079V_W6081A_I6088V |
| D2181 | A6085YF_T6086F | L6079V_W6081A_I6088V |
| D2182 | A6085YY_T6086F | L6079V_W6081A_I6088V |
| D2183 | A6085YM_T6086F | L6079V_W6081A_I6088V |
| D2184 | A6085YW_T6086F | L6079V_W6081A_I6088V |
| D2185 | A6085YH_T6086F | L6079V_W6081A_I6088V |
| D2186 | A6085YF_T6086M | L6079V_W6081A_I6088V |
| D2187 | A6085YY_T6086M | L6079V_W6081A_I6088V |
| D2188 | A6085YM_T6086M | L6079V_W6081A_I6088V |
| D2189 | A6085YW_T6086M | L6079V_W6081A_I6088V |
| D2190 | A6085YH_T6086M | L6079V_W6081A_I6088V |
| D2191 | A6085YF_T6086W | L6079V_W6081A_I6088V |
| D2192 | A6085YY_T6086W | L6079V_W6081A_I6088V |
| D2193 | A6085YM_T6086W | L6079V_W6081A_I6088V |
| D2194 | A6085YW_T6086W | L6079V_W6081A_I6088V |
| D2195 | A6085YH_T6086W | L6079V_W6081A_I6088V |
| D2196 | A6085YF_T6086H | L6079V_W6081A_I6088V |
| D2197 | A6085YY_T6086H | L6079V_W6081A_I6088V |
| D2198 | A6085YM_T6086H | L6079V_W6081A_I6088V |
| D2199 | A6085YW_T6086H | L6079V_W6081A_I6088V |
| D2200 | A6085YH_T6086H | L6079V_W6081A_I6088V |
| D2201 | A6085YF_T6086Y | L6079V_W6081V_I6088V |
| D2202 | A6085YY_T6086Y | L6079V_W6081V_I6088V |
| D2203 | A6085YM_T6086Y | L6079V_W6081V_I6088V |
| D2204 | A6085YW_T6086Y | L6079V_W6081V_I6088V |
| D2205 | A6085YH_T6086Y | L6079V_W6081V_I6088V |
| D2206 | A6085YF_T6086F | L6079V_W6081V_I6088V |
| D2207 | A6085YY_T6086F | L6079V_W6081V_I6088V |
| D2208 | A6085YM_T6086F | L6079V_W6081V_I6088V |
| D2209 | A6085YW_T6086F | L6079V_W6081V_I6088V |
| D2210 | A6085YH_T6086F | L6079V_W6081V_I6088V |
| D2211 | A6085YF_T6086M | L6079V_W6081V_I6088V |
| D2212 | A6085YY_T6086M | L6079V_W6081V_I6088V |
| D2213 | A6085YM_T6086M | L6079V_W6081V_I6088V |
| D2214 | A6085YW_T6086M | L6079V_W6081V_I6088V |
| D2215 | A6085YH_T6086M | L6079V_W6081V_I6088V |
| D2216 | A6085YF_T6086W | L6079V_W6081V_I6088V |
| D2217 | A6085YY_T6086W | L6079V_W6081V_I6088V |
| D2218 | A6085YM_T6086W | L6079V_W6081V_I6088V |
| D2219 | A6085YW_T6086W | L6079V_W6081V_I6088V |
| D2220 | A6085YH_T6086W | L6079V_W6081V_I6088V |
| D2221 | A6085YF_T6086H | L6079V_W6081V_I6088V |
| D2222 | A6085YY_T6086H | L6079V_W6081V_I6088V |
| D2223 | A6085YM_T6086H | L6079V_W6081V_I6088V |
| D2224 | A6085YW_T6086H | L6079V_W6081V_I6088V |
| D2225 | A6085YH_T6086H | L6079V_W6081V_I6088V |
| D2226 | A6085YF_T6086Y | L6079V_W6081I_I6088V |
| D2227 | A6085YY_T6086Y | L6079V_W6081I_I6088V |
| D2228 | A6085YM_T6086Y | L6079V_W6081I_I6088V |
| D2229 | A6085YW_T6086Y | L6079V_W6081I_I6088V |
| D2230 | A6085YH_T6086Y | L6079V_W6081I_I6088V |
| D2231 | A6085YF_T6086F | L6079V_W6081I_I6088V |
| D2232 | A6085YY_T6086F | L6079V_W6081I_I6088V |
| D2233 | A6085YM_T6086F | L6079V_W6081I_I6088V |
| D2234 | A6085YW_T6086F | L6079V_W6081I_I6088V |
| D2235 | A6085YH_T6086F | L6079V_W6081I_I6088V |
| D2236 | A6085YF_T6086M | L6079V_W6081I_I6088V |
| D2237 | A6085YY_T6086M | L6079V_W6081I_I6088V |
| D2238 | A6085YM_T6086M | L6079V_W6081I_I6088V |
| D2239 | A6085YW_T6086M | L6079V_W6081I_I6088V |
| D2240 | A6085YH_T6086M | L6079V_W6081I_I6088V |
| D2241 | A6085YF_T6086W | L6079V_W6081I_I6088V |
| D2242 | A6085YY_T6086W | L6079V_W6081I_I6088V |
| D2243 | A6085YM_T6086W | L6079V_W6081I_I6088V |
| D2244 | A6085YW_T6086W | L6079V_W6081I_I6088V |
| D2245 | A6085YH_T6086W | L6079V_W6081I_I6088V |
| D2246 | A6085YF_T6086H | L6079V_W6081I_I6088V |
| D2247 | A6085YY_T6086H | L6079V_W6081I_I6088V |
| D2248 | A6085YM_T6086H | L6079V_W6081I_I6088V |
| D2249 | A6085YW_T6086H | L6079V_W6081I_I6088V |
| D2250 | A6085YH_T6086H | L6079V_W6081I_I6088V |
| D2251 | A6085YF_T6086Y | L6079T_W6081T_I6088V |
| D2252 | A6085YY_T6086Y | L6079T_W6081T_I6088V |
| D2253 | A6085YM_T6086Y | L6079T_W6081T_I6088V |
| D2254 | A6085YW_T6086Y | L6079T_W6081T_I6088V |
| D2255 | A6085YH_T6086Y | L6079T_W6081T_I6088V |
| D2256 | A6085YF_T6086F | L6079T_W6081T_I6088V |
| D2257 | A6085YY_T6086F | L6079T_W6081T_I6088V |
| D2258 | A6085YM_T6086F | L6079T_W6081T_I6088V |
| D2259 | A6085YW_T6086F | L6079T_W6081T_I6088V |
| D2260 | A6085YH_T6086F | L6079T_W6081T_I6088V |
| D2261 | A6085YF_T6086M | L6079T_W6081T_I6088V |
| D2262 | A6085YY_T6086M | L6079T_W6081T_I6088V |
| D2263 | A6085YM_T6086M | L6079T_W6081T_I6088V |
| D2264 | A6085YW_T6086M | L6079T_W6081T_I6088V |
| D2265 | A6085YH_T6086M | L6079T_W6081T_I6088V |
| D2266 | A6085YF_T6086W | L6079T_W6081T_I6088V |
| D2267 | A6085YY_T6086W | L6079T_W6081T_I6088V |
| D2268 | A6085YM_T6086W | L6079T_W6081T_I6088V |
| D2269 | A6085YW_T6086W | L6079T_W6081T_I6088V |
| D2270 | A6085YH_T6086W | L6079T_W6081T_I6088V |
| D2271 | A6085YF_T6086H | L6079T_W6081T_I6088V |
| D2272 | A6085YY_T6086H | L6079T_W6081T_I6088V |
| D2273 | A6085YM_T6086H | L6079T_W6081T_I6088V |
| D2274 | A6085YW_T6086H | L6079T_W6081T_I6088V |
| D2275 | A6085YH_T6086H | L6079T_W6081T_I6088V |
| D2276 | A6085YF_T6086Y | L6079T_W6081L_I6088V |
| D2277 | A6085YY_T6086Y | L6079T_W6081L_I6088V |
| D2278 | A6085YM_T6086Y | L6079T_W6081L_I6088V |
| D2279 | A6085YW_T6086Y | L6079T_W6081L_I6088V |
| D2280 | A6085YH_T6086Y | L6079T_W6081L_I6088V |
| D2281 | A6085YF_T6086F | L6079T_W6081L_I6088V |
| D2282 | A6085YY_T6086F | L6079T_W6081L_I6088V |
| D2283 | A6085YM_T6086F | L6079T_W6081L_I6088V |
| D2284 | A6085YW_T6086F | L6079T_W6081L_I6088V |
| D2285 | A6085YH_T6086F | L6079T_W6081L_I6088V |
| D2286 | A6085YF_T6086M | L6079T_W6081L_I6088V |
| D2287 | A6085YY_T6086M | L6079T_W6081L_I6088V |
| D2288 | A6085YM_T6086M | L6079T_W6081L_I6088V |
| D2289 | A6085YW_T6086M | L6079T_W6081L_I6088V |
| D2290 | A6085YH_T6086M | L6079T_W6081L_I6088V |
| D2291 | A6085YF_T6086W | L6079T_W6081L_I6088V |
| D2292 | A6085YY_T6086W | L6079T_W6081L_I6088V |
| D2293 | A6085YM_T6086W | L6079T_W6081L_I6088V |
| D2294 | A6085YW_T6086W | L6079T_W6081L_I6088V |
| D2295 | A6085YH_T6086W | L6079T_W6081L_I6088V |
| D2296 | A6085YF_T6086H | L6079T_W6081L_I6088V |
| D2297 | A6085YY_T6086H | L6079T_W6081L_I6088V |
| D2298 | A6085YM_T6086H | L6079T_W6081L_I6088V |
| D2299 | A6085YW_T6086H | L6079T_W6081L_I6088V |
| D2300 | A6085YH_T6086H | L6079T_W6081L_I6088V |
| D2301 | A6085YF_T6086Y | L6079T_W6081A_I6088V |
| D2302 | A6085YY_T6086Y | L6079T_W6081A_I6088V |
| D2303 | A6085YM_T6086Y | L6079T_W6081A_I6088V |
| D2304 | A6085YW_T6086Y | L6079T_W6081A_I6088V |
| D2305 | A6085YH_T6086Y | L6079T_W6081A_I6088V |
| D2306 | A6085YF_T6086F | L6079T_W6081A_I6088V |
| D2307 | A6085YY_T6086F | L6079T_W6081A_I6088V |
| D2308 | A6085YM_T6086F | L6079T_W6081A_I6088V |
| D2309 | A6085YW_T6086F | L6079T_W6081A_I6088V |
| D2310 | A6085YH_T6086F | L6079T_W6081A_I6088V |
| D2311 | A6085YF_T6086M | L6079T_W6081A_I6088V |
| D2312 | A6085YY_T6086M | L6079T_W6081A_I6088V |
| D2313 | A6085YM_T6086M | L6079T_W6081A_I6088V |
| D2314 | A6085YW_T6086M | L6079T_W6081A_I6088V |
| D2315 | A6085YH_T6086M | L6079T_W6081A_I6088V |
| D2316 | A6085YF_T6086W | L6079T_W6081A_I6088V |
| D2317 | A6085YY_T6086W | L6079T_W6081A_I6088V |
| D2318 | A6085YM_T6086W | L6079T_W6081A_I6088V |
| D2319 | A6085YW_T6086W | L6079T_W6081A_I6088V |
| D2320 | A6085YH_T6086W | L6079T_W6081A_I6088V |
| D2321 | A6085YF_T6086H | L6079T_W6081A_I6088V |
| D2322 | A6085YY_T6086H | L6079T_W6081A_I6088V |
| D2323 | A6085YM_T6086H | L6079T_W6081A_I6088V |
| D2324 | A6085YW_T6086H | L6079T_W6081A_I6088V |
| D2325 | A6085YH_T6086H | L6079T_W6081A_I6088V |
| D2326 | A6085YF_T6086Y | L6079T_W6081V_I6088V |
| D2327 | A6085YY_T6086Y | L6079T_W6081V_I6088V |
| D2328 | A6085YM_T6086Y | L6079T_W6081V_I6088V |
| D2329 | A6085YW_T6086Y | L6079T_W6081V_I6088V |
| D2330 | A6085YH_T6086Y | L6079T_W6081V_I6088V |
| D2331 | A6085YF_T6086F | L6079T_W6081V_I6088V |
| D2332 | A6085YY_T6086F | L6079T_W6081V_I6088V |
| D2333 | A6085YM_T6086F | L6079T_W6081V_I6088V |
| D2334 | A6085YW_T6086F | L6079T_W6081V_I6088V |
| D2335 | A6085YH_T6086F | L6079T_W6081V_I6088V |
| D2336 | A6085YF_T6086M | L6079T_W6081V_I6088V |
| D2337 | A6085YY_T6086M | L6079T_W6081V_I6088V |
| D2338 | A6085YM_T6086M | L6079T_W6081V_I6088V |
| D2339 | A6085YW_T6086M | L6079T_W6081V_I6088V |
| D2340 | A6085YH_T6086M | L6079T_W6081V_I6088V |
| D2341 | A6085YF_T6086W | L6079T_W6081V_I6088V |
| D2342 | A6085YY_T6086W | L6079T_W6081V_I6088V |
| D2343 | A6085YM_T6086W | L6079T_W6081V_I6088V |
| D2344 | A6085YW_T6086W | L6079T_W6081V_I6088V |
| D2345 | A6085YH_T6086W | L6079T_W6081V_I6088V |
| D2346 | A6085YF_T6086H | L6079T_W6081V_I6088V |
| D2347 | A6085YY_T6086H | L6079T_W6081V_I6088V |
| D2348 | A6085YM_T6086H | L6079T_W6081V_I6088V |
| D2349 | A6085YW_T6086H | L6079T_W6081V_I6088V |
| D2350 | A6085YH_T6086H | L6079T_W6081V_I6088V |
| D2351 | A6085YF_T6086Y | L6079T_W6081I_I6088V |
| D2352 | A6085YY_T6086Y | L6079T_W6081I_I6088V |
| D2353 | A6085YM_T6086Y | L6079T_W6081I_I6088V |
| D2354 | A6085YW_T6086Y | L6079T_W6081I_I6088V |
| D2355 | A6085YH_T6086Y | L6079T_W6081I_I6088V |
| D2356 | A6085YF_T6086F | L6079T_W6081I_I6088V |
| D2357 | A6085YY_T6086F | L6079T_W6081I_I6088V |
| D2358 | A6085YM_T6086F | L6079T_W6081I_I6088V |
| D2359 | A6085YW_T6086F | L6079T_W6081I_I6088V |
| D2360 | A6085YH_T6086F | L6079T_W6081I_I6088V |
| D2361 | A6085YF_T6086M | L6079T_W6081I_I6088V |
| D2362 | A6085YY_T6086M | L6079T_W6081I_I6088V |
| D2363 | A6085YM_T6086M | L6079T_W6081I_I6088V |
| D2364 | A6085YW_T6086M | L6079T_W6081I_I6088V |
| D2365 | A6085YH_T6086M | L6079T_W6081I_I6088V |
| D2366 | A6085YF_T6086W | L6079T_W6081I_I6088V |
| D2367 | A6085YY_T6086W | L6079T_W6081I_I6088V |
| D2368 | A6085YM_T6086W | L6079T_W6081I_I6088V |
| D2369 | A6085YW_T6086W | L6079T_W6081I_I6088V |
| D2370 | A6085YH_T6086W | L6079T_W6081I_I6088V |
| D2371 | A6085YF_T6086H | L6079T_W6081I_I6088V |
| D2372 | A6085YY_T6086H | L6079T_W6081I_I6088V |
| D2373 | A6085YM_T6086H | L6079T_W6081I_I6088V |
| D2374 | A6085YW_T6086H | L6079T_W6081I_I6088V |
| D2375 | A6085YH_T6086H | L6079T_W6081I_I6088V |
| D2376 | A6085YF_T6086Y | L6079A_W6081T_I6088V |
| D2377 | A6085YY_T6086Y | L6079A_W6081T_I6088V |
| D2378 | A6085YM_T6086Y | L6079A_W6081T_I6088V |
| D2379 | A6085YW_T6086Y | L6079A_W6081T_I6088V |
| D2380 | A6085YH_T6086Y | L6079A_W6081T_I6088V |
| D2381 | A6085YF_T6086F | L6079A_W6081T_I6088V |
| D2382 | A6085YY_T6086F | L6079A_W6081T_I6088V |
| D2383 | A6085YM_T6086F | L6079A_W6081T_I6088V |
| D2384 | A6085YW_T6086F | L6079A_W6081T_I6088V |
| D2385 | A6085YH_T6086F | L6079A_W6081T_I6088V |
| D2386 | A6085YF_T6086M | L6079A_W6081T_I6088V |
| D2387 | A6085YY_T6086M | L6079A_W6081T_I6088V |
| D2388 | A6085YM_T6086M | L6079A_W6081T_I6088V |
| D2389 | A6085YW_T6086M | L6079A_W6081T_I6088V |
| D2390 | A6085YH_T6086M | L6079A_W6081T_I6088V |
| D2391 | A6085YF_T6086W | L6079A_W6081T_I6088V |
| D2392 | A6085YY_T6086W | L6079A_W6081T_I6088V |
| D2393 | A6085YM_T6086W | L6079A_W6081T_I6088V |
| D2394 | A6085YW_T6086W | L6079A_W6081T_I6088V |
| D2395 | A6085YH_T6086W | L6079A_W6081T_I6088V |
| D2396 | A6085YF_T6086H | L6079A_W6081T_I6088V |
| D2397 | A6085YY_T6086H | L6079A_W6081T_I6088V |
| D2398 | A6085YM_T6086H | L6079A_W6081T_I6088V |
| D2399 | A6085YW_T6086H | L6079A_W6081T_I6088V |
| D2400 | A6085YH_T6086H | L6079A_W6081T_I6088V |
| D2401 | A6085YF_T6086Y | L6079A_W6081L_I6088V |
| D2402 | A6085YY_T6086Y | L6079A_W6081L_I6088V |
| D2403 | A6085YM_T6086Y | L6079A_W6081L_I6088V |
| D2404 | A6085YW_T6086Y | L6079A_W6081L_I6088V |
| D2405 | A6085YH_T6086Y | L6079A_W6081L_I6088V |
| D2406 | A6085YF_T6086F | L6079A_W6081L_I6088V |
| D2407 | A6085YY_T6086F | L6079A_W6081L_I6088V |
| D2408 | A6085YM_T6086F | L6079A_W6081L_I6088V |
| D2409 | A6085YW_T6086F | L6079A_W6081L_I6088V |
| D2410 | A6085YH_T6086F | L6079A_W6081L_I6088V |
| D2411 | A6085YF_T6086M | L6079A_W6081L_I6088V |
| D2412 | A6085YY_T6086M | L6079A_W6081L_I6088V |
| D2413 | A6085YM_T6086M | L6079A_W6081L_I6088V |
| D2414 | A6085YW_T6086M | L6079A_W6081L_I6088V |
| D2415 | A6085YH_T6086M | L6079A_W6081L_I6088V |
| D2416 | A6085YF_T6086W | L6079A_W6081L_I6088V |
| D2417 | A6085YY_T6086W | L6079A_W6081L_I6088V |
| D2418 | A6085YM_T6086W | L6079A_W6081L_I6088V |
| D2419 | A6085YW_T6086W | L6079A_W6081L_I6088V |
| D2420 | A6085YH_T6086W | L6079A_W6081L_I6088V |
| D2421 | A6085YF_T6086H | L6079A_W6081L_I6088V |
| D2422 | A6085YY_T6086H | L6079A_W6081L_I6088V |
| D2423 | A6085YM_T6086H | L6079A_W6081L_I6088V |
| D2424 | A6085YW_T6086H | L6079A_W6081L_I6088V |
| D2425 | A6085YH_T6086H | L6079A_W6081L_I6088V |
| D2426 | A6085YF_T6086Y | L6079A_W6081A_I6088V |
| D2427 | A6085YY_T6086Y | L6079A_W6081A_I6088V |
| D2428 | A6085YM_T6086Y | L6079A_W6081A_I6088V |
| D2429 | A6085YW_T6086Y | L6079A_W6081A_I6088V |
| D2430 | A6085YH_T6086Y | L6079A_W6081A_I6088V |
| D2431 | A6085YF_T6086F | L6079A_W6081A_I6088V |
| D2432 | A6085YY_T6086F | L6079A_W6081A_I6088V |
| D2433 | A6085YM_T6086F | L6079A_W6081A_I6088V |
| D2434 | A6085YW_T6086F | L6079A_W6081A_I6088V |
| D2435 | A6085YH_T6086F | L6079A_W6081A_I6088V |
| D2436 | A6085YF_T6086M | L6079A_W6081A_I6088V |
| D2437 | A6085YY_T6086M | L6079A_W6081A_I6088V |
| D2438 | A6085YM_T6086M | L6079A_W6081A_I6088V |
| D2439 | A6085YW_T6086M | L6079A_W6081A_I6088V |
| D2440 | A6085YH_T6086M | L6079A_W6081A_I6088V |
| D2441 | A6085YF_T6086W | L6079A_W6081A_I6088V |
| D2442 | A6085YY_T6086W | L6079A_W6081A_I6088V |
| D2443 | A6085YM_T6086W | L6079A_W6081A_I6088V |
| D2444 | A6085YW_T6086W | L6079A_W6081A_I6088V |
| D2445 | A6085YH_T6086W | L6079A_W6081A_I6088V |
| D2446 | A6085YF_T6086H | L6079A_W6081A_I6088V |
| D2447 | A6085YY_T6086H | L6079A_W6081A_I6088V |
| D2448 | A6085YM_T6086H | L6079A_W6081A_I6088V |
| D2449 | A6085YW_T6086H | L6079A_W6081A_I6088V |
| D2450 | A6085YH_T6086H | L6079A_W6081A_I6088V |
| D2451 | A6085YF_T6086Y | L6079A_W6081V_I6088V |
| D2452 | A6085YY_T6086Y | L6079A_W6081V_I6088V |
| D2453 | A6085YM_T6086Y | L6079A_W6081V_I6088V |
| D2454 | A6085YW_T6086Y | L6079A_W6081V_I6088V |
| D2455 | A6085YH_T6086Y | L6079A_W6081V_I6088V |
| D2456 | A6085YF_T6086F | L6079A_W6081V_I6088V |
| D2457 | A6085YY_T6086F | L6079A_W6081V_I6088V |
| D2458 | A6085YM_T6086F | L6079A_W6081V_I6088V |
| D2459 | A6085YW_T6086F | L6079A_W6081V_I6088V |
| D2460 | A6085YH_T6086F | L6079A_W6081V_I6088V |
| D2461 | A6085YF_T6086M | L6079A_W6081V_I6088V |
| D2462 | A6085YY_T6086M | L6079A_W6081V_I6088V |
| D2463 | A6085YM_T6086M | L6079A_W6081V_I6088V |
| D2464 | A6085YW_T6086M | L6079A_W6081V_I6088V |
| D2465 | A6085YH_T6086M | L6079A_W6081V_I6088V |
| D2466 | A6085YF_T6086W | L6079A_W6081V_I6088V |
| D2467 | A6085YY_T6086W | L6079A_W6081V_I6088V |
| D2468 | A6085YM_T6086W | L6079A_W6081V_I6088V |
| D2469 | A6085YW_T6086W | L6079A_W6081V_I6088V |
| D2470 | A6085YH_T6086W | L6079A_W6081V_I6088V |
| D2471 | A6085YF_T6086H | L6079A_W6081V_I6088V |
| D2472 | A6085YY_T6086H | L6079A_W6081V_I6088V |
| D2473 | A6085YM_T6086H | L6079A_W6081V_I6088V |
| D2474 | A6085YW_T6086H | L6079A_W6081V_I6088V |
| D2475 | A6085YH_T6086H | L6079A_W6081V_I6088V |
| D2476 | A6085YF_T6086Y | L6079A_W6081I_I6088V |
| D2477 | A6085YY_T6086Y | L6079A_W6081I_I6088V |
| D2478 | A6085YM_T6086Y | L6079A_W6081I_I6088V |
| D2479 | A6085YW_T6086Y | L6079A_W6081I_I6088V |
| D2480 | A6085YH_T6086Y | L6079A_W6081I_I6088V |
| D2481 | A6085YF_T6086F | L6079A_W6081I_I6088V |
| D2482 | A6085YY_T6086F | L6079A_W6081I_I6088V |
| D2483 | A6085YM_T6086F | L6079A_W6081I_I6088V |
| D2484 | A6085YW_T6086F | L6079A_W6081I_I6088V |
| D2485 | A6085YH_T6086F | L6079A_W6081I_I6088V |
| D2486 | A6085YF_T6086M | L6079A_W6081I_I6088V |
| D2487 | A6085YY_T6086M | L6079A_W6081I_I6088V |
| D2488 | A6085YM_T6086M | L6079A_W6081I_I6088V |
| D2489 | A6085YW_T6086M | L6079A_W6081I_I6088V |
| D2490 | A6085YH_T6086M | L6079A_W6081I_I6088V |
| D2491 | A6085YF_T6086W | L6079A_W6081I_I6088V |
| D2492 | A6085YY_T6086W | L6079A_W6081I_I6088V |
| D2493 | A6085YM_T6086W | L6079A_W6081I_I6088V |
| D2494 | A6085YW_T6086W | L6079A_W6081I_I6088V |
| D2495 | A6085YH_T6086W | L6079A_W6081I_I6088V |
| D2496 | A6085YF_T6086H | L6079A_W6081I_I6088V |
| D2497 | A6085YY_T6086H | L6079A_W6081I_I6088V |
| D2498 | A6085YM_T6086H | L6079A_W6081I_I6088V |
| D2499 | A6085YW_T6086H | L6079A_W6081I_I6088V |
| D2500 | A6085YH_T6086H | L6079A_W6081I_I6088V |
| D2501 | A6085YF_T6086Y | L6079I_W6081T_I6088V |
| D2502 | A6085YY_T6086Y | L6079I_W6081T_I6088V |
| D2503 | A6085YM_T6086Y | L6079I_W6081T_I6088V |
| D2504 | A6085YW_T6086Y | L6079I_W6081T_I6088V |
| D2505 | A6085YH_T6086Y | L6079I_W6081T_I6088V |
| D2506 | A6085YF_T6086F | L6079I_W6081T_I6088V |
| D2507 | A6085YY_T6086F | L6079I_W6081T_I6088V |
| D2508 | A6085YM_T6086F | L6079I_W6081T_I6088V |
| D2509 | A6085YW_T6086F | L6079I_W6081T_I6088V |
| D2510 | A6085YH_T6086F | L6079I_W6081T_I6088V |
| D2511 | A6085YF_T6086M | L6079I_W6081T_I6088V |
| D2512 | A6085YY_T6086M | L6079I_W6081T_I6088V |
| D2513 | A6085YM_T6086M | L6079I_W6081T_I6088V |
| D2514 | A6085YW_T6086M | L6079I_W6081T_I6088V |
| D2515 | A6085YH_T6086M | L6079I_W6081T_I6088V |
| D2516 | A6085YF_T6086W | L6079I_W6081T_I6088V |
| D2517 | A6085YY_T6086W | L6079I_W6081T_I6088V |
| D2518 | A6085YM_T6086W | L6079I_W6081T_I6088V |
| D2519 | A6085YW_T6086W | L6079I_W6081T_I6088V |
| D2520 | A6085YH_T6086W | L6079I_W6081T_I6088V |
| D2521 | A6085YF_T6086H | L6079I_W6081T_I6088V |
| D2522 | A6085YY_T6086H | L6079I_W6081T_I6088V |
| D2523 | A6085YM_T6086H | L6079I_W6081T_I6088V |
| D2524 | A6085YW_T6086H | L6079I_W6081T_I6088V |
| D2525 | A6085YH_T6086H | L6079I_W6081T_I6088V |
| D2526 | A6085YF_T6086Y | L6079I_W6081L_I6088V |
| D2527 | A6085YY_T6086Y | L6079I_W6081L_I6088V |
| D2528 | A6085YM_T6086Y | L6079I_W6081L_I6088V |
| D2529 | A6085YW_T6086Y | L6079I_W6081L_I6088V |
| D2530 | A6085YH_T6086Y | L6079I_W6081L_I6088V |
| D2531 | A6085YF_T6086F | L6079I_W6081L_I6088V |
| D2532 | A6085YY_T6086F | L6079I_W6081L_I6088V |
| D2533 | A6085YM_T6086F | L6079I_W6081L_I6088V |
| D2534 | A6085YW_T6086F | L6079I_W6081L_I6088V |
| D2535 | A6085YH_T6086F | L6079I_W6081L_I6088V |
| D2536 | A6085YF_T6086M | L6079I_W6081L_I6088V |
| D2537 | A6085YY_T6086M | L6079I_W6081L_I6088V |
| D2538 | A6085YM_T6086M | L6079I_W6081L_I6088V |
| D2539 | A6085YW_T6086M | L6079I_W6081L_I6088V |
| D2540 | A6085YH_T6086M | L6079I_W6081L_I6088V |
| D2541 | A6085YF_T6086W | L6079I_W6081L_I6088V |
| D2542 | A6085YY_T6086W | L6079I_W6081L_I6088V |
| D2543 | A6085YM_T6086W | L6079I_W6081L_I6088V |
| D2544 | A6085YW_T6086W | L6079I_W6081L_I6088V |
| D2545 | A6085YH_T6086W | L6079I_W6081L_I6088V |
| D2546 | A6085YF_T6086H | L6079I_W6081L_I6088V |
| D2547 | A6085YY_T6086H | L6079I_W6081L_I6088V |
| D2548 | A6085YM_T6086H | L6079I_W6081L_I6088V |
| D2549 | A6085YW_T6086H | L6079I_W6081L_I6088V |
| D2550 | A6085YH_T6086H | L6079I_W6081L_I6088V |
| D2551 | A6085YF_T6086Y | L6079I_W6081A_I6088V |
| D2552 | A6085YY_T6086Y | L6079I_W6081A_I6088V |
| D2553 | A6085YM_T6086Y | L6079I_W6081A_I6088V |
| D2554 | A6085YW_T6086Y | L6079I_W6081A_I6088V |
| D2555 | A6085YH_T6086Y | L6079I_W6081A_I6088V |
| D2556 | A6085YF_T6086F | L6079I_W6081A_I6088V |
| D2557 | A6085YY_T6086F | L6079I_W6081A_I6088V |
| D2558 | A6085YM_T6086F | L6079I_W6081A_I6088V |
| D2559 | A6085YW_T6086F | L6079I_W6081A_I6088V |
| D2560 | A6085YH_T6086F | L6079I_W6081A_I6088V |
| D2561 | A6085YF_T6086M | L6079I_W6081A_I6088V |
| D2562 | A6085YY_T6086M | L6079I_W6081A_I6088V |
| D2563 | A6085YM_T6086M | L6079I_W6081A_I6088V |
| D2564 | A6085YW_T6086M | L6079I_W6081A_I6088V |
| D2565 | A6085YH_T6086M | L6079I_W6081A_I6088V |
| D2566 | A6085YF_T6086W | L6079I_W6081A_I6088V |
| D2567 | A6085YY_T6086W | L6079I_W6081A_I6088V |
| D2568 | A6085YM_T6086W | L6079I_W6081A_I6088V |
| D2569 | A6085YW_T6086W | L6079I_W6081A_I6088V |
| D2570 | A6085YH_T6086W | L6079I_W6081A_I6088V |
| D2571 | A6085YF_T6086H | L6079I_W6081A_I6088V |
| D2572 | A6085YY_T6086H | L6079I_W6081A_I6088V |
| D2573 | A6085YM_T6086H | L6079I_W6081A_I6088V |
| D2574 | A6085YW_T6086H | L6079I_W6081A_I6088V |
| D2575 | A6085YH_T6086H | L6079I_W6081A_I6088V |
| D2576 | A6085YF_T6086Y | L6079I_W6081V_I6088V |
| D2577 | A6085YY_T6086Y | L6079I_W6081V_I6088V |
| D2578 | A6085YM_T6086Y | L6079I_W6081V_I6088V |
| D2579 | A6085YW_T6086Y | L6079I_W6081V_I6088V |
| D2580 | A6085YH_T6086Y | L6079I_W6081V_I6088V |
| D2581 | A6085YF_T6086F | L6079I_W6081V_I6088V |
| D2582 | A6085YY_T6086F | L6079I_W6081V_I6088V |
| D2583 | A6085YM_T6086F | L6079I_W6081V_I6088V |
| D2584 | A6085YW_T6086F | L6079I_W6081V_I6088V |
| D2585 | A6085YH_T6086F | L6079I_W6081V_I6088V |
| D2586 | A6085YF_T6086M | L6079I_W6081V_I6088V |
| D2587 | A6085YY_T6086M | L6079I_W6081V_I6088V |
| D2588 | A6085YM_T6086M | L6079I_W6081V_I6088V |
| D2589 | A6085YW_T6086M | L6079I_W6081V_I6088V |
| D2590 | A6085YH_T6086M | L6079I_W6081V_I6088V |
| D2591 | A6085YF_T6086W | L6079I_W6081V_I6088V |
| D2592 | A6085YY_T6086W | L6079I_W6081V_I6088V |
| D2593 | A6085YM_T6086W | L6079I_W6081V_I6088V |
| D2594 | A6085YW_T6086W | L6079I_W6081V_I6088V |
| D2595 | A6085YH_T6086W | L6079I_W6081V_I6088V |
| D2596 | A6085YF_T6086H | L6079I_W6081V_I6088V |
| D2597 | A6085YY_T6086H | L6079I_W6081V_I6088V |
| D2598 | A6085YM_T6086H | L6079I_W6081V_I6088V |
| D2599 | A6085YW_T6086H | L6079I_W6081V_I6088V |
| D2600 | A6085YH_T6086H | L6079I_W6081V_I6088V |
| D2601 | A6085YF_T6086Y | L6079I_W6081I_I6088V |
| D2602 | A6085YY_T6086Y | L6079I_W6081I_I6088V |
| D2603 | A6085YM_T6086Y | L6079I_W6081I_I6088V |
| D2604 | A6085YW_T6086Y | L6079I_W6081I_I6088V |
| D2605 | A6085YH_T6086Y | L6079I_W6081I_I6088V |
| D2606 | A6085YF_T6086F | L6079I_W6081I_I6088V |
| D2607 | A6085YY_T6086F | L6079I_W6081I_I6088V |
| D2608 | A6085YM_T6086F | L6079I_W6081I_I6088V |
| D2609 | A6085YW_T6086F | L6079I_W6081I_I6088V |
| D2610 | A6085YH_T6086F | L6079I_W6081I_I6088V |
| D2611 | A6085YF_T6086M | L6079I_W6081I_I6088V |
| D2612 | A6085YY_T6086M | L6079I_W6081I_I6088V |
| D2613 | A6085YM_T6086M | L6079I_W6081I_I6088V |
| D2614 | A6085YW_T6086M | L6079I_W6081I_I6088V |
| D2615 | A6085YH_T6086M | L6079I_W6081I_I6088V |
| D2616 | A6085YF_T6086W | L6079I_W6081I_I6088V |
| D2617 | A6085YY_T6086W | L6079I_W6081I_I6088V |
| D2618 | A6085YM_T6086W | L6079I_W6081I_I6088V |
| D2619 | A6085YW_T6086W | L6079I_W6081I_I6088V |
| D2620 | A6085YH_T6086W | L6079I_W6081I_I6088V |
| D2621 | A6085YF_T6086H | L6079I_W6081I_I6088V |
| D2622 | A6085YY_T6086H | L6079I_W6081I_I6088V |
| D2623 | A6085YM_T6086H | L6079I_W6081I_I6088V |
| D2624 | A6085YW_T6086H | L6079I_W6081I_I6088V |
| D2625 | A6085YH_T6086H | L6079I_W6081I_I6088V |
| D2626 | A6085YF_T6086Y | L6079V_W6081T_I6088T |
| D2627 | A6085YY_T6086Y | L6079V_W6081T_I6088T |
| D2628 | A6085YM_T6086Y | L6079V_W6081T_I6088T |
| D2629 | A6085YW_T6086Y | L6079V_W6081T_I6088T |
| D2630 | A6085YH_T6086Y | L6079V_W6081T_I6088T |
| D2631 | A6085YF_T6086F | L6079V_W6081T_I6088T |
| D2632 | A6085YY_T6086F | L6079V_W6081T_I6088T |
| D2633 | A6085YM_T6086F | L6079V_W6081T_I6088T |
| D2634 | A6085YW_T6086F | L6079V_W6081T_I6088T |
| D2635 | A6085YH_T6086F | L6079V_W6081T_I6088T |
| D2636 | A6085YF_T6086M | L6079V_W6081T_I6088T |
| D2637 | A6085YY_T6086M | L6079V_W6081T_I6088T |
| D2638 | A6085YM_T6086M | L6079V_W6081T_I6088T |
| D2639 | A6085YW_T6086M | L6079V_W6081T_I6088T |
| D2640 | A6085YH_T6086M | L6079V_W6081T_I6088T |
| D2641 | A6085YF_T6086W | L6079V_W6081T_I6088T |
| D2642 | A6085YY_T6086W | L6079V_W6081T_I6088T |
| D2643 | A6085YM_T6086W | L6079V_W6081T_I6088T |
| D2644 | A6085YW_T6086W | L6079V_W6081T_I6088T |
| D2645 | A6085YH_T6086W | L6079V_W6081T_I6088T |
| D2646 | A6085YF_T6086H | L6079V_W6081T_I6088T |
| D2647 | A6085YY_T6086H | L6079V_W6081T_I6088T |
| D2648 | A6085YM_T6086H | L6079V_W6081T_I6088T |
| D2649 | A6085YW_T6086H | L6079V_W6081T_I6088T |
| D2650 | A6085YH_T6086H | L6079V_W6081T_I6088T |
| D2651 | A6085YF_T6086Y | L6079V_W6081L_I6088T |
| D2652 | A6085YY_T6086Y | L6079V_W6081L_I6088T |
| D2653 | A6085YM_T6086Y | L6079V_W6081L_I6088T |
| D2654 | A6085YW_T6086Y | L6079V_W6081L_I6088T |
| D2655 | A6085YH_T6086Y | L6079V_W6081L_I6088T |
| D2656 | A6085YF_T6086F | L6079V_W6081L_I6088T |
| D2657 | A6085YY_T6086F | L6079V_W6081L_I6088T |
| D2658 | A6085YM_T6086F | L6079V_W6081L_I6088T |
| D2659 | A6085YW_T6086F | L6079V_W6081L_I6088T |
| D2660 | A6085YH_T6086F | L6079V_W6081L_I6088T |
| D2661 | A6085YF_T6086M | L6079V_W6081L_I6088T |
| D2662 | A6085YY_T6086M | L6079V_W6081L_I6088T |
| D2663 | A6085YM_T6086M | L6079V_W6081L_I6088T |
| D2664 | A6085YW_T6086M | L6079V_W6081L_I6088T |
| D2665 | A6085YH_T6086M | L6079V_W6081L_I6088T |
| D2666 | A6085YF_T6086W | L6079V_W6081L_I6088T |
| D2667 | A6085YY_T6086W | L6079V_W6081L_I6088T |
| D2668 | A6085YM_T6086W | L6079V_W6081L_I6088T |
| D2669 | A6085YW_T6086W | L6079V_W6081L_I6088T |
| D2670 | A6085YH_T6086W | L6079V_W6081L_I6088T |
| D2671 | A6085YF_T6086H | L6079V_W6081L_I6088T |
| D2672 | A6085YY_T6086H | L6079V_W6081L_I6088T |
| D2673 | A6085YM_T6086H | L6079V_W6081L_I6088T |
| D2674 | A6085YW_T6086H | L6079V_W6081L_I6088T |
| D2675 | A6085YH_T6086H | L6079V_W6081L_I6088T |
| D2676 | A6085YF_T6086Y | L6079V_W6081A_I6088T |
| D2677 | A6085YY_T6086Y | L6079V_W6081A_I6088T |
| D2678 | A6085YM_T6086Y | L6079V_W6081A_I6088T |
| D2679 | A6085YW_T6086Y | L6079V_W6081A_I6088T |
| D2680 | A6085YH_T6086Y | L6079V_W6081A_I6088T |
| D2681 | A6085YF_T6086F | L6079V_W6081A_I6088T |
| D2682 | A6085YY_T6086F | L6079V_W6081A_I6088T |
| D2683 | A6085YM_T6086F | L6079V_W6081A_I6088T |
| D2684 | A6085YW_T6086F | L6079V_W6081A_I6088T |
| D2685 | A6085YH_T6086F | L6079V_W6081A_I6088T |
| D2686 | A6085YF_T6086M | L6079V_W6081A_I6088T |
| D2687 | A6085YY_T6086M | L6079V_W6081A_I6088T |
| D2688 | A6085YM_T6086M | L6079V_W6081A_I6088T |
| D2689 | A6085YW_T6086M | L6079V_W6081A_I6088T |
| D2690 | A6085YH_T6086M | L6079V_W6081A_I6088T |
| D2691 | A6085YF_T6086W | L6079V_W6081A_I6088T |
| D2692 | A6085YY_T6086W | L6079V_W6081A_I6088T |
| D2693 | A6085YM_T6086W | L6079V_W6081A_I6088T |
| D2694 | A6085YW_T6086W | L6079V_W6081A_I6088T |
| D2695 | A6085YH_T6086W | L6079V_W6081A_I6088T |
| D2696 | A6085YF_T6086H | L6079V_W6081A_I6088T |
| D2697 | A6085YY_T6086H | L6079V_W6081A_I6088T |
| D2698 | A6085YM_T6086H | L6079V_W6081A_I6088T |
| D2699 | A6085YW_T6086H | L6079V_W6081A_I6088T |
| D2700 | A6085YH_T6086H | L6079V_W6081A_I6088T |
| D2701 | A6085YF_T6086Y | L6079V_W6081V_I6088T |
| D2702 | A6085YY_T6086Y | L6079V_W6081V_I6088T |
| D2703 | A6085YM_T6086Y | L6079V_W6081V_I6088T |
| D2704 | A6085YW_T6086Y | L6079V_W6081V_I6088T |
| D2705 | A6085YH_T6086Y | L6079V_W6081V_I6088T |
| D2706 | A6085YF_T6086F | L6079V_W6081V_I6088T |
| D2707 | A6085YY_T6086F | L6079V_W6081V_I6088T |
| D2708 | A6085YM_T6086F | L6079V_W6081V_I6088T |
| D2709 | A6085YW_T6086F | L6079V_W6081V_I6088T |
| D2710 | A6085YH_T6086F | L6079V_W6081V_I6088T |
| D2711 | A6085YF_T6086M | L6079V_W6081V_I6088T |
| D2712 | A6085YY_T6086M | L6079V_W6081V_I6088T |
| D2713 | A6085YM_T6086M | L6079V_W6081V_I6088T |
| D2714 | A6085YW_T6086M | L6079V_W6081V_I6088T |
| D2715 | A6085YH_T6086M | L6079V_W6081V_I6088T |
| D2716 | A6085YF_T6086W | L6079V_W6081V_I6088T |
| D2717 | A6085YY_T6086W | L6079V_W6081V_I6088T |
| D2718 | A6085YM_T6086W | L6079V_W6081V_I6088T |
| D2719 | A6085YW_T6086W | L6079V_W6081V_I6088T |
| D2720 | A6085YH_T6086W | L6079V_W6081V_I6088T |
| D2721 | A6085YF_T6086H | L6079V_W6081V_I6088T |
| D2722 | A6085YY_T6086H | L6079V_W6081V_I6088T |
| D2723 | A6085YM_T6086H | L6079V_W6081V_I6088T |
| D2724 | A6085YW_T6086H | L6079V_W6081V_I6088T |
| D2725 | A6085YH_T6086H | L6079V_W6081V_I6088T |
| D2726 | A6085YF_T6086Y | L6079V_W6081I_I6088T |
| D2727 | A6085YY_T6086Y | L6079V_W6081I_I6088T |
| D2728 | A6085YM_T6086Y | L6079V_W6081I_I6088T |
| D2729 | A6085YW_T6086Y | L6079V_W6081I_I6088T |
| D2730 | A6085YH_T6086Y | L6079V_W6081I_I6088T |
| D2731 | A6085YF_T6086F | L6079V_W6081I_I6088T |
| D2732 | A6085YY_T6086F | L6079V_W6081I_I6088T |
| D2733 | A6085YM_T6086F | L6079V_W6081I_I6088T |
| D2734 | A6085YW_T6086F | L6079V_W6081I_I6088T |
| D2735 | A6085YH_T6086F | L6079V_W6081I_I6088T |
| D2736 | A6085YF_T6086M | L6079V_W6081I_I6088T |
| D2737 | A6085YY_T6086M | L6079V_W6081I_I6088T |
| D2738 | A6085YM_T6086M | L6079V_W6081I_I6088T |
| D2739 | A6085YW_T6086M | L6079V_W6081I_I6088T |
| D2740 | A6085YH_T6086M | L6079V_W6081I_I6088T |
| D2741 | A6085YF_T6086W | L6079V_W6081I_I6088T |
| D2742 | A6085YY_T6086W | L6079V_W6081I_I6088T |
| D2743 | A6085YM_T6086W | L6079V_W6081I_I6088T |
| D2744 | A6085YW_T6086W | L6079V_W6081I_I6088T |
| D2745 | A6085YH_T6086W | L6079V_W6081I_I6088T |
| D2746 | A6085YF_T6086H | L6079V_W6081I_I6088T |
| D2747 | A6085YY_T6086H | L6079V_W6081I_I6088T |
| D2748 | A6085YM_T6086H | L6079V_W6081I_I6088T |
| D2749 | A6085YW_T6086H | L6079V_W6081I_I6088T |
| D2750 | A6085YH_T6086H | L6079V_W6081I_I6088T |
| D2751 | A6085YF_T6086Y | L6079T_W6081T_I6088T |
| D2752 | A6085YY_T6086Y | L6079T_W6081T_I6088T |
| D2753 | A6085YM_T6086Y | L6079T_W6081T_I6088T |
| D2754 | A6085YW_T6086Y | L6079T_W6081T_I6088T |
| D2755 | A6085YH_T6086Y | L6079T_W6081T_I6088T |
| D2756 | A6085YF_T6086F | L6079T_W6081T_I6088T |
| D2757 | A6085YY_T6086F | L6079T_W6081T_I6088T |
| D2758 | A6085YM_T6086F | L6079T_W6081T_I6088T |
| D2759 | A6085YW_T6086F | L6079T_W6081T_I6088T |
| D2760 | A6085YH_T6086F | L6079T_W6081T_I6088T |
| D2761 | A6085YF_T6086M | L6079T_W6081T_I6088T |
| D2762 | A6085YY_T6086M | L6079T_W6081T_I6088T |
| D2763 | A6085YM_T6086M | L6079T_W6081T_I6088T |
| D2764 | A6085YW_T6086M | L6079T_W6081T_I6088T |
| D2765 | A6085YH_T6086M | L6079T_W6081T_I6088T |
| D2766 | A6085YF_T6086W | L6079T_W6081T_I6088T |
| D2767 | A6085YY_T6086W | L6079T_W6081T_I6088T |
| D2768 | A6085YM_T6086W | L6079T_W6081T_I6088T |
| D2769 | A6085YW_T6086W | L6079T_W6081T_I6088T |
| D2770 | A6085YH_T6086W | L6079T_W6081T_I6088T |
| D2771 | A6085YF_T6086H | L6079T_W6081T_I6088T |
| D2772 | A6085YY_T6086H | L6079T_W6081T_I6088T |
| D2773 | A6085YM_T6086H | L6079T_W6081T_I6088T |
| D2774 | A6085YW_T6086H | L6079T_W6081T_I6088T |
| D2775 | A6085YH_T6086H | L6079T_W6081T_I6088T |
| D2776 | A6085YF_T6086Y | L6079T_W6081L_I6088T |
| D2777 | A6085YY_T6086Y | L6079T_W6081L_I6088T |
| D2778 | A6085YM_T6086Y | L6079T_W6081L_I6088T |
| D2779 | A6085YW_T6086Y | L6079T_W6081L_I6088T |
| D2780 | A6085YH_T6086Y | L6079T_W6081L_I6088T |
| D2781 | A6085YF_T6086F | L6079T_W6081L_I6088T |
| D2782 | A6085YY_T6086F | L6079T_W6081L_I6088T |
| D2783 | A6085YM_T6086F | L6079T_W6081L_I6088T |
| D2784 | A6085YW_T6086F | L6079T_W6081L_I6088T |
| D2785 | A6085YH_T6086F | L6079T_W6081L_I6088T |
| D2786 | A6085YF_T6086M | L6079T_W6081L_I6088T |
| D2787 | A6085YY_T6086M | L6079T_W6081L_I6088T |
| D2788 | A6085YM_T6086M | L6079T_W6081L_I6088T |
| D2789 | A6085YW_T6086M | L6079T_W6081L_I6088T |
| D2790 | A6085YH_T6086M | L6079T_W6081L_I6088T |
| D2791 | A6085YF_T6086W | L6079T_W6081L_I6088T |
| D2792 | A6085YY_T6086W | L6079T_W6081L_I6088T |
| D2793 | A6085YM_T6086W | L6079T_W6081L_I6088T |
| D2794 | A6085YW_T6086W | L6079T_W6081L_I6088T |
| D2795 | A6085YH_T6086W | L6079T_W6081L_I6088T |
| D2796 | A6085YF_T6086H | L6079T_W6081L_I6088T |
| D2797 | A6085YY_T6086H | L6079T_W6081L_I6088T |
| D2798 | A6085YM_T6086H | L6079T_W6081L_I6088T |
| D2799 | A6085YW_T6086H | L6079T_W6081L_I6088T |
| D2800 | A6085YH_T6086H | L6079T_W6081L_I6088T |
| D2801 | A6085YF_T6086Y | L6079T_W6081A_I6088T |
| D2802 | A6085YY_T6086Y | L6079T_W6081A_I6088T |
| D2803 | A6085YM_T6086Y | L6079T_W6081A_I6088T |
| D2804 | A6085YW_T6086Y | L6079T_W6081A_I6088T |
| D2805 | A6085YH_T6086Y | L6079T_W6081A_I6088T |
| D2806 | A6085YF_T6086F | L6079T_W6081A_I6088T |
| D2807 | A6085YY_T6086F | L6079T_W6081A_I6088T |
| D2808 | A6085YM_T6086F | L6079T_W6081A_I6088T |
| D2809 | A6085YW_T6086F | L6079T_W6081A_I6088T |
| D2810 | A6085YH_T6086F | L6079T_W6081A_I6088T |
| D2811 | A6085YF_T6086M | L6079T_W6081A_I6088T |
| D2812 | A6085YY_T6086M | L6079T_W6081A_I6088T |
| D2813 | A6085YM_T6086M | L6079T_W6081A_I6088T |
| D2814 | A6085YW_T6086M | L6079T_W6081A_I6088T |
| D2815 | A6085YH_T6086M | L6079T_W6081A_I6088T |
| D2816 | A6085YF_T6086W | L6079T_W6081A_I6088T |
| D2817 | A6085YY_T6086W | L6079T_W6081A_I6088T |
| D2818 | A6085YM_T6086W | L6079T_W6081A_I6088T |
| D2819 | A6085YW_T6086W | L6079T_W6081A_I6088T |
| D2820 | A6085YH_T6086W | L6079T_W6081A_I6088T |
| D2821 | A6085YF_T6086H | L6079T_W6081A_I6088T |
| D2822 | A6085YY_T6086H | L6079T_W6081A_I6088T |
| D2823 | A6085YM_T6086H | L6079T_W6081A_I6088T |
| D2824 | A6085YW_T6086H | L6079T_W6081A_I6088T |
| D2825 | A6085YH_T6086H | L6079T_W6081A_I6088T |
| D2826 | A6085YF_T6086Y | L6079T_W6081V_I6088T |
| D2827 | A6085YY_T6086Y | L6079T_W6081V_I6088T |
| D2828 | A6085YM_T6086Y | L6079T_W6081V_I6088T |
| D2829 | A6085YW_T6086Y | L6079T_W6081V_I6088T |
| D2830 | A6085YH_T6086Y | L6079T_W6081V_I6088T |
| D2831 | A6085YF_T6086F | L6079T_W6081V_I6088T |
| D2832 | A6085YY_T6086F | L6079T_W6081V_I6088T |
| D2833 | A6085YM_T6086F | L6079T_W6081V_I6088T |
| D2834 | A6085YW_T6086F | L6079T_W6081V_I6088T |
| D2835 | A6085YH_T6086F | L6079T_W6081V_I6088T |
| D2836 | A6085YF_T6086M | L6079T_W6081V_I6088T |
| D2837 | A6085YY_T6086M | L6079T_W6081V_I6088T |
| D2838 | A6085YM_T6086M | L6079T_W6081V_I6088T |
| D2839 | A6085YW_T6086M | L6079T_W6081V_I6088T |
| D2840 | A6085YH_T6086M | L6079T_W6081V_I6088T |
| D2841 | A6085YF_T6086W | L6079T_W6081V_I6088T |
| D2842 | A6085YY_T6086W | L6079T_W6081V_I6088T |
| D2843 | A6085YM_T6086W | L6079T_W6081V_I6088T |
| D2844 | A6085YW_T6086W | L6079T_W6081V_I6088T |
| D2845 | A6085YH_T6086W | L6079T_W6081V_I6088T |
| D2846 | A6085YF_T6086H | L6079T_W6081V_I6088T |
| D2847 | A6085YY_T6086H | L6079T_W6081V_I6088T |
| D2848 | A6085YM_T6086H | L6079T_W6081V_I6088T |
| D2849 | A6085YW_T6086H | L6079T_W6081V_I6088T |
| D2850 | A6085YH_T6086H | L6079T_W6081V_I6088T |
| D2851 | A6085YF_T6086Y | L6079T_W6081I_I6088T |
| D2852 | A6085YY_T6086Y | L6079T_W6081I_I6088T |
| D2853 | A6085YM_T6086Y | L6079T_W6081I_I6088T |
| D2854 | A6085YW_T6086Y | L6079T_W6081I_I6088T |
| D2855 | A6085YH_T6086Y | L6079T_W6081I_I6088T |
| D2856 | A6085YF_T6086F | L6079T_W6081I_I6088T |
| D2857 | A6085YY_T6086F | L6079T_W6081I_I6088T |
| D2858 | A6085YM_T6086F | L6079T_W6081I_I6088T |
| D2859 | A6085YW_T6086F | L6079T_W6081I_I6088T |
| D2860 | A6085YH_T6086F | L6079T_W6081I_I6088T |
| D2861 | A6085YF_T6086M | L6079T_W6081I_I6088T |
| D2862 | A6085YY_T6086M | L6079T_W6081I_I6088T |
| D2863 | A6085YM_T6086M | L6079T_W6081I_I6088T |
| D2864 | A6085YW_T6086M | L6079T_W6081I_I6088T |
| D2865 | A6085YH_T6086M | L6079T_W6081I_I6088T |
| D2866 | A6085YF_T6086W | L6079T_W6081I_I6088T |
| D2867 | A6085YY_T6086W | L6079T_W6081I_I6088T |
| D2868 | A6085YM_T6086W | L6079T_W6081I_I6088T |
| D2869 | A6085YW_T6086W | L6079T_W6081I_I6088T |
| D2870 | A6085YH_T6086W | L6079T_W6081I_I6088T |
| D2871 | A6085YF_T6086H | L6079T_W6081I_I6088T |
| D2872 | A6085YY_T6086H | L6079T_W6081I_I6088T |
| D2873 | A6085YM_T6086H | L6079T_W6081I_I6088T |
| D2874 | A6085YW_T6086H | L6079T_W6081I_I6088T |
| D2875 | A6085YH_T6086H | L6079T_W6081I_I6088T |
| D2876 | A6085YF_T6086Y | L6079A_W6081T_I6088T |
| D2877 | A6085YY_T6086Y | L6079A_W6081T_I6088T |
| D2878 | A6085YM_T6086Y | L6079A_W6081T_I6088T |
| D2879 | A6085YW_T6086Y | L6079A_W6081T_I6088T |
| D2880 | A6085YH_T6086Y | L6079A_W6081T_I6088T |
| D2881 | A6085YF_T6086F | L6079A_W6081T_I6088T |
| D2882 | A6085YY_T6086F | L6079A_W6081T_I6088T |
| D2883 | A6085YM_T6086F | L6079A_W6081T_I6088T |
| D2884 | A6085YW_T6086F | L6079A_W6081T_I6088T |
| D2885 | A6085YH_T6086F | L6079A_W6081T_I6088T |
| D2886 | A6085YF_T6086M | L6079A_W6081T_I6088T |
| D2887 | A6085YY_T6086M | L6079A_W6081T_I6088T |
| D2888 | A6085YM_T6086M | L6079A_W6081T_I6088T |
| D2889 | A6085YW_T6086M | L6079A_W6081T_I6088T |
| D2890 | A6085YH_T6086M | L6079A_W6081T_I6088T |
| D2891 | A6085YF_T6086W | L6079A_W6081T_I6088T |
| D2892 | A6085YY_T6086W | L6079A_W6081T_I6088T |
| D2893 | A6085YM_T6086W | L6079A_W6081T_I6088T |
| D2894 | A6085YW_T6086W | L6079A_W6081T_I6088T |
| D2895 | A6085YH_T6086W | L6079A_W6081T_I6088T |
| D2896 | A6085YF_T6086H | L6079A_W6081T_I6088T |
| D2897 | A6085YY_T6086H | L6079A_W6081T_I6088T |
| D2898 | A6085YM_T6086H | L6079A_W6081T_I6088T |
| D2899 | A6085YW_T6086H | L6079A_W6081T_I6088T |
| D2900 | A6085YH_T6086H | L6079A_W6081T_I6088T |
| D2901 | A6085YF_T6086Y | L6079A_W6081L_I6088T |
| D2902 | A6085YY_T6086Y | L6079A_W6081L_I6088T |
| D2903 | A6085YM_T6086Y | L6079A_W6081L_I6088T |
| D2904 | A6085YW_T6086Y | L6079A_W6081L_I6088T |
| D2905 | A6085YH_T6086Y | L6079A_W6081L_I6088T |
| D2906 | A6085YF_T6086F | L6079A_W6081L_I6088T |
| D2907 | A6085YY_T6086F | L6079A_W6081L_I6088T |
| D2908 | A6085YM_T6086F | L6079A_W6081L_I6088T |
| D2909 | A6085YW_T6086F | L6079A_W6081L_I6088T |
| D2910 | A6085YH_T6086F | L6079A_W6081L_I6088T |
| D2911 | A6085YF_T6086M | L6079A_W6081L_I6088T |
| D2912 | A6085YY_T6086M | L6079A_W6081L_I6088T |
| D2913 | A6085YM_T6086M | L6079A_W6081L_I6088T |
| D2914 | A6085YW_T6086M | L6079A_W6081L_I6088T |
| D2915 | A6085YH_T6086M | L6079A_W6081L_I6088T |
| D2916 | A6085YF_T6086W | L6079A_W6081L_I6088T |
| D2917 | A6085YY_T6086W | L6079A_W6081L_I6088T |
| D2918 | A6085YM_T6086W | L6079A_W6081L_I6088T |
| D2919 | A6085YW_T6086W | L6079A_W6081L_I6088T |
| D2920 | A6085YH_T6086W | L6079A_W6081L_I6088T |
| D2921 | A6085YF_T6086H | L6079A_W6081L_I6088T |
| D2922 | A6085YY_T6086H | L6079A_W6081L_I6088T |
| D2923 | A6085YM_T6086H | L6079A_W6081L_I6088T |
| D2924 | A6085YW_T6086H | L6079A_W6081L_I6088T |
| D2925 | A6085YH_T6086H | L6079A_W6081L_I6088T |
| D2926 | A6085YF_T6086Y | L6079A_W6081A_I6088T |
| D2927 | A6085YY_T6086Y | L6079A_W6081A_I6088T |
| D2928 | A6085YM_T6086Y | L6079A_W6081A_I6088T |
| D2929 | A6085YW_T6086Y | L6079A_W6081A_I6088T |
| D2930 | A6085YH_T6086Y | L6079A_W6081A_I6088T |
| D2931 | A6085YF_T6086F | L6079A_W6081A_I6088T |
| D2932 | A6085YY_T6086F | L6079A_W6081A_I6088T |
| D2933 | A6085YM_T6086F | L6079A_W6081A_I6088T |
| D2934 | A6085YW_T6086F | L6079A_W6081A_I6088T |
| D2935 | A6085YH_T6086F | L6079A_W6081A_I6088T |
| D2936 | A6085YF_T6086M | L6079A_W6081A_I6088T |
| D2937 | A6085YY_T6086M | L6079A_W6081A_I6088T |
| D2938 | A6085YM_T6086M | L6079A_W6081A_I6088T |
| D2939 | A6085YW_T6086M | L6079A_W6081A_I6088T |
| D2940 | A6085YH_T6086M | L6079A_W6081A_I6088T |
| D2941 | A6085YF_T6086W | L6079A_W6081A_I6088T |
| D2942 | A6085YY_T6086W | L6079A_W6081A_I6088T |
| D2943 | A6085YM_T6086W | L6079A_W6081A_I6088T |
| D2944 | A6085YW_T6086W | L6079A_W6081A_I6088T |
| D2945 | A6085YH_T6086W | L6079A_W6081A_I6088T |
| D2946 | A6085YF_T6086H | L6079A_W6081A_I6088T |
| D2947 | A6085YY_T6086H | L6079A_W6081A_I6088T |
| D2948 | A6085YM_T6086H | L6079A_W6081A_I6088T |
| D2949 | A6085YW_T6086H | L6079A_W6081A_I6088T |
| D2950 | A6085YH_T6086H | L6079A_W6081A_I6088T |
| D2951 | A6085YF_T6086Y | L6079A_W6081V_I6088T |
| D2952 | A6085YY_T6086Y | L6079A_W6081V_I6088T |
| D2953 | A6085YM_T6086Y | L6079A_W6081V_I6088T |
| D2954 | A6085YW_T6086Y | L6079A_W6081V_I6088T |
| D2955 | A6085YH_T6086Y | L6079A_W6081V_I6088T |
| D2956 | A6085YF_T6086F | L6079A_W6081V_I6088T |
| D2957 | A6085YY_T6086F | L6079A_W6081V_I6088T |
| D2958 | A6085YM_T6086F | L6079A_W6081V_I6088T |
| D2959 | A6085YW_T6086F | L6079A_W6081V_I6088T |
| D2960 | A6085YH_T6086F | L6079A_W6081V_I6088T |
| D2961 | A6085YF_T6086M | L6079A_W6081V_I6088T |
| D2962 | A6085YY_T6086M | L6079A_W6081V_I6088T |
| D2963 | A6085YM_T6086M | L6079A_W6081V_I6088T |
| D2964 | A6085YW_T6086M | L6079A_W6081V_I6088T |
| D2965 | A6085YH_T6086M | L6079A_W6081V_I6088T |
| D2966 | A6085YF_T6086W | L6079A_W6081V_I6088T |
| D2967 | A6085YY_T6086W | L6079A_W6081V_I6088T |
| D2968 | A6085YM_T6086W | L6079A_W6081V_I6088T |
| D2969 | A6085YW_T6086W | L6079A_W6081V_I6088T |
| D2970 | A6085YH_T6086W | L6079A_W6081V_I6088T |
| D2971 | A6085YF_T6086H | L6079A_W6081V_I6088T |
| D2972 | A6085YY_T6086H | L6079A_W6081V_I6088T |
| D2973 | A6085YM_T6086H | L6079A_W6081V_I6088T |
| D2974 | A6085YW_T6086H | L6079A_W6081V_I6088T |
| D2975 | A6085YH_T6086H | L6079A_W6081V_I6088T |
| D2976 | A6085YF_T6086Y | L6079A_W6081I_I6088T |
| D2977 | A6085YY_T6086Y | L6079A_W6081I_I6088T |
| D2978 | A6085YM_T6086Y | L6079A_W6081I_I6088T |
| D2979 | A6085YW_T6086Y | L6079A_W6081I_I6088T |
| D2980 | A6085YH_T6086Y | L6079A_W6081I_I6088T |
| D2981 | A6085YF_T6086F | L6079A_W6081I_I6088T |
| D2982 | A6085YY_T6086F | L6079A_W6081I_I6088T |
| D2983 | A6085YM_T6086F | L6079A_W6081I_I6088T |
| D2984 | A6085YW_T6086F | L6079A_W6081I_I6088T |
| D2985 | A6085YH_T6086F | L6079A_W6081I_I6088T |
| D2986 | A6085YF_T6086M | L6079A_W6081I_I6088T |
| D2987 | A6085YY_T6086M | L6079A_W6081I_I6088T |
| D2988 | A6085YM_T6086M | L6079A_W6081I_I6088T |
| D2989 | A6085YW_T6086M | L6079A_W6081I_I6088T |
| D2990 | A6085YH_T6086M | L6079A_W6081I_I6088T |
| D2991 | A6085YF_T6086W | L6079A_W6081I_I6088T |
| D2992 | A6085YY_T6086W | L6079A_W6081I_I6088T |
| D2993 | A6085YM_T6086W | L6079A_W6081I_I6088T |
| D2994 | A6085YW_T6086W | L6079A_W6081I_I6088T |
| D2995 | A6085YH_T6086W | L6079A_W6081I_I6088T |
| D2996 | A6085YF_T6086H | L6079A_W6081I_I6088T |
| D2997 | A6085YY_T6086H | L6079A_W6081I_I6088T |
| D2998 | A6085YM_T6086H | L6079A_W6081I_I6088T |
| D2999 | A6085YW_T6086H | L6079A_W6081I_I6088T |
| D3000 | A6085YH_T6086H | L6079A_W6081I_I6088T |
| D3001 | A6085YF_T6086Y | L6079I_W6081T_I6088T |
| D3002 | A6085YY_T6086Y | L6079I_W6081T_I6088T |
| D3003 | A6085YM_T6086Y | L6079I_W6081T_I6088T |
| D3004 | A6085YW_T6086Y | L6079I_W6081T_I6088T |
| D3005 | A6085YH_T6086Y | L6079I_W6081T_I6088T |
| D3006 | A6085YF_T6086F | L6079I_W6081T_I6088T |
| D3007 | A6085YY_T6086F | L6079I_W6081T_I6088T |
| D3008 | A6085YM_T6086F | L6079I_W6081T_I6088T |
| D3009 | A6085YW_T6086F | L6079I_W6081T_I6088T |
| D3010 | A6085YH_T6086F | L6079I_W6081T_I6088T |
| D3011 | A6085YF_T6086M | L6079I_W6081T_I6088T |
| D3012 | A6085YY_T6086M | L6079I_W6081T_I6088T |
| D3013 | A6085YM_T6086M | L6079I_W6081T_I6088T |
| D3014 | A6085YW_T6086M | L6079I_W6081T_I6088T |
| D3015 | A6085YH_T6086M | L6079I_W6081T_I6088T |
| D3016 | A6085YF_T6086W | L6079I_W6081T_I6088T |
| D3017 | A6085YY_T6086W | L6079I_W6081T_I6088T |
| D3018 | A6085YM_T6086W | L6079I_W6081T_I6088T |
| D3019 | A6085YW_T6086W | L6079I_W6081T_I6088T |
| D3020 | A6085YH_T6086W | L6079I_W6081T_I6088T |
| D3021 | A6085YF_T6086H | L6079I_W6081T_I6088T |
| D3022 | A6085YY_T6086H | L6079I_W6081T_I6088T |
| D3023 | A6085YM_T6086H | L6079I_W6081T_I6088T |
| D3024 | A6085YW_T6086H | L6079I_W6081T_I6088T |
| D3025 | A6085YH_T6086H | L6079I_W6081T_I6088T |
| D3026 | A6085YF_T6086Y | L6079I_W6081L_I6088T |
| D3027 | A6085YY_T6086Y | L6079I_W6081L_I6088T |
| D3028 | A6085YM_T6086Y | L6079I_W6081L_I6088T |
| D3029 | A6085YW_T6086Y | L6079I_W6081L_I6088T |
| D3030 | A6085YH_T6086Y | L6079I_W6081L_I6088T |
| D3031 | A6085YF_T6086F | L6079I_W6081L_I6088T |
| D3032 | A6085YY_T6086F | L6079I_W6081L_I6088T |
| D3033 | A6085YM_T6086F | L6079I_W6081L_I6088T |
| D3034 | A6085YW_T6086F | L6079I_W6081L_I6088T |
| D3035 | A6085YH_T6086F | L6079I_W6081L_I6088T |
| D3036 | A6085YF_T6086M | L6079I_W6081L_I6088T |
| D3037 | A6085YY_T6086M | L6079I_W6081L_I6088T |
| D3038 | A6085YM_T6086M | L6079I_W6081L_I6088T |
| D3039 | A6085YW_T6086M | L6079I_W6081L_I6088T |
| D3040 | A6085YH_T6086M | L6079I_W6081L_I6088T |
| D3041 | A6085YF_T6086W | L6079I_W6081L_I6088T |
| D3042 | A6085YY_T6086W | L6079I_W6081L_I6088T |
| D3043 | A6085YM_T6086W | L6079I_W6081L_I6088T |
| D3044 | A6085YW_T6086W | L6079I_W6081L_I6088T |
| D3045 | A6085YH_T6086W | L6079I_W6081L_I6088T |
| D3046 | A6085YF_T6086H | L6079I_W6081L_I6088T |
| D3047 | A6085YY_T6086H | L6079I_W6081L_I6088T |
| D3048 | A6085YM_T6086H | L6079I_W6081L_I6088T |
| D3049 | A6085YW_T6086H | L6079I_W6081L_I6088T |
| D3050 | A6085YH_T6086H | L6079I_W6081L_I6088T |
| D3051 | A6085YF_T6086Y | L6079I_W6081A_I6088T |
| D3052 | A6085YY_T6086Y | L6079I_W6081A_I6088T |
| D3053 | A6085YM_T6086Y | L6079I_W6081A_I6088T |
| D3054 | A6085YW_T6086Y | L6079I_W6081A_I6088T |
| D3055 | A6085YH_T6086Y | L6079I_W6081A_I6088T |
| D3056 | A6085YF_T6086F | L6079I_W6081A_I6088T |
| D3057 | A6085YY_T6086F | L6079I_W6081A_I6088T |
| D3058 | A6085YM_T6086F | L6079I_W6081A_I6088T |
| D3059 | A6085YW_T6086F | L6079I_W6081A_I6088T |
| D3060 | A6085YH_T6086F | L6079I_W6081A_I6088T |
| D3061 | A6085YF_T6086M | L6079I_W6081A_I6088T |
| D3062 | A6085YY_T6086M | L6079I_W6081A_I6088T |
| D3063 | A6085YM_T6086M | L6079I_W6081A_I6088T |
| D3064 | A6085YW_T6086M | L6079I_W6081A_I6088T |
| D3065 | A6085YH_T6086M | L6079I_W6081A_I6088T |
| D3066 | A6085YF_T6086W | L6079I_W6081A_I6088T |
| D3067 | A6085YY_T6086W | L6079I_W6081A_I6088T |
| D3068 | A6085YM_T6086W | L6079I_W6081A_I6088T |
| D3069 | A6085YW_T6086W | L6079I_W6081A_I6088T |
| D3070 | A6085YH_T6086W | L6079I_W6081A_I6088T |
| D3071 | A6085YF_T6086H | L6079I_W6081A_I6088T |
| D3072 | A6085YY_T6086H | L6079I_W6081A_I6088T |
| D3073 | A6085YM_T6086H | L6079I_W6081A_I6088T |
| D3074 | A6085YW_T6086H | L6079I_W6081A_I6088T |
| D3075 | A6085YH_T6086H | L6079I_W6081A_I6088T |
| D3076 | A6085YF_T6086Y | L6079I_W6081V_I6088T |
| D3077 | A6085YY_T6086Y | L6079I_W6081V_I6088T |
| D3078 | A6085YM_T6086Y | L6079I_W6081V_I6088T |
| D3079 | A6085YW_T6086Y | L6079I_W6081V_I6088T |
| D3080 | A6085YH_T6086Y | L6079I_W6081V_I6088T |
| D3081 | A6085YF_T6086F | L6079I_W6081V_I6088T |
| D3082 | A6085YY_T6086F | L6079I_W6081V_I6088T |
| D3083 | A6085YM_T6086F | L6079I_W6081V_I6088T |
| D3084 | A6085YW_T6086F | L6079I_W6081V_I6088T |
| D3085 | A6085YH_T6086F | L6079I_W6081V_I6088T |
| D3086 | A6085YF_T6086M | L6079I_W6081V_I6088T |
| D3087 | A6085YY_T6086M | L6079I_W6081V_I6088T |
| D3088 | A6085YM_T6086M | L6079I_W6081V_I6088T |
| D3089 | A6085YW_T6086M | L6079I_W6081V_I6088T |
| D3090 | A6085YH_T6086M | L6079I_W6081V_I6088T |
| D3091 | A6085YF_T6086W | L6079I_W6081V_I6088T |
| D3092 | A6085YY_T6086W | L6079I_W6081V_I6088T |
| D3093 | A6085YM_T6086W | L6079I_W6081V_I6088T |
| D3094 | A6085YW_T6086W | L6079I_W6081V_I6088T |
| D3095 | A6085YH_T6086W | L6079I_W6081V_I6088T |
| D3096 | A6085YF_T6086H | L6079I_W6081V_I6088T |
| D3097 | A6085YY_T6086H | L6079I_W6081V_I6088T |
| D3098 | A6085YM_T6086H | L6079I_W6081V_I6088T |
| D3099 | A6085YW_T6086H | L6079I_W6081V_I6088T |
| D3100 | A6085YH_T6086H | L6079I_W6081V_I6088T |
| D3101 | A6085YF_T6086Y | L6079I_W6081I_I6088T |
| D3102 | A6085YY_T6086Y | L6079I_W6081I_I6088T |
| D3103 | A6085YM_T6086Y | L6079I_W6081I_I6088T |
| D3104 | A6085YW_T6086Y | L6079I_W6081I_I6088T |
| D3105 | A6085YH_T6086Y | L6079I_W6081I_I6088T |
| D3106 | A6085YF_T6086F | L6079I_W6081I_I6088T |
| D3107 | A6085YY_T6086F | L6079I_W6081I_I6088T |
| D3108 | A6085YM_T6086F | L6079I_W6081I_I6088T |
| D3109 | A6085YW_T6086F | L6079I_W6081I_I6088T |
| D3110 | A6085YH_T6086F | L6079I_W6081I_I6088T |
| D3111 | A6085YF_T6086M | L6079I_W6081I_I6088T |
| D3112 | A6085YY_T6086M | L6079I_W6081I_I6088T |
| D3113 | A6085YM_T6086M | L6079I_W6081I_I6088T |
| D3114 | A6085YW_T6086M | L6079I_W6081I_I6088T |
| D3115 | A6085YH_T6086M | L6079I_W6081I_I6088T |
| D3116 | A6085YF_T6086W | L6079I_W6081I_I6088T |
| D3117 | A6085YY_T6086W | L6079I_W6081I_I6088T |
| D3118 | A6085YM_T6086W | L6079I_W6081I_I6088T |
| D3119 | A6085YW_T6086W | L6079I_W6081I_I6088T |
| D3120 | A6085YH_T6086W | L6079I_W6081I_I6088T |
| D3121 | A6085YF_T6086H | L6079I_W6081I_I6088T |
| D3122 | A6085YY_T6086H | L6079I_W6081I_I6088T |
| D3123 | A6085YM_T6086H | L6079I_W6081I_I6088T |
| D3124 | A6085YW_T6086H | L6079I_W6081I_I6088T |
| D3125 | A6085YH_T6086H | L6079I_W6081I_I6088T |

### EXAMPLES

### Example 1: In Silico Selection of Lead IgA Heterodimer Designs

This example describes the in silico analysis and selection of potential IgA Fe Cα3 (CH3) mutations to drive heterodimerization over homodimerization of IgA Fc dimers.

Methods

In an extensive structural analysis of the CH3:CH3 interface of the IgA Fc (PDB ID: 2QEJ, Ramsland et al., 2007, Proc Natl Acad Sci USA 104:15051-15056), residues in the interface were characterized according to their energetic contribution to dimerization. For this, proprietary tools for analysis of connectivity as well as energetics of the structure based on knowledge-based and physics-based potentials were used on a static structure as well as a 50 ns explicit molecular dynamics trajectory. Guided by results from this initial analysis and in a first “negative design” round, residues were selected for the introduction of mutations predicted to be disruptive to dimerization. These mutations were chosen based on two main design concepts illustrated in FIG. 1. Negative electrostatic designs relied on the introduction of same-charge pairs and the associated repulsion across the interface while negative steric designs were based on the introduction of cavities or steric clashes in the interface. These negative designs were modelled and evaluated energetically using proprietary in silico tools. In a second “positive design” step, additional mutations were introduced with the goal of rescuing heterodimerization. The stabilization of the heterodimeric complex was either based on introduction of salt bridges via opposing charges across the interface or the accommodation of residues with large side chains by cavities on the opposite side of the interface. Designs with the largest energetic differences between homodimers and heterodimers were selected to be expressed and evaluated.

Results

The mutations of the lead designs based on the analyzed metrics are shown in Table 11. A select set of in silico metrics for models of homodimeric and heterodimeric lead designs are shown in Table 12. Energies are with respect to wild-type. Negative energies indicate a favourable interaction, positive energies indicate a disfavoured interaction.

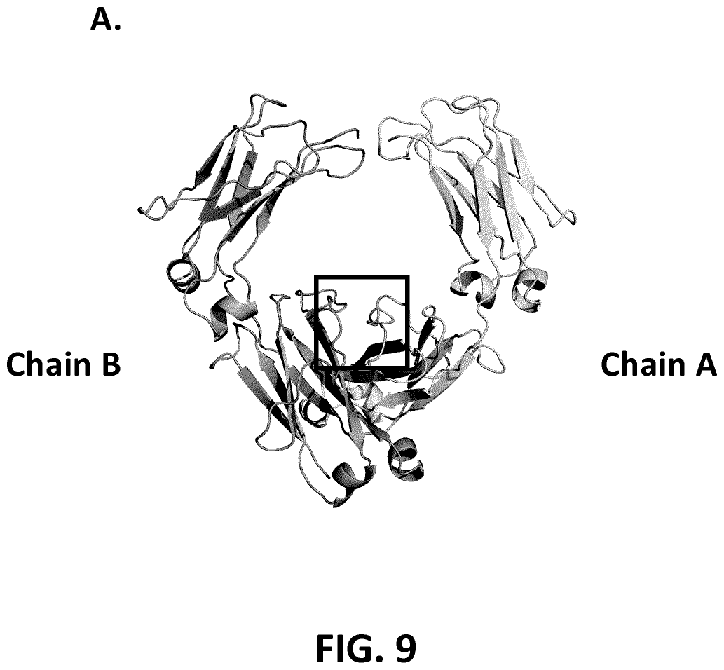

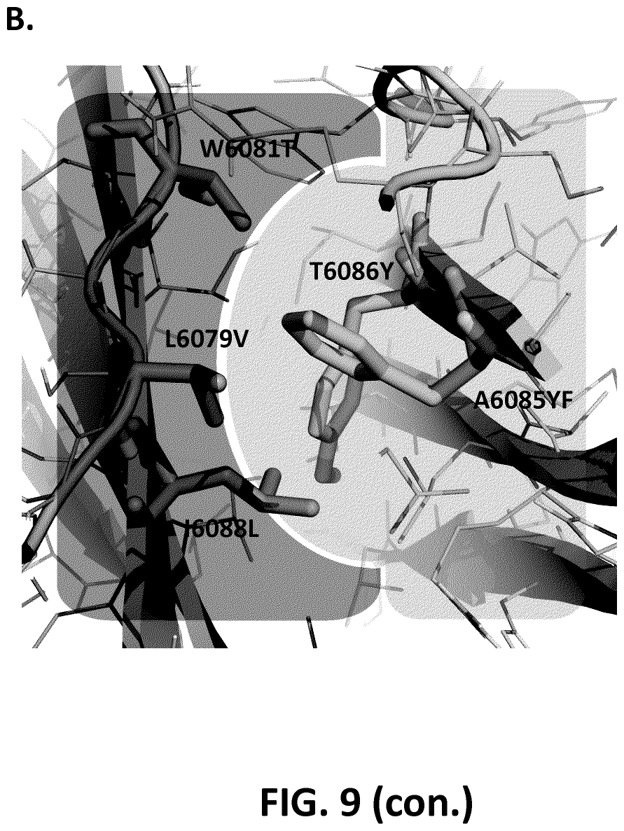

Notably, the steric designs with the largest energetic differences between homo- and heterodimer were centered around mutations to large hydrophobic side chains at positions A6085Y and T6086 in Chain A and a swap of W608I for a small residue on the opposing Chain B. An example of a lead design (Steric 6) is shown in FIG. 9 where large hydrophobic residues were introduced at positions 6085Y and 6086 in Chain A, while a cavity was created by swap of W608I for threonine in Chain B. While Steric 6 includes two additional Chain B mutations, it is the substitution of tryptophan at position 6081 for a residue with a smaller side chain that is responsible for creating the cavity that accommodates the large hydrophobic residues introduced at positions 6085Y and 6086 in Chain A. Together these three mutations are considered to produce the predominant steric design favouring heterodimer formation. As such, mutations at these three positions (A: 6085Y & 6086, B:6081) are considered to constitute a minimal core set of mutations to promote IgA Fc heterodimer formation. Specifically, the core set of mutations is: substitution of each of A6085Y and T6086 in Chain A with residues containing larger and/or more hydrophobic side chains combined with substitution of W608I in Chain B with a residue having a smaller side chain. Larger and/or more hydrophobic residues that are predicted by in silico analysis to be suitable for introduction at positions 6085Y and 6086 include F, Y, M, W and H, and smaller residues predicted by in silico analysis to be suitable for introduction at position 6081 include T, L, A, V and I.

TABLE 11
Mutations in Lead Designs

| | | | |
|---|---|---|---|
| Variant | Design | Chain A Mutations | Chain B Mutations |

Electrostatic Designs

| | | | |
|---|---|---|---|
| 32510 | Electrost. 1 | T6020D_L6024D_R6026D_I6088E | T6020R_L6024R |
| 32511 | Electrost. 2 | T6020E_L6024E_R6026D_I6088D_R6090E | T6020R_L6024K |
| 32512 | Electrost. 3 | T6020E_R6026D_I6088D_R6090D | T6020R_L6024K_I6088R |
| 32513 | Electrost. 4 | T6020E_R6026D_I6088E_R6090E | T6020K_L6024K_R6026K_I6088R |
| 32514 | Electrost. 5 | R6026D_E6084BD_I6088D_R6090E | L6024K_R6026K_E6084BR_I6088K |
| 32515 | Electrost. 6 | R6026D_E6084BD_I6088D_R6090E | L6024K_R6026K_I6088K |

Steric Designs

| | | | |
|---|---|---|---|
| 32516 | Steric 1 | A6085YY_T6086L | L6079T_W6081L_I6088L |
| 32517 | Steric 2 | A6085YY_T6086Y | L6079T_W6081L_I6088L |
| 32518 | Steric 3 | A6085YF_T6086Y | L6079V_W6081L_I6088L |
| 32519 | Steric 4 | L6024M_A6085YF_T6086W | W6081L |
| 32520 | Steric 5 | A6085YY_T6086M | L6079V_W6081L_I6088L |
| 32521 | Steric 6 | A6085YF_T6086Y | L6079V_W6081T_I6088L |
| 33330 | Steric 7 | T6022V_A6085YF_T6086Y | L6079V_W6081T_I6088L |
| 33331 | Steric 8 | T6022L_A6085YF_T6086Y | L6079V_W6081T_I6088L |
| 33332 | Steric 9 | T6022I_A6085YF_T6086Y | L6079V_W6081T_I6088L |
| 33333 | Steric 10 | A6085YF_T6086Y | L6007F_L6079V_W6081T_I6088L |
| 33334 | Steric 11 | H6005Y_A6085YF_T6086Y | H6005Y_L6079V_W6081T_I6088L |

TABLE 12
Exemplary Metrics Used for Lead Design Selection

| | | Δ Physics- | Δ Knowledge- | | Largest |
|---|---|---|---|---|---|
| | | Based Affinity | Based Affinity | Δ SASA | Exceeded vdW |
| Design | Chains¹ | [kcal/mol]^{2, 3} | [kcal/mol]^{2, 3} | [Å²]^{2, 4} | Overlap [Å]⁵ |
| Steric 1 | A/A | 2.3E+02 | 2.3E+02 | −3.1E+01 | 5.1E−01 |
| | B/B | 6.1E+01 | 1.8E+02 | 1.1E+02 | 1.8E−01 |
| | A/B | 2.0E+01 | −7.4E+01 | 3.3E+01 | 1.6E−01 |
| | B/A | 1.7E+01 | −1.0E+02 | 1.0E+01 | 7.0E−02 |
| Steric 2 | A/A | 2.5E+02 | 5.5E+02 | −3.5E+01 | 6.2E−01 |
| | B/B | 6.1E+01 | 1.8E+02 | 1.1E+02 | 1.8E−01 |
| | A/B | 1.6E+01 | −7.3E+01 | 5.0E+01 | 1.8E−01 |
| | B/A | 4.9E+00 | −5.4E+01 | −5.4E+00 | 1.6E−01 |
| Steric 3 | A/A | 1.5E+02 | 7.8E+02 | −3.2E+01 | 6.5E−01 |
| | B/B | 6.3E+01 | 1.3E+02 | 1.2E+02 | 2.2E−01 |
| | A/B | 2.5E+00 | −1.7E+02 | 2.4E+01 | 1.0E−01 |
| | B/A | 5.1E−01 | −1.7E+02 | −3.6E+01 | 1.6E−01 |
| Steric 4 | A/A | 7.9E+01 | 5.8E+02 | −8.0E+01 | 5.1E−01 |
| | B/B | 5.5E+01 | 8.6E+01 | 1.4E+02 | 3.0E−02 |
| | A/B | 2.1E+01 | 3.4E+01 | 1.5E+00 | 4.2E−01 |
| | B/A | 8.5E+00 | −1.3E+02 | 2.0E+01 | 2.6E−01 |
| Steric 5 | A/A | 9.5E+01 | 2.1E+01 | −2.9E+01 | 4.9E−01 |
| | B/B | 6.3E+01 | 1.3E+02 | 1.2E+02 | 2.2E−01 |
| | A/B | 1.6E+01 | −7.7E+01 | 3.5E+01 | 1.3E−01 |
| | B/A | 1.2E+01 | −5.8E+01 | −9.8E+00 | 1.4E−01 |
| Steric 6 | A/A | 1.0E+02 | 6.5E+02 | −2.4E+01 | 6.2E−01 |
| | B/B | 7.2E+01 | 3.0E+02 | 1.4E+02 | 2.2E−01 |
| | A/B | 1.4E+01 | −1.0E+02 | 7.1E+00 | 1.0E−01 |
| | B/A | 1.1E+01 | −6.8E+01 | −1.8E+01 | 1.8E−01 |
| Steric 7 | A/A | −1.2E+01 | 3.1E+02 | −1.3E+00 | 3.5E−01 |
| | B/B | 7.4E+01 | 3.5E+02 | 1.4E+02 | 1.9E−01 |
| | A/B | 1.5E+01 | −1.0E+02 | −8.2E+00 | 2.6E−01 |
| | B/A | 1.1E+01 | −1.0E+02 | 3.1E+01 | 2.1E−01 |
| Steric 8 | A/A | −2.6E+01 | 2.1E+02 | −3.3E+01 | 3.4E−01 |
| | B/B | 7.4E+01 | 3.5E+02 | 1.4E+02 | 1.9E−01 |
| | A/B | 4.2E+00 | −1.6E+02 | −1.1E+01 | 1.6E−01 |
| | B/A | 1.9E+01 | −9.5E+01 | 4.2E+00 | 2.9E−01 |
| Steric 9 | A/A | −1.6E+01 | 2.4E+02 | −3.2E+01 | 2.6E−01 |
| | B/B | 7.4E+01 | 3.5E+02 | 1.4E+02 | 1.9E−01 |
| | A/B | 5.1E+00 | −1.5E+02 | −3.6E+00 | 1.5E−01 |
| | B/A | 1.2E+01 | −1.4E+02 | 1.3E+01 | 1.8E−01 |
| Steric 10 | A/A | −1.2E+01 | 3.3E+02 | 6.4E+00 | 4.6E−01 |
| | B/B | 6.0E+01 | 3.2E+02 | 1.6E+02 | 1.8E−01 |
| | A/B | −2.7E−01 | −8.5E+01 | −2.2E+01 | 1.6E−01 |
| | B/A | 6.6E+00 | 1.3E+02 | 6.7E+00 | 1.9E−01 |
| Steric 11 | A/A | −2.6E+01 | 3.1E+02 | −4.0E+01 | 4.0E−01 |
| | B/B | 6.6E+01 | 2.0E+02 | 1.5E+02 | 1.9E−01 |
| | A/B | 4.8E−01 | −2.0E+02 | −6.4E+00 | 1.2E−01 |
| | B/A | 4.9E+00 | −1.8E+02 | 1.2E+01 | 1.8E−01 |
| Electrostatic | B/B | 9.4E+01 | 1.8E+02 | −3.0E+01 | 2.1E−01 |
| 1 | A/A | 3.1E+01 | 4.4E+02 | 7.7E+01 | 1.0E−01 |
| | B/A | −3.0E−02 | 1.4E+02 | 6.5E+00 | 1.4E−01 |
| | A/B | −1.6E+00 | 1.3E+02 | 3.0E+01 | 1.9E−01 |
| Electrostatic | B/B | 1.2E+02 | 3.2E+02 | −6.7E+00 | 2.7E−01 |
| 2 | A/A | 5.8E+01 | 5.9E+02 | 7.5E+01 | 2.0E−01 |
| | B/A | −5.2E+00 | 2.4E+02 | 5.2E+01 | 1.8E−01 |
| | A/B | −1.0E+01 | 2.2E+02 | 9.1E+00 | 1.6E−01 |
| Electrostatic | B/B | 1.2E+02 | 3.1E+02 | −7.0E+01 | 1.6E−01 |
| 3 | A/A | 5.4E+01 | 5.7E+02 | 5.3E+01 | 1.7E−01 |
| | B/A | 6.4E+00 | 1.9E+02 | 2.3E+01 | 1.1E−01 |
| | A/B | 9.5E−01 | 1.9E+02 | 2.6E+01 | 1.7E−01 |
| Electrostatic | B/B | 8.9E+01 | 3.4E+02 | −2.2E+01 | 3.4E−01 |
| 4 | A/A | 5.1E+01 | 5.8E+02 | 8.8E+01 | 1.7E−01 |
| | B/A | −2.8E+00 | 2.5E+02 | 4.1E+01 | 1.4E−01 |
| | A/B | −3.0E+00 | 2.3E+02 | 8.7E+00 | 2.6E−01 |
| Electrostatic | B/B | 8.1E+01 | 4.1E+02 | −5.5E+00 | 1.8E−01 |
| 5 | A/A | 1.9E+01 | 3.6E+02 | 1.7E+02 | 1.0E−01 |
| | B/A | −1.7E+01 | 2.6E+02 | 6.6E+01 | 5.0E−02 |
| | A/B | −1.5E+01 | 2.5E+02 | 2.0E+01 | 5.0E−02 |
| Electrostatic | B/B | 5.5E+01 | 3.6E+02 | −1.1E+01 | 1.9E−01 |
| 6 | A/A | 1.9E+01 | 3.4E+02 | 1.6E+02 | 1.2E−01 |
| | B/A | −2.2E+01 | 3.4E+02 | 7.7E+01 | 1.8E−01 |
| | A/B | −2.0E+01 | 3.0E+02 | 6.6E+01 | 1.1E−01 |

¹Refers to the chains used in the complex investigated as defined in Table 11. A/A and B/B are homodimers designed to be disfavoured, A/B and B/A refer to heterodimers designed to be the favoured complexes.
²Δ refers to difference in the reported metric compared to wild-type (WT) IgA CH3 homodimer.
³Metrics reporting on the energetics of the interactions of chain A and chain B compared to the WT complex. Negative values indicate a more favourable interaction compared to the WT complex, positive values indicate a less favourable interaction compared to the WT complex.
⁴SASA = solvent accessible surface area. Negative values indicate a loss in SASA compared to the WT complex, generally associated with better packing and a more favourable interaction. Positive values indicate a gain in SASA, generally associated with poorer packing and a less favourable interaction compared to the WT complex.
⁵A metric reporting on the extent of the largest van der Waals (vdW) clash. High values are generally associated with poor structural model quality and are less likely to produce stable complexes while low values are associated with good model quality and high predictive power of the other metrics.

### Example 2: Generation of One-Armed Antibody (OAA) Constructs Using a Heterodimeric IgA Fc

Mutations that were predicted to drive heterodimerization as described in Example 1 were introduced into one-armed antibody constructs containing an IgA Fc to assess their functionality.

Methods

In order to assess mutations designed to drive heterodimeric pairing of an IgA Fc for their effectiveness, an IgA one-armed antibody format with significant weight differences between its two halves was designed. One half-antibody consisted of an IgG1-based anti-Her2 Fab (heavy chain: SEQ ID NO:38, light chain: SEQ ID NO:39, Carter, et al., 1992, Proc Natl Acad Sci USA, 89:4285-4289) that was fused in the heavy chain to an IgA Fc. A chimeric hinge comprising the upper IgG1 hinge (SEQ ID NO: 40) N-terminally attached to an IgA2 hinge (SEQ ID NO:41) was used to connect the IgG Fab to the IgA2 Fc. The sequence of the IgA Fc resembled that of CH2 and CH3 domain of the IgA2m3 allotype (Chintalacharuvu, et al., 1994, J Immunol, 152:5299-5304). Position C5092 (IMGT numbering as shown in Table 2), which attaches to the secretory compartment in WT IgA, and the N5120 glycosylation site were mutated and the α-tailpiece was removed, ending the construct with G6129 as described in Lohse et al., 2016, Cancer Res, 76:403-417 (see SEQ ID NO: 43 in Table 4).

The other half of the one-armed antibody format consisted of just an IgA2 hinge (SEQ ID N2:41) fused to an IgA2m1 CH2 and CH3 without a Fab. The same Fc-mutations as in the heavy chain above were also included. Mutations predicted to drive heterodimeric pairing in Example 1 and listed in Table 11 were introduced into the CH3 domains of the Fc of the one-armed antibody constructs and resulted in the variants described in Table 13. Chain A mutations were introduced in the heavy chain including VH and CH1 (H1) and Chain B mutations were introduced in the Fc-only heavy chain.

TABLE 13
Heterodimeric IgA Variants in OAA Format

| | | Clone | Clone | Clone |
|---|---|---|---|---|
| Variant | Design | No. H1 | No. L1 | No. H2 |
| 32595 | WT IgA | 21755 | 11150 | 21715 |
| 32510 | Electrostatic 1 | 23773 | 11150 | 23767 |
| 32511 | Electrostatic 2 | 23774 | 11150 | 23768 |
| 32512 | Electrostatic 3 | 23775 | 11150 | 23769 |
| 32513 | Electrostatic 4 | 23776 | 11150 | 23770 |
| 32514 | Electrostatic 5 | 23777 | 11150 | 23771 |
| 32515 | Electrostatic 6 | 23777 | 11150 | 23772 |
| 32516 | Steric 1 | 23778 | 11150 | 23783 |
| 32517 | Steric 2 | 23779 | 11150 | 23783 |
| 32518 | Steric 3 | 23780 | 11150 | 23784 |
| 32519 | Steric 4 | 23781 | 11150 | 23785 |
| 32520 | Steric 5 | 23782 | 11150 | 23784 |
| 32521 | Steric 6 | 23780 | 11150 | 23786 |
| 33330 | Steric 7 | 24674 | 11150 | 23786 |
| 33331 | Steric 8 | 24675 | 11150 | 23786 |
| 33332 | Steric 9 | 24676 | 11150 | 23786 |
| 33333 | Steric 10 | 23780 | 11150 | 24677 |
| 33334 | Steric 11 | 24678 | 11150 | 24679 |

### Example 3: Production of Heterodimeric IgA One Armed Antibodies

Sequences of heavy and light chains of modified IgA OAA variants designed in Examples 1 and 2 were cloned into expression vectors and expressed and purified as described below.

Methods

Vector inserts comprising a signal peptide (EFATMRPTWAWWLFLVLLLALWAPARG [SEQ ID NO:49]) (Barash et al., 2002, Biochem and Biophys Res. Comm., 294:835-842) and the heavy and light chain sequences described in Example 2 were ligated into a pTT5 vector to produce heavy and light chain expression vectors. Vectors were sequenced to confirm correct reading frame and sequence of the coding DNA.

Heavy and light chains and the Fc-only chains of the modified IgA OAA variants were co-expressed in 25 mL cultures of Expi293F™ cells (Thermo Fisher, Waltham, MA). Expi293™ cells were cultured at 37° C. in Expi293™ Expression Medium (Thermo Fisher, Waltham, MA) on an orbital shaker rotating at 125 rpm in a humidified atmosphere of 8% CO₂. A volume of 25 mL with a total cell count of 7.5×10⁷ cells was transfected with a total of 25 μg DNA at a transfection ratio of 30:40:30 for H1:L1:H2. Prior to transfection the DNA was diluted in 1.5 mL Opti-MEM™ I Reduced Serum Medium (Thermo Fisher, Waltham, MA). In a volume of 1.42 mL Opti-MEM™ I Reduced Serum Medium, 80 μL of ExpiFectamine™ 293 reagent (Thermo Fisher, Waltham, MA) were diluted and, after incubation for five minutes, combined with the DNA transfection mix to a total volume of 3 mL. After 10 to 20 minutes the DNA-ExpiFectamine™293 reagent mixture was added to the cell culture. After incubation at 37° C. for 18-22 hours, 150 μL of ExpiFectamine™ 293 Enhancer 1 and 1.5 mL of ExpiFectamine™ 293 Enhancer 2 (Thermo Fisher, Waltham, MA) were added to each culture. Cells were incubated for five to seven days, and supernatants were harvested for protein purification.

Clarified supernatant samples were diluted 1:1 with PBS and applied to 2 mL of CaptureSelect™ IgA Affinity Matrix (ThermoFisher, Waltham, MA) packed in-house in a Millipore Vantage L×250 column on AKTA™ Pure FPLC System (GE Life Sciences). The column was equilibrated in PBS. After loading, the column was washed with PBS and protein eluted with 0.1 M glycine, pH 2.5. The eluted samples were pH adjusted by adding 10% (v/v) 1 M Tris, pH 9 to yield a final pH of 6-7. The variants were assessed for heterodimeric purity after affinity chromatography by non-reducing CE-SDS and UPLC-SEC as described in Example 4.

After concentration and to separate heterodimeric from homodimeric Fc species and other impurities, the material of variants with significant amounts of heterodimeric species was injected into an AKTA™ Pure FPLC System (GE Life Sciencies) and run on a Superdex 200 Increase 10/300 GL (GE Life Sciences) column pre-equilibrated with PBS pH 7.4. The protein was eluted from the column at a rate of 0.75 mL/min and collected in 0.5 mL fractions. Peak fractions with concentrations of >0.5 mg/mL of target protein and a CE-SDS purity of >95% were pooled and concentrated using Vivaspin™ 20, 30 kDa MWCO polyethersulfone concentrators (MilliporeSigma, Burlington, MA). After sterile filtering through 0.2 μm PALL Acrodisc™ Syringe Filters with Supor™ Membrane, proteins were quantitated based on A280 nm (Nanodrop), frozen and stored at −80° C. until further use.

Results

Inclusion of electrostatic design mutations did not result in variants with detectable expression, pointing to a disruptive nature of these mutations. Conversely, all steric designs showed expression under the conditions tested and ten designs were purified and investigated further (Steric 1-4, Steric 6-11). While some samples of these variants showed highly pure, heterodimeric species after affinity chromatography, preparative SEC was required in order to obtain samples of high purity for most due to the presence of homodimeric Fc species as well as other impurities such as half antibodies and aggregates (see Example 4). After preparative SEC was performed on Steric 1-3 and Steric 6-11 designs as well as the WT IgA Fc OAA, yields ranged from 30-200 mg/L of expression culture. The assessment of sample purity and stability is described in Example 4, Example 5 and Example 6.

### Example 4: Assessment of Heterodimeric Purity of Lead Designs after Affinity Chromatography

OAA variants were assessed for heterodimeric purity and sample homogeneity by non-reducing CE-SDS and UPLC-SEC after CaptureSelect IgA affinity purification and before SEC purification.

Methods

Following CaptureSelect IgA affinity purification, purity of samples was assessed by non-reducing and reducing High Throughput Protein Express assay using CE-SDS LabChip® GXII (Perkin Elmer, Waltham, MA). Procedures were carried out according to HT Protein Express LabChip® User Guide version 2 with the following modifications. Antibody samples, at either 2 ul or 5 ul (concentration range 5-2000 ng/ul), were added to separate wells in 96 well plates (BioRad, Hercules, CA) along with 7 ul of HT Protein Express Sample Buffer (Perkin Elmer #760328). Samples were then denatured at 90° C. for 5 mins and 35 μl of water was added to each sample well. The LabChip® instrument was operated using the HT Protein Express Chip (Perkin Elmer #760499) and the HT Protein Express 200 assay setting (14 kDa-200 kDa).

UPLC-SEC was performed on an Agilent Technologies 1260 Infinity LC system using an Agilent Technologies AdvanceBio SEC 300A column at 25° C. Before injection, samples were centrifuged at 10000 g for 5 minutes, and 5 μL was injected into the column. Samples were run for 7 min at a flow rate of 1 mL/min in PBS, pH 7.4 and elution was monitored by UV absorbance at 190-400 nm. Chromatograms were extracted at 280 nm. Peak integration was performed using the OpenLAB CDS ChemStation software.

Results

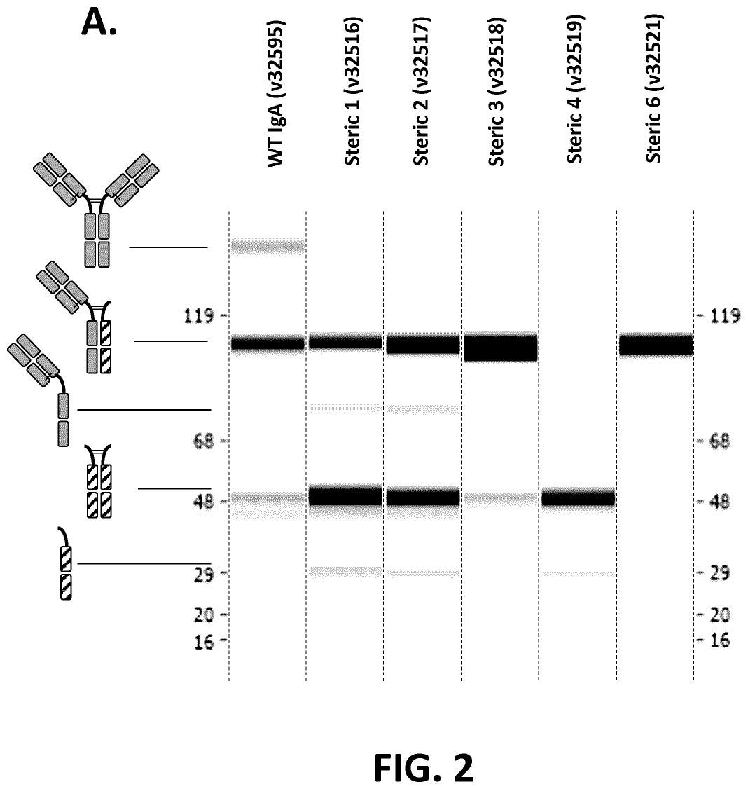

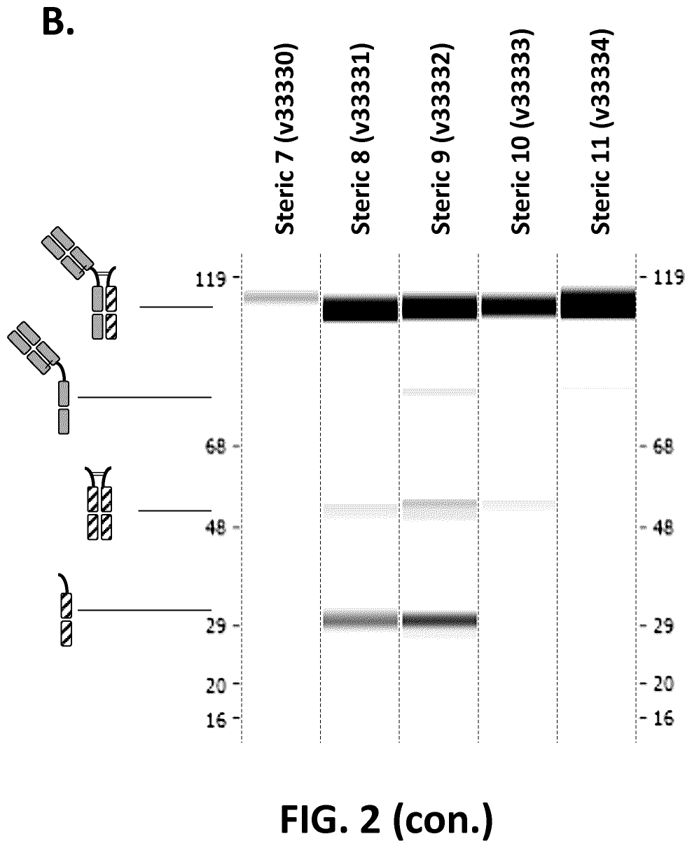

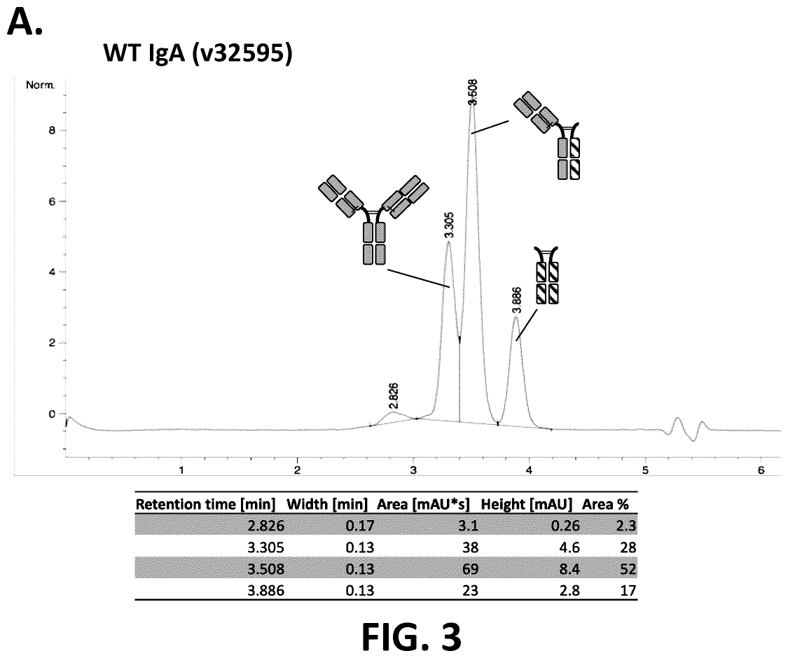

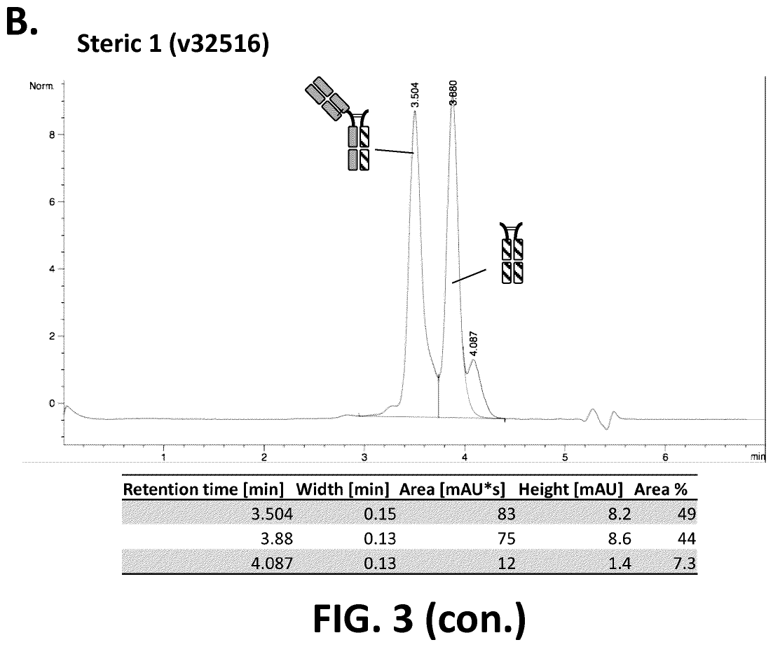

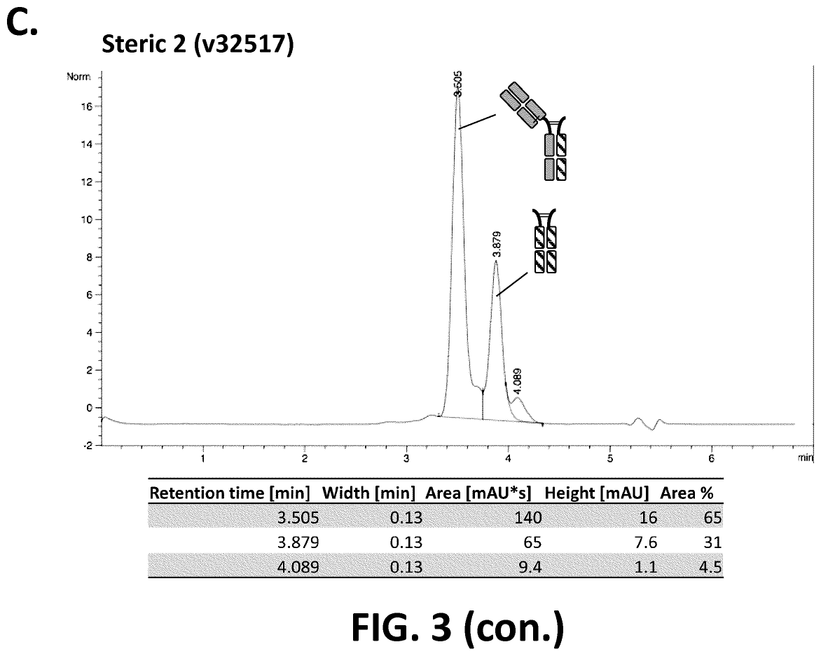

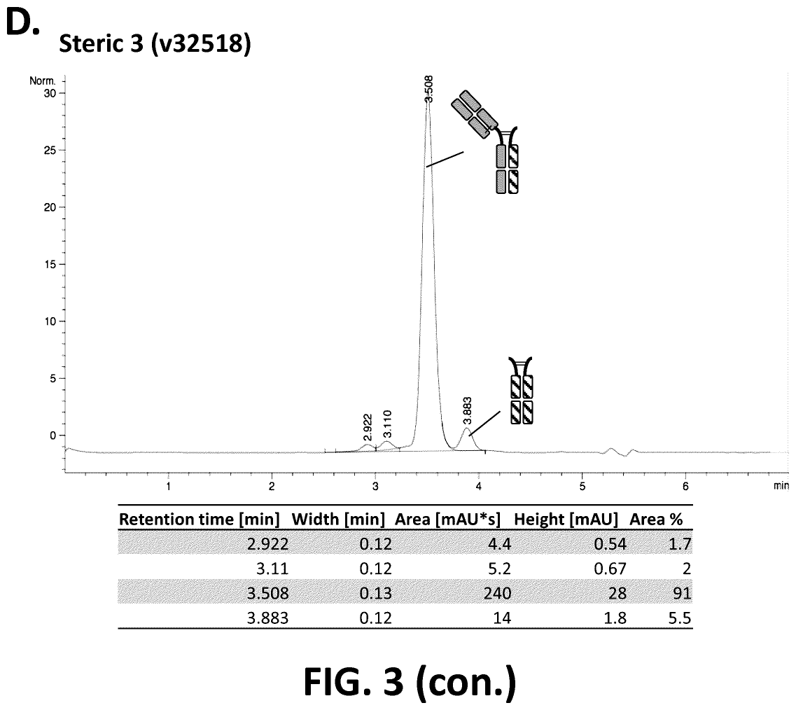

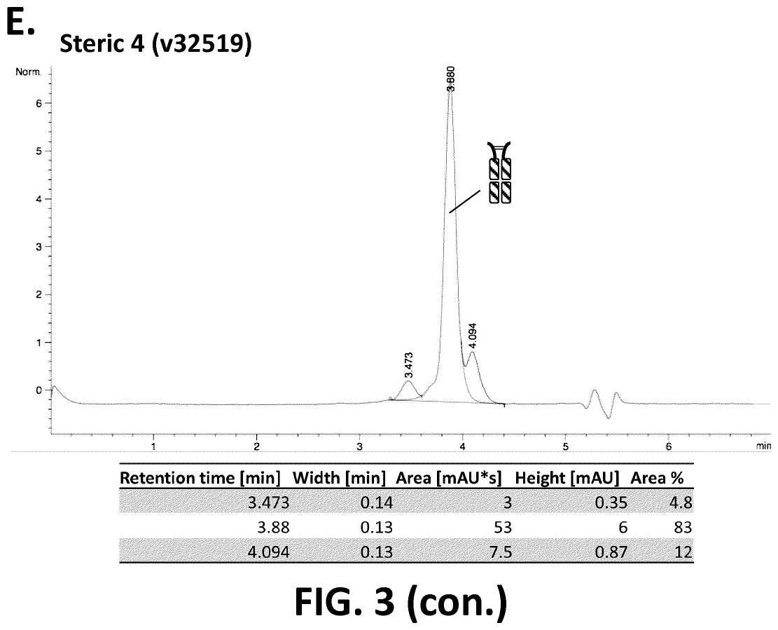

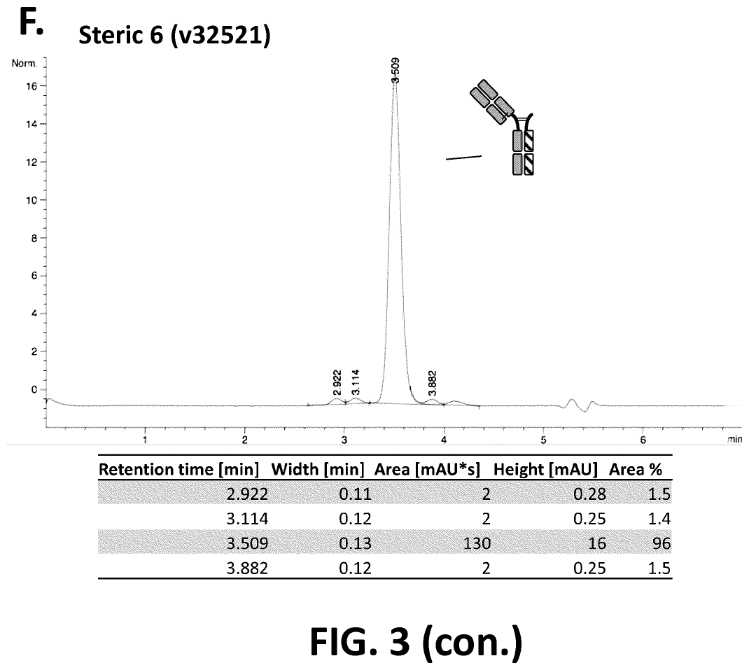

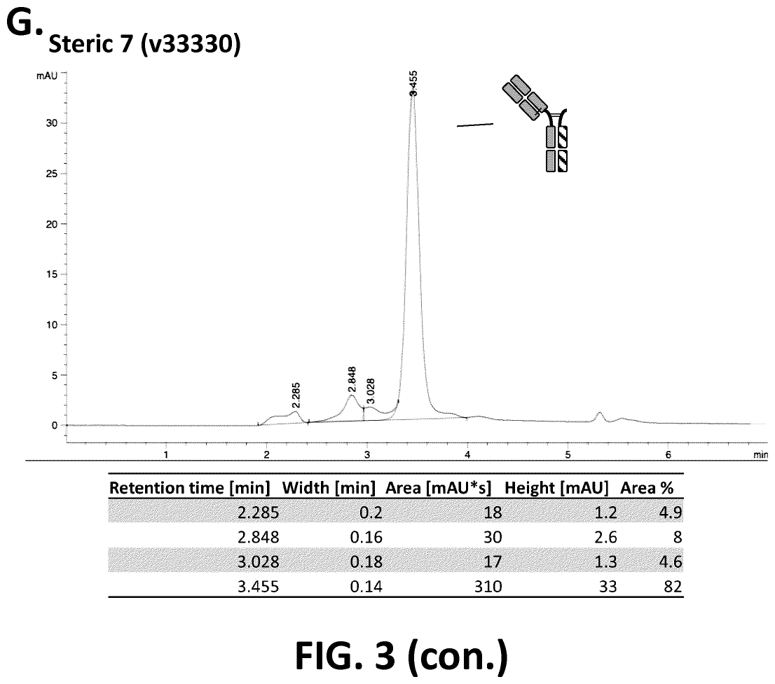

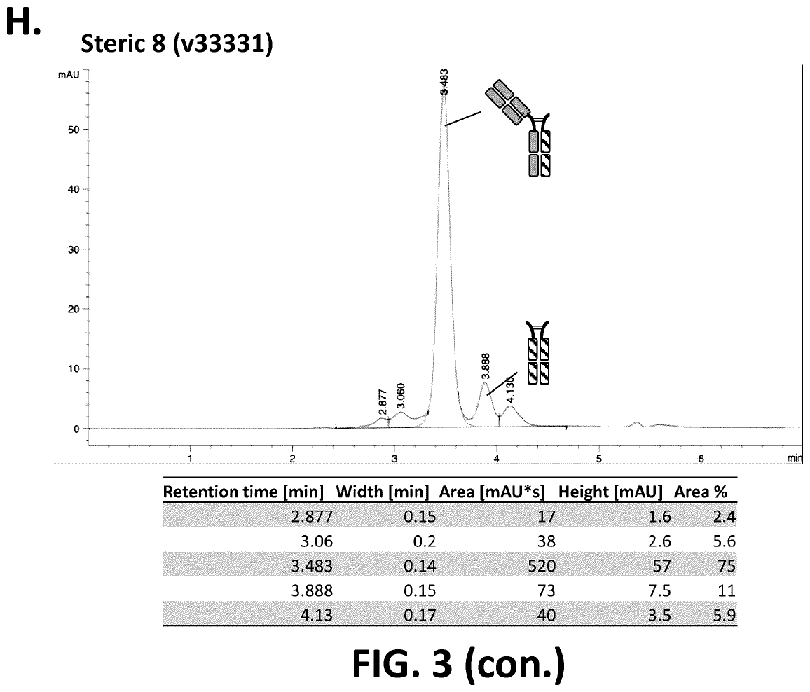

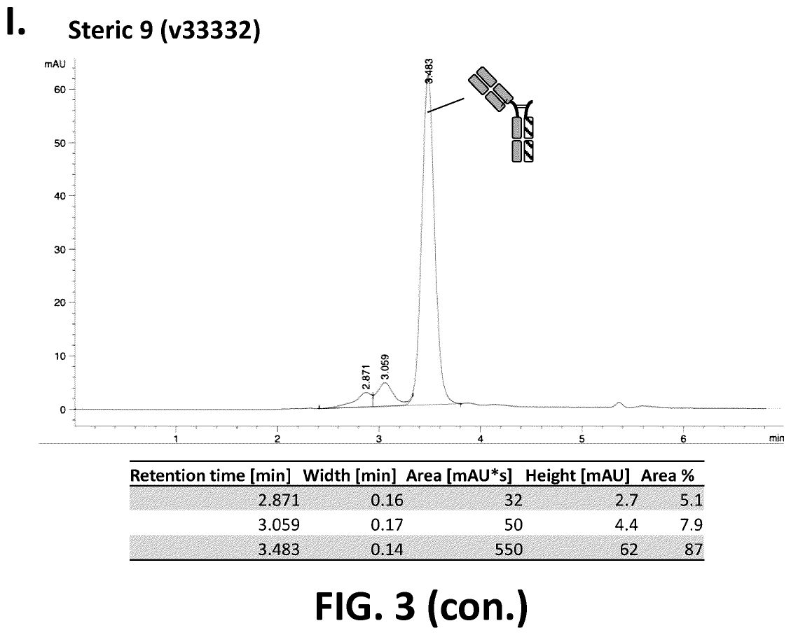

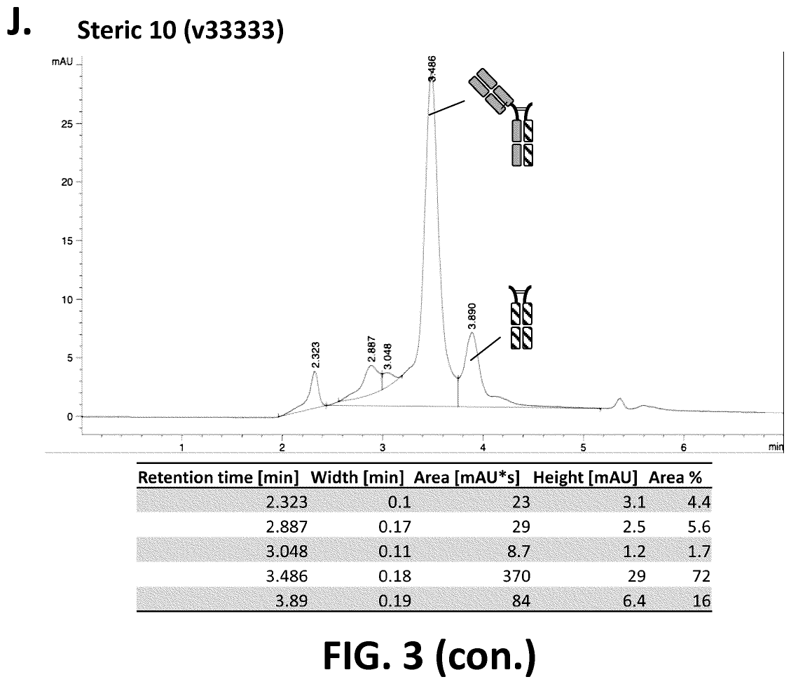

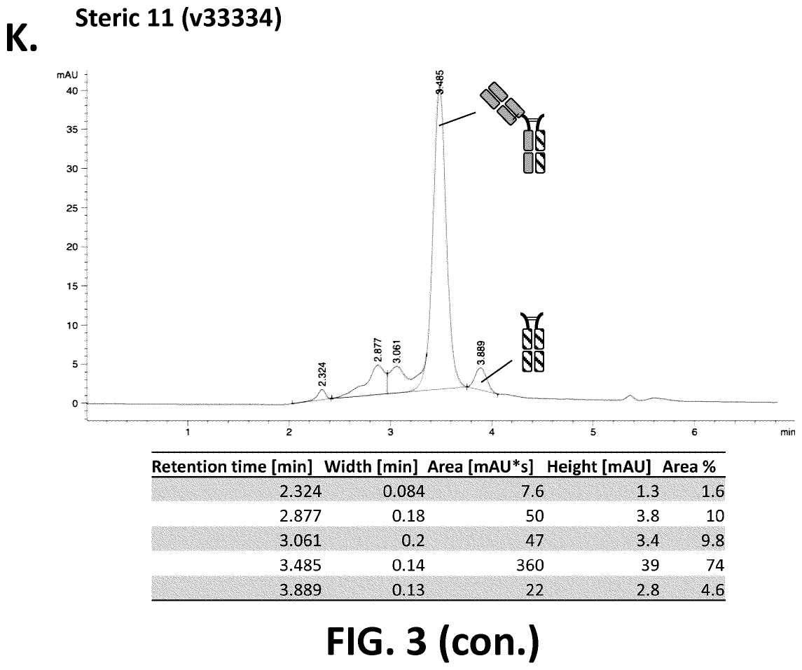

Analysis of non-reducing CE-SDS of the WT IgA OAA (v32595) showed a mix of homodimeric Full Sized Antibody (FSA) together with Fc and heterodimeric OAA species (FIG. 2). The heterodimeric species was the most prominent with less of each homodimeric species present. This is the expected distribution of species at equimolar expression of both Fc chains without any mutations promoting heterodimer formation present (Ridgway, et al., 1996, Protein Eng, 9:617-621) and was also seen by UPLC-SEC (FIG. 3A).

Variants including mutations promoting heterodimer formation showed notably different distribution of species in both non-reducing CE-SDS (FIG. 2) and UPLC-SEC (FIG. 3) as compared to WT IgA OAA. While FSA homodimers were not present for any of the steric designs shown in FIGS. 2 and 3, varying levels of Fc homodimers and half antibody species could be found in addition to OAA heterodimers. Most notably, Steric 3 (v32518; FIG. 3D) and Steric 6 (v32521; FIG. 3F) designs showed significantly increased purity of OAA heterodimeric species with Steric 6 reaching heterodimeric purity of >95% by both CE-SDS and UPLC-SEC. Conversely, Steric 4 (v32519; FIG. 3E) contained no OAA heterodimer or FSA homodimer species but only Fc homodimer and the corresponding half-antibody, pointing to a problem in the expression of the other heavy chain likely caused by the introduced mutations. The presence of small peaks at retention times <3 min indicated the presence of small amounts of high molecular weight species such as oligomers and aggregates in all samples.

### Example 5: Assessment of Heterodimeric Purity of Lead Designs after Size Exclusion Chromatography

After SEC purification of select designs, samples were assessed for homogeneity of the sample by non-reducing as well as reducing CE-SDS and UPLC-SEC as described below.

Methods

Non-reducing CE-SDS and UPLC-SEC were performed as described in Example 4. For electrophoretic analysis under reducing conditions, the CE-SDS protocol was modified by adding 3.5 μL of DTT(1M) to 100 μL of HT Protein Express Sample Buffer.

Results

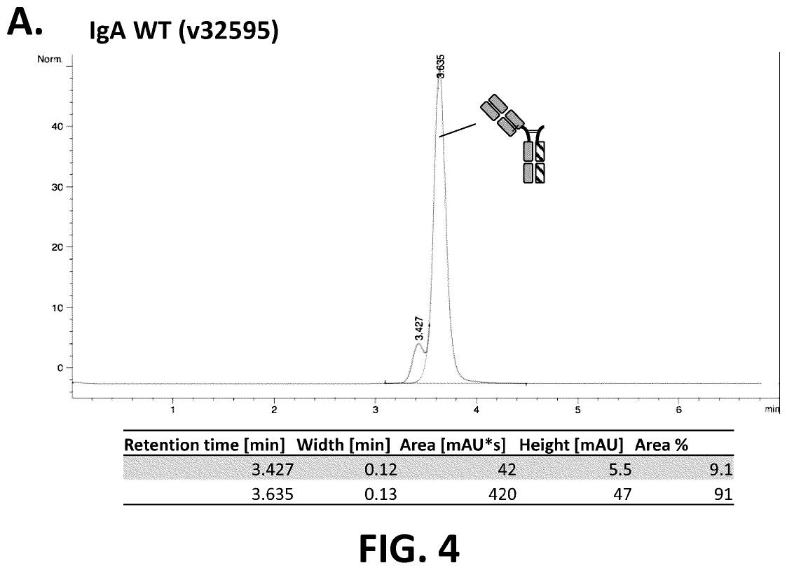

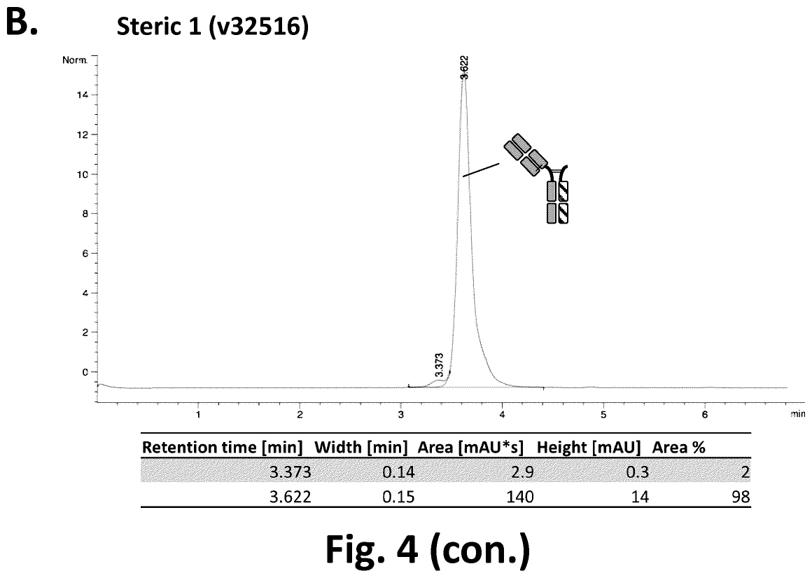

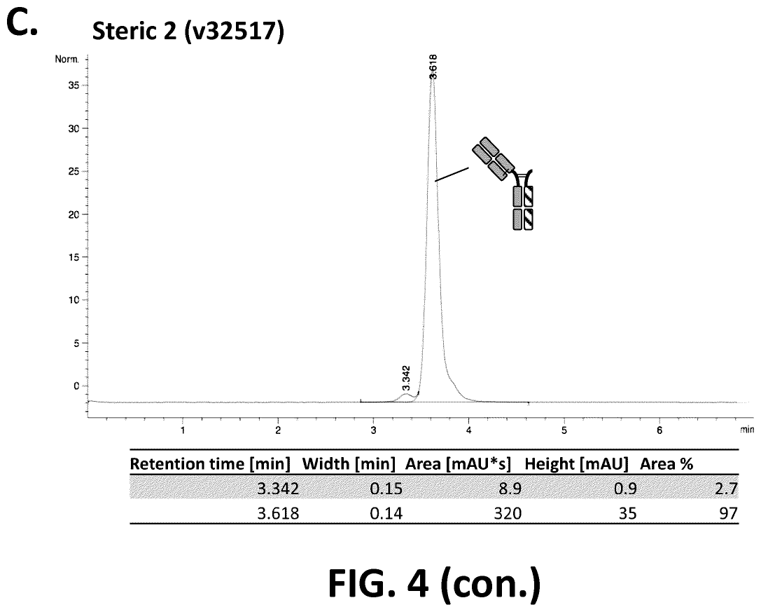

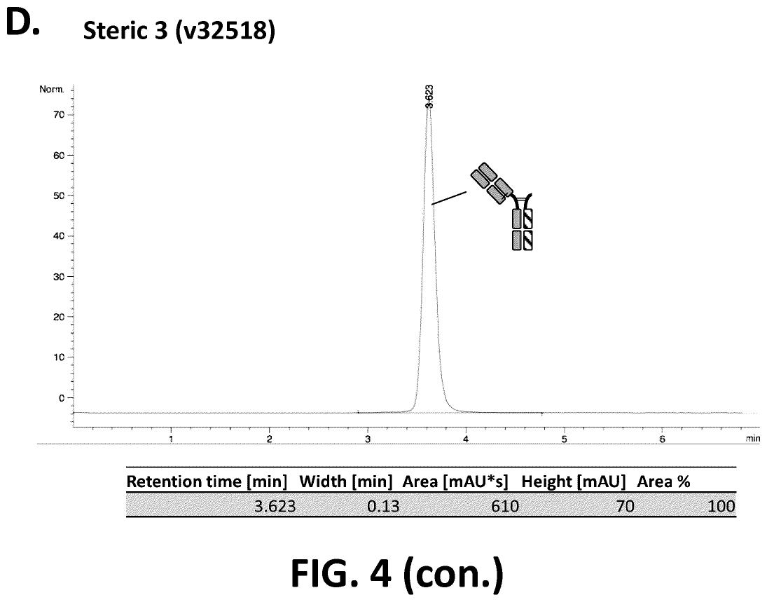

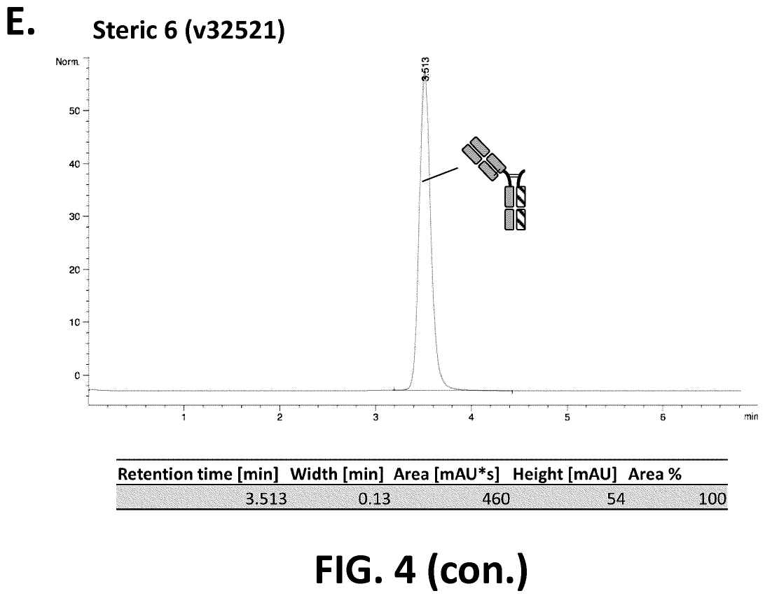

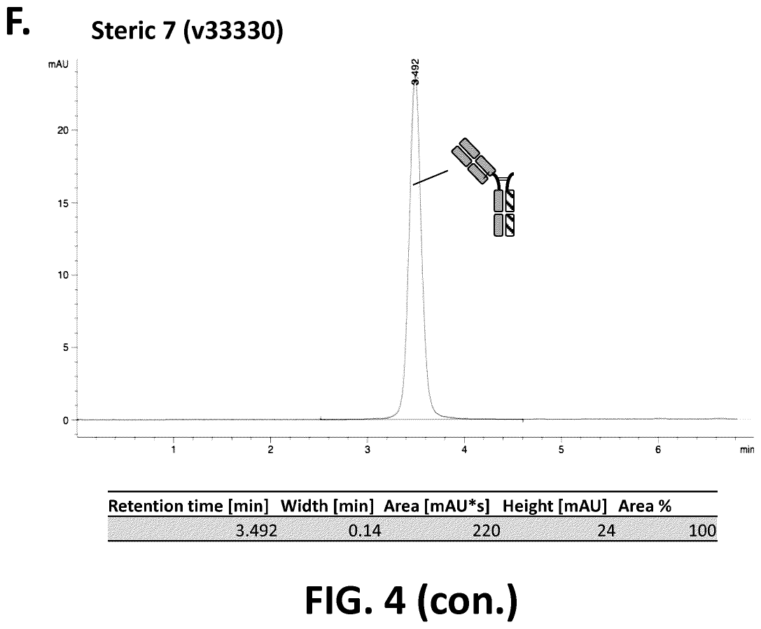

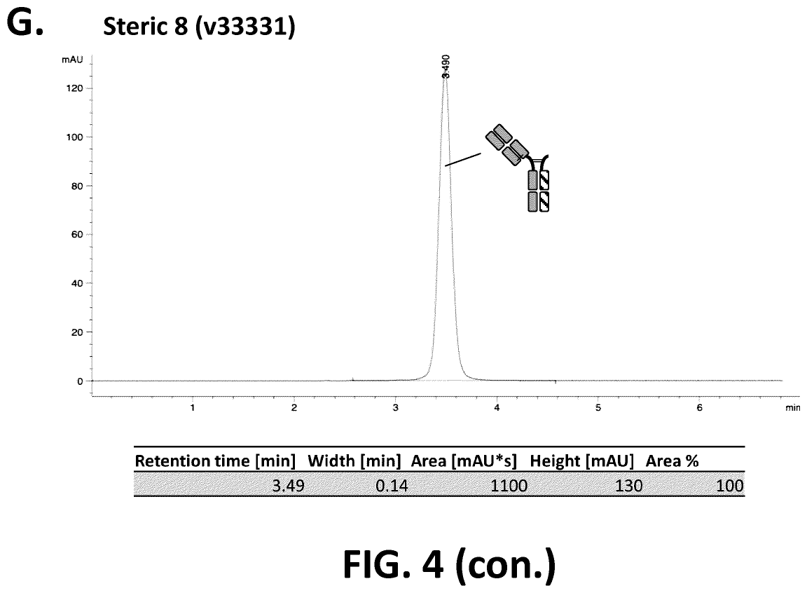

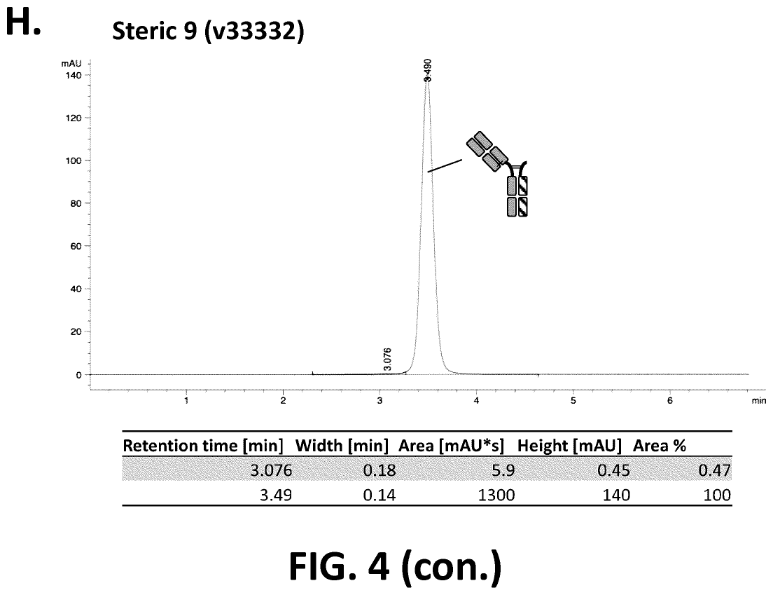

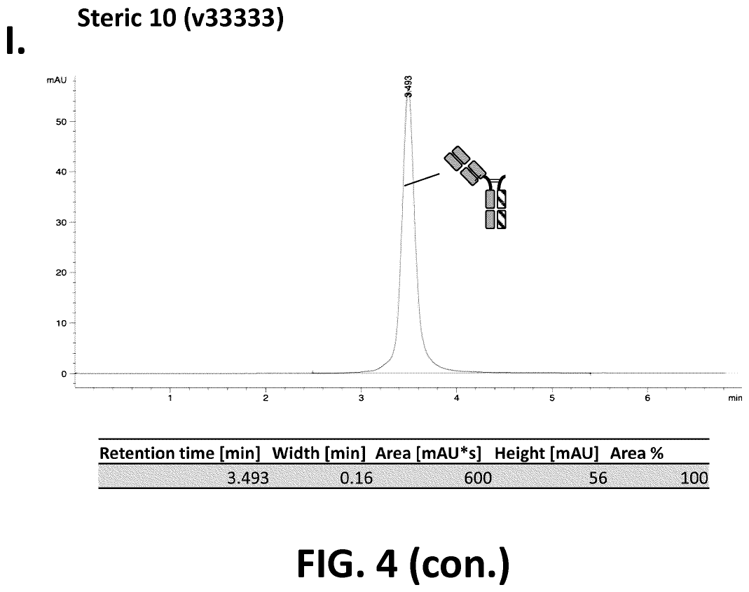

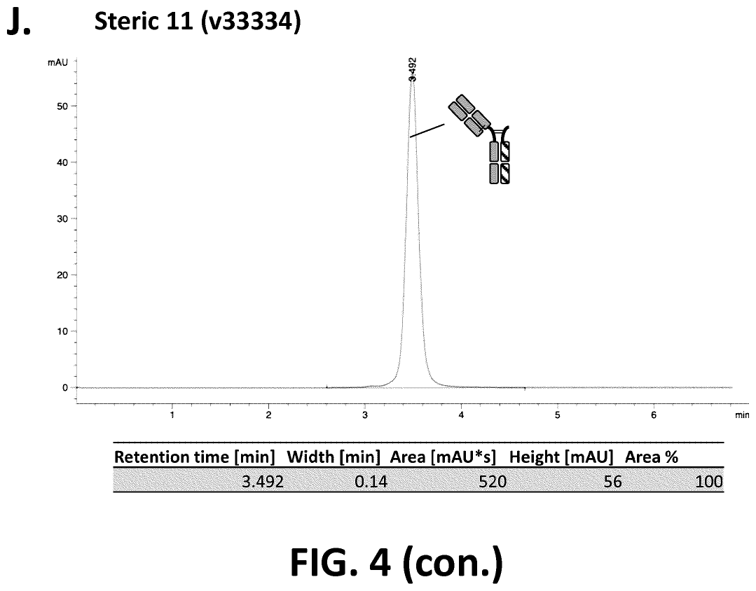

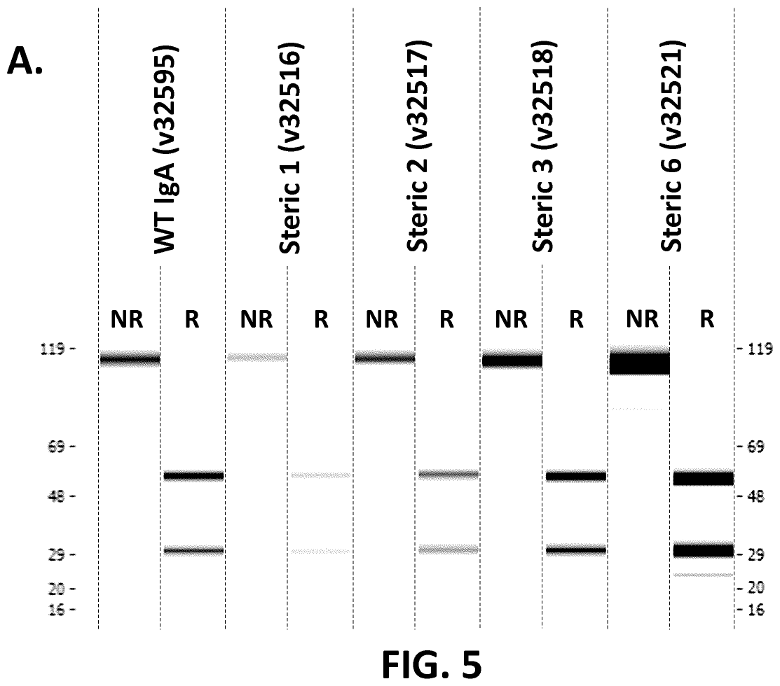

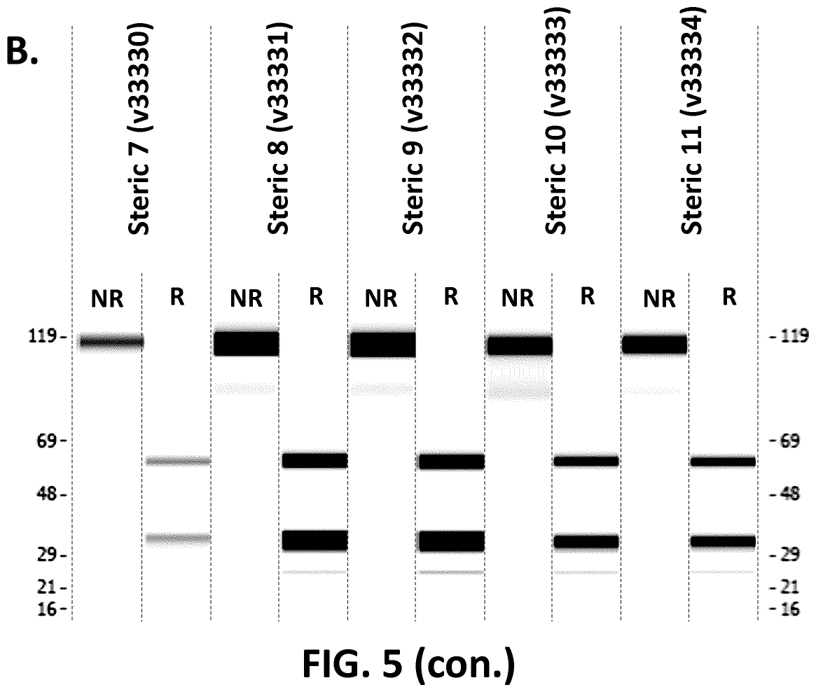

UPLC-SEC traces and CE-SDS electrophoresis profiles (reducing and non-reducing) of heterodimeric OAA samples purified by SEC as described in Example 3 are shown in FIG. 4 and FIG. 5, respectively. Analysis of UPLC-SEC showed highly homogeneous samples that contained 90%-100% of heterodimeric OAA species. The presence of a small peak at a low retention time and a shoulder at higher retention time compared to the main species indicates the presence of small amounts of homodimers in WT IgA (FIG. 4A), Steric 1 (FIG. 4B) and Steric 2 (FIG. 4C) designs. After SEC purification, non-reducing CE-SDS showed a single predominant species for all variants investigated. Only bands corresponding to the three intact chains of all variants were observed by reducing CE-SDS. Notably, light chain as well as the Fc-only heavy chain have a similar molecular weight (23.4 kDa and 23.7 kDa) and appear as one band in the reducing CE-SDS profile.

### Example 6: Thermal Stability of Lead IgA Heterodimer Designs

Purified samples of heterodimeric OAA variants after preparative SEC were assessed for thermal stability by Differential Scanning Calorimetry (DSC) as described below.

Methods

After preparative SEC as described in Example 3, samples of heterodimeric OAA designs were diluted in PBS to 0.5-1 mg/ml. For DSC analysis using NanoDSC (TA Instruments, New Castle, DE, USA), 950 ul of sample and matching buffer (PBS) were added to sample and reference 96 well plates, respectively. At the start of the DSC run, a buffer (PBS) blank injection was performed to stabilize the baseline. Each sample was then injected and scanned from 25° C. to 95° C. at 1° C./min with 60 psi nitrogen pressure. Thermograms were analyzed using the NanoAnalyze software. The matching buffer thermogram was subtracted from sample thermogram and baseline fit using a sigmoidal curve. Data was then fit with a two-state scaled DSC model.

Results

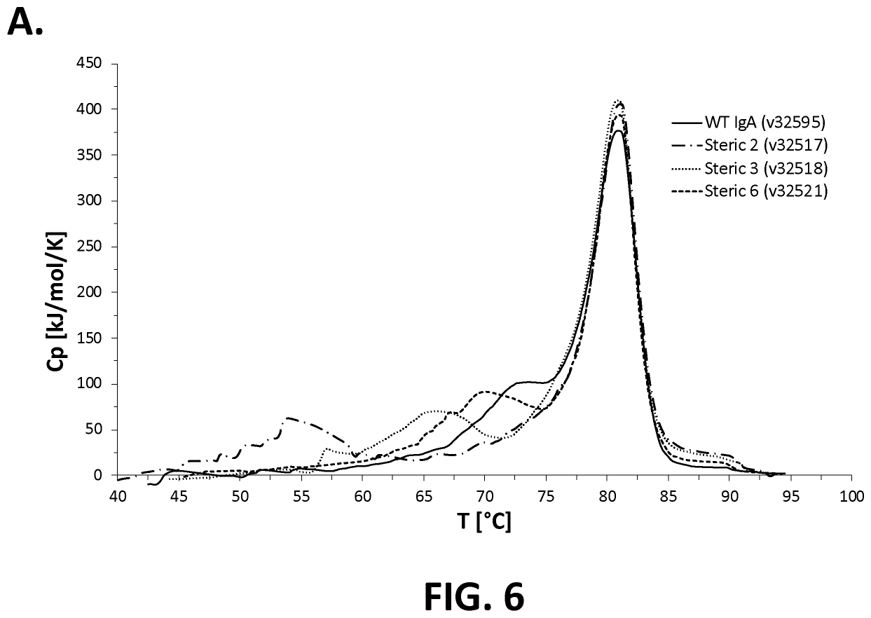

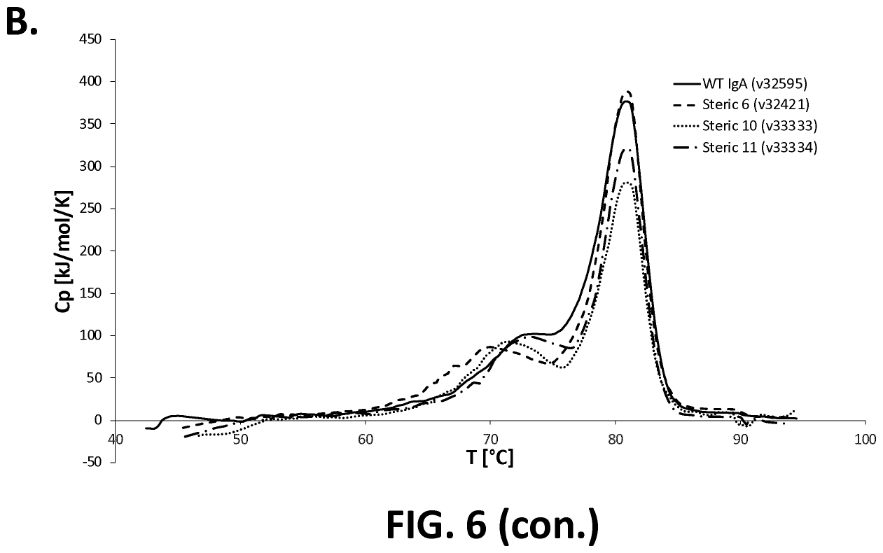

The DSC thermogram of WT IgA OAA with an unmodified IgA CH3-CH3 interface (v32595) showed two transitions at 74° C. and 81° C. (FIG. 6A). The more dominant transition at 81° C. was present for all investigated designs and was attributed to the unfolding of the Fab overlapped with unfolding of the CH2 domain, neither of which was mutated in the designs. Conversely, a transition was observed to change across designs and was attributed to the unfolding of the CH3 domain (FIG. 6A-B). While the modified CH3 in Steric 2 (v32517) was significantly destabilized compared to WT (Tm of 55° C. vs 74° C.), the designs with the highest heterodimeric purity show CH3 stabilities close to WT. Transitions were observed at 65.9° C. and 71.9° C. for Steric 3 (v32518) and Steric 6 (v32521), respectively. The two designs that showed the highest thermal stability were Steric 10 (v33333) and Steric 11 (v33334) with CH3 unfolding transitions observed at 72.0° C. and 73.6° C., respectively. This higher thermal stability was observed while the heterodimeric purity of these two designs as assessed by CE-SDS and UPLC-SEC in Example 4 was lower than that of Steric 3 and Steric 6.

In summary, combinations of mutations were identified in the IgA CH3 domain that significantly drove heterodimer formation of the IgA Fc. The thermal stability of the CH3 domain of heterodimeric variants bearing these mutations was within −2° C. of the WT IgA CH3 for the Steric 6 (v32521), Steric 10 (v33333) and Steric 11 (v33334) designs. The properties of the Steric designs tested are summarized in Table 14.

TABLE 14
Summary of Properties of Steric Designs 1-3 and 6-11

| | | | | Post CSIgA Purification | | | Post prepSEC Purification | | | |
|---|---|---|---|---|---|---|---|---|---|---|
| | | | | UPLC-SEC | | Yield | UPLC-SEC | | Yield | |
| | | HetFc Mutations | | purity | CE-SDS | (mg/L | purity | CE-SDS | (mg/L | Tm* |
| Variant | Design | Chain A | Chain B | (%) | purity (%) | culture) | (%) | purity (%) | culture) | (° C.) |
| 32595 | WT IgA | — | — | 52 | 49 | 324 | 91 | 92 | 76 | 74.2 |
| 32516 | Steric 1 | A6085YY_T6086L | L6079T_W6081L_ | 49 | 36 | 148 | 98 | 93 | 36 | 71.1 |
| | | | I6088L | | | | | | | |
| 32517 | Steric 2 | A6085YY_T6086Y | L6079T_W6081L_ | 65 | 55 | 240 | 97 | 96 | 76 | 55 |
| | | | I6088L | | | | | | | |
| 32518 | Steric 3 | A6085YF_T6086Y | L6079V_W6081L_ | 91 | 89 | 328 | 100 | 98 | 136 | 65.9 |
| | | | I6088L | | | | | | | |
| 32519 | Steric 4 | L6024M_A6085YF_ | W6081L | 5 | 3.7 | 60 | ND | ND | ND | ND |
| | | T6086W | | | | | | | | |
| 32521 | Steric 6 | A6085YF_T6086Y | L6079V_W6081T_ | 96 | 92 | 320 | 100 | 97 | 100 | 71.9 |
| | | | I6088L | | | | | | | |
| 33330 | Steric 7 | T6022V_A6085YF_ | L6079V_W6081T_ | 82 | 31 | 130 | 100 | 99 | 52 | 69.2 |
| | | T6086Y | I6088L | | | | | | | |
| 33331 | Steric 8 | T6022L_A6085YF_ | L6079V_W6081T_ | 75 | 97 | 370 | 100 | 95 | 140 | 67.6 |
| | | T6086Y | I6088L | | | | | | | |
| 33332 | Steric 9 | T60221_A6085YF_ | L6079V_W6081T_ | 87 | 97 | 390 | 100 | 95 | 210 | 69 |
| | | T6086Y | I6088L | | | | | | | |
| 33333 | Steric 10 | A6085YF_T6086Y | L6007F_L6079V_ | 72 | 88 | 370 | 100 | 85 | 82 | 72 |
| | | | W6081T_I6088L | | | | | | | |
| 33334 | Steric 11 | H6005Y_A6085YF_ | H6005Y_L6079V_ | 74 | 95 | 440 | 100 | 93 | 71 | 73.6 |
| | | T6086Y | W6081T_I6088L | | | | | | | |

*CH3 domain Tm determined by DSC

### Example 7: Further Stabilization of IgA HetFc Designs

To increase the thermal stability and heterodimeric purity of lead IgA HetFc designs via covalent disulfide bridges across the interface, cysteine mutations were introduced in the CH3 interface of the IgA Fc.

Methods

Residue pairs in the interface of the IgA Fc were selected based on Cα and Cβ distances determined to be sufficient to accommodate the geometry of a disulfide bond. The selected residues were then substituted with cysteine residues and the resultant covalent disulfide bonds were modelled. The resulting structures were evaluated energetically using proprietary in silico tools.

Results

Cysteine substitutions were introduced into the Steric 6 design and evaluated by proprietary in silico tools. Exemplary metrics for select designs are shown in Table 15. The cysteine substitutions were then introduced as single and double disulfide designs in an OAA format of Steric 6 as well as a single disulfide design in a WT OAA (Table 16).

The variants shown in Table 16 will be expressed and evaluated for heterodimeric purity and thermal stability. While the high heterodimeric purity of Steric 6 based designs (34688-34690) as assessed by UPLC-SEC and CE-SDS is expected to be preserved when compared to that of Steric 6 (>90% as assessed by UPLC-SEC and CE-SDS after CaptureSelect IgA purification, see example 6), the thermal stability of these designs, as measured by DSC, is predicted to be significantly increased when compared to that of Steric 6 (>71° C., see example 6) due to the addition of one or two covalent disulfide bonds in the interface. When introduced as a single disulfide design in an asymmetric manner in an otherwise unchanged WT IgA Fc (34691), heterodimeric purity as assessed by UPLC-SEC and CE-SDS is expected to be significantly improved compared to WT IgA (>50% as assessed by UPLC-SEC and CE-SDS after CaptureSelect IgA purification, see example 6) and thermal stability is predicted to be at or above WT (>74° C., see example 6).

The identified disulfide designs may also be combined with other lead HetFc designs identified in Examples 1-6, expressed in OAA format, purified and assessed for heterodimeric purity as well as thermal stability as described in Examples 2-6.

TABLE 15
Exemplary Metrics used for Disulfide Bond Design Selection

| Chain A | Chain B | Δ Physics- | Δ Knowledge- | Disulfide Dihedral | Number |
|---|---|---|---|---|---|
| Cysteine | Cysteine | Based Affinity | Based Affinity | Angle Energy | of |
| Mutation | Mutation | [kcal/mol]^{1, 2} | [kcal/mol]^{1, 2} | [KJ/mol]³ | Clashes⁴ |
| H6005C | P6010C | 2.5E+01 | −5.6E+01 | 1.3E+01 | 0 |
| P6010C | H6005C | 1.4E+01 | −5.7E+01 | 1.4E+01 | 0 |

¹Δ refers to difference in the reported metric compared to WT IgA CH3 homodimer.
²Metrics reporting on the non-covalent energetics of the interactions of Chain A and Chain B compared to the WT complex. Negative values indicate a more favourable interaction compared to the WT complex, positive values indicate a less favourable interaction compared to the WT complex. The energy difference afforded by the formation of the covalent disulfide bridge is not included.
³Metric reporting on the dihedral angle strain in the disulfide bond. Smaller values indicate less angle strain.
⁴Clashes are flagged for distances between heavy atoms that fall below distance cut-offs defined for different types of interactions.

TABLE 16
Selected Heterodimeric IgA Variants Including a Disulfide Bond

| | | Clone | Clone | Clone |
|---|---|---|---|---|
| Variant | Description | No. H1 | No. L1 | No. H2 |
| 34688 | IgA Het_Fc OAA Steric 6 | 25880 | 11150 | 25881 |
| | A H6005C B P6010C | | | |
| 34689 | IgA Het_Fc OAA Steric 6 | 25882 | 11150 | 25883 |
| | A P6010C B H6005C | | | |
| 34690 | IgA Het_Fc OAA Steric 6 | 25884 | 11150 | 25885 |
| | A H6005C P6010C | | | |
| | B H6005C P6010C | | | |
| 34691 | WT IgA OAA | 25886 | 11150 | 25887 |
| | A H6005C B P6010C | | | |

### Example 8: Multimeric, Multispecific Formats Based on IgA HetFc

Mutations driving heterodimeric pairing of the IgA Fe described in Example 1-7 can be used to construct multimeric, multispecific variants, which may then be tested for target binding and functionality.

Methods

The two chains of an IgA1, IgA2m1 or IgA2m2 Fc including a C-terminal tailpiece (SEQ ID NO:46 or 47) are equipped with mutations in the CH3 domain that drive heterodimer formation as described in Examples 1-6 and Table 11, to form the core IgA HetFc scaffold. A binding domain (e.g. Fab, scFv, VHH, Immunomodulatory Ig domain, non-Ig viral receptor decoy, and as described elsewhere herein) specific for one target is linked to the N-terminus of one of the IgA HetFc chains via an IgA1, IgA2 or IgG1/IgA2 chimeric hinge while the same hinges are used to link a second binding domain specific for another target to the N-terminus of the other chain of the IgA HetFc. The resulting two chains are then transiently expressed in a mammalian expression system together with a joining chain (J-chain) as well as any additional polypeptide chains needed to complete the IgA HetFc construct (e.g. other chains to complete Fabs used as targeting domains). Depending on the IgA allotype used for the Fc and the ratio of J chain to IgA Fc chains, this results in the formation of dimeric, tetrameric or pentameric molecules (Lombana et al., 2019, MAbs, 11:1122-1138, Kumar, et al., 2020, Science, 367:1008-1014) in which each IgA HetFc binding unit of the dimeric, tetrameric or pentameric IgA HetFc multimer possesses two binding domains (see FIG. 8). After purification by CaptureSelect™ IgA affinity chromatography, samples are assessed for purity and homogeneity of particle sizes by one or more of non-reducing and reducing SDS-PAGE or CE-SDS, UPLC-SEC, multi-angle light scattering (MALS) or dynamic light scattering (DLS). If needed, samples are further purified by SEC as described in Example 3 and their sample quality assessed as described before. Samples are then tested for target binding by one or more of surface plasmon resonance (SPR), flow cytometry or functional assays specific to the target.

Results

While IgA HetFc multimer variants based on an IgA1 and IgA2m1 HetFc will be predominately dimeric, those based on an IgA2m2 HetFc will show dimeric, tetrameric and pentameric species that can be separated by SEC. In binding studies to the individual targets, an increased apparent affinity compared to monovalent binding is expected due to the avidity provided by the multimeric scaffold. This avidity effect on the apparent affinity is expected to be further enhanced when both targets are present in the binding assay. When compared to IgG-based, monomeric and bispecific antibodies, IgA HetFc multimers with increasing valency (monomer<dimer<tetramer<pentamer) should demonstrate a sequentially enhanced apparent affinity. Taken together, this avidity effect is expected to lead to high specificity and high efficacy for binding targets which is reflected in functional studies as seen previously (Slaga et al., 2018, Sci Transl Med, 10(463):eaat5775; International Patent Publication Nos. WO 2016/141303 and WO 2016/118641). When used to target viral or bacterial pathogens, the high valency of IgA HetFc multimers is expected to lead to agglutination and clearance of the target(s), while multi-specificity limits mutational escape and assures a consistently high level of neutralization.

### Example 9: A Heterodimeric IgA Fc Including a Mutations to Eliminate Binding to FcαRI

To assess the impact of valency of FcαRI engagement via the IgA Fc on its functionality, a heterodimeric IgA Fc based on mutations described in Examples 1-7 was used to construct an IgA Fc with a single FcαRI binding site.

Methods

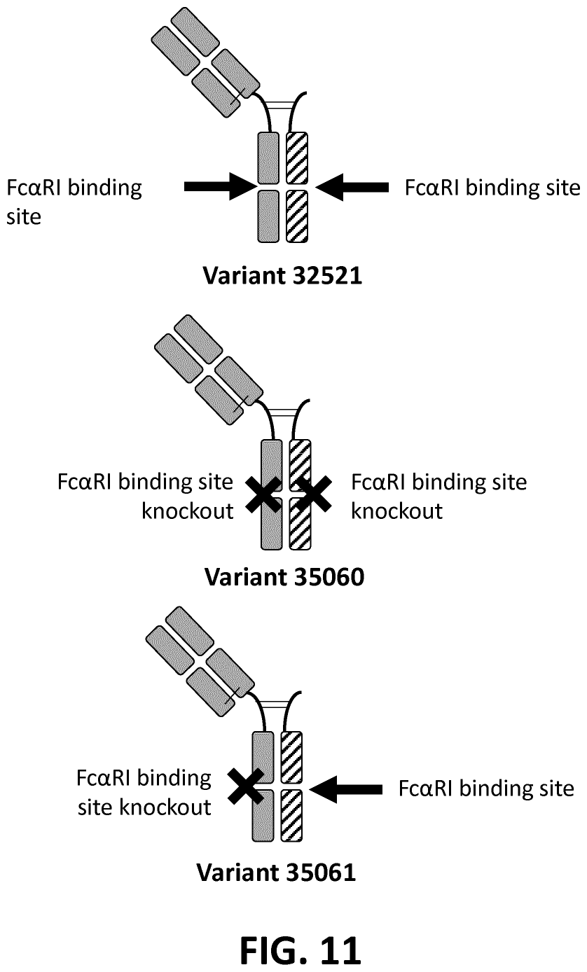

A mutation that has been identified to disrupt the IgA Fc:FcαRI interaction (F6116A, Posgai, M. T. et al., 2018, Proc Natl Acad Sci USA 115:E8882-E8891) was introduced into either one or both heavy chains of OAA variants of the Steric 6 design (Table 17). These variants as well as a wild-type Steric 6 OAA (32521) were then expressed and purified as described in examples 3-6. Other constructs may include combinations of mutations achieving differing FcαRI affinities on the two chains of a heterodimeric IgA Fc. Possible combinations are shown in Table 18. These variants can be evaluated for binding to FcαRI and neutrophil activation. Schematics of the variants containing two, one or no FcαRI binding sites are shown in FIG. 11.

TABLE 17
Heterodimeric IgA Variants based on Steric 6
OAA Including Mutations in FcαR Binding Site

| | Additional | Additional | | | |
|---|---|---|---|---|---|
| | mutations | mutations | Clone | Clone | Clone |
| Variant | chain A | chain B | No. H1 | No. L1 | No. H2 |
| 35060 | F6116A | F6116A | 26235 | 11150 | 26236 |
| 35061 | F6116A | | 26235 | 11150 | 23786 |

TABLE 18
Possible Combinations of FcαRI Affinities in IgA HetFc

| Chain A FcαRI affinity | Chain B FcαR affinity |
|---|---|
| Increased compared to WT | Increased compared to WT |
| Increased compared to WT | WT |
| Increased compared to WT | Decreased compared to WT |
| Increased compared to WT | Eliminated |
| WT | Increased compared to WT |
| WT | WT |
| WT | Decreased compared to WT |
| WT | Eliminated |
| Decreased compared to WT | Increased compared to WT |
| Decreased compared to WT | WT |
| Decreased compared to WT | Decreased compared to WT |
| Decreased compared to WT | Eliminated |
| Eliminated | Increased compared to WT |
| Eliminated | WT |
| Eliminated | Decreased compared to WT |
| Eliminated | Eliminated |

Results

Variants with modified FcαRI binding sites aimed at increasing, lowering or eliminating binding are expected to show a range of affinities to FcαRI and a range of activities in neutrophil activation assays compared to a WT IgA Fc. While knockout mutations in both chains are expected to eliminate binding and neutrophil activation, mutations aimed at increasing FcαRI binding in both chains are expected to increase binding and neutrophil activation and constitute the highest possible activity. All other combinations shown in Table 18 are expected show binding and neutrophil activation at a level between these limits.

### Example 10: A Heterodimeric IgA Fc Including FcaRI and FcRn Binding Sites

Mutations driving the assembly of a heterodimeric IgA Fc described in Examples 1-7 are used to construct IgA-based variants capable of activating neutrophils via the FcαRI as well as having an increased half-life due to the presence of a FcRn binding site.

Methods

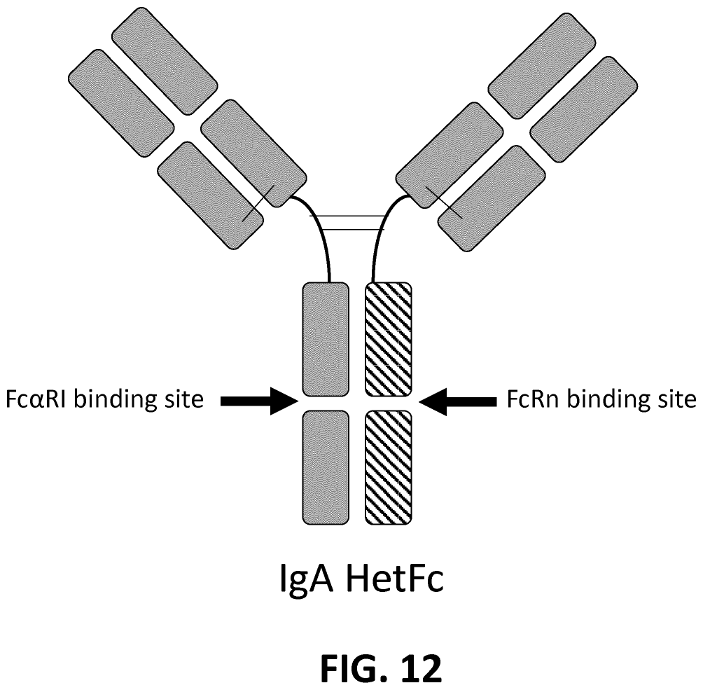

Residues important for binding of an IgG Fc to the Neonatal Fc Receptor (FcRn) (Oganesyan, V. et al., 2014, J Biol Chem 289:7812-7824) are grafted onto heterodimeric IgA variants to create constructs capable engaging FcRn as well as FcαRI. A heterodimeric Fc is necessary since FcαRI and FcRn binding sites are located in structurally equivalent locations at the CH2/CH3 interfaces in IgA and IgG, respectively (Kelton, W. et al., 2014, Chem Biol 21:1603-1609). Grafting of the FcRn binding site is achieved by an overlay of peptide backbone atoms of IgA and IgG Fc and identification of structurally equivalent residues in IgA to the IgG:FcRn binding patch. These are then swapped for their IgG counterpart. Alternatively, mutations can be included that are known to modify FcRn affinity in IgG (Robbie, G. J. et al., 2013, Antimicrob Agents Chemother 57:6147-6153, Yeung, Y. A. et al., 2009, J Immunol 182:7663-7671, Hinton, P. R. et al., 2006, J Immunol 176:346-356, Hinton, P. R. et al., 2004, J Biol Chem 279:6213-6216, 1 Dall'Acqua, W. F., Kiener, P. A. & Wu, H., 2006, J Biol Chem 281:23514-23524). Multiple designs are evaluated energetically using proprietary in silico tools. They are expressed, purified and then assessed for their binding to FcαRI and FcRn as well as neutrophil activation in vitro and half-life in vivo. A schematic of such a variant is shown in FIG. 12.

Results

Variants where binding to both FcαRI and FcRn is achieved are expected to show activity in a neutrophil ADCC assay as well as significantly increased half-life in FcRn in in vivo models when compared to an IgA Fc without a FcRn binding site.

### Sequence Tables

A brief description of the SEQ ID NOs for the clones described herein is provided in Table A. Amino acid sequences for each SEQ ID NO. are provided in Table B.

TABLE A
Brief Description of the Clones used to Prepare IgA
HetFc Constructs (see also Table 13 and Table 16)

| SEQ | | |
|---|---|---|
| ID | Clone | |
| NO | ID | Domain structure |
| 1 | 11150 | Tras* VL - IgKCL |
| 2 | 21715 | IgA2 hinge - IgA2m1 CH2 - IgA2m1 CH3 |
| 3 | 21755 | Tras VH - IgG1 CH1 - IgG1/IgA2 hinge - IgA2m1 CH2 - IgA2m1 CH3 |
| 4 | 23767 | IgA2 hinge - IgA2m1 CH2 - IgA2m1 CH3 |
| 5 | 23768 | IgA2 hinge - IgA2m1 CH2 - IgA2m1 CH3 |
| 6 | 23769 | IgA2 hinge - IgA2m1 CH2 - IgA2m1 CH3 |
| 7 | 23770 | IgA2 hinge - IgA2m1 CH2 - IgA2m1 CH3 |
| 8 | 23771 | IgA2 hinge - IgA2m1 CH2 - IgA2m1 CH3 |
| 9 | 23772 | IgA2 hinge - IgA2m1 CH2 - IgA2m1 CH3 |
| 10 | 23773 | Tras VH - IgG1 CH1 - IgG1/IgA2 hinge - IgA2m1 CH2 - IgA2m1 CH3 |
| 11 | 23774 | Tras VH - IgG1 CH1 - IgG1/IgA2 hinge - IgA2m1 CH2 - IgA2m1 CH3 |
| 12 | 23775 | Tras VH - IgG1 CH1 - IgG1/IgA2 hinge - IgA2m1 CH2 - IgA2m1 CH3 |
| 13 | 23776 | Tras VH - IgG1 CH1 - IgG1/IgA2 hinge - IgA2m1 CH2 - IgA2m1 CH3 |
| 14 | 23777 | Tras VH - IgG1 CH1 - IgG1/IgA2 hinge - IgA2m1 CH2 - IgA2m1 CH3 |
| 15 | 23778 | Tras VH - IgG1 CH1 - IgG1/IgA2 hinge - IgA2m1 CH2 - IgA2m1 CH3 |
| 16 | 23779 | Tras VH - IgG1 CH1 - IgG1/IgA2 hinge - IgA2m1 CH2 - IgA2m1 CH3 |
| 17 | 23780 | Tras VH - IgG1 CH1 - IgG1/IgA2 hinge - IgA2m1 CH2 - IgA2m1 CH3 |
| 18 | 23781 | Tras VH - IgG1 CH1 - IgG1/IgA2 hinge - IgA2m1 CH2 - IgA2m1 CH3 |
| 19 | 23782 | Tras VH - IgG1 CH1 - IgG1/IgA2 hinge - IgA2m1 CH2 - IgA2m1 CH3 |
| 20 | 23783 | IgA2 hinge - IgA2m1 CH2 - IgA2m1 CH3 |
| 21 | 23784 | IgA2 hinge - IgA2m1 CH2 - IgA2m1 CH3 |
| 22 | 23785 | IgA2 hinge - IgA2m1 CH2 - IgA2m1 CH3 |
| 23 | 23786 | IgA2 hinge - IgA2m1 CH2 - IgA2m1 CH3 |
| 24 | 24674 | Tras VH - IgG1 CH1 - IgG1/IgA2 hinge - IgA2m1 CH2 - IgA2m1 CH3 |
| 25 | 24675 | Tras VH - IgG1 CH1 - IgG1/IgA2 hinge - IgA2m1 CH2 - IgA2m1 CH3 |
| 26 | 24676 | Tras VH - IgG1 CH1 - IgG1/IgA2 hinge - IgA2m1 CH2 - IgA2m1 CH3 |
| 27 | 24677 | IgA2 hinge - IgA2m1 CH2 - IgA2m1 CH3 |
| 28 | 24678 | Tras VH - IgG1 CH1 - IgG1/IgA2 hinge - IgA2m1 CH2 - IgA2m1 CH3 |
| 29 | 24679 | IgA2 hinge - IgA2m1 CH2 - IgA2m1 CH3 |
| 30 | 25880 | Tras VH - IgG1 CH1 - IgG1/IgA2 hinge - IgA2m1 CH2 - IgA2m1 CH3 |
| 31 | 25881 | IgA2 hinge - IgA2m1 CH2 - IgA2m1 CH3 |
| 32 | 25882 | Tras VH - IgG1 CH1 - IgG1/IgA2 hinge - IgA2m1 CH2 - IgA2m1 CH3 |
| 33 | 25883 | IgA2 hinge - IgA2m1 CH2 - IgA2m1 CH3 |
| 34 | 25884 | Tras VH - IgG1 CH1 - IgG1/IgA2 hinge - IgA2m1 CH2 - IgA2m1 CH3 |
| 35 | 25885 | IgA2 hinge - IgA2m1 CH2 - IgA2m1 CH3 |
| 36 | 25886 | Tras VH - IgG1 CH1 - IgG1/IgA2 hinge - IgA2m1 CH2 - IgA2m1 CH3 |
| 37 | 25887 | IgA2 hinge - IgA2m1 CH2 - IgA2m1 CH3 |

*Tras—Trastuzumab

TABLE B
Amino Acid Sequences

| SEQ | Clone | | |
|---|---|---|---|
| ID NO | No. | Description | Sequence |
| 1 | 11150 | Full | DIQMTQSPSSLSASVGDRVTITCRASQDVNTAVAW |
| | | | YQQKPGKAPKLLIYSASFLYSGVPSRFSGSRSGTDF |
| | | | TLTISSLQPEDFATYYCQQHYTTPPTFGQGTKVEIK |
| | | | RTVAAPSVFIFPPSDEQLKSGTASVVCLLNNFYPRE |
| | | | AKVQWKVDNALQSGNSQESVTEQDSKDSTYSLSS |
| | | | TLTLSKADYEKHKVYACEVTHQGLSSPVTKSFNR |
| | | | GEC |
| 2 | 21715 | Full | RVPPPPPCCHPRLSLHRPALEDLLLGSEANLTCTLT |
| | | | GLRDASGATFTWTPSSGKSAVQGPPERDLCGCYS |
| | | | VSSVLPGSAQPWNHGETFTCTAAHPELKTPLTATL |
| | | | SKSGNTFRPEVHLLPPPSEELALNELVTLTCLARGF |
| | | | SPKDVLVRWLQGSQELPREKYLTWASRQEPSQGT |
| | | | TTFAVTSILRVAAEDWKKGDTFSCMVGHEALPLA |
| | | | FTQKTIDRLAG |
| 3 | 21755 | Full | EVQLVESGGGLVQPGGSLRLSCAASGFNIKDTYIH |
| | | | WVRQAPGKGLEWVARIYPTNGYTRYADSVKGRF |
| | | | TISADTSKNTAYLQMNSLRAEDTAVYYCSRWGGD |
| | | | GFYAMDYWGQGTLVTVSSASTKGPSVFPLAPSSK |
| | | | STSGGTAALGCLVKDYFPEPVTVSWNSGALTSGV |
| | | | HTFPAVLQSSGLYSLSSVVTVPSSSLGTQTYICNVN |
| | | | HKPSNTKVDKKVEPKSCRVPPPPPCCHPRLSLHRP |
| | | | ALEDLLLGSEANLTCTLTGLRDASGATFTWTPSSG |
| | | | KSAVQGPPERDLCGCYSVSSVLPGSAQPWNHGET |
| | | | FTCTAAHPELKTPLTATLSKSGNTFRPEVHLLPPPS |
| | | | EELALNELVTLTCLARGFSPKDVLVRWLQGSQELP |
| | | | REKYLTWASRQEPSQGTTTFAVTSILRVAAEDWK |
| | | | KGDTFSCMVGHEALPLAFTQKTIDRLAG |
| 4 | 23767 | Full | RVPPPPPCCHPRLSLHRPALEDLLLGSEANLTCTLT |
| | | | GLRDASGATFTWTPSSGKSAVQGPPERDLCGCYS |
| | | | VSSVLPGSAQPWNHGETFTCTAAHPELKTPLTATL |
| | | | SKSGNTFRPEVHLLPPPSEELALNELVRLTCRARGF |
| | | | SPKDVLVRWLQGSQELPREKYLTWASRQEPSQGT |
| | | | TTFAVTSILRVAAEDWKKGDTFSCMVGHEALPLA |
| | | | FTQKTIDRLAG |
| 5 | 23768 | Full | RVPPPPPCCHPRLSLHRPALEDLLLGSEANLTCTLT |
| | | | GLRDASGATFTWTPSSGKSAVQGPPERDLCGCYS |
| | | | VSSVLPGSAQPWNHGETFTCTAAHPELKTPLTATL |
| | | | SKSGNTFRPEVHLLPPPSEELALNELVRLTCKARGF |
| | | | SPKDVLVRWLQGSQELPREKYLTWASRQEPSQGT |
| | | | TTFAVTSILRVAAEDWKKGDTFSCMVGHEALPLA |
| | | | FTQKTIDRLAG |
| 6 | 23769 | Full | RVPPPPPCCHPRLSLHRPALEDLLLGSEANLTCTLT |
| | | | GLRDASGATFTWTPSSGKSAVQGPPERDLCGCYS |
| | | | VSSVLPGSAQPWNHGETFTCTAAHPELKTPLTATL |
| | | | SKSGNTFRPEVHLLPPPSEELALNELVRLTCKARGF |
| | | | SPKDVLVRWLQGSQELPREKYLTWASRQEPSQGT |
| | | | TTFAVTSRLRVAAEDWKKGDTFSCMVGHEALPLA |
| | | | FTQKTIDRLAG |
| 7 | 23770 | Full | RVPPPPPCCHPRLSLHRPALEDLLLGSEANLTCTLT |
| | | | GLRDASGATFTWTPSSGKSAVQGPPERDLCGCYS |
| | | | VSSVLPGSAQPWNHGETFTCTAAHPELKTPLTATL |
| | | | SKSGNTFRPEVHLLPPPSEELALNELVKLICKAKGF |
| | | | SPKDVLVRWLQGSQELPREKYLTWASRQEPSQGT |
| | | | TTFAVTSRLRVAAEDWKKGDTFSCMVGHEALPLA |
| | | | FTQKTIDRLAG |
| 8 | 23771 | Full | RVPPPPPCCHPRLSLHRPALEDLLLGSEANLTCTLT |
| | | | GLRDASGATFTWTPSSGKSAVQGPPERDLCGCYS |
| | | | VSSVLPGSAQPWNHGETFTCTAAHPELKTPLTATL |
| | | | SKSGNTFRPEVHLLPPPSEELALNELVTLTCKAKGF |
| | | | SPKDVLVRWLQGSQELPREKYLTWASRQRPSQGT |
| | | | TTFAVTSKLRVAAEDWKKGDTFSCMVGHEALPLA |
| | | | FTQKTIDRLAG |
| 9 | 23772 | Full | RVPPPPPCCHPRLSLHRPALEDLLLGSEANLTCTLT |
| | | | GLRDASGATFTWTPSSGKSAVQGPPERDLCGCYS |
| | | | VSSVLPGSAQPWNHGETFTCTAAHPELKTPLTATL |
| | | | SKSGNTFRPEVHLLPPPSEELALNELVTLTCKAKGF |
| | | | SPKDVLVRWLQGSQELPREKYLTWASRQEPSQGT |
| | | | TTFAVTSKLRVAAEDWKKGDTFSCMVGHEALPLA |
| | | | FTQKTIDRLAG |
| 10 | 23773 | Full | EVQLVESGGGLVQPGGSLRLSCAASGFNIKDTYIH |
| | | | WVRQAPGKGLEWVARIYPTNGYTRYADSVKGRF |
| | | | TISADTSKNTAYLQMNSLRAEDTAVYYCSRWGGD |
| | | | GFYAMDYWGQGTLVTVSSASTKGPSVFPLAPSSK |
| | | | STSGGTAALGCLVKDYFPEPVTVSWNSGALTSGV |
| | | | HTFPAVLQSSGLYSLSSVVTVPSSSLGTQTYICNVN |
| | | | HKPSNTKVDKKVEPKSCRVPPPPPCCHPRLSLHRP |
| | | | ALEDLLLGSEANLTCTLTGLRDASGATFTWTPSSG |
| | | | KSAVQGPPERDLCGCYSVSSVLPGSAQPWNHGET |
| | | | FTCTAAHPELKTPLTATLSKSGNTFRPEVHLLPPPS |
| | | | EELALNELVDLTCDADGFSPKDVLVRWLQGSQEL |
| | | | PREKYLTWASRQEPSQGTTTFAVTSELRVAAEDW |
| | | | KKGDTFSCMVGHEALPLAFTQKTIDRLAG |
| 11 | 23774 | Full | EVQLVESGGGLVQPGGSLRLSCAASGFNIKDTYIH |
| | | | WVRQAPGKGLEWVARIYPTNGYTRYADSVKGRF |
| | | | TISADTSKNTAYLQMNSLRAEDTAVYYCSRWGGD |
| | | | GFYAMDYWGQGTLVTVSSASTKGPSVFPLAPSSK |
| | | | STSGGTAALGCLVKDYFPEPVTVSWNSGALTSGV |
| | | | HTFPAVLQSSGLYSLSSVVTVPSSSLGTQTYICNVN |
| | | | HKPSNTKVDKKVEPKSCRVPPPPPCCHPRLSLHRP |
| | | | ALEDLLLGSEANLTCTLTGLRDASGATFTWTPSSG |
| | | | KSAVQGPPERDLCGCYSVSSVLPGSAQPWNHGET |
| | | | FTCTAAHPELKTPLTATLSKSGNTFRPEVHLLPPPS |
| | | | EELALNELVELTCEADGFSPKDVLVRWLQGSQELP |
| | | | REKYLTWASRQEPSQGTTTFAVTSDLEVAAEDWK |
| | | | KGDTFSCMVGHEALPLAFTQKTIDRLAG |
| 12 | 23775 | Full | EVQLVESGGGLVQPGGSLRLSCAASGFNIKDTYIH |
| | | | WVRQAPGKGLEWVARIYPTNGYTRYADSVKGRF |
| | | | TISADTSKNTAYLQMNSLRAEDTAVYYCSRWGGD |
| | | | GFYAMDYWGQGTLVTVSSASTKGPSVFPLAPSSK |
| | | | STSGGTAALGCLVKDYFPEPVTVSWNSGALTSGV |
| | | | HTFPAVLQSSGLYSLSSVVTVPSSSLGTQTYICNVN |
| | | | HKPSNTKVDKKVEPKSCRVPPPPPCCHPRLSLHRP |
| | | | ALEDLLLGSEANLTCTLTGLRDASGATFTWTPSSG |
| | | | KSAVQGPPERDLCGCYSVSSVLPGSAQPWNHGET |
| | | | FTCTAAHPELKTPLTATLSKSGNTFRPEVHLLPPPS |
| | | | EELALNELVELTCLADGFSPKDVLVRWLQGSQELP |
| | | | REKYLTWASRQEPSQGTTTFAVTSDLDVAAEDWK |
| | | | KGDTFSCMVGHEALPLAFTQKTIDRLAG |
| 13 | 23776 | Full | EVQLVESGGGLVQPGGSLRLSCAASGFNIKDTYIH |
| | | | WVRQAPGKGLEWVARIYPTNGYTRYADSVKGRF |
| | | | TISADTSKNTAYLQMNSLRAEDTAVYYCSRWGGD |
| | | | GFYAMDYWGQGTLVTVSSASTKGPSVFPLAPSSK |
| | | | STSGGTAALGCLVKDYFPEPVTVSWNSGALTSGV |
| | | | HTFPAVLQSSGLYSLSSVVTVPSSSLGTQTYICNVN |
| | | | HKPSNTKVDKKVEPKSCRVPPPPPCCHPRLSLHRP |
| | | | ALEDLLLGSEANLTCTLTGLRDASGATFTWTPSSG |
| | | | KSAVQGPPERDLCGCYSVSSVLPGSAQPWNHGET |
| | | | FTCTAAHPELKTPLTATLSKSGNTFRPEVHLLPPPS |
| | | | EELALNELVELTCLADGFSPKDVLVRWLQGSQELP |
| | | | REKYLTWASRQEPSQGTTTFAVTSELEVAAEDWK |
| | | | KGDTFSCMVGHEALPLAFTQKTIDRLAG |
| 14 | 23777 | Full | EVQLVESGGGLVQPGGSLRLSCAASGFNIKDTYIH |
| | | | WVRQAPGKGLEWVARIYPTNGYTRYADSVKGRF |
| | | | TISADTSKNTAYLQMNSLRAEDTAVYYCSRWGGD |
| | | | GFYAMDYWGQGTLVTVSSASTKGPSVFPLAPSSK |
| | | | STSGGTAALGCLVKDYFPEPVTVSWNSGALTSGV |
| | | | HTFPAVLQSSGLYSLSSVVTVPSSSLGTQTYICNVN |
| | | | HKPSNTKVDKKVEPKSCRVPPPPPCCHPRLSLHRP |
| | | | ALEDLLLGSEANLTCTLTGLRDASGATFTWTPSSG |
| | | | KSAVQGPPERDLCGCYSVSSVLPGSAQPWNHGET |
| | | | FTCTAAHPELKTPLTATLSKSGNTFRPEVHLLPPPS |
| | | | EELALNELVTLTCLADGFSPKDVLVRWLQGSQELP |
| | | | REKYLTWASRQDPSQGTTTFAVTSDLEVAAEDWK |
| | | | KGDTFSCMVGHEALPLAFTQKTIDRLAG |
| 15 | 23778 | Full | EVQLVESGGGLVQPGGSLRLSCAASGFNIKDTYIH |
| | | | WVRQAPGKGLEWVARIYPTNGYTRYADSVKGRF |
| | | | TISADTSKNTAYLQMNSLRAEDTAVYYCSRWGGD |
| | | | GFYAMDYWGQGTLVTVSSASTKGPSVFPLAPSSK |
| | | | STSGGTAALGCLVKDYFPEPVTVSWNSGALTSGV |
| | | | HTFPAVLQSSGLYSLSSVVTVPSSSLGTQTYICNVN |
| | | | HKPSNTKVDKKVEPKSCRVPPPPPCCHPRLSLHRP |
| | | | ALEDLLLGSEANLTCTLTGLRDASGATFTWTPSSG |
| | | | KSAVQGPPERDLCGCYSVSSVLPGSAQPWNHGET |
| | | | FTCTAAHPELKTPLTATLSKSGNTFRPEVHLLPPPS |
| | | | EELALNELVTLTCLARGFSPKDVLVRWLQGSQELP |
| | | | REKYLTWASRQEPSQGTTTFYVLSILRVAAEDWK |
| | | | KGDTFSCMVGHEALPLAFTQKTIDRLAG |
| 16 | 23779 | Full | EVQLVESGGGLVQPGGSLRLSCAASGFNIKDTYIH |
| | | | WVRQAPGKGLEWVARIYPTNGYTRYADSVKGRF |
| | | | TISADTSKNTAYLQMNSLRAEDTAVYYCSRWGGD |
| | | | GFYAMDYWGQGTLVTVSSASTKGPSVFPLAPSSK |
| | | | STSGGTAALGCLVKDYFPEPVTVSWNSGALTSGV |
| | | | HTFPAVLQSSGLYSLSSVVTVPSSSLGTQTYICNVN |
| | | | HKPSNTKVDKKVEPKSCRVPPPPPCCHPRLSLHRP |
| | | | ALEDLLLGSEANLTCTLTGLRDASGATFTWTPSSG |
| | | | KSAVQGPPERDLCGCYSVSSVLPGSAQPWNHGET |
| | | | FTCTAAHPELKTPLTATLSKSGNTFRPEVHLLPPPS |
| | | | EELALNELVTLTCLARGFSPKDVLVRWLQGSQELP |
| | | | REKYLTWASRQEPSQGTTTFYVYSILRVAAEDWK |
| | | | KGDTFSCMVGHEALPLAFTQKTIDRLAG |
| 17 | 23780 | Full | EVQLVESGGGLVQPGGSLRLSCAASGFNIKDTYIH |
| | | | WVRQAPGKGLEWVARIYPTNGYTRYADSVKGRF |
| | | | TISADTSKNTAYLQMNSLRAEDTAVYYCSRWGGD |
| | | | GFYAMDYWGQGTLVTVSSASTKGPSVFPLAPSSK |
| | | | STSGGTAALGCLVKDYFPEPVTVSWNSGALTSGV |
| | | | HTFPAVLQSSGLYSLSSVVTVPSSSLGTQTYICNVN |
| | | | HKPSNTKVDKKVEPKSCRVPPPPPCCHPRLSLHRP |
| | | | ALEDLLLGSEANLTCTLTGLRDASGATFTWTPSSG |
| | | | KSAVQGPPERDLCGCYSVSSVLPGSAQPWNHGET |
| | | | FTCTAAHPELKTPLTATLSKSGNTFRPEVHLLPPPS |
| | | | EELALNELVTLTCLARGFSPKDVLVRWLQGSQELP |
| | | | REKYLTWASRQEPSQGTTTFFVYSILRVAAEDWK |
| | | | KGDTFSCMVGHEALPLAFTQKTIDRLAG |
| 18 | 23781 | Full | EVQLVESGGGLVQPGGSLRLSCAASGFNIKDTYIH |
| | | | WVRQAPGKGLEWVARIYPTNGYTRYADSVKGRF |
| | | | TISADTSKNTAYLQMNSLRAEDTAVYYCSRWGGD |
| | | | GFYAMDYWGQGTLVTVSSASTKGPSVFPLAPSSK |
| | | | STSGGTAALGCLVKDYFPEPVTVSWNSGALTSGV |
| | | | HTFPAVLQSSGLYSLSSVVTVPSSSLGTQTYICNVN |
| | | | HKPSNTKVDKKVEPKSCRVPPPPPCCHPRLSLHRP |
| | | | ALEDLLLGSEANLTCTLTGLRDASGATFTWTPSSG |
| | | | KSAVQGPPERDLCGCYSVSSVLPGSAQPWNHGET |
| | | | FTCTAAHPELKTPLTATLSKSGNTFRPEVHLLPPPS |
| | | | EELALNELVTLTCMARGFSPKDVLVRWLQGSQEL |
| | | | PREKYLTWASRQEPSQGTTTFFVWSILRVAAEDW |
| | | | KKGDTFSCMVGHEALPLAFTQKTIDRLAG |
| 19 | 23782 | Full | EVQLVESGGGLVQPGGSLRLSCAASGFNIKDTYIH |
| | | | WVRQAPGKGLEWVARIYPTNGYTRYADSVKGRF |
| | | | TISADTSKNTAYLQMNSLRAEDTAVYYCSRWGGD |
| | | | GFYAMDYWGQGTLVTVSSASTKGPSVFPLAPSSK |
| | | | STSGGTAALGCLVKDYFPEPVTVSWNSGALTSGV |
| | | | HTFPAVLQSSGLYSLSSVVTVPSSSLGTQTYICNVN |
| | | | HKPSNTKVDKKVEPKSCRVPPPPPCCHPRLSLHRP |
| | | | ALEDLLLGSEANLTCTLTGLRDASGATFTWTPSSG |
| | | | KSAVQGPPERDLCGCYSVSSVLPGSAQPWNHGET |
| | | | FTCTAAHPELKTPLTATLSKSGNTFRPEVHLLPPPS |
| | | | EELALNELVTLTCLARGFSPKDVLVRWLQGSQELP |
| | | | REKYLTWASRQEPSQGTTTFYVMSILRVAAEDWK |
| | | | KGDTFSCMVGHEALPLAFTQKTIDRLAG |
| 20 | 23783 | Full | RVPPPPPCCHPRLSLHRPALEDLLLGSEANLTCTLT |
| | | | GLRDASGATFTWTPSSGKSAVQGPPERDLCGCYS |
| | | | VSSVLPGSAQPWNHGETFTCTAAHPELKTPLTATL |
| | | | SKSGNTFRPEVHLLPPPSEELALNELVTLTCLARGF |
| | | | SPKDVLVRWLQGSQELPREKYTTLASRQEPSQGTT |
| | | | TFAVTSLLRVAAEDWKKGDTFSCMVGHEALPLAF |
| | | | TQKTIDRLAG |
| 21 | 23784 | Full | RVPPPPPCCHPRLSLHRPALEDLLLGSEANLTCTLT |
| | | | GLRDASGATFTWTPSSGKSAVQGPPERDLCGCYS |
| | | | VSSVLPGSAQPWNHGETFTCTAAHPELKTPLTATL |
| | | | SKSGNTFRPEVHLLPPPSEELALNELVTLTCLARGF |
| | | | SPKDVLVRWLQGSQELPREKYVTLASRQEPSQGTT |
| | | | TFAVTSLLRVAAEDWKKGDTFSCMVGHEALPLAF |
| | | | TQKTIDRLAG |
| 22 | 23785 | Full | RVPPPPPCCHPRLSLHRPALEDLLLGSEANLTCTLT |
| | | | GLRDASGATFTWTPSSGKSAVQGPPERDLCGCYS |
| | | | VSSVLPGSAQPWNHGETFTCTAAHPELKTPLTATL |
| | | | SKSGNTFRPEVHLLPPPSEELALNELVTLTCLARGF |
| | | | SPKDVLVRWLQGSQELPREKYLTLASRQEPSQGTT |
| | | | TFAVTSILRVAAEDWKKGDTFSCMVGHEALPLAF |
| | | | TQKTIDRLAG |
| 23 | 23786 | Full | RVPPPPPCCHPRLSLHRPALEDLLLGSEANLTCTLT |
| | | | GLRDASGATFTWTPSSGKSAVQGPPERDLCGCYS |
| | | | VSSVLPGSAQPWNHGETFTCTAAHPELKTPLTATL |
| | | | SKSGNTFRPEVHLLPPPSEELALNELVTLTCLARGF |
| | | | SPKDVLVRWLQGSQELPREKYVTTASRQEPSQGTT |
| | | | TFAVTSLLRVAAEDWKKGDTFSCMVGHEALPLAF |
| | | | TQKTIDRLAG |
| 24 | 24674 | Full | EVQLVESGGGLVQPGGSLRLSCAASGFNIKDTYIH |
| | | | WVRQAPGKGLEWVARIYPTNGYTRYADSVKGRF |
| | | | TISADTSKNTAYLQMNSLRAEDTAVYYCSRWGGD |
| | | | GFYAMDYWGQGTLVTVSSASTKGPSVFPLAPSSK |
| | | | STSGGTAALGCLVKDYFPEPVTVSWNSGALTSGV |
| | | | HTFPAVLQSSGLYSLSSVVTVPSSSLGTQTYICNVN |
| | | | HKPSNTKVDKKVEPKSCRVPPPPPCCHPRLSLHRP |
| | | | ALEDLLLGSEANLTCTLTGLRDASGATFTWTPSSG |
| | | | KSAVQGPPERDLCGCYSVSSVLPGSAQPWNHGET |
| | | | FTCTAAHPELKTPLTATLSKSGNTFRPEVHLLPPPS |
| | | | EELALNELVTLVCLARGFSPKDVLVRWLQGSQELP |
| | | | REKYLTWASRQEPSQGTTTFFVYSILRVAAEDWK |
| | | | KGDTFSCMVGHEALPLAFTQKTIDRLAG |
| 25 | 24675 | Full | EVQLVESGGGLVQPGGSLRLSCAASGFNIKDTYIH |
| | | | WVRQAPGKGLEWVARIYPTNGYTRYADSVKGRF |
| | | | TISADTSKNTAYLQMNSLRAEDTAVYYCSRWGGD |
| | | | GFYAMDYWGQGTLVTVSSASTKGPSVFPLAPSSK |
| | | | STSGGTAALGCLVKDYFPEPVTVSWNSGALTSGV |
| | | | HTFPAVLQSSGLYSLSSVVTVPSSSLGTQTYICNVN |
| | | | HKPSNTKVDKKVEPKSCRVPPPPPCCHPRLSLHRP |
| | | | ALEDLLLGSEANLTCTLTGLRDASGATFTWTPSSG |
| | | | KSAVQGPPERDLCGCYSVSSVLPGSAQPWNHGET |
| | | | FTCTAAHPELKTPLTATLSKSGNTFRPEVHLLPPPS |
| | | | EELALNELVTLLCLARGFSPKDVLVRWLQGSQELP |
| | | | REKYLTWASRQEPSQGTTTFFVYSILRVAAEDWK |
| | | | KGDTFSCMVGHEALPLAFTQKTIDRLAG |
| 26 | 24676 | Full | EVQLVESGGGLVQPGGSLRLSCAASGFNIKDTYIH |
| | | | WVRQAPGKGLEWVARIYPTNGYTRYADSVKGRF |
| | | | TISADTSKNTAYLQMNSLRAEDTAVYYCSRWGGD |
| | | | GFYAMDYWGQGTLVTVSSASTKGPSVFPLAPSSK |
| | | | STSGGTAALGCLVKDYFPEPVTVSWNSGALTSGV |
| | | | HTFPAVLQSSGLYSLSSVVTVPSSSLGTQTYICNVN |
| | | | HKPSNTKVDKKVEPKSCRVPPPPPCCHPRLSLHRP |
| | | | ALEDLLLGSEANLTCTLTGLRDASGATFTWTPSSG |
| | | | KSAVQGPPERDLCGCYSVSSVLPGSAQPWNHGET |
| | | | FTCTAAHPELKTPLTATLSKSGNTFRPEVHLLPPPS |
| | | | EELALNELVTLICLARGFSPKDVLVRWLQGSQELP |
| | | | REKYLTWASRQEPSQGTTTFFVYSILRVAAEDWK |
| | | | KGDTFSCMVGHEALPLAFTQKTIDRLAG |
| 27 | 24677 | Full | RVPPPPPCCHPRLSLHRPALEDLLLGSEANLTCTLT |
| | | | GLRDASGATFTWTPSSGKSAVQGPPERDLCGCYS |
| | | | VSSVLPGSAQPWNHGETFTCTAAHPELKTPLTATL |
| | | | SKSGNTFRPEVHLFPPPSEELALNELVTLTCLARGF |
| | | | SPKDVLVRWLQGSQELPREKYVTTASRQEPSQGTT |
| | | | TFAVTSLLRVAAEDWKKGDTFSCMVGHEALPLAF |
| | | | TQKTIDRLAG |
| 28 | 24678 | Full | EVQLVESGGGLVQPGGSLRLSCAASGFNIKDTYIH |
| | | | WVRQAPGKGLEWVARIYPTNGYTRYADSVKGRF |
| | | | TISADTSKNTAYLQMNSLRAEDTAVYYCSRWGGD |
| | | | GFYAMDYWGQGTLVTVSSASTKGPSVFPLAPSSK |
| | | | STSGGTAALGCLVKDYFPEPVTVSWNSGALTSGV |
| | | | HTFPAVLQSSGLYSLSSVVTVPSSSLGTQTYICNVN |
| | | | HKPSNTKVDKKVEPKSCRVPPPPPCCHPRLSLHRP |
| | | | ALEDLLLGSEANLTCTLTGLRDASGATFTWTPSSG |
| | | | KSAVQGPPERDLCGCYSVSSVLPGSAQPWNHGET |
| | | | FTCTAAHPELKTPLTATLSKSGNTFRPEVYLLPPPS |
| | | | EELALNELVTLTCLARGFSPKDVLVRWLQGSQELP |
| | | | REKYLTWASRQEPSQGTTTFFVYSILRVAAEDWK |
| | | | KGDTFSCMVGHEALPLAFTQKTIDRLAG |
| 29 | 24679 | Full | RVPPPPPCCHPRLSLHRPALEDLLLGSEANLTCTLT |
| | | | GLRDASGATFTWTPSSGKSAVQGPPERDLCGCYS |
| | | | VSSVLPGSAQPWNHGETFTCTAAHPELKTPLTATL |
| | | | SKSGNTFRPEVYLLPPPSEELALNELVTLTCLARGF |
| | | | SPKDVLVRWLQGSQELPREKYVTTASRQEPSQGTT |
| | | | TFAVTSLLRVAAEDWKKGDTFSCMVGHEALPLAF |
| | | | TQKTIDRLAG |
| 30 | 25880 | Full | EVQLVESGGGLVQPGGSLRLSCAASGFNIKDTYIH |
| | | | WVRQAPGKGLEWVARIYPTNGYTRYADSVKGRF |
| | | | TISADTSKNTAYLQMNSLRAEDTAVYYCSRWGGD |
| | | | GFYAMDYWGQGTLVTVSSASTKGPSVFPLAPSSK |
| | | | STSGGTAALGCLVKDYFPEPVTVSWNSGALTSGV |
| | | | HTFPAVLQSSGLYSLSSVVTVPSSSLGTQTYICNVN |
| | | | HKPSNTKVDKKVEPKSCRVPPPPPCCHPRLSLHRP |
| | | | ALEDLLLGSEANLTCTLTGLRDASGATFTWTPSSG |
| | | | KSAVQGPPERDLCGCYSVSSVLPGSAQPWNHGET |
| | | | FTCTAAHPELKTPLTATLSKSGNTFRPEVCLLPPPS |
| | | | EELALNELVTLTCLARGFSPKDVLVRWLQGSQELP |
| | | | REKYLTWASRQEPSQGTTTFFVYSILRVAAEDWK |
| | | | KGDTFSCMVGHEALPLAFTQKTIDRLAG |
| 31 | 25881 | Full | RVPPPPPCCHPRLSLHRPALEDLLLGSEANLTCTLT |
| | | | GLRDASGATFTWTPSSGKSAVQGPPERDLCGCYS |
| | | | VSSVLPGSAQPWNHGETFTCTAAHPELKTPLTATL |
| | | | SKSGNTFRPEVHLLPPCSEELALNELVTLTCLARGF |
| | | | SPKDVLVRWLQGSQELPREKYVTTASRQEPSQGTT |
| | | | TFAVTSLLRVAAEDWKKGDTFSCMVGHEALPLAF |
| | | | TQKTIDRLAG |
| 32 | 25882 | Full | EVQLVESGGGLVQPGGSLRLSCAASGFNIKDTYIH |
| | | | WVRQAPGKGLEWVARIYPTNGYTRYADSVKGRF |
| | | | TISADTSKNTAYLQMNSLRAEDTAVYYCSRWGGD |
| | | | GFYAMDYWGQGTLVTVSSASTKGPSVFPLAPSSK |
| | | | STSGGTAALGCLVKDYFPEPVTVSWNSGALTSGV |
| | | | HTFPAVLQSSGLYSLSSVVTVPSSSLGTQTYICNVN |
| | | | HKPSNTKVDKKVEPKSCRVPPPPPCCHPRLSLHRP |
| | | | ALEDLLLGSEANLTCTLTGLRDASGATFTWTPSSG |
| | | | KSAVQGPPERDLCGCYSVSSVLPGSAQPWNHGET |
| | | | FTCTAAHPELKTPLTATLSKSGNTFRPEVHLLPPCS |
| | | | EELALNELVTLTCLARGFSPKDVLVRWLQGSQELP |
| | | | REKYLTWASRQEPSQGTTTFFVYSILRVAAEDWK |
| | | | KGDTFSCMVGHEALPLAFTQKTIDRLAG |
| 33 | 25883 | Full | RVPPPPPCCHPRLSLHRPALEDLLLGSEANLTCTLT |
| | | | GLRDASGATFTWTPSSGKSAVQGPPERDLCGCYS |
| | | | VSSVLPGSAQPWNHGETFTCTAAHPELKTPLTATL |
| | | | SKSGNTFRPEVCLLPPPSEELALNELVTLTCLARGF |
| | | | SPKDVLVRWLQGSQELPREKYVTTASRQEPSQGTT |
| | | | TFAVTSLLRVAAEDWKKGDTFSCMVGHEALPLAF |
| | | | TQKTIDRLAG |
| 34 | 25884 | Full | EVQLVESGGGLVQPGGSLRLSCAASGFNIKDTYIH |
| | | | WVRQAPGKGLEWVARIYPTNGYTRYADSVKGRF |
| | | | TISADTSKNTAYLQMNSLRAEDTAVYYCSRWGGD |
| | | | GFYAMDYWGQGTLVTVSSASTKGPSVFPLAPSSK |
| | | | STSGGTAALGCLVKDYFPEPVTVSWNSGALTSGV |
| | | | HTFPAVLQSSGLYSLSSVVTVPSSSLGTQTYICNVN |
| | | | HKPSNTKVDKKVEPKSCRVPPPPPCCHPRLSLHRP |
| | | | ALEDLLLGSEANLTCTLTGLRDASGATFTWTPSSG |
| | | | KSAVQGPPERDLCGCYSVSSVLPGSAQPWNHGET |
| | | | FTCTAAHPELKTPLTATLSKSGNTFRPEVCLLPPCS |
| | | | EELALNELVTLTCLARGFSPKDVLVRWLQGSQELP |
| | | | REKYLTWASRQEPSQGTTTFFVYSILRVAAEDWK |
| | | | KGDTFSCMVGHEALPLAFTQKTIDRLAG |
| 35 | 25885 | Full | RVPPPPPCCHPRLSLHRPALEDLLLGSEANLTCTLT |
| | | | GLRDASGATFTWTPSSGKSAVQGPPERDLCGCYS |
| | | | VSSVLPGSAQPWNHGETFTCTAAHPELKTPLTATL |
| | | | SKSGNTFRPEVCLLPPCSEELALNELVTLTCLARGF |
| | | | SPKDVLVRWLQGSQELPREKYVTTASRQEPSQGTT |
| | | | TFAVTSLLRVAAEDWKKGDTFSCMVGHEALPLAF |
| | | | TQKTIDRLAG |
| 36 | 25886 | Full | EVQLVESGGGLVQPGGSLRLSCAASGFNIKDTYIH |
| | | | WVRQAPGKGLEWVARIYPTNGYTRYADSVKGRF |
| | | | TISADTSKNTAYLQMNSLRAEDTAVYYCSRWGGD |
| | | | GFYAMDYWGQGTLVTVSSASTKGPSVFPLAPSSK |
| | | | STSGGTAALGCLVKDYFPEPVTVSWNSGALTSGV |
| | | | HTFPAVLQSSGLYSLSSVVTVPSSSLGTQTYICNVN |
| | | | HKPSNTKVDKKVEPKSCRVPPPPPCCHPRLSLHRP |
| | | | ALEDLLLGSEANLTCTLTGLRDASGATFTWTPSSG |
| | | | KSAVQGPPERDLCGCYSVSSVLPGSAQPWNHGET |
| | | | FTCTAAHPELKTPLTATLSKSGNTFRPEVCLLPPPS |
| | | | EELALNELVTLTCLARGFSPKDVLVRWLQGSQELP |
| | | | REKYLTWASRQEPSQGTTTFAVTSILRVAAEDWK |
| | | | KGDTFSCMVGHEALPLAFTQKTIDRLAG |
| 37 | 25887 | Full | RVPPPPPCCHPRLSLHRPALEDLLLGSEANLTCTLT |
| | | | GLRDASGATFTWTPSSGKSAVQGPPERDLCGCYS |
| | | | VSSVLPGSAQPWNHGETFTCTAAHPELKTPLTATL |
| | | | SKSGNTFRPEVHLLPPCSEELALNELVTLTCLARGF |
| | | | SPKDVLVRWLQGSQELPREKYLTWASRQEPSQGT |
| | | | TTFAVTSILRVAAEDWKKGDTFSCMVGHEALPLA |
| | | | FTQKTIDRLAG |
| 38 | N/A | Anti-Her2 | EVQLVESGGGLVQPGGSLRLSCAASGFNIKDTYIH |
| | | Fab HC | WVRQAPGKGLEWVARIYPTNGYTRYADSVKGRF |
| | | | TISADTSKNTAYLQMNSLRAEDTAVYYCSRWGGD |
| | | | GFYAMDYWGQGTLVTVSSASTKGPSVFPLAPSSK |
| | | | STSGGTAALGCLVKDYFPEPVTVSWNSGALTSGV |
| | | | HTFPAVLQSSGLYSLSSVVTVPSSSLGTQTYICNVN |
| | | | HKPSNTKVDKKV |
| 39 | N/A | Anti-Her2 | DIQMTQSPSSLSASVGDRVTITCRASQDVNTAVAW |
| | | Fab LC | YQQKPGKAPKLLIYSASFLYSGVPSRFSGSRSGTDF |
| | | | TLTISSLQPEDFATYYCQQHYTTPPTFGQGTKVEIK |
| | | | RTVAAPSVFIFPPSDEQLKSGTASVVCLLNNFYPRE |
| | | | AKVQWKVDNALQSGNSQESVTEQDSKDSTYSLSS |
| | | | TLTLSKADYEKHKVYACEVTHQGLSSPVTKSFNR |
| | | | GEC |
| 40 | N/A | IgG1 upper | EPKSC |
| | | hinge | |
| 41 | N/A | IgA2 hinge | RVPPPPP |
| 42 | N/A | IgA2m1 | CCHPRLSLHRPALEDLLLGSEANLTCTLTGLRDAS |
| | | wild type | GATFTWTPSSGKSAVQGPPERDLCGCYSVSSVLPG |
| | | sequence | CAQPWNHGETFTCTAAHPELKTPLTANITKSGNTF |
| | | | RPEVHLLPPPSEELALNELVTLTCLARGFSPKDVLV |
| | | | RWLQGSQELPREKYLTWASRQEPSQGTTTFAVTSI |
| | | | LRVAAEDWKKGDTFSCMVGHEALPLAFTQKTIDR |
| | | | LAG |
| 43 | N/A | IgA2m1 | CCHPRLSLHRPALEDLLLGSEANLTCTLTGLRDAS |
| | | C5092S/N5120T/ | GATFTWTPSSGKSAVQGPPERDLCGCYSVSSVLPG |
| | | I5121L/ | SAQPWNHGETFTCTAAHPELKTPLTATLSKSGNTF |
| | | T5122S/Δ | RPEVHLLPPPSEELALNELVTLTCLARGFSPKDVLV |
| | | α-tailpiece | RWLQGSQELPREKYLTWASRQEPSQGTTTFAVTSI |
| | | | LRVAAEDWKKGDTFSCMVGHEALPLAFTQKTIDR |
| | | | LAG |
| 44 | N/A | IgA1 wild | CCHPRLSLHRPALEDLLLGSEANLTCTLTGLRDAS |
| | | type | GVTFTWTPSSGKSAVQGPPERDLCGCYSVSSVLPG |
| | | | CAEPWNHGKTFTCTAAYPESKTPLTATLSKSGNTF |
| | | | RPEVHLLPPPSEELALNELVTLTCLARGFSPKDVLV |
| | | | RWLQGSQELPREKYLTWASRQEPSQGTTTFAVTSI |
| | | | LRVAAEDWKKGDTFSCMVGHEALPLAFTQKTIDR |
| | | | LAG |
| 45 | N/A | IgA2m2 | CCHPRLSLHRPALEDLLLGSEANLTCTLTGLRDAS |
| | | wild type | GATFTWTPSSGKSAVQGPPERDLCGCYSVSSVLPG |
| | | | CAQPWNHGETFTCTAAHPELKTPLTANITKSGNTF |
| | | | RPEVHLLPPPSEELALNELVTLTCLARGFSPKDVLV |
| | | | RWLQGSQELPREKYLTWASRQEPSQGTTTYAVTSI |
| | | | LRVAAEDWKKGETFSCMVGHEALPLAFTQKTIDR |
| | | | LAG |
| 46 | N/A | α-tailpiece | KPTHVNVSVVMAEVDGTCY |
| | | IgA1 | |
| | | IgA2m1 | |
| 47 | N/A | α-tailpiece | KPTHINVSVVMAEADGTCY |
| | | IgA2m2 | |
| 48 | N/A | J Chain | EFATMRPTWAWWLFLVLLLALWAPARGQEDERI |
| | | | VLVDNKCKCARITSRIIRSSEDPNEDIVERNIRIIVPL |
| | | | NNRENISDPTSPLRTRFVYHLSDLCKKCDPTEVELD |
| | | | NQIVTATQSNICDEDSATETCYTYDRNKCYTAVVP |
| | | | LVYGGETKMVETALTPDACYPD |
| 49 | N/A | Signal | EFATMRPTWAWWLFLVLLLALWAPARG |
| | | peptide | |

It is to be understood that the methods and compositions described herein are not limited to the particular methodology, protocols, cell lines, constructs, and reagents described herein and as such may vary. It is also to be understood that the terminology used herein is for the purpose of describing particular embodiments only and is not intended to limit the scope of the methods and compositions described herein, which will be limited only by the appended claims.

The disclosures of all patents, patent applications, publications and database entries referenced in this specification are hereby specifically incorporated by reference in their entirety to the same extent as if each such individual patent, patent application, publication and database entry were specifically and individually indicated to be incorporated by reference.

We claim:

1. An IgA heterodimeric Fc (IgA HetFc) construct comprising a first Fc polypeptide and a second Fc polypeptide, the first Fc polypeptide comprising a first CH3 domain sequence and the second Fc polypeptide comprising an second CH3 domain sequence, the first and second CH3 domain sequences forming a modified CH3 domain,
wherein the first and second CH3 domain sequences comprise amino acid mutations that promote formation of a heterodimeric Fc over a homodimeric Fc,
wherein:
the amino acid mutations in the first CH3 domain sequence comprise an amino acid substitution at position A6085Y selected from A6085YF, A6085YY, A6085YM, A6085YW and A6085YH, and an amino acid substitution at position T6086 selected from T6086Y, T6086F, T6086M, T6086W and T6086H, and
the amino acid mutations in the second CH3 domain sequence comprise an amino acid substitution at position W608I selected from W6081T, W6081L, W6081A, W6081V and W6081I,
wherein the heterodimeric Fc is formed with a purity of 70% or higher,
and wherein the numbering of amino acid positions is according to IMGT numbering.

2. The IgA HetFc construct according to claim 1, wherein the modified CH3 domain has a melting temperature (Tm) that is 60° C. or higher.

3. The IgA HetFc construct according to claim 1, wherein the modified CH3 domain has a melting temperature (Tm) that is ±10° C. of the Tm of a corresponding wild-type IgA CH3 domain.

4. The IgA HetFc construct according to any one of claims 1 to 3, wherein the amino acid substitution at position A6085Y is A6085YF, A6085YY or A6085YW.

5. The IgA HetFc construct according to any one of claims 1 to 3, wherein the amino acid substitution at position A6085Y is A6085YF or A6085YY.

6. The IgA HetFc construct according to any one of claims 1 to 5, wherein the amino acid substitution at position T6086 is T6086Y, T6086F or T6086W.

7. The IgA HetFc construct according to any one of claims 1 to 5, wherein the amino acid substitution at position T6086 is T6086Y.

8. The IgA HetFc construct according to any one of claims 1 to 7, wherein the amino acid substitution at position W608I is W6081T or W6081L.

9. The IgA HetFc construct according to any one of claims 1 to 3, wherein the amino acid mutations in the first CH3 domain sequence comprise the amino acid substitutions A6085YF, and T6086W, and the amino acid mutations in the second CH3 domain sequence comprise the amino acid substitution W6081T or W6081L.

10. The IgA HetFc construct according to claim 9, wherein the amino acid mutations in the second CH3 domain sequence comprise the amino acid substitution W6081T.

11. The IgA HetFc construct according to any one of claims 1 to 10, wherein the amino acid mutations in the second CH3 domain sequence further comprise an amino acid substitution at position L6079 selected from L6079V, L6079T, L6079A and L6079I.

12. The IgA HetFc construct according to any one of claims 1 to 10, wherein the amino acid mutations in the second CH3 domain sequence further comprise an amino acid substitution at position L6079 selected from L6079V and L6079T.

13. The IgA HetFc construct according to any one of claims 1 to 12, wherein the amino acid mutations in the second CH3 domain sequence further comprise an amino acid substitution at position 16088 selected from I6088L, I6088A, L6088V and L6088T.

14. The IgA HetFc construct according to any one of claims 1 to 12, wherein the amino acid mutations in the second CH3 domain sequence further comprise the amino acid substitution I6088L.

15. The IgA HetFc construct according to any one of claims 1 to 14, wherein the amino acid mutations in the first CH3 domain sequence further comprise an amino acid substitution at position T6022 selected from T6022V, T6022I, T6022L and T6022A.

16. The IgA HetFc construct according to any one of claims 1 to 14, wherein the amino acid mutations in the first CH3 domain sequence further comprise an amino acid substitution at position T6022 selected from T6022V, T6022I and T6022L.

17. The IgA HetFc construct according to any one of claims 1 to 16, wherein the amino acid mutations in the second CH3 domain sequence further comprise an amino acid substitution at position L6007 selected from L6007F, L6007Y, L6007M, L6007W, L6007H and L6007I.

18. The IgA HetFc construct according to any one of claims 1 to 16, wherein the amino acid mutations in the second CH3 domain sequence further comprise the amino acid substitution L6007F.

19. The IgA HetFc construct according to any one of claims 1 to 18, wherein the amino acid mutations in the first CH3 domain sequence further comprise an amino acid substitution at position H6005 selected from H6005Y, H6005F, H6005M and H6005W.

20. The IgA HetFc construct according to any one of claims 1 to 18, wherein the amino acid mutations in the first CH3 domain sequence further comprise the amino acid substitution H6005Y.

21. The IgA HetFc construct according to any one of claims 1 to 20, wherein the amino acid mutations in the second CH3 domain sequence further comprise an amino acid substitution at position H6005 selected from H6005Y, H6005F, H6005M and H6005W.

22. The IgA HetFc construct according to any one of claims 1 to 20, wherein the amino acid mutations in the second CH3 domain sequence further comprise the amino acid substitution H6005Y.

23. The IgA HetFc construct according to any one of claims 1 to 10, wherein the modified CH3 domain further comprises amino acid substitutions to introduce cysteine residues capable of forming a disulfide bond.

24. The IgA HetFc construct according to claim 23, wherein the modified CH3 domain comprises two amino acid substitutions to introduce cysteine residues that form one disulfide bond in the modified CH3 domain, or four amino acid substitutions to introduce cysteine residues that form two disulfide bonds in the modified CH3 domain.

25. The IgA Het Fc construct according to claim 23, wherein the amino acid substitutions to introduce cysteine residues comprise the mutation H6005C in one CH3 domain sequence and the mutation P6010C in the other CH3 domain sequence.

26. The IgA HetFc construct according to claim 23, wherein the amino acid substitutions to introduce cysteine residues comprise the mutations H6005C and P6010C in one CH3 domain sequence and the mutations P6010C and H6005C in the other CH3 domain sequence.

27. An IgA heterodimeric Fc (IgA HetFc) construct comprising a first Fc polypeptide and a second Fc polypeptide, the first Fc polypeptide comprising a first CH3 domain sequence and the second Fc polypeptide comprising an second CH3 domain sequence, the first and second CH3 domain sequences forming a modified CH3 domain,
wherein the first and second CH3 domain sequences comprise amino acid mutations that promote formation of a heterodimeric Fc over a homodimeric Fc,
wherein:
(a) the amino acid mutations in the first CH3 domain sequence comprise the amino acid substitutions: A6085YY and T6086L, and the amino acid mutations in the second CH3 domain sequence comprise the amino acid substitutions: L6079T, W6081L and I6088L; or
(b) the amino acid mutations in the first CH3 domain sequence comprise the amino acid substitutions: A6085YY and T6086Y, and the amino acid mutations in the second CH3 domain sequence comprise the amino acid substitutions: L6079T, W6081L and I6088L; or
(c) the amino acid mutations in the first CH3 domain sequence comprise the amino acid substitutions: A6085YF and T6086Y, and the amino acid mutations in the second CH3 domain sequence comprise the amino acid substitutions: L6079V, W6081L and I6088L; or
(d) the amino acid mutations in the first CH3 domain sequence comprise the amino acid substitutions: A6085YF and T6086Y, and the amino acid mutations in the second CH3 domain sequence comprise the amino acid substitutions: L6079V, W6081T and I6088L; or
(e) the amino acid mutations in the first CH3 domain sequence comprise the amino acid substitutions: T6022V, A6085YF and T6086Y, and the amino acid mutations in the second CH3 domain sequence comprise the amino acid substitutions: L6079V, W6081T and I6088L; or
(f) the amino acid mutations in the first CH3 domain sequence comprise the amino acid substitutions: T6022L, A6085YF and T6086Y, and the amino acid mutations in the second CH3 domain sequence comprise the amino acid substitutions: L6079V, W6081T and I6088L; or
(g) the amino acid mutations in the first CH3 domain sequence comprise the amino acid substitutions: T6022I, A6085YF and T6086Y, and the amino acid mutations in the second CH3 domain sequence comprise the amino acid substitutions: L6079V, W6081T and I6088L; or
(h) the amino acid mutations in the first CH3 domain sequence comprise the amino acid substitutions: A6085YF and T6086Y, and the amino acid mutations in the second CH3 domain sequence comprise the amino acid substitutions: L6007F, L6079V, W6081T and I6088L
(i) the amino acid mutations in the first CH3 domain sequence comprise the amino acid substitutions: H6005Y, A6085YF and T6086Y, and the amino acid mutations in the second CH3 domain sequence comprise the amino acid substitutions: H6005Y, L6079V, W6081T and I6088L; or
(j) the amino acid mutations in the first CH3 domain sequence comprise the amino acid substitutions: H6005C, A6085YF and T6086Y, and the amino acid mutations in the second CH3 domain sequence comprise the amino acid substitutions: P6010C, L6079V, W6081T and I6088L; or
(k) the amino acid mutations in the first CH3 domain sequence comprise the amino acid substitutions: P6010C, A6085YF and T6086Y, and the amino acid mutations in the second CH3 domain sequence comprise the amino acid substitutions: H6005C, L6079V, W6081T and I6088L; or
(l) the amino acid mutations in the first CH3 domain sequence comprise the amino acid substitutions: H6005C, P6010C, A6085YF and T6086Y, and the amino acid mutations in the second CH3 domain sequence comprise the amino acid substitutions: H6005C, P6010C, L6079V, W6081T and I6088L,
wherein the heterodimeric Fc is formed with a purity of 70% or higher,
and wherein the numbering of amino acid positions is according to IMGT numbering.

28. The IgA HetFc construct according to claim 27, wherein the modified CH3 domain has a melting temperature (Tm) that is 60° C. or higher.

29. The IgA HetFc construct according to claim 27, wherein the modified CH3 domain has a melting temperature (Tm) that is ±10° C. of the Tm of a corresponding wild-type IgA CH3 domain.

30. The IgA HetFc construct according to any one of claims 1 to 29 further comprising one or more target binding domains.

31. The IgA HetFc construct according to claim 30, wherein the one or more target binding domains are antigen-binding antibody fragments.

32. The IgA HetFc construct according to claim 31, wherein each of the one or more antigen-binding antibody fragments are independently selected from a Fab and an scFv.

33. The IgA HetFc construct according to any one of claims 30 to 32, wherein the IgA HetFc construct comprises two target binding domains and is bispecific.

34. The IgA HetFc construct according to any one of claims 1 to 33, wherein the modified IgA CH3 domain comprises an α-tailpiece.

35. The IgA HetFc construct according to any one of claims 1 to 33, wherein the modified IgA CH3 domain lacks an α-tailpiece.

36. A conjugate comprising the IgA HetFc construct according to any one of claims 1 to 35 and one or more therapeutic, diagnostic or labeling agents.

37. An IgA HetFc multimer comprising two or more IgA HetFc constructs according to any one of claims 1 to 34 and a J chain, wherein two of the IgA HetFc constructs are joined by the J chain.

38. A pharmaceutical composition comprising the IgA HetFc construct according to any one of claims 1 to 35 and a pharmaceutically acceptable carrier or diluent.

39. A pharmaceutical composition comprising the conjugate according to claim 36 and a pharmaceutically acceptable carrier or diluent.

40. A pharmaceutical composition comprising the IgA HetFc multimer according to claim 37 and a pharmaceutically acceptable carrier or diluent.

41. An isolated polynucleotide or set of polynucleotides encoding the IgA HetFc construct according to any one of claims 1 to 35.

42. A vector set or set of vectors comprising one or more polynucleotides encoding the IgA HetFc according to any one of claims 1 to 35.

43. A host cell comprising one or more polynucleotides encoding the IgA HetFc according to any one of claims 1 to 35.

44. A method of preparing the IgA HetFc construct according to any one of claims 1 to 35 comprising transfecting a host cell with one or more polynucleotides encoding the IgA HetFc construct, and culturing the host cell under conditions suitable for expression of the IgA HetFc construct.

45. A method of preparing the IgA HetFc multimer according to claim 37 comprising transfecting a host cell with one or more polynucleotides encoding the IgA HetFc construct according to claim 34 and a polynucleotide encoding a J chain, and culturing the host cell under conditions suitable for expression of the IgA HetFc construct and the J chain.

46. An IgA HetFc construct of any one of claims 1 to 35, wherein the IgA Het Fc includes one or more mutations to eliminate binding to a binding target.

47. An IgA HetFc construct of any one of claims 1 to 35, wherein the IgA HetFc includes one or more mutations to introduce binding to the Neonatal Fc Receptor (FcRn).
